(19) United States
(12) Patent Application Publication
(10) Pub. No.: US 2024/0309344 A1
(43) Pub. Date: Sep. 19, 2024

(54) COMPOSITIONS AND METHODS FOR THE TARGETING OF C9orf72

(71) Applicant: Scribe Therapeutics Inc., Alameda, CA (US)

(72) Inventors: Benjamin OAKES, El Cerrito, CA (US); Hannah SPINNER, Boston, MA (US); Sarah DENNY, San Francisco, CA (US); Brett T. STAAHL, Tiburon, CA (US); Kian TAYLOR, Atlanta, GA (US); Katherine BANEY, Berkeley, CA (US); Isabel COLIN, Oakland, CA (US); Maroof ADIL, Davis, CA (US); Cole URNES, Los Angeles, CA (US); Sean HIGGINS, Alameda, CA (US)

(21) Appl. No.: 17/932,798

(22) Filed: Sep. 16, 2022

Related U.S. Application Data
(63) Continuation of application No. PCT/US21/22840, filed on Mar. 17, 2021.
(60) Provisional application No. 62/991,403, filed on Mar. 18, 2020.

(51) Int. Cl. C12N 9/22; C12N 15/11; C12N 15/86

(52) U.S. Cl. CPC C12N 9/22; C12N 15/111; C12N 15/86; C07K 2319/09; C12N 2310/531; C12N 2750/14141

(57) ABSTRACT

Provided herein are Class 2 Type V systems comprising nucleases, guide nucleic acids (gNA), and optionally donor template nucleic acids useful in the modification of a C9orf72 gene. The systems are also useful for introduction into cells, for example eukaryotic cells having mutations or duplications in the C9orf72 gene. Also provided are methods of using such systems to modify cells having such mutations or duplications.

### CROSS REFERENCE TO RELATED APPLICATIONS

This application claims priority to U.S. provisional patent application No. 62/991,403 filed on Mar. 18, 2020, the contents of which are incorporated by reference in their entirety herein.

### INCORPORATION BY REFERENCE OF SEQUENCE LISTING

The contents of the text file submitted electronically herewith are incorporated herein by reference in their entirety: A computer readable format copy of the Sequence Listing (filename: SCRB-025-01WO_SeqList_ST25.txt; date recorded: Mar. 12, 2021; file size: 5.61 Megabytes).

### BACKGROUND

Amyotrophic lateral sclerosis (ALS) and frontotemporal dementia (FTD) are forms of progressive neurological diseases with devastating outcomes. ALS is a fatal neurodegenerative disease characterized clinically by progressive paralysis leading to death from respiratory failure, typically within two to three years of symptom onset, and is the third most common neurodegenerative disease in the Western world (Rowland and Shneider, N. Engl. J. Med., 2001, 344, 1688-1700; Hirtz et al., Neurology, 2007, 68, 326-337). FTD is the second most common cause of pre-senile dementia in which degeneration of the frontal and temporal lobes of the brain results in progressive changes in personality, behavior, and language with relative preservation of perception and memory (Graff-Radford, N and Woodruff, B. Frontotemporal dementia. Semin. Neurol. 27(1):48 (2007)).

Chromosome 9 open reading frame 72 protein is a protein encoded by the C9orf72 gene (sometimes also referred to as C9orf72-SMCR8 complex subunit). Forms of disease related to mutations or abnormalities in the C9orf72 gene include FTD and ALS. The protein is found in many regions of the brain, including the cytoplasm of neurons, as well as in presynaptic terminals. In particular, the relevant mutation in the C9orf72 gene in connection with FTD and ALS is a hexanucleotide repeat expansion of the six letter string of nucleotides GGGGCC that occurs is located either in intron 1, between two 5′-untranslated region (5′-UTR) exons, or in the promoter region of the C9orf72 gene (DeJesus-Hernandez, M., et al. Expanded GGGGCC hexanucleotide repeat in noncoding region of C9ORF72 causes chromosome 9p-linked FTD and ALS. Neuron 72: 245 (2011); Niblock, M., et al. Retention of hexanucleotide repeat-containing intron in C9orf72 mRNA: implications for the pathogenesis of ALS/FTD. Acta Neuropathologica Communications 4:18 (2016)). Accordingly, the presence of the hexanucleotide repeat sequence (HRS) expansion does not alter the coding sequence of the resulting C9orf72 protein. In healthy individuals, there are few repeats of this hexanucleotide, typically 30 or less, but in people with the diseased phenotype, the repeat units range from approximately 700 to 1600 (Mori K., et al. The C9orf72 GGGGCC repeat is translated into aggregating dipeptide-repeat proteins in FTLD/ALS. Science. 339:1335 (2013)). It is believed that the repeat hexanucleotide expansion leads to the loss of one alternatively spliced C9orf72 transcript and to formation and the accumulation of insoluble dipeptide repeat protein aggregates by repeat-associated non-AUG-initiated (RAN) translation, most containing poly-(Gly-Ala) and, to a lesser extent, poly-(Gly-Pro) and poly-(Gly-Arg) dipeptide-repeat proteins (DPR) that are extremely hydrophobic and could be pathogenic in FTD-ALS patients (Mori K., et al. 2013; Niblock, M., et al. 2016). Furthermore, three main disease mechanisms have been proposed: loss of function of the C9orf72 protein, toxic gain of function from C9orf72 repeat RNA through accumulation in the frontal cortex and spinal cord of RNA transcripts containing the repeat as well as the antisense GGCCCC RNA, or from accumulation of DPR produced by repeat-associated non-ATG translation (Balendra R, Isaacs A M. C9orf72-mediated ALS and FTD: multiple pathways to disease. Nat Rev Neurol. 14:544 (2018)). The inheritance of the C9orf72 mutation is autosomal dominant (Iyer et al. C9orf72, a protein associated with amyotrophic lateral sclerosis (ALS) is a guanine nucleotide exchange factor. PeerJ 6:e5815 (2018)).

The advent of CRISPR/Cas systems and the programmable nature of these minimal systems has facilitated their use as a versatile technology for genomic manipulation and engineering. However, efforts to correct C9orf72-related diseases such as FTD and ALS by genetic engineering have received limited attention. Thus, there is a need for compositions and methods to regulate C9orf72 in subjects with C9orf72-related diseases. Provided herein are compositions and methods for targeting the C9orf72 gene to the address this need.

### SUMMARY

The present disclosure provides compositions of modified Class 2, Type V CRISPR proteins and guide nucleic acids used in the editing of chromosome 9 open reading frame 72 (C9orf72) gene target nucleic acid sequences. The Class 2 Type V CRISPR proteins and guide nucleic acids are modified for passive entry into target cells. The Class 2 Type V CRISPR proteins and guide nucleic acids are useful in a variety of methods for target nucleic acid modification of C9orf72-related diseases, which methods are also provided.

In one aspect, the present disclosure relates to CasX:guide nucleic acid systems (CasX:gNA system) and methods used to alter a target nucleic acid comprising the C9orf72 gene with one or more mutations or that comprises the hexanucleotide repeat sequence expansion (HRS) in cells. In some embodiments of the disclosure, the CasX:gNA system has utility in knocking-down or knocking-out a C9orf72 gene with one or more mutations or that comprises the hexanucleotide repeat sequence expansion (HRS), in order to reduce or eliminate expression of the C9orf72 gene product, accumulation of the RNA from the HRS, and/or the DPR in subjects having a C9orf72-related disease. In other embodiments, the CasX:gNA system has utility in correcting the C9orf72 gene comprising the HRS.

In some embodiments of the system, the gNA is a gRNA, or a gDNA, or a chimera of RNA and DNA, and may be a single-molecule gNA or a dual-molecule gNA. In other embodiments, the CasX:gNA system gNA has a targeting sequence complementary to a target nucleic acid sequence comprising a region within the C9orf72 gene. In some embodiments, the targeting sequence of the gNA is selected from the group consisting of SEQ ID NOS: 309-343, 363-2100, 2295-2185, or a sequence having at least about 65%, at least about 75%, at least about 85%, or at least about 95% identity thereto. The gNA can comprise a targeting sequence comprising 14 to 30 consecutive nucleotides. In some embodiments, the targeting sequence of the gNA consists of 21 nucleotides. In other embodiments, the targeting sequence of the gNA consists of 20 nucleotides. In other embodiments, the targeting sequence consists of 19 nucleotides, the targeting sequence of the gNA having a sequence selected from the group consisting of SEQ ID NOS: 309-343, 363-2100, 2295-2185 with a single nucleotide removed from the 3′ end of the sequence. In other embodiments, the targeting sequence consists of 18 nucleotides, having a sequence selected from the group consisting of SEQ ID NOS: 309-343, 363-2100, 2295-2185 with two nucleotides removed from the 3′ end of the sequence. In other embodiments, the targeting sequence consists of 17 nucleotides, having a sequence selected from the group consisting of SEQ ID NOS: 309-343, 363-2100, 2295-2185 with three nucleotides removed from the 3′ end of the sequence. In other embodiments, the targeting sequence consists of 16 nucleotides, having a sequence selected from the group consisting of SEQ ID NOS: 309-343, 363-2100, 2295-2185 with four nucleotides removed from the 3′ end of the sequence. In other embodiments, the targeting sequence consists of 15 nucleotides, having a sequence selected from the group consisting of SEQ ID NOS: 309-343, 363-2100, 2295-2185 with five nucleotides removed from the 3′ end of the sequence.

In some embodiments of the system, the gNA has a scaffold comprising a sequence selected from the group consisting of SEQ ID NOS: 4-16 and 2101-2294, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto.

In some embodiments of the system, the Class 2, Type V CRISPR protein comprises a reference CasX protein having a sequence of any one of SEQ ID NOS: 1-3, a CasX variant protein having a sequence selected from the group consisting of SEQ ID NOS: 49-150, 233-235, 238-239, 240-242, and 272-281, or a sequence having at least about 50%, at least about 60%, at least about 70%, at least about 80%, at least about 90%, at least about 95%, at least about 96%, at least about 97%, at least about 98%, or at least about 99% sequence identity thereto. In these embodiments, a CasX variant exhibits one or more improved characteristics relative to the reference CasX protein. In some embodiments, the CasX protein has binding affinity for a protospacer adjacent motif (PAM) sequence selected from the group consisting of TTC, ATC, GTC, and CTC. In some embodiments, the CasX protein has binding affinity for the PAM sequence that is at least 1.5-fold greater compared to the binding affinity of any one of the CasX proteins of SEQ ID NOS: 1-3 for the PAM sequences selected from the group consisting of TTC, ATC, GTC, and CTC.

In some embodiments of the system, the CasX molecule and the gNA molecule are associated together in a ribonuclear protein complex (RNP). In a particular embodiment, the RNP comprising the CasX variant and the gNA variant exhibits greater editing efficiency and/or binding of a target sequence in the target DNA when any one of the PAM sequences TTC, ATC, GTC, or CTC is located 1 nucleotide 5′ to the non-target strand sequence having identity with the targeting sequence of the gNA in a cellular assay system compared to the editing efficiency and/or binding of an RNP comprising a reference CasX protein and a reference gNA in a comparable assay system.

In some embodiments, the system further comprises a donor template comprising a nucleic acid comprising at least a portion of a C9orf72 gene, wherein the C9orf72 gene portion is selected from the group consisting of a C9orf72 exon, a C9orf72 intron, a C9orf72 intron-exon junction, a C9orf72 regulatory element, or combinations thereof, wherein the donor template is used to knock down or knock out the C9orf72 gene or is used to correct the mutation in the C9orf72 gene. In some embodiments, the donor template comprises a hexanucleotide repeat of a GGGGCC sequence wherein the number of repeats ranges from 10 to about 30 repeats and is utilized to replace the hexanucleotide repeat expansion of the mutant C9orf72 gene. In some cases, the donor sequence is a single-stranded DNA template or a single stranded RNA template. In other cases, the donor template is a double-stranded DNA template.

In other embodiments, the disclosure relates to nucleic acids encoding the systems of any of the embodiments described herein, as well as vectors comprising the nucleic acids. In some embodiments, the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, and an RNA vector. In other embodiments, the vector is a virus-like particle (VLP) comprising an RNP of a CasX and gNA of any of the embodiments described herein and, optionally, a donor template nucleic acid and a targeting moiety such as a viral-derived glycoprotein.

In other embodiments, the disclosure provides a method of modifying a C9orf72 target nucleic acid sequence of cells of a population, wherein said method comprises introducing into the cell: a) CasX:gNA system of any of the embodiments disclosed herein; b) the nucleic acid of any of the embodiments disclosed herein; c) the vector of any of the embodiments disclosed herein; d) the VLP of any of the embodiments disclosed herein; or e) a combination of the foregoing, wherein the C9orf72 gene target nucleic acid sequence of the cells targeted by the first gNA is modified by the CasX protein, introducing a single- or double-stranded break in the target nucleic acid sequence. In some embodiments of the method, the method further comprises a second gNA or a nucleic acid encoding the second gNA, wherein the second gNA has a targeting sequence complementary to a different portion of the target nucleic acid sequence. In some embodiments of the method, the modifying comprises introducing an insertion, deletion, substitution, duplication, or inversion of one or more nucleotides in the target nucleic acid sequence as compared to the wild-type sequence. In some cases, the method further comprises contacting the target nucleic acid with a donor template nucleic acid of any of the embodiments disclosed herein. In some embodiments, the target C9orf72 gene for modification comprises more than 30, more than 100, more than 500, more than 700, more than 1000, or more than 1600 copies of a hexanucleotide repeat sequence GGGGCC. In some embodiments of the method, the donor template comprises a nucleic acid comprising at least a portion of a C9orf72 gene for correcting (by knocking in) the mutation of the C9orf72 gene, or comprises a sequence comprising a mutation or heterologous sequence for knocking-down or knocking out the mutant C9orf72 such that expression of the HRS or the DPR by the cells of the population is reduced by at least about 10%, at least about 20%, at least about 30%, at least about 40%, at least about 50%, at least about 60%, at least about 70%, at least about 80%, or at least about 90% in comparison to a cell where the that has not been modified. In some cases, the modifying of the target nucleic acid sequence occurs in vivo. In some embodiments, the cell is a eukaryotic cell selected from the group consisting of a rodent cell, a mouse cell, a rat cell, a primate cell, a and a non-human primate cell. In some embodiments, the cell is a human cell. In some embodiments, the cell is a selected from the group consisting of a Purkinje cell, frontal cortex neuron, motor cortex neuron, hippocampus neuron, cerebellum neuron, upper motor neuron, spinal cord neuron, spinal cord motor neuron, glial cell, and astrocytes.

In other embodiments, the disclosure provides methods of modifying a C9orf72 target nucleic acid in a population of cells of a subject wherein the target cells are contacted using vectors encoding the CasX protein and one or more gNAs comprising a targeting sequence complementary to the C9orf72 gene, and optionally further comprising a donor template. In some cases, the vector is an Adeno-Associated Viral (AAV) vector selected from AAV1, AAV2, AAV3, AAV4, AAV5, AAV6, AAV7, AAV8, AAV9, AAV10, AAV11, AAV12, AAV 44.9, AAV-Rh74, or AAVRh10. In other cases, the vector is a lentiviral vector. In other embodiments, the disclosure provides methods wherein the target cells are contacted using a vector wherein the vector is a virus-like particle (VLP) comprising an RNP of a CasX and gNA of any of the embodiments described herein and, optionally, a donor template nucleic acid. In some embodiments of the method, the vector is administered to a subject at a therapeutically effective dose. The subject can be a mouse, rat, pig, non-human primate, or a human. The dose can be administered by a route of administration selected from the group consisting of subcutaneous, intradermal, intraneural, intranodal, intramedullary, intramuscular, intralumbar, intrathecal, subarachnoid, intraventricular, intracapsular, intravenous, intralymphatic, or intraperitoneal routes, wherein the administering method is injection, transfusion, or implantation.

In other embodiments, the disclosure provides a method of treating a C9orf72-related disorder in a subject, comprising modifying a gene encoding C9orf72 gene in cells of the subject, the modifying comprising contacting said cells with: a) CasX:gNA system of any of the embodiments disclosed herein; b) the nucleic acid of any of the embodiments disclosed herein; c) the vector of any of the embodiments disclosed herein; d) the VLP of any of the embodiments disclosed herein; or e) a combination of the foregoing wherein the C9orf72 gene of the cells targeted by the first gNA is modified by the CasX protein. In some embodiments, the subject is selected from the group consisting of mouse, rat, pig, non-human primate, and human. In some embodiments, the C9orf72-related disorder is ALS or FTD. In some cases, the methods of treating a subject with a C9orf72-related disease result in improvement in at least one clinically-relevant parameter. In other cases, the methods of treating a subject with a C9orf72-related disease result in improvement in at least two clinically-relevant parameters.

In other embodiments, the disclosure provides compositions for use in a method of treating a C9orf72-related disorder in a subject. In some embodiments, the method comprises modifying a gene encoding C9orf72 gene in cells of the subject, the modifying comprising contacting said cells with compositions selected from: a) CasX:gNA system of any of the embodiments disclosed herein; b) the nucleic acid of any of the embodiments disclosed herein; c) the vector of any of the embodiments disclosed herein; d) the VLP of any of the embodiments disclosed herein; or e) a combination of the foregoing wherein the C9orf72 gene of the cells targeted by the first gNA is modified by the CasX protein. In some embodiments, the subject is selected from the group consisting of mouse, rat, pig, non-human primate, and human. In some embodiments, the C9orf72-related disorder is ALS or FTD. In some cases, the methods of treating a subject with a C9orf72-related disease result in improvement in at least one clinically-relevant parameter. In other cases, the methods of treating a subject with a C9orf72-related disease result in improvement in at least two clinically-relevant parameters.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of WO 2020/247882, filed on Jun. 5, 2020, and U.S. provisional applications 63/121,196 filed Dec. 3, 2020, and 63/162,346 filed on Mar. 17, 2021, which disclose CasX variants and gNA variants, are hereby incorporated by reference in their entireties.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an SDS-PAGE gel of CasX StX2 purification fractions visualized bycolloidal Coomassie staining, as described in Example 1.

FIG. 2 shows a chromatogram from a size exclusion chromatography assay of the CasX StX2, using of Superdex 200 16/600 pg Gel Filtration, as described in Example 1.

FIG. 3 shows an SDS-PAGE gel of CasX StX2 purification fractions visualized by colloidal Coomassie staining, as described in Example 1.

FIG. 4 is a schematic showing the organization of the components in the pSTX34 plasmid used to assemble the CasX constructs, as described in Example 2.

FIG. 5 is a schematic showing the steps of generating the pSTX34 plasmid with a CasX 119 variant, as described in Example 2.

FIG. 6 shows an SDS-PAGE gel of purification samples, visualized on a Bio-Rad Stain-Free™ gel, as described in Example 2.

FIG. 7 shows the chromatogram of Superdex 200 16/600 pg Gel Filtration, as described in Example 2.

FIG. 8 shows an SDS-PAGE gel of gel filtration samples, stained with colloidal Coomassie, as described in Example 2.

FIG. 9 is a graph of the results of an assay for the quantification of active fractions of RNP formed by sgRNA174 and the CasX variants 119, 457, 488 and 491, as described in Example 13. Equimolar amounts of RNP and target were co-incubated and the amount of cleaved target was determined at the indicated timepoints. Mean and standard deviation of three independent replicates are shown for each timepoint. The biphasic fit of the combined replicates is shown. “2” refers to the reference CasX protein of SEQ ID NO: 2.

FIG. 10 shows the quantification of active fractions of RNP formed by CasX2 (reference CasX protein of SEQ ID NO:2) and the modified sgRNAs, as described in Example 13. Equimolar amounts of RNP and target were co-incubated and the amount of cleaved target was determined at the indicated timepoints. Mean and standard deviation of three independent replicates are shown for each timepoint. The biphasic fit of the combined replicates is shown.

FIG. 11 shows the quantification of active fractions of RNP formed by CasX 491 and the modified sgRNAs under guide-limiting conditions, as described in Example 13. Equimolar amounts of RNP and target were co-incubated and the amount of cleaved target was determined at the indicated timepoints. The biphasic fit of the data is shown.

FIG. 12 shows the quantification of cleavage rates of RNP formed by sgRNA174 and the CasX variants, as described in Example 13. Target DNA was incubated with a 20-fold excess of the indicated RNP and the amount of cleaved target was determined at the indicated time points. Mean and standard deviation of three independent replicates are shown for each timepoint, except for 488 and 491 where a single replicate is shown. The monophasic fit of the combined replicates is shown.

FIG. 13 shows the quantification of cleavage rates of RNP formed by CasX2 and the sgRNA variants, as described in Example 13. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 14 shows the quantification of initial velocities of RNP formed by CasX2 and the sgRNA variants, as described in Example 13. The first two time-points of the previous cleavage experiment were fit with a linear model to determine the initial cleavage velocity.

FIG. 15 shows the quantification of cleavage rates of RNP formed by CasX491 and the sgRNA variants, as described in Example 13. Target DNA was incubated with a 20-fold excess of the indicated RNP at 10° C. and the amount of cleaved target was determined at the indicated time points. The monophasic fit of the timepoints is shown.

FIGS. 16A-16D show the quantification of cleavage rates of CasX variants on NTC PAMs, as described in Example 14. Target DNA substrates with identical spacers and the indicated PAM sequence were incubated with a 20-fold excess of the indicated RNP at 37° C. and the amount of cleaved target was determined at the indicated time points. Monophasic fit of a single replicate is shown. FIG. 16A shows the results for sequences having a TTC PAM. FIG. 16B shows the results for sequences having a CTC PAM. FIG. 16C shows the results for sequences having a GTC PAM. FIG. 16D shows the results for sequences having a ATC PAM.

FIG. 17 is a schematic showing an example of CasX protein and scaffold DNA sequence for packaging in adeno-associated virus (AAV), as described in Example 23. The DNA segment between the AAV inverted terminal repeats (ITRs), comprised of a CasX-encoding DNA and its promoter, and scaffold-encoding DNA and its promoter gets packaged within an AAV capsid during AAV production.

FIG. 18 shows the results of an editing assay comparing gRNA scaffolds 229-237 to scaffold 174 in mouse neural progenitor cells (mNPC) isolated from the Ai9-tdtomato transgenic mice. Cells were nucleofected with the indicated doses of p59 plasmids encoding CasX 491, the scaffold, and spacer 11.30 (5′ AAGGGGCUCCGCACCACGCC 3′, SEQ ID NO: 361) targeting mRHO. Editing at the mRHO locus was assessed 5 days post-transfection by NGS, and show that editing with constructs with scaffolds 230, 231, 234 and 235 demonstrated greater editing compared to constructs with scaffold 174 at both doses.

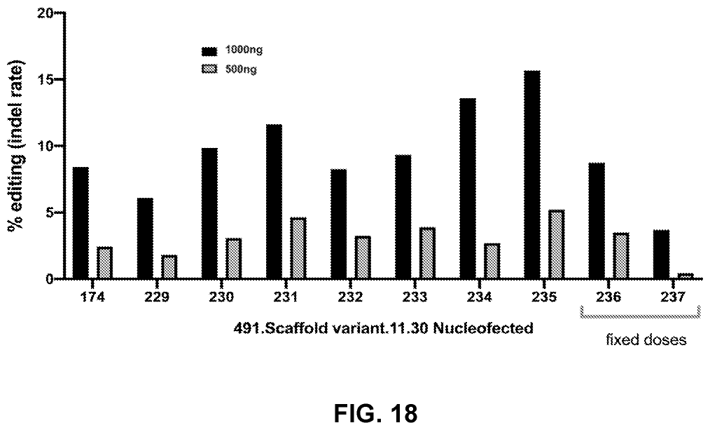

FIG. 19 shows the results of an editing assay comparing gRNA scaffolds 229-237 to scaffold 174 in mNPC cells. Cells were nucleofected with the indicated doses of p59 plasmids encoding CasX 491, the scaffold, and spacer 12.7 (5′ CUGCAUUCUAGUUGUGGUUU 3′, SEQ ID NO: 362) targeting repeat elements preventing expression of the tdtomato fluorescent protein. Editing was assessed 5 days post-transfection by FACS, to quantify the fraction of tdTomato positive cells. Cells nucleofected with scaffolds 231-235 displayed approximately 35% greater editing compared to constructs with scaffold 174 at the high dose, and approximately 25% greater editing at the low dose.

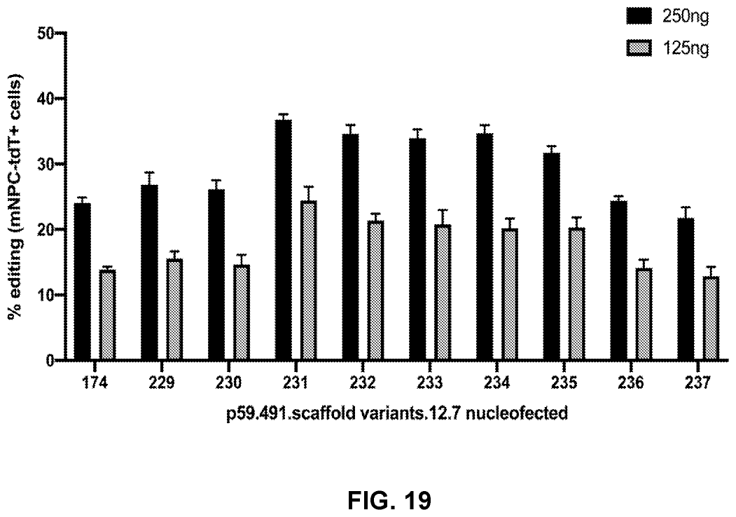

FIG. 20 shows the results of an editing assay comparing CasX nucleases 2, 119, 491, 515, 527, 528, 529, 530, and 531 in a custom HEK293 cell line, PASS_V1.01. Cells were lipofected with 2 μg of p67 plasmid encoding the indicated CasX protein. After five days, cell genomic DNA was extracted. PCR amplification and Next-Generation Sequencing was performed to isolate and quantify the fraction of edited cells at custom designed on-target editing sites. For each sample, editing was evaluated at target sites (individual points) consisting of the following PAM sequences: 48 TTC, 14 ATC, 22 CTC, 11 GTC individual sites, and percent editing was normalized to a vehicle control. Cells lipofected with any nuclease displayed higher mean editing at TTC PAM target sites (horizontal bar) than that of the wild-type nuclease CasX 2, except CasX 528. The relative preference of any given nuclease for the four different PAM sequences is also represented by the violin plots. In particular, CasX nucleases 527, 528, and 529 exhibit substantially different PAM preferences than that of the wild-type nuclease CasX 2.

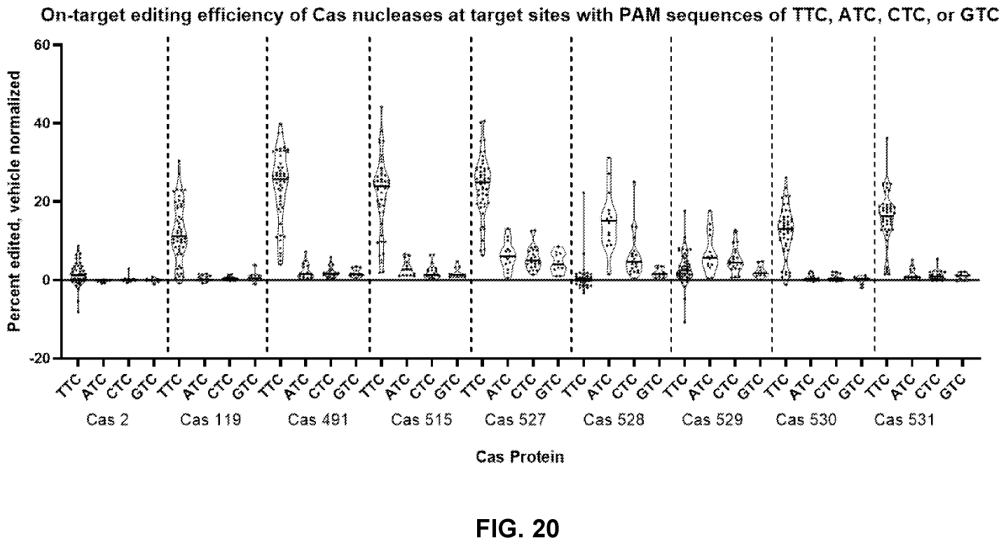

FIG. 21 shows the results of an editing assay comparing improved CasX nuclease 491 to improved nucleases 532 and 533 in a custom HEK293 cell line, PASS_V1.01. Cells were lipofected, in duplicate, with 2 μg of p67 plasmid encoding the indicated CasX protein and a puromycin resistance gene, and grown under puromycin selection. After three days, cell genomic DNA was extracted. PCR amplification and Next-Generation Sequencing was performed to isolate and quantify the fraction of edited cells at custom designed on-target editing sites. For each sample, editing was evaluated at target sites consisting of the following PAM sequences: 48 TTC, 14 ATC, 22 CTC, 11 GTC individual sites, and fraction editing was normalized to a vehicle control. Cells lipofected with CasX 532 or 533 displayed higher mean editing than Cas 491 at each of the PAM sequences, with the exception of CasX 533 at TTC PAM target sites. Error bars represent standard error of the mean for n=2 biological samples.

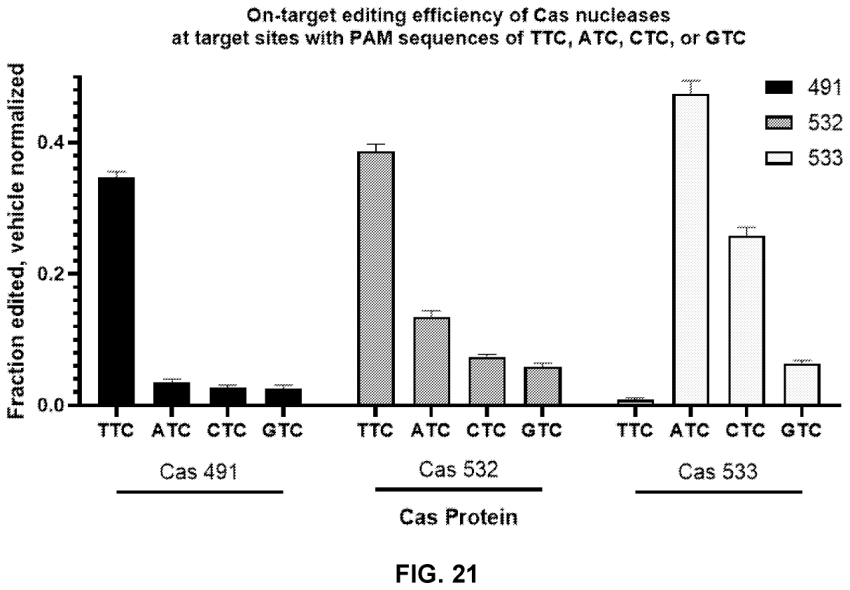

FIG. 22 is a schematic of a portion of the 5′ region of the C9orf72 locus. The top diagram shows the relative locations of the exon 1a and exon 1b, which flank the hexanucleotide repeat element (HRE) while the open boxes indicate downstream exons. The lower diagram shows the regions of the locus that are targeted by (complementary to) the targeting segments (spacers) of the guide RNA of Table 15, as described in Example 18.

FIG. 23 is a graph showing results of a single cut experiment using targeting sequence 164 to introduce edits in Exon 1a, as described in Example 18. The black deletion trace indicates for every position in the amplicon and the fraction of reads that have a deletion at that position. Gray bars at the bottom of the graph indicate the sgRNA binding site position. Quantification window indicates region used for quantification of deletions. Predicted cleavage position is the location of CasX induced double-stranded break. The deletion traces illustrate rates and extent of genetic deletions generated by the single guides delivered, resulting in an overall deletion efficiency of 65.4%. The data are representative of the results observed for single cuts (Table 15).

FIG. 24 is a graph showing results of a double cut experiment using targeting sequences (spacers) 138 and 151, which targeting sequences that flank the hexanucleotide repeat element (HRE, also sometimes referred to herein as the hexanucleotide repeat sequence expansion, or HRS) at positions 193-248 in the reference amplicon, as described in Example 18. The black deletion trace indicates for every position in the amplicon the fraction of reads that have a deletion at that position. Gray bars at the bottom of the graph indicate the sgRNA binding site position. The quantification window indicates the region used for quantification of deletions. The predicted cleavage position is the location of CasX induced double-stranded break. In this experiment, the overall deletion efficiency was 45.4%, and was representative of the results observed for double cuts (Table 16), supporting that, under the conditions of the experiment, the HRE can be deleted using the double-cut design.

FIG. 25 is a pair of graphs of an experiment testing the effects of spacer length on ability to edits target nucleic acid in Jurkat cells, as described in Example 26. The results demonstrate that shorter spacers of 18 or 19 support increased activity compared to a spacer of 20 bases in ex vivo editing by RNP.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid sequence to be specifically hybridizable; it can have at least about 70%, at least about 80%, or at least about 90%, or at least about 95% sequence identity and still hybridize to the target nucleic acid sequence. Moreover, a polynucleotide may hybridize over one or more segments such that intervening or adjacent segments are not involved in the hybridization event (e.g., a loop structure or hairpin structure, a ‘bulge’, ‘bubble’ and the like).

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include regulatory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “regulatory element” is used interchangeably herein with the term “regulatory sequence,” and is intended to include promoters, enhancers, and other expression regulatory elements (e.g. transcription termination signals, such as polyadenylation signals and poly-U sequences). Exemplary regulatory elements include a transcription promoter such as, but not limited to, CMV, CMV+intron A, SV40, RSV, HIV-Ltr, elongation factor 1 alpha (EF1α), MMLV-ltr, internal ribosome entry site (IRES) or P2A peptide to permit translation of multiple genes from a single transcript, metallothionein, a transcription enhancer element, a transcription termination signal, polyadenylation sequences, sequences for optimization of initiation of translation, and translation termination sequences. In the case of systems utilized for exon skipping, regulatory elements include exonic splicing enhancers. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “promoter” refers to a DNA sequence that contains an RNA polymerase binding site, transcription start site, TATA box, and/or B recognition element and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid sequence with a guide nucleic acid means that the target nucleic acid sequence and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L][P]/[LP], where [P], [L] and [LP] represent molar concentrations of the protein, ligand and complex, respectively.

The disclosure provides compositions and methods useful for editing a target nucleic acid sequence. As used herein “editing” is used interchangeably with “modifying” and includes but is not limited to cleaving, nicking, deleting, knocking in, knocking out, and the like.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor (e.g., such as the donor template) to the target, resulting in a transgene of interest. Homology-directed repair can result in an alteration of the sequence of the target nucleic acid sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide (or protein) has a certain percent “sequence similarity” or “sequence identity” to another polynucleotide or polypeptide, meaning that, when aligned, that percentage of bases or amino acids are the same, and in the same relative position, when comparing the two sequences. Sequence similarity (interchangeably referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, virus-like particle, or cosmid, to which another DNA segment, i.e., an “insert”, may be attached so as to bring about the replication or expression of the attached segment in a cell.

The term “naturally-occurring” or “unmodified” or “wild-type” as used herein as applied to a nucleic acid, a polypeptide, a cell, or an organism, refers to a nucleic acid, polypeptide, cell, or organism that is found in nature. Thus, “wild-type” can refer to more than one naturally occurring variants of a nucleic acid, polypeptide, cell or organism. With respect to genes, “wild-type” can also be used to refer to naturally-occurring, non-disease causing variants of the gene.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disease.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” is meant as a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, rabbits, mice, rats and other rodents.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Genetic Editing of C9orf72 Genes

In a first aspect, the present disclosure provides systems comprising a Class 2, Type V CRISPR nuclease protein and one or more guide nucleic acids (gNA) for use in modifying a C9orf72 gene with one or more mutations or that comprises the HRS, in order to reduce or eliminate expression of the C9orf72 gene product, the RNA from the transcription of the HRS, and/or the DPR (collectively referred to herein as the “target nucleic acid”, inclusive of coding and non-coding regions).

The human C9orf72 gene (HGNC:28337) encodes a protein (Q01453) having the sequence MSTLCPPPSPAVAKTEIALSGKSPLLAATFAYWDNILGPRVRHIWAPKTEQVLLSDGEIT FLANHTLNGEILRNAESGAIDVKFFVLSEKGVIIVSLIFDGNWNGDRSTYGLSIILPQTELS FYLPLHRVCVDRLTHIIRKGRIWMHKERQENVQKIILEGTERMEDQGQ SIIPMLTGEVIP VMELLSSMKSHSVPEEIDIADTVLNDDDIGDSCHEGFLLNAISSHLQTCGCSVVVGSSAE KVNKIVRTLCLFLTPAERKCSRLCEAESSFKYESGLFVQGLLKDSTGSFVLPFRQVMYAP YPTTHIDVDVNTVKQMPPCHEHIYNQRRYMRSELTAFWRATSEEDMAQDTIIYTDESFT PDLNIFQDVLHRDTLVKAFLDQVFQLKPGLSLRSTFLAQFLLVLHRKALTLIKYIEDDTQ KGKKPFKSLRNLKIDLDLTAEGDLNIIMALAEKIKPGLHSFIFGRPFYTSVQERDVLMTF (SEQ ID NO: 227). The C9orf72 gene is defined as the sequence that spans chr9: 27,546,546-27,573,866 of the human genome on chromosome 9 (Homo sapiens Updated Annotation Release 109.20191205, GRCh38.p13 (NCBI). The human C9orf72 gene is described in part in the NCBI database (ncbi.nlm.nih.gov) as Reference Sequence NC_000009.12, which is incorporated by reference herein. The C9orf72 locus contains 12 exons, including 2 alternate noncoding first exons (exons 1a and 1b) (DeJesus-Hernandez, M., et al. 2011). In the case of the hexanucleotide repeats, the translated DPR proteins include poly-(Gly-Ala) and, to a lesser extent, poly-(Gly-Pro) and poly-(Gly-Arg). A shorter isoform b (NP_659442.2) has the sequence

(SEQ ID NO: 228)
MSTLCPPPSPAVAKTEIALSGKSPLLAATFAYWDNILGPRVRHIWAPKTE
QVLLSDGEITFLANHTLNGEILRNAESGAIDVKFFVLSEKGVIIVSLIFD
GNWNGDRSTYGLSIILPQTELSFYLPLHRVCVDRLTHIIRKGRIWMHKER
QENVQKIILEGTERMEDQGQSIIPMLTGEVIPVMELLSSMKSHSVPEEID
IADTVLNDDDIGDSCHEGFLLK.

In some embodiments, the disclosure provides systems specifically designed to modify the C9orf72 gene in eukaryotic cells. In some cases the systems are designed to knock-down or knock-out the C9orf72 gene. In other cases, the systems are designed to correct one or more mutations in the C9orf72 gene. In some embodiments, the systems are designed to excise the hexanucleotide repeat sequence and restore the ability of the cell to express a functional C9orf72 protein. In some embodiments, the systems are designed to correct the hexanucleotide repeat sequence GGGGCC mutation of the C9orf72 gene that encodes the RNA transcripts of the HRS and/or the DPR and restore the ability of the cell to express a functional C9orf72 protein.

Generally, any portion of the C9orf72 gene can be targeted using the programmable compositions and methods provided herein. In some embodiments, the CRISPR nuclease is a Class 2, Type V nuclease. In some embodiments, the Class 2, Type V nuclease is selected from the group consisting of Cas12a, Cas12b, Cas12c, Cas12d (CasY), Cas12J, and CasX. In some embodiments, the Class 2, Type V nuclease is a CasX. In some embodiments, the disclosure provides systems comprising one or more CasX proteins and one or more guide nucleic acids (gNA) as a CasX:gNA system and, optionally, one or more donor template nucleic acids. Each of these components and their use in the editing of the C9orf72 gene is described herein, below.

In some embodiments, the disclosure provides gene editing pairs of a CasX and a gNA of any of the embodiments described herein that are capable of being bound together prior to their use for gene editing and, thus, are “pre-complexed” as a ribonuclear protein complex (RNP). The use of a pre-complexed RNP confers advantages in the delivery of the system components to a cell or target nucleic acid sequence for editing of the target nucleic acid sequence. In some embodiments, the functional RNP can be delivered ex vivo to a cell by electrophoresis or by chemical means. In other embodiments, the functional RNP can be delivered either ex vivo or in vivo by a vector in their functional form, or are expressed and then complex together as the RNP. The gNA can provide target specificity to the complex by including a targeting sequence (or “spacer”) having a nucleotide sequence that is complementary to a sequence of the target nucleic acid sequence while the CasX protein of the pre-complexed CasX:gNA provides the site-specific activity such as cleavage or nicking of the target sequence that is guided to a target site (e.g., stabilized at a target site) within a target nucleic acid sequence (e.g., a C9orf72 gene to be modified) by virtue of its association with the gNA. The CasX proteins and gNA components of the CasX:gNA systems and their sequences, features and functions are described more fully, below.

In some embodiments, the CasX:gNA systems utilized in the editing of the C9orf72 gene can optionally further comprise a donor template comprising all or at least a portion of a gene encoding a C9orf72 protein, a non-coding region, or a C9orf72 regulatory element wherein the donor template comprises one or more mutations compared to a wild-type C9orf72 gene utilized for insertion for either knocking out or knocking down (described more fully, below) the target nucleic acid sequence with one or more mutations or the HRS. In other cases, the CasX:gNA systems can optionally further comprise a donor template for the introduction (or knocking in) of all or a portion of gene encoding the physiologically-normal number of hexanucleotide repeats, or a sequence for the production of a wild-type C9orf72 protein (SEQ ID NO:227 or 228), or a sequence for the production of physiologically-normal levels of C9orf72 in the target cell. In some embodiments, the donor template comprises at least about 20, at least about 50, at least about 100, at least about 200, at least about 300, at least about 400, at least about 500, at least about 600, at least about 700, at least about 800, at least about 900, at least about 1000, at least about 10,000, at least 15,000, or at least 25,000 nucleotides of a wild-type C9orf72 gene, wherein the C9orf72 gene portion is selected from the group consisting of a C9orf72 exon, a C9orf72 intron, a C9orf72 intron-exon junction, a C9orf72 regulatory element, a C9orf72 coding region, a C9orf72 non-coding region, or the entirety of the C9orf72 gene. In some embodiments, the C9orf72 gene portion comprises a combination of any of a C9orf72 exon sequence, a C9orf72 intron sequence, a C9orf72 intron-exon junction sequence, a C9orf72 non-coding region, or a C9orf72 regulatory element sequence. In particular embodiments, the donor template comprises a sequence having a physiologically-normal number of hexanucleotide repeats of the GGGGCC sequence wherein upon insertion of the donor template, the hexanucleotide repeat sequence expansion of the C9orf72 gene is replaced. In other embodiments, the donor polynucleotide comprises at least about 10 to about 15,000 nucleotides, at least about 100 to about 10,000 nucleotides, at least about 400 to about 6000 nucleotides, at least about 600 to about 4000 nucleotides, or at least about 1000 to about 2000 nucleotides of a wild-type C9orf72 gene. In some embodiments, the donor template is a single stranded DNA template or a single stranded RNA template. In other embodiments, the donor template is a double stranded DNA template.

### III. Guide Nucleic Acids of the Systems for Genetic Editing

In another aspect, the disclosure relates to guide nucleic acids (gNA) comprising a targeting sequence complementary to a target nucleic acid sequence of a C9orf72 gene, wherein the gNA is capable of forming a complex with a CRISPR protein that has specificity to a protospacer adjacent motif (PAM) sequence comprising a TC motif in the complementary non-target strand, and wherein the PAM sequence is located 1 nucleotide 5′ of the sequence in the non-target strand that is complementary to the target nucleic acid sequence in the target strand of the target nucleic acid. In some embodiments, the gNA is capable of forming a complex with a Class 2, Type V CRISPR nuclease. In a particular embodiment, the gNA is capable of forming a complex with a CasX nuclease.

In some embodiments, the disclosure provides gNAs utilized in the CasX:gNA systems that have utility in genome editing in a cell that have utility in editing of the C9orf72 gene. The present disclosure provides specifically-designed guide nucleic acids (“gNAs”) with targeting sequences that are complementary to (and are therefore able to hybridize with) the C9orf72 gene as a component of the gene editing CasX:gNA systems. Representative, but non-limiting examples of targeting sequences to the C9orf72 target nucleic acid that can be utilized in the gNA of the embodiments are presented as SEQ ID NOS: 309-343, 363-2100 and 2295-21835. In some embodiments, the gNA is a deoxyribonucleic acid molecule (“gDNA”); in some embodiments, the gNA is a ribonucleic acid molecule (“gRNA”); and in other embodiments, the gNA is a chimera, and comprises both DNA and RNA. As used herein, the terms gNA, gRNA, and gDNA cover naturally-occurring molecules, as well as sequence variants.

It is envisioned that in some embodiments, multiple gNAs (e.g., multiple gRNAs) are delivered in the CasX:gNA system for the modification of a gene encoding one or more regions of a C9orf72 protein, a non-coding region of the C9orf72 gene, or a C9orf72 regulatory element. For example, when a deletion of a regulatory element or the HRS of the gene is desired, a pair of gNAs with targeting sequences to different or overlapping regions of the target nucleic acid sequence can be used in order to bind and cleave at two different or overlapping sites within the gene. In other cases, wherein the hexanucleotide repeat region is to be deleted, a pair of gNAs can be used in order to bind and cleave at two different sites 5′ and 3′ of the hexanucleotide repeats within the C9orf72 gene, resulting in the excision of the HRS that is then edited by non-homologous end joining (NHEJ), homology-directed repair (HDR), homology-independent targeted integration (HITI), micro-homology mediated end joining (MMEJ), single strand annealing (SSA) or base excision repair (BER). Exemplary pairs of gNAs that can be used to edit the HRS are shown in Table 16, below, and exemplary methods to effect the editing are described in Example 18, below.

a. Reference gNA and gNA Variants.

In some embodiments, a gNA of the present disclosure comprises a sequence of a naturally-occurring gNA (a “reference gNA”). In other cases, a reference gNA of the disclosure may be subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping, in order to generate one or more gNA variants with enhanced or varied properties relative to the reference gNA. gNA variants also include variants comprising one or more exogenous sequences, for example fused to either the 5′ or 3′ end, or inserted internally. The activity of reference gNAs may be used as a benchmark against which the activity of gNA variants are compared, thereby measuring improvements in function or other characteristics of the gNA variants. In other embodiments, a reference gNA may be subjected to one or more deliberate, specifically-targeted mutations in order to produce a gNA variant, for example a rationally designed variant.

The gNAs of the disclosure comprise two segments: a targeting sequence and a protein-binding segment. The targeting segment of a gNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a target ssRNA, a target ssDNA, a strand of a double stranded target DNA, etc.), described more fully below. The targeting sequence of a gNA is capable of binding to a target nucleic acid sequence, including a coding sequence, a complement of a coding sequence, a non-coding sequence, and to regulatory elements. The protein-binding segment (or “activator” or “protein-binding sequence”) interacts with (e.g., binds to) a CasX protein as a complex, forming an RNP (described more fully, below).

In the case of a dual guide RNA (dgRNA), the targeter and the activator portions each have a duplex-forming segment, where the duplex forming segment of the targeter and the duplex-forming segment of the activator have complementarity with one another and hybridize to one another to form a double stranded duplex (dsRNA duplex for a gRNA). When the gNA is a gRNA, the term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. Thus, for example, a guide RNA (dgRNA or sgRNA) comprises a guide sequence and a duplex-forming segment of a crRNA, which can also be referred to as a crRNA repeat. A corresponding tracrRNA-like molecule (activator) also comprises a duplex-forming stretch of nucleotides that forms the other half of the dsRNA duplex of the protein-binding segment of the guide RNA. Thus, a targeter and an activator, as a corresponding pair, hybridize to form a dual guide NA, referred to herein as a “dual guide NA”, a “dual-molecule gNA”, a “dgNA”, a “double-molecule guide NA”, or a “two-molecule guide NA”. Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gNA and the target nucleic acid sequence. Thus, for example, the gNA of the disclosure have sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. Thus, in some cases, the sequence of a targeter may be a non-naturally occurring sequence. In other cases, the sequence of a targeter may be a naturally-occurring sequence, derived from the gene to be edited. In other embodiments, the activator and targeter of the gNA are covalently linked to one another (rather than hybridizing to one another) and comprise a single molecule, referred to herein as a “single-molecule gNA,” “one-molecule guide NA,” “single guide NA”, “single guide RNA”, a “single-molecule guide RNA,” a “one-molecule guide RNA”, a “single guide DNA”, a “single-molecule DNA”, or a “one-molecule guide DNA”, (“sgNA”, “sgRNA”, or a “sgDNA”). In some embodiments, the sgNA includes an “activator” or a “targeter” and thus can be an “activator-RNA” and a “targeter-RNA,” respectively.

Collectively, the assembled gNAs of the disclosure comprise four distinct regions, or domains: the RNA triplex, the scaffold stem, the extended stem, and the targeting sequence that, in the embodiments of the disclosure is specific for a target nucleic acid and is located on the 3′ end of the gNA. The RNA triplex, the scaffold stem, and the extended stem, together, are referred to as the “scaffold” of the gNA.

b. RNA Triplex

In some embodiments of the guide NAs provided herein (including reference sgNAs), there is a RNA-triplex, and the RNA triplex comprises the sequence of a UUU-nX(˜4-15)-UUU stem loop (SEQ ID NO: 19) that ends with an AAAG after 2 intervening stem loops (the scaffold stem loop and the extended stem loop), forming a pseudoknot that may also extend past the triplex into a duplex pseudoknot. The UU-UUU-AAA sequence of the triplex forms as a nexus between the spacer, scaffold stem, and extended stem. In exemplary reference CasX sgNAs, the UUU-loop-UUU region is coded for first, then the scaffold stem loop, and then the extended stem loop, which is linked by the tetraloop, and then an AAAG closes off the triplex before becoming the spacer.

c. Scaffold Stem Loop

In some embodiments of sgNAs of the disclosure, the triplex region is followed by the scaffold stem loop. The scaffold stem loop is a region of the gNA that is bound by CasX protein (such as a reference or CasX variant protein). In some embodiments, the scaffold stem loop is a fairly short and stable stem loop. In some cases, the scaffold stem loop does not tolerate many changes, and requires some form of an RNA bubble. In some embodiments, the scaffold stem is necessary for CasX sgNA function. While it is perhaps analogous to the nexus stem of Cas9 as being a critical stem loop, the scaffold stem of a CasX sgNA, in some embodiments, has a necessary bulge (RNA bubble) that is different from many other stem loops found in CRISPR/Cas systems. In some embodiments, the presence of this bulge is conserved across sgNA that interact with different CasX proteins. An exemplary sequence of a scaffold stem loop sequence of a gNA comprises the sequence CCAGCGACUAUGUCGUAUGG (SEQ ID NO: 20). In other embodiments, the disclosure provides gNA variants wherein the scaffold stem loop is replaced with an RNA stem loop sequence from a heterologous RNA source with proximal 5′ and 3′ ends, such as, but not limited to stem loop sequences selected from MS2, Q β, U1 hairpin II, Uvsx, or PP7 stem loops. In some cases, the heterologous RNA stem loop of the gNA is capable of binding a protein, an RNA structure, a DNA sequence, or a small molecule.

d. Extended Stem Loop

In some embodiments of the CasX sgNAs of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA tetraloop linker or a GAGAAA linker between the tracr and crRNA in the extended stem loop. In some cases, the targeter and activator of a CasX sgNA are linked to one another by intervening nucleotides and the linker can have a length of from 3 to 20 nucleotides. In some embodiments of the CasX sgNAs of the disclosure, the extended stem is a large 32-bp loop that sits outside of the CasX protein in the ribonucleoprotein complex. An exemplary sequence of an extended stem loop sequence of a sgNA comprises the sequence GCGCUUAUUUAUCGGAGAGAAAUCCGAUAAAUAAGAAGC (SEQ ID NO: 21). In some embodiments, the extended stem loop comprises a GAGAAA spacer sequence. In some embodiments, the disclosure provides gNA variants wherein the extended stem loop is replaced with an RNA stem loop sequence from a heterologous RNA source with proximal 5′ and 3′ ends, such as, but not limited to stem loop sequences selected from MS2, Qβ, U1 hairpin II, Uvsx, or PP7 stem loops. In such cases, the heterologous RNA stem loop increases the stability of the gNA. In other embodiments, the disclosure provides gNA variants having an extended stem loop region comprising at least 10, at least 100, at least 500, at least 1000, or at least 10,000 nucleotides, or at least 10-10,000, at least 10-1000, or at least 10-100 nucleotides.

e. Targeting Sequence

In some embodiments of the gNAs of the disclosure, the extended stem loop is followed by a region that forms part of the triplex, and then the targeting sequence. The targeting sequence targets the CasX ribonucleoprotein holo complex to a specific region of the target nucleic acid sequence of the C9orf72 gene. Thus, for example, CasX gNA targeting sequences of the disclosure have sequences complementarity to, and therefore can hybridize to, a portion of the C9orf72 gene in a nucleic acid in a eukaryotic cell (e.g., a eukaryotic chromosome, chromosomal sequence, a eukaryotic RNA, etc.) as a component of the RNP when the TC PAM motif or any one of the PAM sequences TTC, ATC, GTC, or CTC is located 1 nucleotide 5′ to the non-target strand sequence complementary to the target sequence. The targeting sequence of a gNA can be modified so that the gNA can target a desired sequence of any desired target nucleic acid sequence, so long as the PAM sequence location is taken into consideration. In some embodiments, the gNA scaffold is 5′ of the targeting sequence, with the targeting sequence on the 3′ end of the gNA. In some embodiments, the PAM motif sequence recognized by the nuclease of the RNP is TC. In other embodiments, the PAM sequence recognized by the nuclease of the RNP is NTC.

In some embodiments, the targeting sequence of the gNA is specific for a portion of a gene encoding a C9orf72 protein comprising one or more mutations. In some embodiments, the targeting sequence of a gNA is specific for a C9orf72 exon. In some embodiments, the targeting sequence of a gNA is specific for a C9orf72 intron. In some embodiments, the targeting sequence of the gNA is specific for a C9orf72 intron-exon junction. In some embodiments, the targeting sequence of the gNA has a sequence that hybridizes with a C9orf72 regulatory element, a C9orf72 coding region, a C9orf72 non-coding region, or combinations thereof. In particular embodiments, the targeting sequence of the gNA hybridizes with a sequence that is 5′ to the HRS. In some embodiments, wherein two or more gNA are used, the first gNA targeting sequence of the gNA hybridizes with a sequence that is 5′ to the HRS and the second gNA hybridizes with a sequence that is 3′ to the HRS. In some embodiments, the targeting sequence of the gNA is complementary to a sequence comprising one or more single nucleotide polymorphisms (SNPs) of the C9orf72 gene or its complement. SNPs that are within C9orf72 coding sequence or within C9orf72 non-coding sequence are both within the scope of the instant disclosure. In other embodiments, the targeting sequence of the gNA is complementary to a sequence of an intergenic region of the C9orf72 gene or a sequence complementary to an intergenic region of the C9orf72 gene.

In some embodiments, the targeting sequence of a gNA is specific for a regulatory element that regulates expression of C9orf72. Such C9orf72 regulatory elements include, but are not limited to promoter regions, enhancer regions, intergenic regions, 5′ untranslated regions (5′ UTR), 3′ untranslated regions (3′ UTR), intergenic regions, gene enhancer elements, conserved elements, and regions comprising cis-regulatory elements. The promoter region is intended to encompass nucleotides within 100 kb of the C9orf72 initiation point or, in the case of gene enhancer elements or conserved elements, can be 1 Mb or more distal to the C9orf72 gene. In some embodiments, the disclosure provides a gNA with a targeting sequence that hybridizes with a C9orf72 regulatory element. In the foregoing, the targets are those in which the encoding gene of the target is intended to be knocked out or knocked down such that the C9orf72 protein comprising mutations or the hexanucleotide duplication of the C9orf72 gene product is not expressed or is expressed at a lower level in a cell. In some embodiments, the disclosure provides a CasX:gNA system wherein the targeting sequence (or spacer) of the gNA is complementary to a nucleic acid sequence encoding C9orf72, a portion of the C9orf72 protein, a portion of a C9orf72 regulatory element, or the complement of a portion of the C9orf72 gene. In some embodiments, the targeting sequence of the gNA has between 14 and 35 consecutive nucleotides. In some embodiments, the targeting sequence has 14, 15, 16, 18, 18, 19, 20, 21, 22, 23, 24, 25, 26, 27, 28, 29, 30, 31, 32, 33, 34 or 35 consecutive nucleotides. In some embodiments, the targeting sequence consists of 21 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the targeting sequence has 14, 15, 16, 17, 18, 19, 20, or 21 consecutive nucleotides and the targeting sequence can comprise 0 to 5, 0 to 4, 0 to 3, or 0 to 2 mismatches relative to the target nucleic acid sequence and retain sufficient binding specificity such that the RNP comprising the gNA comprising the targeting sequence can form a complementary bond with respect to the target nucleic acid.

Representative, but non-limiting examples of targeting sequences to wild-type C9orf72 nucleic acid are presented in Tables 3 and 15 as SEQ ID NOS: 309-343, 363-2100 and 2295-21835. In some embodiments, the disclosure provides targeting sequences comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence in Tables 3 and 15 as SEQ ID NOS: 309-343, 363-2100 and 2295-21835. In some embodiments, the targeting sequence of the gNA comprises a sequence of SEQ ID NOS: 2281-159093 with a single nucleotide removed from the 3′ end of the sequence. In other embodiments, the targeting sequence of the gNA comprises a sequence of SEQ ID NOS: 309-343, 363-2100 and 2295-21835 with two nucleotides removed from the 3′ end of the sequence. In other embodiments, the targeting sequence of the gNA comprises a sequence of SEQ ID NOS: 309-343, 363-2100 and 2295-21835 with three nucleotides removed from the 3′ end of the sequence. In other embodiments, the targeting sequence of the gNA comprises a sequence of SEQ ID NOS: 309-343, 363-2100 and 2295-21835 with four nucleotides removed from the 3′ end of the sequence. In other embodiments, the targeting sequence of the gNA comprises a sequence of SEQ ID NOS: 309-343, 363-2100 and 2295-21835 with five nucleotides removed from the 3′ end of the sequence. In the foregoing embodiments of the paragraph, thymine (T) nucleotides can be substituted for one or more or all of the uracil (U) nucleotides in any of the targeting sequences such that the gNA can be a gDNA or a gRNA, or a chimera of RNA and DNA, or in those cases where the encoding sequence for the spacer is incorporated into an expression vector. In some embodiments, a targeting sequence of SEQ ID NOS: 309-343, 363-2100 and 2295-21835 has at least 1, 2, 3, 4, 5, or 6 or more thymine nucleotides substituted for uracil nucleotides. In other embodiments, a gNA, gRNA, or gDNA of the disclosure comprises 1, 2, 3 or more targeting sequences of SEQ ID NOS: 309-343, 363-2100 and 2295-21835, or targeting sequences that are at least 50% identical, at least 55% identical, at least 60% identical, at least 65% identical, at least 70% identical, at least 75% identical, at least 80% identical, at least 85% identical, at least 90% identical, at least 95% identical, or 100% identical to one or more sequences of SEQ ID NOS: 309-343, 363-2100 and 2295-21835.

In some embodiments, the targeting sequence is complementary to a nucleic acid sequence encoding a mutation of the C9orf72 protein of SEQ ID NO: 227 or 228 or hexanucleotide duplications that disrupt the function or expression of the C9orf72 protein.

In some embodiments, the CasX:gNA system comprises a first gNA and further comprises a second (and optionally a third, fourth or fifth) gNA, wherein the second gNA or additional gNA has a targeting sequence complementary a different portion of the target nucleic acid sequence or its complement compared to the targeting sequence of the first gNA; e.g., the first gNA targets 5′ to the hexanucleotide repeat and the second gNA targets 3′ to the hexanucleotide repeat. By selection of the targeting sequences of the gNA, defined regions of the target nucleic acid sequence can be modified or edited using the CasX:gNA systems described herein.

### f. gNA Scaffolds

In some embodiments, a CasX reference gRNA comprises a sequence isolated or derived from Deltaproteobacter. In some embodiments, the sequence is a CasX tracrRNA sequence. Exemplary CasX reference tracrRNA sequences isolated or derived from Deltaproteobacter may include: ACAUCUGGCGCGUUUAUUCCAUUACUUUGGAGCCAGUCCCAGCGACUAUGUCGU AUGGACGAAGCGCUUAUUUAUCGGAGA (SEQ ID NO: 22) and ACAUCUGGCGCGUUUAUUCCAUUACUUUGGAGCCAGUCCCAGCGACUAUGUCGU AUGGACGAAGCGCUUAUUUAUCGG (SEQ ID NO: 23). Exemplary crRNA sequences isolated or derived from Deltaproteobacter may comprise a sequence of CCGAUAAGUAAAACGCAUCAAAG (SEQ ID NO: 24). In some embodiments, a CasX reference gNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Deltaproteobacter.

In some embodiments, a CasX reference guide RNA comprises a sequence isolated or derived from Planctomycetes. In some embodiments, the sequence is a CasX tracrRNA sequence. Exemplary CasX reference tracrRNA sequences isolated or derived from Planctomycetes may include: UACUGGCGCUUUUAUCUCAUUACUUUGAGAGCCAUCACCAGCGACUAUGUCGUA UGGGUAAAGCGCUUAUUUAUCGGAGA (SEQ ID NO: 25) and UACUGGCGCUUUUAUCUCAUUACUUUGAGAGCCAUCACCAGCGACUAUGUCGUA UGGGUAAAGCGCUUAUUUAUCGG (SEQ ID NO: 26). Exemplary crRNA sequences isolated or derived from Planctomycetes may comprise a sequence of UCUCCGAUAAAUAAGAAGCAUCAAAG (SEQ ID NO: 27). In some embodiments, a CasX reference gNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Planctomycetes.

In some embodiments, a CasX reference gNA comprises a sequence isolated or derived from Candidatus sungbacteria. In some embodiments, the sequence is a CasX tracrRNA sequence. Exemplary CasX reference tracrRNA sequences isolated or derived from Candidatus sungbacteria may comprise sequences of: GUUUACACACUCCCUCUCAUAGGGU (SEQ ID NO: 28), GUUUACACACUCCCUCUCAUGAGGU (SEQ ID NO: 29), UUUUACAUACCCCCUCUCAUGGGAU (SEQ ID NO: 30) and GUUUACACACUCCCUCUCAUGGGGG (SEQ ID NO: 31). In some embodiments, a CasX reference guide RNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Candidatus sungbacteria.

Table 1 provides the sequences of reference gRNAs tracr and scaffold sequences. In some embodiments, the disclosure provides gNA sequences wherein the gNA has a scaffold comprising a sequence having at least one nucleotide modification relative to a reference gNA sequence having a sequence of any one of SEQ ID NOS:4-16 of Table 1. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein, including the sequences of Table 1 and Table 2.

TABLE 1
Reference gRNA tracr and scaffold sequences

| SEQ | |
|---|---|
| ID | |
| NO. | Nucleotide Sequence |
| 4 | ACAUCUGGCGCGUUUAUUCCAUUACUUUGGAGCCAGUCCCAGCG |
| | ACUAUGUCGUAUGGACGAAGCGCUUAUUUAUCGGAGAGAAACCG |
| | AUAAGUAAAACGCAUCAAAG |
| 5 | UACUGGCGCUUUUAUCUCAUUACUUUGAGAGCCAUCACCAGCGA |
| | CUAUGUCGUAUGGGUAAAGCGCUUAUUUAUCGGAGAGAAAUCCG |
| | AUAAAUAAGAAGCAUCAAAG |
| 6 | ACAUCUGGCGCGUUUAUUCCAUUACUUUGGAGCCAGUCCCAGCG |
| | ACUAUGUCGUAUGGACGAAGCGCUUAUUUAUCGGAGA |
| 7 | ACAUCUGGCGCGUUUAUUCCAUUACUUUGGAGCCAGUCCCAGCG |
| | ACUAUGUCGUAUGGACGAAGCGCUUAUUUAUCGG |
| 8 | UACUGGCGCUUUUAUCUCAUUACUUUGAGAGCCAUCACCAGCGA |
| | CUAUGUCGUAUGGGUAAAGCGCUUAUUUAUCGGAGA |
| 9 | UACUGGCGCUUUUAUCUCAUUACUUUGAGAGCCAUCACCAGCGA |
| | CUAUGUCGUAUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAU |
| | GUCGUAUGGGUAAAGCGCUUAUUUAUCGGA |

### g. gNA Variants

In another aspect, the disclosure relates to guide nucleic acid variants (referred to herein alternatively as “gNA variant” or “gRNA variant”), which comprise one or more modifications relative to a reference gRNA scaffold. As used herein, “scaffold” refers to all parts to the gNA necessary for gNA function with the exception of the spacer sequence.

In some embodiments, a gNA variant comprises one or more nucleotide substitutions, insertions, deletions, or swapped or replaced regions relative to a reference gRNA sequence of the disclosure. In some embodiments, a mutation can occur in any region of a reference gRNA to produce a gNA variant. In some embodiments, the scaffold of the gNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO:4 or SEQ ID NO:5.

In some embodiments, a gNA variant comprises one or more nucleotide changes within one or more regions of the reference gRNA that improve a characteristic of the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gNA variant scaffold comprises a scaffold stem loop having at least 60% sequence identity to SEQ ID NO:14. In another embodiment, the gNA variant comprises a scaffold stem loop having the sequence of CCAGCGACUAUGUCGUAGUGG (SEQ ID NO: 32). In another embodiment, the disclosure provides a gNA scaffold comprising, relative to SEQ ID NO:5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G64U. In the foregoing embodiment, the gNA scaffold comprises the sequence

(SEQ ID NO: 33)
ACUGGCGCUUUUAUCUGAUUACUUUGAGAGCCAUCACCAGCGACUAUGUC
GUAGUGGGUAAAGCUCCCUCUUCGGAGGGAGCAUCAAAG.

All gNA variants that have one or more improved functions or characteristics, or add one or more new functions when the variant gNA is compared to a reference gRNA described herein, are envisaged as within the scope of the disclosure. A representative example of such a gNA variant is guide 174 (SEQ ID NO:2238), the design of which is described in the Examples. In some embodiments, the gNA variant adds a new function to the RNP comprising the gNA variant. In some embodiments, the gNA variant has an improved characteristic selected from: improved stability; improved solubility; improved transcription of the gNA; improved resistance to nuclease activity; increased folding rate of the gNA; decreased side product formation during folding; increased productive folding; improved binding affinity to a CasX protein; improved binding affinity to a target DNA when complexed with a CasX protein; improved gene editing when complexed with a CasX protein; improved specificity of editing when complexed with a CasX protein; and improved ability to utilize a greater spectrum of one or more PAM sequences, including ATC, CTC, GTC, or TTC, in the editing of target DNA when complexed with a CasX protein, or any combination thereof. In some cases, the one or more of the improved characteristics of the gNA variant is at least about 1.1 to about 100,000-fold improved relative to the reference gNA of SEQ ID NO:4 or SEQ ID NO:5. In other cases, the one or more improved characteristics of the gNA variant is at least about 1.1, at least about 10, at least about 100, at least about 1000, at least about 10,000, at least about 100,000-fold or more improved relative to the reference gNA of SEQ ID NO:4 or SEQ ID NO:5. In other cases, the one or more of the improved characteristics of the gNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the reference gNA of SEQ ID NO:4 or SEQ ID NO:5. In other cases, the one or more improved characteristics of the gNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to the reference gNA of SEQ ID NO:4 or SEQ ID NO:5.

In some embodiments, a gNA variant can be created by subjecting a reference gRNA to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gNA variants of the disclosure. The activity of reference gRNAs may be used as a benchmark against which the activity of gNA variants are compared, thereby measuring improvements in function of gNA variants. In other embodiments, a reference gRNA may be subjected to one or more deliberate, targeted mutations, substitutions, or domain swaps in order to produce a gNA variant, for example a rationally designed variant. Exemplary gRNA variants produced by such methods are described in the Examples and representative sequences of gNA scaffolds are presented in Table 2.

In some embodiments, the gNA variant comprises one or more modifications compared to a reference guide nucleic acid scaffold sequence, wherein the one or more modification is selected from: at least one nucleotide substitution in a region of the gNA variant; at least one nucleotide deletion in a region of the gNA variant; at least one nucleotide insertion in a region of the gNA variant; a substitution of all or a portion of a region of the gNA variant; a deletion of all or a portion of a region of the gNA variant; or any combination of the foregoing. In some cases, the modification is a substitution of 1 to 15 consecutive or non-consecutive nucleotides in the gNA variant in one or more regions. In other cases, the modification is a deletion of 1 to 10 consecutive or non-consecutive nucleotides in the gNA variant in one or more regions. In other cases, the modification is an insertion of 1 to 10 consecutive or non-consecutive nucleotides in the gNA variant in one or more regions. In other cases, the modification is a substitution of the scaffold stem loop or the extended stem loop with an RNA stem loop sequence from a heterologous RNA source with proximal 5′ and 3′ ends. In some cases, a gNA variant of the disclosure comprises two or more modifications in one region. In other cases, a gNA variant of the disclosure comprises modifications in two or more regions. In other cases, a gNA variant comprises any combination of the foregoing modifications described in this paragraph.

In some embodiments, a 5′ G is added to a gNA variant sequence for expression in vivo, as transcription from a U6 promoter is more efficient and more consistent with regard to the start site when the +1 nucleotide is a G. In other embodiments, two 5′ Gs are added to a gNA variant sequence for in vitro transcription to increase production efficiency, as T7 polymerase strongly prefers a G in the +1 position and a purine in the +2 position. In some cases, the 5′ G bases are added to the reference scaffolds of Table 1. In other cases, the 5′ G bases are added to the variant scaffolds of Table 2.

Table 2 provides exemplary gNA variant scaffold sequences. In Table 2, (−) indicates a deletion at the specified position(s) relative to the reference sequence of SEQ ID NO:5, (+) indicates an insertion of the specified base(s) at the position indicated relative to SEQ ID NO:5, (:) indicates the range of bases at the specified start:stop coordinates of a deletion or substitution relative to SEQ ID NO:5, and multiple insertions, deletions or substitutions are separated by commas; e.g., A14C, U17G. In some embodiments, the gNA variant scaffold comprises any one of the sequences listed in Table 2 as SEQ ID NOS:2101-2294, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein.

TABLE 2
Exemplary gNA Scaffold Sequences

| SEQ | | |
|---|---|---|
| ID | NAME or | |
| NO: | Modification | NUCLEOTIDE SEQUENCE |
| 2101 | phage | UACUGGCGCUUUUAUCUCAUUACUUUGAGAGCCAUCACCAGCGACUA |
| | replication | UGUCGUAUGGGUAAAGCGCAGGUGGGACGACCUCUCGGUCGUCCUAU |
| | stable | CUGAAGCAUCAAAG |
| 2102 | Kissing | UACUGGCGCUUUUAUCUCAUUACUUUGAGAGCCAUCACCAGCGACUA |
| | loop_b1 | UGUCGUAUGGGUAAAGCGCUGCUCGACGCGUCCUCGAGCAGAAGCAU |
| | | CAAAG |
| 2103 | Kissing | UACUGGCGCUUUUAUCUCAUUACUUUGAGAGCCAUCACCAGCGACUA |
| | loop_a | UGUCGUAUGGGUAAAGCGCUGCUCGCUCCGUUCGAGCAGAAGCAUCA |
| | | AAG |
| 2104 | 32: uvsX | GUACUGGCGCUUUUAUCUCAUUACUUUGAGAGCCAUCACCAGCGACU |
| | hairpin | AUGUCGUAUGGGUAAAGCGCCCUCUUCGGAGGGAAGCAUCAAAG |
| 2105 | PP7 | UACUGGCGCUUUUAUCUCAUUACUUUGAGAGCCAUCACCAGCGACUA |
| | | UGUCGUAUGGGUAAAGCGCAGGAGUUUCUAUGGAAACCCUGAAGCAU |
| | | CAAAG |
| 2106 | 64: trip mut, | GUACUGGCGCCUUUAUCUCAUUACUUUGAGAGCCAUCACCAGCGACU |
| | extended stem | AUGUCGUAUGGGUAAAGCGCUUACGGACUUCGGUCCGUAAGAAGCAU |
| | truncation | CAAAG |
| 2107 | hyperstable | UACUGGCGCUUUUAUCUCAUUACUUUGAGAGCCAUCACCAGCGACUA |
| | tetraloop | UGUCGUAUGGGUAAAGCGCUGCGCUUGCGCAGAAGCAUCAAAG |
| 2108 | C18G | UACUGGCGCUUUUAUCUGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2109 | U17G | UACUGGCGCUUUUAUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2110 | CUUCGG | UACUGGCGCUUUUAUCUCAUUACUUUGAGAGCCAUCACCAGCGACUA |
| | loop | UGUCGUAUGGGUAAAGCGCUUAUUUAUCGGAGACUUCGGUCCGAUAA |
| | | AUAAGAAGCAUCAAAG |
| 2111 | MS2 | UACUGGCGCUUUUAUCUCAUUACUUUGAGAGCCAUCACCAGCGACUA |
| | | UGUCGUAUGGGUAAAGCGCACAUGAGGAUUACCCAUGUGAAGCAUCA |
| | | AAG |
| 2112 | −1, A2G, −78, | GCUGGCGCUUUUAUCUCAUUACUUUGAGAGCCAUCACCAGCGACUAU |
| | G77U | GUCGUAUGGGUAAAGCGCUUAUUUAUCGUGAGAAAUCCGAUAAAUAA |
| | | GAAGCAUCAAAG |
| 2113 | QB | UACUGGCGCUUUUAUCUCAUUACUUUGAGAGCCAUCACCAGCGACUA |
| | | UGUCGUAUGGGUAAAGCGCUGCAUGUCUAAGACAGCAGAAGCAUCAA |
| | | AG |
| 2114 | 45, 44 hairpin | UACUGGCGCUUUUAUCUCAUUACUUUGAGAGCCAUCACCAGCGACUA |
| | | UGUCGUAUGGGUAAAGCGCAGGGCUUCGGCCGAAGCAUCAAAG |
| 2115 | U1A | UACUGGCGCUUUUAUCUCAUUACUUUGAGAGCCAUCACCAGCGACUA |
| | | UGUCGUAUGGGUAAAGCGCAAUCCAUUGCACUCCGGAUUGAAGCAUC |
| | | AAAG |
| 2116 | A14C, U17G | UACUGGCGCUUUUCUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2117 | CUUCGG | UACUGGCGCUUUUAUCUCAUUACUUUGAGAGCCAUCACCAGCGACUA |
| | loop modified | UGUCGUAUGGGUAAAGCGCUUAUUUAUCGGACUUCGGUCCGAUAAAU |
| | | AAGAAGCAUCAAAG |
| 2118 | Kissing | UACUGGCGCUUUUAUCUCAUUACUUUGAGAGCCAUCACCAGCGACUA |
| | loop_b2 | UGUCGUAUGGGUAAAGCGCUGCUCGUUUGCGGCUACGAGCAGAAGCA |
| | | UCAAAG |
| 2119 | −76:78, −83:87 | UACUGGCGCUUUUAUCUCAUUACUUUGAGAGCCAUCACCAGCGACUA |
| | | UGUCGUAUGGGUAAAGCGCUUAUUUAUCGAGAGAUAAAUAAGAAGCA |
| | | UCAAAG |
| 2120 | −4 | UACGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2121 | extended stem | UACUGGCGCCUUUUAUCUCAUUACUUUGAGAGCCAUCACCAGCGACU |
| | truncation | AUGUCGUAUGGGUAAAGCGCUUACGGACUUCGGUCCGUAAGAAGCAU |
| | | CAAAG |
| 2122 | C55 | UACUGGCGCUUUUAUCUCAUUACUUUGAGAGCCAUCACCAGCGACUA |
| | | UGUCGUAUCGGUAAAGCGCUUAUUUAUCGGAGAGAAAUCCGAUAAAU |
| | | AAGAAGCAUCAAAG |
| 2123 | trip mut | UACUGGCGCCUUUAUCUCAUUACUUUGAGAGCCAUCACCAGCGACUA |
| | | UGUCGUAUGGGUAAAGCGCUUAUUUAUCGGACUUCGGUCCGAUAAAU |
| | | AAGAAGCAUCAAAG |
| 2124 | −76:78 | UACUGGCGCUUUUAUCUCAUUACUUUGAGAGCCAUCACCAGCGACUA |
| | | UGUCGUAUGGGUAAAGCGCUUAUUUAUCGAGAAAUCCGAUAAAUAAG |
| | | AAGCAUCAAAG |
| 2125 | −1:5 | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2126 | −83:87 | UACUGGCGCUUUUAUCUCAUUACUUUGAGAGCCAUCACCAGCGACUA |
| | | UGUCGUAUGGGUAAAGCGCUUAUUUAUCGGAGAGAGAUAAAUAAGAA |
| | | GCAUCAAAG |
| 2127 | =+G28, | UACUGGCGCUUUUAUCUCAUUACUUUGGAGAGCCAUCACCAGCGACU |
| | A82U, −84, | AUGUCGUAUGGGUAAAGCGCUUAUUUAUCGGAGAGUAUCCGAUAAAU |
| | | AAGAAGCAUCAAAG |
| 2128 | =+51U | UACUGGCGCUUUUAUCUCAUUACUUUGAGAGCCAUCACCAGCGACUA |
| | | UGUUCGUAUGGGUAAAGCGCUUAUUUAUCGGAGAGAAAUCCGAUAAA |
| | | UAAGAAGCAUCAAAG |
| 2129 | −1: 4, +G5A, | AGCGCUUUUAUCUCAUUACUUUGAGAGCCAUCACCAGCGACUAUGUC |
| | +G86, | GUAUGGGUAAAGCGCUUAUUUAUCGGAGAGAAAUGCCGAUAAAUAAG |
| | | AAGCAUCAAAG |
| 2130 | =+A94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | UAAGAAGCAUCAAAG |
| 2131 | =+G72 | UACUGGCGCUUUUAUCUCAUUACUUUGAGAGCCAUCACCAGCGACUA |
| | | UGUCGUAUGGGUAAAGCGCUUAUUGUAUCGGAGAGAAAUCCGAUAAA |
| | | UAAGAAGCAUCAAAG |
| 2132 | shorten front, | GCGCUUUUAUCUCAUUACUUUGAGAGCCAUCACCAGCGACUAUGUCG |
| | CUUCGG | UAUGGGUAAAGCGCUUAUUUAUCGGACUUCGGUCCGAUAAAUAAGCG |
| | loop modified. | CAUCAAAG |
| | extend | |
| | extended | |
| 2133 | A14C | UACUGGCGCUUUUCUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2134 | −1: 3, +G3 | GUGGCGCUUUUAUCUCAUUACUUUGAGAGCCAUCACCAGCGACUAUG |
| | | UCGUAUGGGUAAAGCGCUUAUUUAUCGGAGAGAAAUCCGAUAAAUAA |
| | | GAAGCAUCAAAG |
| 2135 | =+C45, +U46 | UACUGGCGCUUUUAUCUCAUUACUUUGAGAGCCAUCACCAGCGACCU |
| | | UAUGUCGUAUGGGUAAAGCGCUUAUUUAUCGGAGAGAAAUCCGAUAA |
| | | AUAAGAAGCAUCAAAG |
| 2136 | CUUCGG | GAUGGCGCUUUUAUCUCAUUACUUUGAGAGCCAUCACCAGCGACUAU |
| | loop modified, | GUCGUAUGGGUAAAGCGCUUAUUUAUCGGACUUCGGUCCGAUAAAUA |
| | fun start | AGAAGCAUCAAAG |
| 2137 | −93: 94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | GAAGCAUCAAAG |
| 2138 | =+U45 | UACUGGCGCUUUUAUCUCAUUACUUUGAGAGCCAUCACCAGCGAUCU |
| | | AUGUCGUAUGGGUAAAGCGCUUAUUUAUCGGAGAGAAAUCCGAUAAA |
| | | UAAGAAGCAUCAAAG |
| 2139 | −69, −94 | UACUGGCGCUUUUAUCUCAUUACUUUGAGAGCCAUCACCAGCGACUA |
| | | UGUCGUAUGGGUAAAGGCUUAUUUAUCGGAGAGAAAUCCGAUAAAAA |
| | | GAAGCAUCAAAG |
| 2140 | -94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | AGAAGCAUCAAAG |
| 2141 | modified | UACUGGCGCUUUAUCUCAUUACUUUGAGAGCCAUCACCAGCGACUAU |
| | CUUCGG, | GUCGUAUGGGUAAAGCGCUUAUUUAUCGGACUUCGGUCCGAUAAAUA |
| | minus U in 1st | AGAAGCAUCAAAG |
| | triplex | |
| 2142 | −1: 4, +C4, | CGGCGCUUUUCUCGCAUUACUUUGAGAGCCAUCACCAGCGACUAUGU |
| | A14C, U17G, | CGUAUGGGUAAAGCGCUUAUUGUAUCGAGAGAUAAAUAAGAAGCAUC |
| | +G72, −76:78, | AAAG |
| | −83:87 | |
| 2143 | U1C, −73 | CACUGGCGCUUUUAUCUCAUUACUUUGAGAGCCAUCACCAGCGACUA |
| | | UGUCGUAUGGGUAAAGCGCUUAUUUUCGGAGAGAAAUCCGAUAAAUA |
| | | AGAAGCAUCAAAG |
| 2144 | Scaffold | UACUGGCGCUUUAUCUCAUUACUUUGAGAGCCAUCACCAGCGACUUC |
| | uuCG, stem | GGUCGUAUGGGUAAAGCGCUUAUGUAUCGGCUUCGGCCGAUACAUAA |
| | uuCG. Stem | GAAGCAUCAAAG |
| | swap, t | |
| | shorten | |
| 2145 | Scaffold | UACUGGCGCUUUUAUCUCAUUACUUUGAGAGCCAUCACCAGCGACUU |
| | uuCG, stem | CGGUCGUAUGGGUAAAGCGCUUAUGUAUCGGCUUCGGCCGAUACAUA |
| | uuCG. Stem | AGAAGCAUCAAAG |
| | swap | |
| 2146 | =+G60 | UACUGGCGCUUUUAUCUCAUUACUUUGAGAGCCAUCACCAGCGACUA |
| | | UGUCGUAUGGGUGAAAGCGCUUAUUUAUCGGAGAGAAAUCCGAUAAA |
| | | UAAGAAGCAUCAAAG |
| 2147 | no stem | UACUGGCGCUUUUAUCUCAUUACUUUGAGAGCCAUCACCAGCGACUU |
| | Scaffold | CGGUCGUAUGGGUAAAG |
| | uuCG | |
| 2148 | no stem | GAUGGGCUUUUAUCUCAUUACUUUGAGAGCCAUCACCAGCGACUUCG |
| | Scaffold | GUCGUAUGGGUAAAG |
| | uuCG, fun | |
| | start | |
| 2149 | Scaffold | GAUGGGCUUUUAUCUCAUUACUUUGAGAGCCAUCACCAGCGACUUCG |
| | uuCG, stem | GUCGUAUGGGUAAAGCGCUUAUUUAUCGGCUUCGGCCGAUAAAUAAG |
| | uuCG, fun | AAGCAUCAAAG |
| | start | |
| 2150 | Pseudoknots | UACUGGCGCUUUUAUCUCAUUACUUUGAGAGCCAUCACCAGCGACUA |
| | | UGUCGUAUGGGUAAAGCGCUACACUGGGAUCGCUGAAUUAGAGAUCG |
| | | GCGUCCUUUCAUUCUAUAUACUUUGGAGUUUUAAAAUGUCUCUAAGU |
| | | ACAGAAGCAUCAAAG |
| 2151 | Scaffold | GGCGCUUUUAUCUCAUUACUUUGAGAGCCAUCACCAGCGACUUCGGU |
| | uuCG, stem | CGUAUGGGUAAAGCGCUUAUUUAUCGGCUUCGGCCGAUAAAUAAGAA |
| | uuCG | GCAUCAAAG |
| 2152 | Scaffold | GCUGGCGCUUUUAUCUCAUUACUUUGAGAGCCAUCACCAGCGACUUC |
| | uuCG, stem | GGUCGUAUGGGUAAAGCGCUUAUUUAUCGGCUUCGGCCGAUAAAUAA |
| | uuCG, no start | GAAGCAUCAAAG |
| 2153 | Scaffold | UACUGGCGCUUUUAUCUCAUUACUUUGAGAGCCAUCACCAGCGACUU |
| | uuCG | CGGUCGUAUGGGUAAAGCGCUUAUUUAUCGGAGAGAAAUCCGAUAAA |
| | | UAAGAAGCAUCAAAG |
| 2154 | =+GCUC36 | UACUGGCGCUUUUAUCUCAUUACUUUGAGAGCCAUGCUCCACCAGCG |
| | | ACUAUGUCGUAUGGGUAAAGCGCUUAUUUAUCGGAGAGAAAUCCGAU |
| | | AAAUAAGAAGCAUCAAAG |
| 2155 | G quadriplex | UACUGGCGCUUUUAUCUCAUUACUUUGAGAGCCAUCACCAGCGACUA |
| | telomere | UGUCGUAUGGGUAAAGCGGGGUUAGGGUUAGGGUUAGGGAAGCAUCA |
| | basket + ends | AAG |
| 2156 | G quadriplex | UACUGGCGCUUUUAUCUCAUUACUUUGAGAGCCAUCACCAGCGACUA |
| | M3q | UGUCGUAUGGGUAAAGCGGAGGGAGGGAGGGAGAGGGAAAGCAUCAA |
| | | AG |
| 2157 | G quadriplex | UACUGGCGCUUUUAUCUCAUUACUUUGAGAGCCAUCACCAGCGACUA |
| | telomere | UGUCGUAUGGGUAAAGCGUUGGGUUAGGGUUAGGGUUAGGGAAAAGC |
| | basket no ends | AUCAAAG |
| 2158 | 45, 44 hairpin | UACUGGCGCUUUUAUCUCAUUACUUUGAGAGCCAUCACCAGCGACUA |
| | (old version) | UGUCGUAUGGGUAAAGCGC--------AGGGCUUCGGCCG------- |
| | | --GAAGCAUCAAAG |
| 2159 | Sarcin-ricin | UACUGGCGCUUUUAUCUCAUUACUUUGAGAGCCAUCACCAGCGACUA |
| | loop | UGUCGUAUGGGUAAAGCGCCUGCUCAGUACGAGAGGAACCGCAGGAA |
| | | GCAUCAAAG |
| 2160 | uvsX, C18G | UACUGGCGCUUUUAUCUGAUUACUUUGAGAGCCAUCACCAGCGACUA |
| | | UGUCGUAUGGGUAAAGCGCCCUCUUCGGAGGGAAGCAUCAAAG |
| 2161 | truncated stem | UACUGGCGCCUUUAUCUGAUUACUUUGAGAGCCAUCACCAGCGACUA |
| | loop, C18G, | UGUCGUAUGGGUAAAGCGCUUACGGACUUCGGUCCGUAAGAAGCAUC |
| | trip mut | AAAG |
| | (U10C) | |
| 2162 | short phage | UACUGGCGCUUUUAUCUGAUUACUUUGAGAGCCAUCACCAGCGACUA |
| | rep, C18G | UGUCGUAUGGGUAAAGCGCGGACGACCUCUCGGUCGUCCGAAGCAUC |
| | | AAAG |
| 2163 | phage rep | UACUGGCGCUUUUAUCUGAUUACUUUGAGAGCCAUCACCAGCGACUA |
| | loop, C18G | UGUCGUAUGGGUAAAGCGCAGGUGGGACGACCUCUCGGUCGUCCUAU |
| | | CUGAAGCAUCAAAG |
| 2164 | =+G18, | UACUGGCGCCUUUAUCUGCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2165 | truncated stem | GCUGGCGCUUUUAUCUGAUUACUUUGAGAGCCAUCACCAGCGACUAU |
| | loop, C18G, | GUCGUAUGGGUAAAGCGCUUACGGACUUCGGUCCGUAAGAAGCAUCA |
| | −1 A2G | AAG |
| 2166 | phage rep | UACUGGCGCCUUUAUCUGAUUACUUUGAGAGCCAUCACCAGCGACUA |
| | loop, C18G, | UGUCGUAUGGGUAAAGCGCAGGUGGGACGACCUCUCGGUCGUCCUAU |
| | trip mut | CUGAAGCAUCAAAG |
| | (U10C) | |
| 2167 | short phage | UACUGGCGCCUUUAUCUGAUUACUUUGAGAGCCAUCACCAGCGACUA |
| | rep, C18G, | UGUCGUAUGGGUAAAGCGCGGACGACCUCUCGGUCGUCCGAAGCAUC |
| | trip mut | AAAG |
| | (U10C) | |
| 2168 | uvsX, trip mut | UACUGGCGCCUUUAUCUCAUUACUUUGAGAGCCAUCACCAGCGACUA |
| | (U10C) | UGUCGUAUGGGUAAAGCGCCCUCUUCGGAGGGAAGCAUCAAAG |
| 2169 | truncated stem | UACUGGCGCUUUUAUCUCAUUACUUUGAGAGCCAUCACCAGCGACUA |
| | loop | UGUCGUAUGGGUAAAGCGCUUACGGACUUCGGUCCGUAAGAAGCAUC |
| | | AAAG |
| 2170 | =+A17, | UACUGGCGCCUUUAUCAUCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2171 | 3′ HDV | UACUGGCGCUUUUAUCUCAUUACUUUGAGAGCCAUCACCAGCGACUA |
| | genomic | UGUCGUAUGGGUAAAGCGCUUAUUUAUCGGAGAGAAAUCCGAUAAAU |
| | ribozyme | AAGAAGCAUCAAAGGGCCGGCAUGGUCCCAGCCUCCUCGCUGGCGCC |
| | | GGCUGGGCAACAUUCCGAGGGGACCGUCCCCUCGGUAAUGGCGAAUG |
| | | GGACCC |
| 2172 | phage rep | UACUGGCGCCUUUAUCUCAUUACUUUGAGAGCCAUCACCAGCGACUA |
| | loop, trip mut | UGUCGUAUGGGUAAAGCGCAGGUGGGACGACCUCUCGGUCGUCCUAU |
| | (U10C) | CUGAAGCAUCAAAG |
| 2173 | -79:80 | UACUGGCGCUUUUAUCUCAUUACUUUGAGAGCCAUCACCAGCGACUA |
| | | UGUCGUAUGGGUAAAGCGCUUAUUUAUCGGAGAAAUCCGAUAAAUAA |
| | | GAAGCAUCAAAG |
| 2174 | short phage | UACUGGCGCCUUUAUCUCAUUACUUUGAGAGCCAUCACCAGCGACUA |
| | rep, trip mut | UGUCGUAUGGGUAAAGCGCGGACGACCUCUCGGUCGUCCGAAGCAUC |
| | (U10C) | AAAG |
| 2175 | extra | UACUGGCGCUUUUAUCUCAUUACUUUGAGAGCCAUCACCAGCGACUA |
| | truncated stem | UGUCGUAUGGGUAAAGCGCCGGACUUCGGUCCGGAAGCAUCAAAG |
| | loop | |
| 2176 | U17G, C18G | UACUGGCGCUUUUAUCGGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2177 | short phage | UACUGGCGCUUUUAUCUCAUUACUUUGAGAGCCAUCACCAGCGACUA |
| | rep | UGUCGUAUGGGUAAAGCGCGGACGACCUCUCGGUCGUCCGAAGCAUC |
| | | AAAG |
| 2178 | uvsX, C18G, | GCUGGCGCUUUUAUCUGAUUACUUUGAGAGCCAUCACCAGCGACUAU |
| | −1 A2G | GUCGUAUGGGUAAAGCGCCCUCUUCGGAGGGAAGCAUCAAAG |
| 2179 | uvsX, C18G, | GCUGGCGCCUUUAUCUGAUUACUUUGAGAGCCAUCACCAGCGACUAU |
| | trip mut | GUCGUAUGGGUAAAGCUCCCUCUUCGGAGGGAGCAUCAAAG |
| | (U10C), −1 | |
| | A2G, HDV | |
| | −99 G65U | |
| 2180 | 3′ HDV | UACUGGCGCUUUUAUCUCAUUACUUUGAGAGCCAUCACCAGCGACUA |
| | antigenomic | UGUCGUAUGGGUAAAGCGCUUAUUUAUCGGAGAGAAAUCCGAUAAAU |
| | ribozyme | AAGAAGCAUCAAAGGGGUCGGCAUGGCAUCUCCACCUCCUCGCGGUC |
| | | CGACCUGGGCAUCCGAAGGAGGACGCACGUCCACUCGGAUGGCUAAG |
| | | GGAGAGCCA |
| 2181 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGCGCAUCAAAG |
| | (U10C), −1 | |
| | A2G, HDV | |
| | AA(98: 99)C | |
| 2182 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Lior Nissim, | AAGAAGCAUCAAAGUUUUGGCCGGCAUGGUCCCAGCCUCCUCGCUGG |
| | Timothy Lu) | CGCCGGCUGGGCAACAUGCUUCGGCAUGGCGAAUGGGACCCCGGG |
| 2183 | TAC(1: 3)GA, | GAUGGCGCCUUUAUCUCAUUACUUUGAGAGCCAUCACCAGCGACUAU |
| | stacked onto | GUCGUAUGGGUAAAGCGCUUACGGACUUCGGUCCGUAAGAAGCAUCA |
| | 64 | AAG |
| 2184 | uvsX, −1 A2G | GCUGGCGCUUUUAUCUCAUUACUUUGAGAGCCAUCACCAGCGACUAU |
| | | GUCGUAUGGGUAAAGCGCCCUCUUCGGAGGGAAGCAUCAAAG |
| 2185 | truncated stem | GCUGGCGCCUUUAUCUGAUUACUUUGAGAGCCAUCACCAGCGACUAU |
| | loop, C18G, | GUCGUAUGGGUAAAGCUCUUACGGACUUCGGUCCGUAAGAGCAUCAA |
| | trip mut | AG |
| | (U10C), −1 | |
| | A2G, HDV | |
| | −99 G65U | |
| 2186 | short phage | GCUGGCGCCUUUAUCUGAUUACUUUGAGAGCCAUCACCAGCGACUAU |
| | rep, C18G, | GUCGUAUGGGUAAAGCUCGGACGACCUCUCGGUCGUCCGAGCAUCAA |
| | trip mut | AG |
| | (U10C), −1 | |
| | A2G, HDV | |
| | −99 G65U | |
| 2187 | 3′ sTRSV WT | UACUGGCGCUUUUAUCUCAUUACUUUGAGAGCCAUCACCAGCGACUA |
| | viral | UGUCGUAUGGGUAAAGCGCUUAUUUAUCGGAGAGAAAUCCGAUAAAU |
| | Hammerhead | AAGAAGCAUCAAAGCCUGUCACCGGAUGUGCUUUCCGGUCUGAUGAG |
| | ribozyme | UCCGUGAGGACGAAACAGG |
| 2188 | short phage | GCUGGCGCUUUUAUCUGAUUACUUUGAGAGCCAUCACCAGCGACUAU |
| | rep, C18G, −1 | GUCGUAUGGGUAAAGCGCGGACGACCUCUCGGUCGUCCGAAGCAUCA |
| | A2G | AAG |
| 2189 | short phage | GCUGGCGCCUUUAUCUGAUUACUUUGAGAGCCAUCACCAGCGACUAU |
| | rep, C18G, | GUCGUAUGGGUAAAGCGCGGACGACCUCUCGGUCGUCCGAAGCAUCA |
| | trip mut | AAG |
| | (U10C), −1 | |
| | A2G, 3′ | |
| | genomic HDV | |
| 2190 | phage rep | GCUGGCGCCUUUAUCUGAUUACUUUGAGAGCCAUCACCAGCGACUAU |
| | loop, C18G, | GUCGUAUGGGUAAAGCUCAGGUGGGACGACCUCUCGGUCGUCCUAUC |
| | trip mut | UGAGCAUCAAAG |
| | (U10C), −1 | |
| | A2G, HDV | |
| | −99 G65U | |
| 2191 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Owen Ryan, | AAGAAGCAUCAAAGGAUGGCCGGCAUGGUCCCAGCCUCCUCGCUGGC |
| | Jamie Cate) | GCCGGCUGGGCAACACCUUCGGGUGGCGAAUGGGAC |
| 2192 | phage rep | GCUGGCGCUUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | −1 A2G | UGAAGCAUCAAAG |
| 2193 | 0.14 | UACUGGCGCUUUUAUCUCAUUACUUUGAGAGCCAUCACCAGCGACUA |
| | | UGUCGUACUGGGUAAAGCGCUUAUUUAUCGGAGAGAAAUCCGAUAAA |
| | | UAAGAAGCAUCAAAG |
| 2194 | −78, G77U | UACUGGCGCUUUUAUCUCAUUACUUUGAGAGCCAUCACCAGCGACUA |
| | | UGUCGUAUGGGUAAAGCGCUUAUUUAUCGUGAGAAAUCCGAUAAAUA |
| | | AGAAGCAUCAAAG |
| 2195 | | GUACUGGCGCUUUUAUCUCAUUACUUUGAGAGCCAUCACCAGCGACU |
| | | AUGUCGUAUGGGUAAAGCGCUUAUUUAUCGGAGAGAAAUCCGAUAAA |
| | | UAAGAAGCAUCAAAG |
| 2196 | short phage | GCUGGCGCUUUUAUCUCAUUACUUUGAGAGCCAUCACCAGCGACUAU |
| | rep, −1 A2G | GUCGUAUGGGUAAAGCGCGGACGACCUCUCGGUCGUCCGAAGCAUCA |
| | | AAG |
| 2197 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGAAGCAUCA |
| | trip mut | AAG |
| | (U10C), −1 | |
| | A2G | |
| 2198 | −1, A2G | GCUGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2199 | truncated stem | GCUGGCGCCUUUAUCUCAUUACUUUGAGAGCCAUCACCAGCGACUAU |
| | loop, trip mut | GUCGUAUGGGUAAAGCGCUUACGGACUUCGGUCCGUAAGAAGCAUCA |
| | (U10C), −1 | AAG |
| | A2G | |
| 2200 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGAAGCAUCAAAG |
| | (U10C), −1 | |
| | A2G | |
| 2201 | phage rep | GCUGGCGCUUUUAUCUCAUUACUUUGAGAGCCAUCACCAGCGACUAU |
| | loop, −1 A2G | GUCGUAUGGGUAAAGCGCAGGUGGGACGACCUCUCGGUCGUCCUAUC |
| | | UGAAGCAUCAAAG |
| 2202 | phage rep | GCUGGCGCCUUUAUCUCAUUACUUUGAGAGCCAUCACCAGCGACUAU |
| | loop, trip mut | GUCGUAUGGGUAAAGCGCAGGUGGGACGACCUCUCGGUCGUCCUAUC |
| | (U10C), −1 | UGAAGCAUCAAAG |
| | A2G | |
| 2203 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGAAGCAUCAAAG |
| | (U10C), −1 | |
| | A2G | |
| 2204 | truncated stem | UACUGGCGCUUUUAUCUGAUUACUUUGAGAGCCAUCACCAGCGACUA |
| | loop, C18G | UGUCGUAUGGGUAAAGCGCUUACGGACUUCGGUCCGUAAGAAGCAUC |
| | | AAAG |
| 2205 | uvsX, trip mut | GCUGGCGCCUUUAUCUCAUUACUUUGAGAGCCAUCACCAGCGACUAU |
| | (U10C), −1 | GUCGUAUGGGUAAAGCGCCCUCUUCGGAGGGAAGCAUCAAAG |
| | A2G | |
| 2206 | truncated stem | GCUGGCGCUUUUAUCUCAUUACUUUGAGAGCCAUCACCAGCGACUAU |
| | loop, −1 A2G | GUCGUAUGGGUAAAGCGCUUACGGACUUCGGUCCGUAAGAAGCAUCA |
| | | AAG |
| 2207 | short phage | GCUGGCGCCUUUAUCUCAUUACUUUGAGAGCCAUCACCAGCGACUAU |
| | rep, trip mut | GUCGUAUGGGUAAAGCGCGGACGACCUCUCGGUCGUCCGAAGCAUCA |
| | (U10C), −1 | AAG |
| | A2G | |
| 2208 | 5′HDV | GAUGGCCGGCAUGGUCCCAGCCUCCUCGCUGGCGCCGGCUGGGCAAC |
| | ribozyme | ACCUUCGGGUGGCGAAUGGGACUACUGGCGCUUUUAUCUCAUUACUU |
| | (Owen Ryan, | UGAGAGCCAUCACCAGCGACUAUGUCGUAUGGGUAAAGCGCUUAUUU |
| | Jamie Cate) | AUCGGAGAGAAAUCCGAUAAAUAAGAAGCAUCAAAG |
| 2209 | 5′HDV | GGCCGGCAUGGUCCCAGCCUCCUCGCUGGCGCCGGCUGGGCAACAUU |
| | genomic | CCGAGGGGACCGUCCCCUCGGUAAUGGCGAAUGGGACCCUACUGGCG |
| | ribozyme | CUUUUAUCUCAUUACUUUGAGAGCCAUCACCAGCGACUAUGUCGUAU |
| | | GGGUAAAGCGCUUAUUUAUCGGAGAGAAAUCCGAUAAAUAAGAAGCA |
| | | UCAAAG |
| 2210 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGCGCAUCAA |
| | trip mut | AG |
| | (U10C), −1 | |
| | A2G, HDV | |
| | AA(98: 99)C | |
| 2211 | 5′env25 pistol | CGUGGUUAGGGCCACGUUAAAUAGUUGCUUAAGCCCUAAGCGUUGAU |
| | ribozyme | CUUCGGAUCAGGUGCAAUACUGGCGCUUUUAUCUCAUUACUUUGAGA |
| | (with an added | GCCAUCACCAGCGACUAUGUCGUAUGGGUAAAGCGCUUAUUUAUCGG |
| | CUUCGG | AGAGAAAUCCGAUAAAUAAGAAGCAUCAAAG |
| | loop) | |
| 2212 | 5′HDV | GGGUCGGCAUGGCAUCUCCACCUCCUCGCGGUCCGACCUGGGCAUCC |
| | antigenomic | GAAGGAGGACGCACGUCCACUCGGAUGGCUAAGGGAGAGCCAUACUG |
| | ribozyme | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2213 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCCAGUACUGAUGAGUCCGUGAGGACGAAACGAG |
| | (Lior Nissim, | UAAGCUCGUCUACUGGCGCUUUUAUCUCAU |
| | Timothy Lu) | |
| | guide scaffold | |
| | scar | |
| 2214 | =+A27, | UACUGGCGCCUUUAUCUCAUUACUUUAGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2215 | 5′Hammerhead | CGACUACUGAUGAGUCCGUGAGGACGAAACGAGUAAGCUCGUCUAGU |
| | ribozyme | CGUACUGGCGCUUUUAUCUCAUUACUUUGAGAGCCAUCACCAGCGAC |
| | (Lior Nissim, | UAUGUCGUAUGGGUAAAGCGCUUAUUUAUCGGAGAGAAAUCCGAUAA |
| | Timothy Lu) | AUAAGAAGCAUCAAAG |
| | smaller scar | |
| 2216 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGCGCAUCAAAG |
| | (U10C), −1 | |
| | A2G, HDV | |
| | AA(98: 99)C | |
| 2217 | −27, stacked | UACUGGCGCCUUUAUCUCAUUACUUUAGAGCCAUCACCAGCGACUAU |
| | onto 64 | GUCGUAUGGGUAAAGCGCUUACGGACUUCGGUCCGUAAGAAGCAUCA |
| | | AAG |
| 2218 | 3′ Hatchet | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAGCAUUCCUCAGAAAAUGACAAACCUGUGGGGCGU |
| | | AAGUAGAUCUUCGGAUCUAUGAUCGUGCAGACGUUAAAAUCAGGU |
| 2219 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCGCGUGUAGCGAAGCA |
| | Timothy Lu) | |
| 2220 | 5′ Hatchet | CAUUCCUCAGAAAAUGACAAACCUGUGGGGCGUAAGUAGAUCUUCGG |
| | | AUCUAUGAUCGUGCAGACGUUAAAAUCAGGUUACUGGCGCUUUUAUC |
| | | UCAUUACUUUGAGAGCCAUCACCAGCGACUAUGUCGUAUGGGUAAAG |
| | | CGCUUAUUUAUCGGAGAGAAAUCCGAUAAAUAAGAAGCAUCAAAG |
| 2221 | 5′ HDV | UUUUGGCCGGCAUGGUCCCAGCCUCCUCGCUGGCGCCGGCUGGGCAA |
| | ribozyme | CAUGCUUCGGCAUGGCGAAUGGGACCCCGGGUACUGGCGCUUUUAUC |
| | (Lior Nissim, | UCAUUACUUUGAGAGCCAUCACCAGCGACUAUGUCGUAUGGGUAAAG |
| | Timothy Lu) | CGCUUAUUUAUCGGAGAGAAAUCCGAUAAAUAAGAAGCAUCAAAG |
| 2222 | 5′ | CGACUACUGAUGAGUCCGUGAGGACGAAACGAGUAAGCUCGUCUAGU |
| | Hammerhead | CGCGUGUAGCGAAGCAUACUGGCGCUUUUAUCUCAUUACUUUGAGAG |
| | ribozyme | CCAUCACCAGCGACUAUGUCGUAUGGGUAAAGCGCUUAUUUAUCGGA |
| | (Lior Nissim, | GAGAAAUCCGAUAAAUAAGAAGCAUCAAAG |
| | Timothy Lu) | |
| 2223 | 3′ HH15 | UACUGGCGCUUUUAUCUCAUUACUUUGAGAGCCAUCACCAGCGACUA |
| | Minimal | UGUCGUAUGGGUAAAGCGCUUAUUUAUCGGAGAGAAAUCCGAUAAAU |
| | Hammerhead | AAGAAGCAUCAAAGGGGAGCCCCGCUGAUGAGGUCGGGGAGACCGAA |
| | ribozyme | AGGGACUUCGGUCCCUACGGGGCUCCC |
| 2224 | 5′ RBMX | CCACCCCCACCACCACCCCCACCCCCACCACCACCCUACUGGCGCUU |
| | recruiting | UUAUCUCAUUACUUUGAGAGCCAUCACCAGCGACUAUGUCGUAUGGG |
| | motif | UAAAGCGCUUAUUUAUCGGAGAGAAAUCCGAUAAAUAAGAAGCAUCA |
| | | AAG |
| 2225 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCG |
| | Timothy Lu) | |
| | smaller scar | |
| 2226 | 3′ env25 pistol | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (with an added | AAGAAGCAUCAAAGCGUGGUUAGGGCCACGUUAAAUAGUUGCUUAAG |
| | CUUCGG | CCCUAAGCGUUGAUCUUCGGAUCAGGUGCAA |
| | loop) | |
| 2227 | 3′ Env-9 | UACUGGCGCUUUUAUCUCAUUACUUUGAGAGCCAUCACCAGCGACUA |
| | Twister | UGUCGUAUGGGUAAAGCGCUUAUUUAUCGGAGAGAAAUCCGAUAAAU |
| | | AAGAAGCAUCAAAGGGCAAUAAAGCGGUUACAAGCCCGCAAAAAUAG |
| | | CAGAGUAAUGUCGCGAUAGCGCGGCAUUAAUGCAGCUUUAUUG |
| 2228 | =+AUUAUC | UACUGGCGCUUUUAUCUCAUUACUAUUAUCUCAUUACUUUGAGAGCC |
| | UCAUUACU | AUCACCAGCGACUAUGUCGUAUGGGUAAAGCGCUUAUUUAUCGGAGA |
| | 25 | GAAAUCCGAUAAAUAAGAAGCAUCAAAG |
| 2229 | 5′ Env-9 | GGCAAUAAAGCGGUUACAAGCCCGCAAAAAUAGCAGAGUAAUGUCGC |
| | Twister | GAUAGCGCGGCAUUAAUGCAGCUUUAUUGUACUGGCGCUUUUAUCUC |
| | | AUUACUUUGAGAGCCAUCACCAGCGACUAUGUCGUAUGGGUAAAGCG |
| | | CUUAUUUAUCGGAGAGAAAUCCGAUAAAUAAGAAGCAUCAAAG |
| 2230 | 3′ Twisted | UACUGGCGCUUUUAUCUCAUUACUUUGAGAGCCAUCACCAGCGACUA |
| | Sister 1 | UGUCGUAUGGGUAAAGCGCUUAUUUAUCGGAGAGAAAUCCGAUAAAU |
| | | AAGAAGCAUCAAAGACCCGCAAGGCCGACGGCAUCCGCCGCCGCUGG |
| | | UGCAAGUCCAGCCGCCCCUUCGGGGGCGGGCGCUCAUGGGUAAC |
| 2231 | no stem | UACUGGCGCUUUUAUCUCAUUACUUUGAGAGCCAUCACCAGCGACUA |
| | | UGUCGUAUGGGUAAAG |
| 2232 | 5′ HH15 | GGGAGCCCCGCUGAUGAGGUCGGGGAGACCGAAAGGGACUUCGGUCC |
| | Minimal | CUACGGGGCUCCCUACUGGCGCUUUUAUCUCAUUACUUUGAGAGCCA |
| | Hammerhead | UCACCAGCGACUAUGUCGUAUGGGUAAAGCGCUUAUUUAUCGGAGAG |
| | ribozyme | AAAUCCGAUAAAUAAGAAGCAUCAAAG |
| 2233 | 5′ | CCAGUACUGAUGAGUCCGUGAGGACGAAACGAGUAAGCUCGUCUACU |
| | Hammerhead | GGCGCUUUUAUCUCAUUACUGGCGCUUUUAUCUCAUUACUUUGAGAG |
| | ribozyme | CCAUCACCAGCGACUAUGUCGUAUGGGUAAAGCGCUUAUUUAUCGGA |
| | (Lior Nissim, | GAGAAAUCCGAUAAAUAAGAAGCAUCAAAG |
| | Timothy Lu) | |
| | guide scaffold | |
| | scar | |
| 2234 | 5′ Twisted | ACCCGCAAGGCCGACGGCAUCCGCCGCCGCUGGUGCAAGUCCAGCCG |
| | Sister 1 | CCCCUUCGGGGGCGGGCGCUCAUGGGUAACUACUGGCGCUUUUAUCU |
| | | CAUUACUUUGAGAGCCAUCACCAGCGACUAUGUCGUAUGGGUAAAGC |
| | | GCUUAUUUAUCGGAGAGAAAUCCGAUAAAUAAGAAGCAUCAAAG |
| 2235 | 5′ sTRSV WT | CCUGUCACCGGAUGUGCUUUCCGGUCUGAUGAGUCCGUGAGGACGAA |
| | viral | ACAGGUACUGGCGCUUUUAUCUCAUUACUUUGAGAGCCAUCACCAGC |
| | Hammerhead | GACUAUGUCGUAUGGGUAAAGCGCUUAUUUAUCGGAGAGAAAUCCGA |
| | ribozyme | UAAAUAAGAAGCAUCAAAG |
| 2236 | 148: =+G55, | GUACUGGCGCCUUUAUCUCAUUACUUUGAGAGCCAUCACCAGCGACU |
| | stacked onto | AUGUCGUAGUGGGUAAAGCGCUUACGGACUUCGGUCCGUAAGAAGCA |
| | 64 | UCAAAG |
| 2237 | 158: | GUACUGGCGCUUUUAUCUGAUUACUUUGAGAGCCAUCACCAGCGACU |
| | 103 + 148(+G55) | AUGUCGUAGUGGGUAAAGCUCCCUCUUCGGAGGGAGCAUCAAAG |
| | −99, G65U | |
| 2238 | 174: Uvsx | ACUGGCGCUUUUAUCUGAUUACUUUGAGAGCCAUCACCAGCGACUAU |
| | Extended stem | GUCGUAGUGGGUAAAGCUCCCUCUUCGGAGGGAGCAUCAAAG |
| | with [A99] | |
| | G65U), | |
| | C18G, \hat{ }G55, | |
| | [GU−1] | |
| 2239 | 175: extended | ACUGGCGCCUUUAUCUCAUUACUUUGAGAGCCAUCACCAGCGACUAU |
| | stem | GUCGUAUGGGUAAAGCGCUUACGGACUUCGGUCCGUAAGAAGCAUCA |
| | truncation, | AAG |
| | U10C, [GU−1] | |
| 2240 | 176: 174 with | GCUGGCGCUUUUAUCUGAUUACUUUGAGAGCCAUCACCAGCGACUAU |
| | A1G | GUCGUAGUGGGUAAAGCUCCCUCUUCGGAGGGAGCAUCAAAG |
| | substitution | |
| | for T7 | |
| | transcription | |
| 2241 | 177: 174 with | ACUGGCGCUUUUAUCUGAUUACUUUGAGAGCCAUCACCAGCGACUAU |
| | bubble (+G55) | GUCGUAUGGGUAAAGCUCCCUCUUCGGAGGGAGCAUCAAAG |
| | removed | |
| 2242 | 181: stem 42 | ACUGGCGCCUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | U10C,C18G, | |
| | [GU−1] | |
| | (95 + [GU−1]) | |
| 2243 | 182: stem 42 | ACUGGCGCUUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | C18G, [GU−1] | |
| 2244 | 183: stem 42 | ACUGGCGCUUUUAUCUGAUUACUUUGAGAGCCAUCACCAGCGACUAU |
| | (truncated | GUCGUAGUGGGUAAAGCGCUUACGGACUUCGGUCCGUAAGAAGCAUC |
| | stem loop); | AAAG |
| | C18G, \hat{ }G55, | |
| | [GU−1] | |
| 2245 | 184: stem 48 | ACUGGCGCUUUUAUCUGAUUACUUUGAGAGCCAUCACCAGCGACUAU |
| | (uvsx, −99 | GUCGUAUUGGGUAAAGCUCCCUCUUCGGAGGGAGCAUCAAAG |
| | g65t); | |
| | C18G, \hat{ }T55, | |
| | [GU−1] | |
| 2246 | 185: stem 42 | ACUGGCGCUUUUAUCUGAUUACUUUGAGAGCCAUCACCAGCGACUAU |
| | (truncated | GUCGUAUUGGGUAAAGCGCUUACGGACUUCGGUCCGUAAGAAGCAUC |
| | stem loop); | AAAG |
| | C18G, \hat{ }U55, | |
| | [GU−1] | |
| 2247 | 186: stem 42 | ACUGGCGCCUUUAUCAUCAUUACUUUGAGAGCCAUCACCAGCGACUA |
| | (truncated | UGUCGUAUGGGUAAAGCGCUUACGGACUUCGGUCCGUAAGAAGCAUC |
| | stem loop); | AAAG |
| | U10C, \hat{ }A17, | |
| | [GU−1] | |
| 2248 | 187: stem 46 | ACUGGCGCUUUUAUCUGAUUACUUUGAGAGCCAUCACCAGCGACUAU |
| | (uvsx); | GUCGUAGUGGGUAAAGCGCCCUCUUCGGAGGGAAGCAUCAAAG |
| | C18G, \hat{ }G55, | |
| | [GU−1] | |
| 2249 | 188: stem 50 | ACUGGCGCUUUUAUCUGAUUACUUUGAGAGCCAUCACCAGCGACUAU |
| | (ms2 U15C, | GUCGUAGUGGGUAAAGCUCACAUGAGGAUCACCCAUGUGAGCAUCAA |
| | −99, g65t); | AG |
| | C18G, \hat{ }G55, | |
| | [GU−1] | |
| 2250 | 189: 174 + | ACUGGCACUUUUACCUGAUUACUUUGAGAGCCAACACCAGCGACUAU |
| | G8A; U15C; | GUCGUAGUGGGUAAAGCUCCCUCUUCGGAGGGAGCAUCAAAG |
| | U35A | |
| 2251 | 190: 174 + | ACUGGCACUUUUAUCUGAUUACUUUGAGAGCCAUCACCAGCGACUAU |
| | G8A | GUCGUAGUGGGUAAAGCUCCCUCUUCGGAGGGAGCAUCAAAG |
| 2252 | 191: 174 + | ACUGGCCCUUUUAUCUGAUUACUUUGAGAGCCAUCACCAGCGACUAU |
| | G8C | GUCGUAGUGGGUAAAGCUCCCUCUUCGGAGGGAGCAUCAAAG |
| 2253 | 192: 174 + | ACUGGCGCUUUUACCUGAUUACUUUGAGAGCCAUCACCAGCGACUAU |
| | U15C | GUCGUAGUGGGUAAAGCUCCCUCUUCGGAGGGAGCAUCAAAG |
| 2254 | 193, 174 + | ACUGGCGCUUUUAUCUGAUUACUUUGAGAGCCAACACCAGCGACUAU |
| | U35A | GUCGUAGUGGGUAAAGCUCCCUCUUCGGAGGGAGCAUCAAAG |
| 2255 | 195: 175 + | ACUGGCACCUUUACCUGAUUACUUUGAGAGCCAACACCAGCGACUAU |
| | C18G + | GUCGUAUGGGUAAAGCGCUUACGGACUUCGGUCCGUAAGAAGCAUCA |
| | G8A; U15C; | AAG |
| | U35A | |
| 2256 | 196: 175 + | ACUGGCACCUUUAUCUGAUUACUUUGAGAGCCAUCACCAGCGACUAU |
| | C18G + G8A | GUCGUAUGGGUAAAGCGCUUACGGACUUCGGUCCGUAAGAAGCAUCA |
| | | AAG |
| 2257 | 197: 175 + | ACUGGCCCCUUUAUCUGAUUACUUUGAGAGCCAUCACCAGCGACUAU |
| | C18G + G8C | GUCGUAUGGGUAAAGCGCUUACGGACUUCGGUCCGUAAGAAGCAUCA |
| | | AAG |
| 2258 | 198: 175 + | ACUGGCGCCUUUAUCUGAUUACUUUGAGAGCCAACACCAGCGACUAU |
| | C18G + U35A | GUCGUAUGGGUAAAGCGCUUACGGACUUCGGUCCGUAAGAAGCAUCA |
| | | AAG |
| 2259 | 199: 174 + | GCUGGCGCUUUUAUCUGAUUACUUUGAGAGCCAUCACCAGCGACUAU |
| | A2G (test G | GUCGUAGUGGGUAAAGCUCCCUCUUCGGAGGGAGCAUCAAAG |
| | transcription | |
| | at start; | |
| | ccGCT . . .) | |
| 2260 | 200: 174 + | GACUGGCGCUUUUAUCUGAUUACUUUGAGAGCCAUCACCAGCGACUA |
| | \hat{ }G1 | UGUCGUAGUGGGUAAAGCUCCCUCUUCGGAGGGAGCAUCAAAG |
| | (ccGACU . . .) | |
| 2261 | 201: 174 + | ACUGGCGCCUUUAUCUGAUUACUUUGGAGAGCCAUCACCAGCGACUA |
| | U10C; \hat{ }G28 | UGUCGUAGUGGGUAAAGCUCCCUCUUCGGAGGGAGCAUCAAAG |
| 2262 | 202: 174 + | ACUGGCGCAUUUAUCUGAUUACUUUGUGAGCCAUCACCAGCGACUAU |
| | U10A; A28U | GUCGUAGUGGGUAAAGCUCCCUCUUCGGAGGGAGCAUCAAAG |
| 2263 | 203: 174 + | ACUGGCGCCUUUAUCUGAUUACUUUGAGAGCCAUCACCAGCGACUAU |
| | U10C | GUCGUAGUGGGUAAAGCUCCCUCUUCGGAGGGAGCAUCAAAG |
| 2264 | 204: 174 + | ACUGGCGCUUUUAUCUGAUUACUUUGGAGAGCCAUCACCAGCGACUA |
| | \hat{ }G28 | UGUCGUAGUGGGUAAAGCUCCCUCUUCGGAGGGAGCAUCAAAG |
| 2265 | 205: 174 + | ACUGGCGCAUUUAUCUGAUUACUUUGAGAGCCAUCACCAGCGACUAU |
| | U10A | GUCGUAGUGGGUAAAGCUCCCUCUUCGGAGGGAGCAUCAAAG |
| 2266 | 206, 174 + | ACUGGCGCUUUUAUCUGAUUACUUUGUGAGCCAUCACCAGCGACUAU |
| | A28U | GUCGUAGUGGGUAAAGCUCCCUCUUCGGAGGGAGCAUCAAAG |
| 2267 | 207: 174 + | ACUGGCGCUUUUAUUCUGAUUACUUUGAGAGCCAUCACCAGCGACUA |
| | \hat{ }U15 | UGUCGUAGUGGGUAAAGCUCCCUCUUCGGAGGGAGCAUCAAAG |
| 2268 | 208: 174 + | ACGGCGCUUUUAUCUGAUUACUUUGAGAGCCAUCACCAGCGACUAUG |
| | [U4] | UCGUAGUGGGUAAAGCUCCCUCUUCGGAGGGAGCAUCAAAG |
| 2269 | 209: 174 + | ACUGGCGCUUUUAUAUGAUUACUUUGAGAGCCAUCACCAGCGACUAU |
| | C16A | GUCGUAGUGGGUAAAGCUCCCUCUUCGGAGGGAGCAUCAAAG |
| 2270 | 210: 174 + | ACUGGCGCUUUUAUCUUGAUUACUUUGAGAGCCAUCACCAGCGACUA |
| | \hat{ }U17 | UGUCGUAGUGGGUAAAGCUCCCUCUUCGGAGGGAGCAUCAAAG |
| 2271 | 211: 174 + | ACUGGCGCUUUUAUCUGAUUACUUUGAGAGCCAGCACCAGCGACUAU |
| | U35G | GUCGUAGUGGGUAAAGCUCCCUCUUCGGAGGGAGCAUCAAAG |
| | (compare with | |
| | 174 + U35A | |
| | above) | |
| 2272 | 212: 174 + | ACUGGCGCUGUUAUCUGAUUACUUCGAGAGCCAUCACCAGCGACUAU |
| | U11G, | GUCGUAGUGGGUAAAGCUCCCUCUUCGGAGGGAGCAUCGAAG |
| | A105G | |
| | (A86G), | |
| | U26C | |
| 2273 | 213: 174 + | ACUGGCGCUCUUAUCUGAUUACUUCGAGAGCCAUCACCAGCGACUAU |
| | U11C, | GUCGUAGUGGGUAAAGCUCCCUCUUCGGAGGGAGCAUCGAAG |
| | A105G | |
| | (A86G), | |
| | U26C | |
| 2274 | 214: | ACUGGCGCUUGUAUCUGAUUACUCUGAGAGCCAUCACCAGCGACUAU |
| | 174 + U12G; | GUCGUAGUGGGUAAAGCUCCCUCUUCGGAGGGAGCAUCAGAG |
| | A106G | |
| | (A87G), | |
| | U25C | |
| 2275 | 215: | ACUGGCGCUUCUAUCUGAUUACUCUGAGAGCCAUCACCAGCGACUAU |
| | 174 + U12C; | GUCGUAGUGGGUAAAGCUCCCUCUUCGGAGGGAGCAUCAGAG |
| | A106G | |
| | (A87G), | |
| | U25C | |
| 2276 | 216: | ACUGGCGCUUUGAUCUGAUUACCUUGAGAGCCAUCACCAGCGACUAU |
| | 174_tx_11.G, | GUCGUAGUGGGUAAAGCUCCCUCUUCGGAGGGAGCAUCAAGG |
| | 87.G, 22.C | |
| 2277 | 217: | ACUGGCGCUUUCAUCUGAUUACCUUGAGAGCCAUCACCAGCGACUAU |
| | 174_tx_11.C, | GUCGUAGUGGGUAAAGCUCCCUCUUCGGAGGGAGCAUCAAGG |
| | 87.G, 22.C | |
| 2278 | 218: 174 + | ACUGGCGCUGUUAUCUGAUUACUUUGAGAGCCAUCACCAGCGACUAU |
| | U11G | GUCGUAGUGGGUAAAGCUCCCUCUUCGGAGGGAGCAUCAAAG |
| 2279 | 219: 174 + | ACUGGCGCUUUUAUCUGAUUACUUUGAGAGCCAUCACCAGCGACUAU |
| | A105G | GUCGUAGUGGGUAAAGCUCCCUCUUCGGAGGGAGCAUCGAAG |
| | (A86G) | |
| 2280 | 220: 174 + | ACUGGCGCUUUUAUCUGAUUACUUCGAGAGCCAUCACCAGCGACUAU |
| | U26C | GUCGUAGUGGGUAAAGCUCCCUCUUCGGAGGGAGCAUCAAAG |
| 2281 | 221: 182: + | ACUGGCACUUCUAUCUGAUUACUCUGAGAGCCAUCACCAGCGACUAU |
| | G7A, T11C, | GUCGUAUGGGUAAAGCCGCUUACGGACUUCGGUCCGUAAGAGGCAUC |
| | T24C, A95G + | AGAG |
| | \hat{ }C63, A88G | |
| 2282 | 222: 174: + | ACUGGCACUUCUAUCUGAUUACUCUGAGAGCCAUCACCAGCGACUAU |
| | G7A, T11C, | GUCGUAGUGGGUAAAGCUCCCUCUUCGGAGGGAGCAUCAGAG |
| | T24C, A87G | |
| 2283 | 223: 175: + | ACUGGCACCUUUAUCUGAUUACUUUGAGAGCCAUCACCAGCGACUAU |
| | G7A, C17G + | GUCGUAUGGGUAAAGCCGCUUACGGACUUCGGUCCGUAAGAGGCAUC |
| | \hat{ }C63, A88G | AAAG |
| 2284 | 224: 182: + | ACUGGCACUUGUAUCUGAUUACUCUGAGAGCCAUCACCAGCGACUAU |
| | G7A, T11G, | GUCGUAUGGGUAAAGCCGCUUACGGACUUCGGUCCGUAAGAGGCAUC |
| | T24C, A95G + | AGAG |
| | \hat{ }C63, A88G | |
| 2285 | 225: 174: + | ACUGGCACUUGUAUCUGAUUACUCUGAGAGCCAUCACCAGCGACUAU |
| | G7A, T11G, | GUCGUAGUGGGUAAAGCUCCCUCUUCGGAGGGAGCAUCAGAG |
| | T24C, A87G | |
| 2286 | 229: 196 + | ACUGGCACUUUUAUCUGAUUACUUUGAGAGCCAUCACCAGCGACUAU |
| | C9T | GUCGUAUGGGUAAAGCGCUUACGGACUUCGGUCCGUAAGAAGCAUCA |
| | | AAG |
| 2287 | 230: 221 + | ACUGGCACUUCUAUCUGAUUACUCUGAGAGCCAUCACCAGCGACUAU |
| | [C64], G89A | GUCGUAUGGGUAAAGCGCUUACGGACUUCGGUCCGUAAGAAGCAUCA |
| | | GAG |
| 2288 | 231: 221 + | ACUGGCGCUUCUAUCUGAUUACUCUGAGAGCCAUCACCAGCGACUAU |
| | A7G | GUCGUAUGGGUAAAGCCGCUUACGGACUUCGGUCCGUAAGAGGCAUC |
| | | AGAG |
| 2289 | 232: 221 + | ACUGGCACUUCUAUCUGAUUACUCUGAGCGCCAUCACCAGCGACUAU |
| | A29C | GUCGUAUGGGUAAAGCCGCUUACGGACUUCGGUCCGUAAGAGGCAUC |
| | | AGAG |
| 2290 | 233: 221 + | ACUGGCGCUUCUAUCUGAUUACUCUGAGCGCCAUCACCAGCGACUAU |
| | A7G, A29C | GUCGUAUGGGUAAAGCCGCUUACGGACUUCGGUCCGUAAGAGGCAUC |
| | | AGAG |
| 2291 | 234: 221 + | ACUGGCGCUUCUAUCUGAUUACUCUGAGCGCCAUCACCAGCGACUAU |
| | A7G,A29C + | GUCGUAUGGGUAAAGCGCCUUACGGACUUCGGUCCGUAAGGAGCAUC |
| | CG64GC, | AGAG |
| | AG88GA | |
| 2292 | 235: 221 + | ACUGGCGCUUCUAUCUGAUUACUCUGAGCGCCAUCACCAGCGACUAU |
| | A7G, A29C, | GUCGUAGUGGGUAAAGCCGCUUACGGACUUCGGUCCGUAAGAGGCAU |
| | \hat{ }G54 | CAGAG |
| 2293 | 236: 221 + | ACGGGACUUUCUAUCUGAUUACUCUGAAGUCCCUCACCAGCGACUAU |
| | pseudoknot | GUCGUAUGGGUAAAGCCGCUUACGGACUUCGGUCCGUAAGAGGCAUC |
| | GGGACT_A | AGAG |
| | AGTCCC | |
| 2294 | 237: 221 + | ACCUGUAGUUCUAUCUGAUUACUCUGACUACAGUCACCAGCGACUAU |
| | pseudoknot | GUCGUAUGGGUAAAGCCGCUUACGGACUUCGGUCCGUAAGAGGCAUC |
| | CTGTAG_ | AGAG |
| | CTACAG | |

In some embodiments, the gNA variant comprises a tracrRNA stem loop comprising the sequence -UUU-N4-25-UUU- (SEQ ID NO: 34). For example, the gNA variant comprises a scaffold stem loop or a replacement thereof, flanked by two triplet U motifs that contribute to the triplex region. In some embodiments, the scaffold stem loop or replacement thereof comprises at least 4 nucleotides, at least 5 nucleotides, at least 6 nucleotides, at least 7 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides.

In some embodiments, the gNA variant comprises a crRNA sequence with -AAAG- in a location 5′ to the spacer region. In some embodiments, the -AAAG- sequence is immediately 5′ to the spacer region.

In some embodiments, the at least one nucleotide modification to a reference gNA to produce a gNA variant comprises at least one nucleotide deletion in the CasX variant gNA relative to the reference gRNA. In some embodiments, a gNA variant comprises a deletion of 1, 2, 3, 4, 5, 6, 7, 8, 9, 10, 11, 12, 13, 14, 15, 16, 17, 18, 19 or 20 consecutive or non-consecutive nucleotides relative to a reference gNA. In some embodiments, the at least one deletion comprises a deletion of 1, 2, 3, 4, 5, 6, 7, 8, 9, 10, 11, 12, 13, 14, 15, 16, 17, 18, 19 or 20 or more consecutive nucleotides relative to a reference gNA. In some embodiments, the gNA variant comprises 2, 3, 4, 5, 6, 7, 8, 9, 10, 11, 12, 13, 14, 15, 16, 17, 18, 19 or 20 or more nucleotide deletions relative to the reference gNA, and the deletions are not in consecutive nucleotides. In those embodiments where there are two or more non-consecutive deletions in the gNA variant relative to the reference gRNA, any length of deletions, and any combination of lengths of deletions, as described herein, are contemplated as within the scope of the disclosure. In some embodiments, a gNA variant comprises at least two deletions in different regions of the reference gRNA. In some embodiments, a gNA variant comprises at least two deletions in the same region of the reference gRNA. For example, the regions may be the extended stem loop, scaffold stem loop, scaffold stem bubble, triplex loop, pseudoknot, triplex, or a 5′ end of the gNA variant. The deletion of any nucleotide in a reference gRNA is contemplated as within the scope of the disclosure.

In some embodiments, the at least one nucleotide modification of a reference gRNA to generate a gNA variant comprises at least one nucleotide insertion. In some embodiments, a gNA variant comprises an insertion of 1, 2, 3, 4, 5, 6, 7, 8, 9 or 10 consecutive or non-consecutive nucleotides relative to a reference gRNA. In some embodiments, the at least one nucleotide insertion comprises an insertion of 1, 2, 3, 4, 5, 6, 7, 8, 9, 10, 11, 12, 13, 14, 15, 16, 17, 18, 19 or 20 or more consecutive nucleotides relative to a reference gRNA. In some embodiments, the gNA variant comprises 2 or more insertions relative to the reference gRNA, and the insertions are not consecutive. In those embodiments where there are two or more non-consecutive insertions in the gNA variant relative to the reference gRNA, any length of insertions, and any combination of lengths of insertions, as described herein, are contemplated as within the scope of the disclosure. For example, in some embodiments, a gNA variant may comprise a first insertion of one nucleotide, and a second insertion of two nucleotides and the two insertions are not consecutive. In some embodiments, a gNA variant comprises at least two insertions in different regions of the reference gRNA. In some embodiments, a gNA variant comprises at least two insertions in the same region of the reference gRNA. For example, the regions may be the extended stem loop, scaffold stem loop, scaffold stem bubble, triplex loop, pseudoknot, triplex, or a 5′ end of the gNA variant. Any insertion of A, G, C, U (or T, in the corresponding DNA) or combinations thereof at any location in the reference gRNA is contemplated as within the scope of the disclosure.

In some embodiments, the at least one nucleotide modification of a reference gRNA to generate a gNA variant comprises at least one nucleic acid substitution. In some embodiments, a gNA variant comprises 1, 2, 3, 4, 5, 6, 7, 8, 9, 10, 11, 12, 13, 14, 15, 16, 17, 18, 19 or 20 or more consecutive or non-consecutive substituted nucleotides relative to a reference gRNA. In some embodiments, a gNA variant comprises 1-4 nucleotide substitutions relative to a reference gRNA. In some embodiments, the at least one substitution comprises a substitution of 1, 2, 3, 4, 5, 6, 7, 8, 9, 10, 11, 12, 13, 14, 15, 16, 17, 18, 19 or 20 or more consecutive nucleotides relative to a reference gRNA. In some embodiments, the gNA variant comprises 2 or more substitutions relative to the reference gRNA, and the substitutions are not consecutive. In those embodiments where there are two or more non-consecutive substitutions in the gNA variant relative to the reference gRNA, any length of substituted nucleotides, and any combination of lengths of substituted nucleotides, as described herein, are contemplated as within the scope of the disclosure. For example, in some embodiments, a gNA variant may comprise a first substitution of one nucleotide, and a second substitution of two nucleotides and the two substitutions are not consecutive. In some embodiments, a gNA variant comprises at least two substitutions in different regions of the reference gRNA. In some embodiments, a gNA variant comprises at least two substitutions in the same region of the reference gRNA. For example, the regions may be the triplex, the extended stem loop, scaffold stem loop, scaffold stem bubble, triplex loop, pseudoknot, triplex, or a 5′ end of the gNA variant. Any substitution of A, G, C, U (or T, in the corresponding DNA) or combinations thereof at any location in the reference gRNA is contemplated as within the scope of the disclosure.

Any of the substitutions, insertions and deletions described herein can be combined to generate a gNA variant of the disclosure. For example, a gNA variant can comprise at least one substitution and at least one deletion relative to a reference gRNA, at least one substitution and at least one insertion relative to a reference gRNA, at least one insertion and at least one deletion relative to a reference gRNA, or at least one substitution, one insertion and one deletion relative to a reference gRNA.

In some embodiments, the gNA variant comprises a scaffold region at least 20% identical, at least 30% identical, at least 40% identical, at least 50% identical, at least 60% identical, at least 65% identical, at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to any one of SEQ ID NOS:4-16. In some embodiments, the gNA variant comprises a scaffold region at least 60% homologous (or identical) to any one of SEQ ID NOS:4-16.

In some embodiments, the gNA variant comprises a tracr stem loop at least 60% identical, at least 65% identical, at least 70% identical, at least 75% identical, at least 80% identical, at least 85% identical, at least 90% identical, or at least 95% identical to SEQ ID NO:14. In some embodiments, the gNA variant comprises a tracr stem loop at least 60% homologous (or identical) to SEQ ID NO: 14.

In some embodiments, the gNA variant comprises an extended stem loop at least 60% identical, at least 65% identical, at least 70% identical, at least 75% identical, at least 80% identical, at least 85% identical, at least 90% identical, or at least 95% identical to SEQ ID NO:15. In some embodiments, the gNA variant comprises an extended stem loop at least 60% homologous (or identical) to SEQ ID NO: 15.

In some embodiments, the gNA variant comprises an exogenous extended stem loop, with such differences from a reference gNA described as follows. In some embodiments, an exogenous extended stem loop has little or no identity to the reference stem loop regions disclosed herein (e.g., SEQ ID NO:15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, at least 2,000 bp, at least 3,000 bp, at least 4,000 bp, at least 5,000 bp, at least 6,000 bp, at least 7,000 bp, at least 8,000 bp, at least 9,000 bp, at least 10,000 bp, at least 12,000 bp, at least 15,000 bp or at least 20,000 bp. In some embodiments, the gNA variant comprises an extended stem loop region comprising at least 10, at least 100, at least 500, at least 1000, or at least 10,000 nucleotides. In some embodiments, the heterologous stem loop increases the stability of the gNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gNA has increased stability and, depending on the choice of loop, can interact with certain cellular proteins or RNA. Such exogenous extended stem loops can comprise, for example a thermostable RNA such as MS2 (ACAUGAGGAUCACCCAUGU (SEQ ID NO: 35)), Qβ (UGCAUGUCUAAGACAGCA (SEQ ID NO: 36)), U1 hairpin II (AAUCCAUUGCACUCCGGAUU (SEQ ID NO: 37)), Uvsx (CCUCUUCGGAGG (SEQ ID NO: 38)), PP7 (AGGAGUUUCUAUGGAAACCCU (SEQ ID NO: 39)), Phage replication loop (AGGUGGGACGACCUCUCGGUCGUCCUAUCU (SEQ ID NO: 40)), Kissing loop_a (UGCUCGCUCCGUUCGAGCA (SEQ ID NO: 41)), Kissing loop_b1 (UGCUCGACGCGUCCUCGAGCA (SEQ ID NO: 42)), Kissing loop_b2 (UGCUCGUUUGCGGCUACGAGCA (SEQ ID NO: 43)), G quadriplex M3q (AGGGAGGGAGGGAGAGG (SEQ ID NO: 44)), G quadriplex telomere basket (GGUUAGGGUUAGGGUUAGG (SEQ ID NO: 45)), Sarcin-ricin loop (CUGCUCAGUACGAGAGGAACCGCAG (SEQ ID NO: 46)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 47)). In some embodiments, an exogenous stem loop comprises a long non-coding RNA (lncRNA). As used herein, a lncRNA refers to a non-coding RNA that is longer than approximately 200 bp in length. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired. In some embodiments, the at least one nucleotide modification comprises: (a) substitution of 1 to 15 consecutive or non-consecutive nucleotides in the gNA variant in one or more regions; (b) a deletion of 1 to 10 consecutive or non-consecutive nucleotides in the gNA variant in one or more regions; (c) an insertion of 1 to 10 consecutive or non-consecutive nucleotides in the gNA variant in one or more regions; (d) a substitution of the scaffold stem loop or the extended stem loop with an RNA stem loop sequence from a heterologous RNA source with proximal 5′ and 3′ ends; or any combination of (a)-(d).

In some embodiments, the gNA variant comprises a scaffold stem loop sequence of CCAGCGACUAUGUCGUAGUGG (SEQ ID NO: 32). In some embodiments, the gNA variant comprises a scaffold stem loop sequence of CCAGCGACUAUGUCGUAGUGG (SEQ ID NO: 32) with at least 1, 2, 3, 4, or 5 mismatches thereto.

In some embodiments, the gNA variant comprises an extended stem loop region comprising less than 32 nucleotides, less than 31 nucleotides, less than 30 nucleotides, less than 29 nucleotides, less than 28 nucleotides, less than 27 nucleotides, less than 26 nucleotides, less than 25 nucleotides, less than 24 nucleotides, less than 23 nucleotides, less than 22 nucleotides, less than 21 nucleotides, or less than 20 nucleotides. In some embodiments, the gNA variant comprises an extended stem loop region comprising less than 32 nucleotides. In some embodiments, the gNA variant further comprises a thermostable stem loop.

In some embodiments, a sgRNA variant comprises a sequence of SEQ ID NOS:2104, SEQ ID NO:2106, SEQ ID NO:2163, SEQ ID NO:2107, SEQ ID NO:2164, SEQ ID NO:2165, SEQ ID NO:2166, SEQ ID NO:2103, SEQ ID NO:2167, SEQ ID NO:2105, SEQ ID NO:2108, SEQ ID NO:2112, SEQ ID NO:2160, SEQ ID NO:2170, SEQ ID NO:2114, SEQ ID NO:2171, SEQ ID NO:2112, SEQ ID NO:2173, SEQ ID NO:2102, SEQ ID NO:2174, SEQ ID NO:2175, SEQ ID NO:2109, SEQ ID NO:2176, SEQ ID NO:2238, SEQ ID NO:2239, SEQ ID NO:2240, SEQ ID NO:2241, SEQ ID NO:2256, SEQ ID NO:2274, SEQ ID NO:2275, SEQ ID NO:2279, or SEQ ID NO:2281. In some embodiments, a sgRNA variant comprises a sequence of SEQ ID NOS: 2238, 2246, 2256, 2274 or 2275.

In some embodiments, the gNA variant comprises the sequence of any one of SEQ ID NOS: 2236, 2237, 2238, 2241, 2244, 2248, 2249, 2256, or 2259-2294, or having at least about 80%, at least about 90%, at least about 95%, at least about 96%, at least about 97%, at least about 98%, at least about 99% identity thereto. In some embodiments, the gNA variant comprises one or more additional changes to a sequence of any one of SEQ ID NOs: 2201-2294. In some embodiments, the gNA variant comprises the sequence of any one of SEQ ID NOS:2236, 2237, 2238, 2241, 2244, 2248, 2249, or 2259-2294.

In some embodiments, a sgRNA variant comprises one or more additional changes to a sequence of SEQ ID NO:2104, SEQ ID NO:2163, SEQ ID NO:2107, SEQ ID NO:2164, SEQ ID NO:2165, SEQ ID NO:2166, SEQ ID NO:2103, SEQ ID NO:2167, SEQ ID NO:2105, SEQ ID NO:2108, SEQ ID NO:2112, SEQ ID NO:2160, SEQ ID NO:2170, SEQ ID NO:2114, SEQ ID NO:2171, SEQ ID NO:2112, SEQ ID NO:2173, SEQ ID NO:2102, SEQ ID NO:2174, SEQ ID NO:2175, SEQ ID NO:2109, SEQ ID NO:2176, SEQ ID NO:2238, SEQ ID NO:2239, SEQ ID NO:2240, SEQ ID NO:2241, SEQ ID NO:2243, SEQ ID NO:2256, SEQ ID NO:2274, SEQ ID NO:2275, SEQ ID NO:2279, or SEQ ID NO:2281.

In some embodiments of the gNA variants of the disclosure, the gNA variant comprises at least one modification, wherein the at least one modification compared to the reference guide scaffold of SEQ ID NO:5 is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired. In such embodiments, the gNA variant comprises the sequence of any one of SEQ ID NOS:2236, 2237, 2238, 2241, 2244, 2248, 2249, 2256 or 2259-2294.

In the embodiments of the gNA variants, the gNA variant further comprises a spacer (or targeting sequence) region located at the 3′ end of the gNA, specific to a C9orf72 sequence. Exemplary spacers, and their cognate PAM sequences, are shown in Table 3 below.

TABLE 3
gNA Targeting Sequences for the C9orf72 Gene

| PAM Sequence | SEQ ID NO |
|---|---|
| ATC | 363-2100, 2295-5426 |
| TTC | 5427-12893 |
| GTC | 12894-16202 |
| CTC | 16203-21835 |

In the embodiments of the gNA variants, the gNA variant further comprises a spacer (or targeting sequence) region located at the 3′ end of the gNA, described more fully, supra, which comprises at least 14 to about 35 nucleotides wherein the spacer is designed with a sequence that is complementary to a target nucleic acid. In some embodiments, the gNA variant comprises a targeting sequence of at least 10 to 30 nucleotides complementary to a target nucleic acid. In some embodiments, the targeting sequence has 14, 15, 16, 17, 18, 19, 20, 21, 22, 23, 24, 25, 26, 27, 28, 29, 30, 31, 32, 33, 34 or 35 nucleotides. In some embodiments, the gNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides. In some embodiments, the disclosure provides targeting sequences for inclusion in the gNA variants of the disclosure comprising a sequence that is at least 50% identical, at least 55% identical, at least 60% identical, at least 65% identical, at least 70% identical, at least 75% identical, at least 80% identical, at least 85% identical, at least 90% identical, at least 95% identical, or 100% identical to a sequence of SEQ ID NOs: 309-343, 363-2100 and 2295-21835. In some embodiments, the targeting sequence of the gNA variant comprises a sequence a sequence of SEQ ID NOs: 309-343, 363-2100 and 2295-21835 with a single nucleotide removed from the 3′ end of the sequence. In other embodiments, the targeting sequence of the gNA variant comprises a sequence a sequence of SEQ ID NOs: 309-343, 363-2100 and 2295-21835 with two nucleotides removed from the 3′ end of the sequence. In other embodiments, the targeting sequence of the gNA variant comprises a sequence a sequence of SEQ ID NOs: 309-343, 363-2100 and 2295-21835 with three nucleotides removed from the 3′ end of the sequence. In other embodiments, the targeting sequence of the gNA variant comprises a sequence a sequence of SEQ ID NOs: 309-343, 363-2100 and 2295-21835 with four nucleotides removed from the 3′ end of the sequence. In other embodiments, the targeting sequence of the gNA variant comprises a sequence a sequence of SEQ ID NOs: 309-343, 363-2100 and 2295-21835 with five nucleotides removed from the 3′ end of the sequence.

In some embodiments, the gNA variant further comprises a spacer (targeting) region located at the 3′ end of the gNA, wherein the spacer is designed with a sequence that is complementary to a target nucleic acid. In some embodiments, the target nucleic acid comprises a PAM sequence located 5′ of the spacer with at least a single nucleotide separating the PAM from the first nucleotide of the spacer. In some embodiments, the PAM is located on the non-targeted strand of the target region, i.e. the strand that is complementary to the target nucleic acid. In some embodiments, the PAM sequence is ATC. In some embodiments, the targeting sequence for an ATC PAM comprises SEQ ID NOs: 363-2100 or 2295-5426, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOs: 363-2100 or 2295-5426. In some embodiments, the targeting sequence for an ATC PAM is selected from the group consisting of SEQ ID NOs: 363-2100 or 2295-5426. In some embodiments, the PAM sequence is CTC. In some embodiments, the targeting sequence for a CTC PAM comprises SEQ ID NOs: 16203-21835, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOs: 16203-21835. In some embodiments, the targeting sequence for a CTC PAM is selected from the group consisting of SEQ ID NOs: 16203-21835. In some embodiments, the PAM sequence is GTC. In some embodiments, the targeting sequences for a GTC PAM comprises SEQ ID NOs: 12894-16202 or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOs: 12894-16202. In some embodiments, the targeting sequence for a GTC PAM is selected from the group consisting of SEQ ID NOs: 12894-16202. In some embodiments, the PAM sequence is TTC. In some embodiments, a targeting sequences for a TTC PAM comprises SEQ ID NOs: 5427-12893, or a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to SEQ ID NOs: 5427-12893. In some embodiments, a targeting sequence for a TTC PAM is selected from the group consisting of SEQ ID NOs: 5427-12893.

In some embodiments, the scaffold of the gNA variant is part of an RNP with a reference CasX protein comprising SEQ ID NO:1, SEQ ID NO:2, or SEQ ID NO:3. In other embodiments, the scaffold of the gNA variant is part of an RNP with a CasX variant protein comprising any one of the sequences of Tables 4, 6, 7, 8, or 10 or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In the foregoing embodiments, the gNA further comprises a spacer sequence.

In some embodiments, the scaffold of the gNA variant is a variant comprising one or more additional changes to a sequence of a reference gRNA that comprises SEQ ID NO:4 or SEQ ID NO:5. In those embodiments where the scaffold of the reference gRNA is derived from SEQ ID NO:4 or SEQ ID NO:5, the one or more improved or added characteristics of the gNA variant are improved compared to the same characteristic in SEQ ID NO:4 or SEQ ID NO:5.

m. Complex Formation with CasX Protein

In some embodiments, a gNA variant has an improved ability to form a complex with a CasX protein (such as a reference CasX or a CasX variant protein) when compared to a reference gRNA. In some embodiments, a gNA variant has an improved affinity for a CasX protein (such as a reference or variant protein) when compared to a reference gRNA, thereby improving its ability to form a ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gNA variant and its spacer are competent for gene editing of a target nucleic acid.

h. Complex Formation with CasX Protein

In some embodiments, a gNA variant has an improved ability to form a complex with a CasX protein (such as a reference CasX or a CasX variant protein) when compared to a reference gRNA. In some embodiments, a gNA variant has an improved affinity for a CasX protein (such as a reference or variant protein) when compared to a reference gRNA, thereby improving its ability to form a ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gNA variant and its spacer are competent for gene editing of a target nucleic acid.

Exemplary nucleotide changes that can improve the ability of gNA variants to form a complex with CasX protein may, in some embodiments, include replacing the scaffold stem with a thermostable stem loop. Without wishing to be bound by any theory, replacing the scaffold stem with a thermostable stem loop could increase the overall binding stability of the gNA variant with the CasX protein. Alternatively, or in addition, removing a large section of the stem loop could change the gNA variant folding kinetics and make a functional folded gNA easier and quicker to structurally-assemble, for example by lessening the degree to which the gNA variant can get “tangled” in itself. In some embodiments, choice of scaffold stem loop sequence could change with different spacers that are utilized for the gNA. In some embodiments, scaffold sequence can be tailored to the spacer and therefore the target sequence. Biochemical assays can be used to evaluate the binding affinity of CasX protein for the gNA variant to form the RNP, including the assays of the Examples. For example, a person of ordinary skill can measure changes in the amount of a fluorescently tagged gNA that is bound to an immobilized CasX protein, as a response to increasing concentrations of an additional unlabeled “cold competitor” gNA. Alternatively, or in addition, fluorescence signal can be monitored to or seeing how it changes as different amounts of fluorescently labeled gNA are flowed over immobilized CasX protein. Alternatively, the ability to form an RNP can be assessed using in vitro cleavage assays against a defined target nucleic acid sequence.

### i. gNA Stability

In some embodiments, a gNA variant has improved stability when compared to a reference gRNA. Increased stability and efficient folding may, in some embodiments, increase the extent to which a gNA variant persists inside a target cell, which may thereby increase the chance of forming a functional RNP capable of carrying out CasX functions such as gene editing. Increased stability of gNA variants may also, in some embodiments, allow for a similar outcome with a lower amount of gNA delivered to a cell, which may in turn reduce the chance of off-target effects during gene editing. Guide RNA stability can be assessed in a variety of ways, including for example in vitro by assembling the guide, incubating for varying periods of time in a solution that mimics the intracellular environment, and then measuring functional activity via the in vitro cleavage assays described herein. Alternatively, or in addition, gNAs can be harvested from cells at varying time points after initial transfection/transduction of the gNA to determine how long gNA variants persist relative to reference gRNAs.

j. Solubility

In some embodiments, a gNA variant has improved solubility when compared to a reference gRNA. In some embodiments, a gNA variant has improved solubility of the CasX protein:gNA RNP when compared to a reference gRNA. In some embodiments, solubility of the CasX protein:gNA RNP is improved by the addition of a ribozyme sequence to a 5′ or 3′ end of the gNA variant, for example the 5′ or 3′ of a reference sgRNA. Some ribozymes, such as the M1 ribozyme, can increase solubility of proteins through RNA mediated protein folding. Increased solubility of CasX RNPs comprising a gNA variant as described herein can be evaluated through a variety of means known to one of skill in the art, such as by taking densitometry readings on a gel of the soluble fraction of lysed E. coli in which the CasX and gNA variants are expressed.

k. Resistance to Nuclease Activity

In some embodiments, a gNA variant has improved resistance to nuclease activity compared to a reference gRNA that may, for example, increase the persistence of a variant gNA in an intracellular environment, thereby improving gene editing. Resistance to nuclease activity may be evaluated through a variety of methods known to one of skill in the art. For example, in vitro methods of measuring resistance to nuclease activity may include for example contacting reference gNA and variants with one or more exemplary RNA nucleases and measuring degradation. Alternatively, or in addition, measuring persistence of a gNA variant in a cellular environment using the methods described herein can indicate the degree to which the gNA variant is nuclease resistant.

l. Binding Affinity to a Target DNA

In some embodiments, a gNA variant has improved affinity for the target DNA relative to a reference gRNA. In certain embodiments, a ribonucleoprotein complex comprising a gNA variant has improved affinity for the target DNA, relative to the affinity of an RNP comprising a reference gRNA. In some embodiments, the improved affinity of the RNP for the target DNA comprises improved affinity for the target sequence, improved affinity for the PAM sequence, improved ability of the RNP to search DNA for the target sequence, or any combinations thereof. In some embodiments, the improved affinity for the target DNA is the result of increased overall DNA binding affinity.

Without wishing to be bound by theory, it is possible that nucleotide changes in the gNA variant that affect the function of the OBD in the CasX protein may increase the affinity of CasX variant protein binding to the protospacer adjacent motif (PAM), as well as the ability to bind or utilize an increased spectrum of PAM sequences other than the canonical TTC PAM recognized by the reference CasX protein of SEQ ID NO:2, including PAM sequences selected from the group consisting of TTC, ATC, GTC, and CTC, thereby increasing the affinity and diversity of the CasX variant protein for target DNA sequences, resulting in a substantial increase in the target nucleic acid sequences that can be edited and/or bound, compared to a reference CasX. As described more fully, below, increasing the sequences of the target nucleic acid that can be edited, compared to a reference CasX, refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with at least a single nucleotide separating the PAM from the first nucleotide of the protospacer. Alternatively, or in addition, changes in the gNA that affect function of the helical I and/or helical II domains that increase the affinity of the CasX variant protein for the target DNA strand can increase the affinity of the CasX RNP comprising the variant gNA for target DNA.

m. Adding or Changing gNA Function

In some embodiments, gNA variants can comprise larger structural changes that change the topology of the gNA variant with respect to the reference gRNA, thereby allowing for different gNA functionality. For example, in some embodiments a gNA variant has swapped an endogenous stem loop of the reference gRNA scaffold with a previously identified stable RNA structure or a stem loop that can interact with a protein or RNA binding partner to recruit additional moieties to the CasX or to recruit CasX to a specific location, such as the inside of a viral capsid, that has the binding partner to the said RNA structure. In other scenarios the RNAs may be recruited to each other, as in Kissing loops, such that two CasX proteins can be co-localized for more effective gene editing at the target DNA sequence. Such RNA structures may include MS2, Qβ, U1 hairpin II, Uvsx, PP7, Phage replication loop, Kissing loop_a, Kissing loop_b1, Kissing loop_b2, G quadriplex M3q, G quadriplex telomere basket, Sarcin-ricin loop, or a Pseudoknot.

In some embodiments, a gNA variant comprises a terminal fusion partner. Exemplary terminal fusions may include fusion of the gRNA to a self-cleaving ribozyme or protein binding motif. As used herein, a “ribozyme” refers to an RNA or segment thereof with one or more catalytic activities similar to a protein enzyme. Exemplary ribozyme catalytic activities may include, for example, cleavage and/or ligation of RNA, cleavage and/or ligation of DNA, or peptide bond formation. In some embodiments, such fusions could either improve scaffold folding or recruit DNA repair machinery. For example, a gRNA may in some embodiments be fused to a hepatitis delta virus (HDV) antigenomic ribozyme, HDV genomic ribozyme, hatchet ribozyme (from metagenomic data), env25 pistol ribozyme (representative from Aliistipes putredinis), HH15 Minimal Hammerhead ribozyme, tobacco ringspot virus (TRSV) ribozyme, WT viral Hammerhead ribozyme (and rational variants), or Twisted Sister 1 or RBMX recruiting motif. Hammerhead ribozymes are RNA motifs that catalyze reversible cleavage and ligation reactions at a specific site within an RNA molecule. Hammerhead ribozymes include type I, type II and type III hammerhead ribozymes. The HDV, pistol, and hatchet ribozymes have self-cleaving activities. gNA variants comprising one or more ribozymes may allow for expanded gNA function as compared to a gRNA reference. For example, gNAs comprising self-cleaving ribozymes can, in some embodiments, be transcribed and processed into mature gNAs as part of polycistronic transcripts. Such fusions may occur at either the 5′ or the 3′ end of the gNA. In some embodiments, a gNA variant comprises a fusion at both the 5′ and the 3′ end, wherein each fusion is independently as described herein. In some embodiments, a gNA variant comprises a phage replication loop or a tetraloop. In some embodiments, a gNA comprises a hairpin loop that is capable of binding a protein. For example, in some embodiments the hairpin loop is an MS2, Qβ, U1 hairpin II, Uvsx, or PP7 hairpin loop.

In some embodiments, a gNA variant comprises one or more RNA aptamers. As used herein, an “RNA aptamer” refers to an RNA molecule that binds a target with high affinity and high specificity. In some embodiments, a gNA variant comprises one or more riboswitches. As used herein, a “riboswitch” refers to an RNA molecule that changes state upon binding a small molecule. In some embodiments, the gNA variant further comprises one or more protein binding motifs. Adding protein binding motifs to a reference gRNA or gNA variant of the disclosure may, in some embodiments, allow a CasX RNP to associate with additional proteins, which can, for example, add the functionality of those proteins to the CasX RNP.

n. Chemically Modified gNA

In some embodiments, the disclosure relates to chemically-modified gNA. In some embodiments, the present disclosure provides a chemically-modified gNA that has guide RNA functionality and has reduced susceptibility to cleavage by a nuclease. A gNA that comprises any nucleotide other than the four canonical ribonucleotides A, C, G, and U, or a deoxynucleotide, is a chemically modified gNA. In some cases, a chemically-modified gNA comprises any backbone or internucleotide linkage other than a natural phosphodiester internucleotide linkage. In certain embodiments, the retained functionality includes the ability of the modified gNA to bind to a CasX of any of the embodiments described herein. In certain embodiments, the retained functionality includes the ability of the modified gNA to bind to a C9orf72 target nucleic acid sequence. In certain embodiments, the retained functionality includes targeting a CasX protein or the ability of a pre-complexed CasX protein-gNA to bind to a target nucleic acid sequence. In certain embodiments, the retained functionality includes the ability to nick a target polynucleotide by a CasX-gNA. In certain embodiments, the retained functionality includes the ability to cleave a target nucleic acid sequence by a CasX-gNA. In certain embodiments, the retained functionality is any other known function of a gNA in a CasX system with a CasX protein of the embodiments of the disclosure.

In some embodiments, the disclosure provides a chemically-modified gNA in which a nucleotide sugar modification is incorporated into the gNA selected from the group consisting of 2′-OC₁₋₄alkyl such as 2′-O-methyl (2′-OMe), 2′-deoxy (2′-H), 2′-OC₁₋₃alkyl-O—C₁₋₃alkyl such as 2′-methoxyethyl (“2′-MOE”), 2′-fluoro (“2′-F”), 2′-amino (“2′-NH₂”), 2′-arabinosyl (“2′-arabino”) nucleotide, 2′-F-arabinosyl (“2′-F-arabino”) nucleotide, 2′-locked nucleic acid (“LNA”) nucleotide, 2′-unlocked nucleic acid (“ULNA”) nucleotide, a sugar in L form (“L-sugar”), and 4′-thioribosyl nucleotide. In other embodiments, an internucleotide linkage modification incorporated into the guide RNA is selected from the group consisting of: phosphorothioate “P(S)” (P(S)), phosphonocarboxylate (P(CH₂)COOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂)ₙCOO⁻)), alkylphosphonate (P(C₁₋₃alkyl) such as methylphosphonate —P(CH₃), boranophosphonate (P(BH₃)), and phosphorodithioate (P(S)₂).

In certain embodiments, the disclosure provides a chemically-modified gNA in which a nucleobase (“base”) modification is incorporated into the gNA selected from the group consisting of: 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”), 5-methyl-2-pyrimidine, x(A,G,C,T) and y(A,G,C,T).

In other embodiments, the disclosure provides a chemically-modified gNA in which one or more isotopic modifications are introduced on the nucleotide sugar, the nucleobase, the phosphodiester linkage and/or the nucleotide phosphates, including nucleotides comprising one or more ¹⁵N, ¹³C, ¹⁴C, deuterium, ³H, ³²P, ¹²⁵I, ¹³¹I atoms or other atoms or elements used as tracers.

In some embodiments, an “end” modification incorporated into the gNA is selected from the group consisting of: PEG (polyethyleneglycol), hydrocarbon linkers (including: heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers), spermine linkers, dyes including fluorescent dyes (for example fluoresceins, rhodamines, cyanines) attached to linkers such as, for example, 6-fluorescein-hexyl, quenchers (for example dabcyl, BHQ) and other labels (for example biotin, digoxigenin, acridine, streptavidin, avidin, peptides and/or proteins). In some embodiments, an “end” modification comprises a conjugation (or ligation) of the gNA to another molecule comprising an oligonucleotide of deoxynucleotides and/or ribonucleotides, a peptide, a protein, a sugar, an oligosaccharide, a steroid, a lipid, a folic acid, a vitamin and/or other molecule. In certain embodiments, the disclosure provides a chemically-modified gNA in which an “end” modification (described above) is located internally in the gNA sequence via a linker such as, for example, a 2-(4-butylamidofluorescein)propane-1,3-diol bis(phosphodiester) linker, which is incorporated as a phosphodiester linkage and can be incorporated anywhere between two nucleotides in the gNA.

In some embodiments, the disclosure provides a chemically-modified gNA having an end modification comprising a terminal functional group such as an amine, a thiol (or sulfhydryl), a hydroxyl, a carboxyl, carbonyl, thionyl, thiocarbonyl, a carbamoyl, a thiocarbamoyl, a phoshoryl, an alkene, an alkyne, an halogen or a functional group-terminated linker that can be subsequently conjugated to a desired moiety selected from the group consisting of a fluorescent dye, a non-fluorescent label, a tag (for ¹⁴C, example biotin, avidin, streptavidin, or moiety containing an isotopic label such as ¹⁵N, ¹³C, deuterium, ³H, ³²P, ¹²⁵I and the like), an oligonucleotide (comprising deoxynucleotides and/or ribonucleotides, including an aptamer), an amino acid, a peptide, a protein, a sugar, an oligosaccharide, a steroid, a lipid, a folic acid, and a vitamin. The conjugation employs standard chemistry well-known in the art, including but not limited to coupling via N-hydroxysuccinimide, isothiocyanate, DCC (or DCI), and/or any other standard method as described in “Bioconjugate Techniques” by Greg T. Hermanson, Publisher Eslsevier Science, 3^{rd} ed. (2013), the contents of which are incorporated herein by reference in its entirety.

### IV. Proteins for Modifying a Target Nucleic Acid

The present disclosure provides systems comprising a CRISPR nuclease that have utility in genome editing of eukaryotic cells. In some embodiments, the CRISPR nuclease employed in the genome editing systems is a Class 2 Type V nuclease. Although members of Class 2 Type V CRISPR-Cas systems have differences, they share some common characteristics that distinguish them from the Cas9 systems. Firstly, the Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a, Cas12b, Cas12c, Cas12d (CasY), and CasX. In some embodiments, the Type V nuclease is a CasX nuclease. In some embodiments, the present disclosure provides systems comprising a CasX protein and one or more gNA acids (CasX:gNA system) that are specifically designed to modify a target nucleic acid sequence in eukaryotic cells.

The term “CasX protein”, as used herein, refers to a family of proteins, and encompasses all naturally occurring CasX proteins, proteins that share at least 50% identity to naturally occurring CasX proteins, as well as CasX variants exhibiting one or more improved characteristics relative to a naturally-occurring reference CasX protein.

Exemplary improved characteristics of the CasX variant embodiments include, but are not limited to improved folding of the variant, improved binding affinity to the gNA, improved binding affinity to the target nucleic acid, improved ability to utilize a greater spectrum of PAM sequences in the editing and/or binding of target DNA, improved unwinding of the target DNA, increased editing activity, improved editing efficiency, improved editing specificity, increased percentage of a eukaryotic genome that can be efficiently edited, increased activity of the nuclease, increased target strand loading for double strand cleavage, decreased target strand loading for single strand nicking, decreased off-target cleavage, improved binding of the non-target strand of DNA, improved protein stability, improved protein:gNA (RNP) complex stability, improved protein solubility, improved protein:gNA (RNP) complex solubility, improved protein yield, improved protein expression, and improved fusion characteristics, as described more fully, below. In some embodiments, the RNP of the CasX variant and the gNA variant exhibit one or more of the improved characteristics that are at least about 1.1 to about 100,000-fold improved relative to an RNP of the reference CasX protein of SEQ ID NO:1, SEQ ID NO:2, or SEQ ID NO:3 and the gNA of Table 1, when assayed in a comparable fashion. In other cases, the one or more improved characteristics of an RNP of the CasX variant and the gNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the gNA of Table 1. In other cases, the one or more of the improved characteristics of an RNP of the CasX variant and the gNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to an RNP of the reference CasX protein of SEQ ID NO:1, SEQ ID NO:2, or SEQ ID NO:3 and the gNA of Table 1, when assayed in a comparable fashion. In other cases, the one or more improved characteristics of an RNP of the CasX variant and the gNA variant are about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to an RNP of the reference CasX protein of SEQ ID NO:1, SEQ ID NO:2, or SEQ ID NO:3 and the gNA of Table 1, when assayed in a comparable fashion.

The term “CasX variant” is inclusive of variants that are fusion proteins; i.e., the CasX is “fused to” a heterologous sequence. This includes CasX variants comprising CasX variant sequences and N-terminal, C-terminal, or internal fusions of the CasX to a heterologous protein or domain thereof.

CasX proteins of the disclosure comprise at least one of the following domains: a non-target strand binding (NTSB) domain, a target strand loading (TSL) domain, a helical I domain, a helical II domain, an oligonucleotide binding domain (OBD), and a RuvC DNA cleavage domain (the last of which may be modified or deleted in a catalytically dead CasX variant), described more fully, below. Additionally, the CasX variant proteins of the disclosure have an enhanced ability to efficiently edit and/or bind target DNA, when complexed with a gNA as an RNP, utilizing PAM TC motif, including PAM sequences selected from TTC, ATC, GTC, or CTC, compared to an RNP of a reference CasX protein and reference gNA. In the foregoing, the PAM sequence is located at least 1 nucleotide 5′ to the non-target strand of the protospacer having identity with the targeting sequence of the gNA in a assay system compared to the editing efficiency and/or binding of an RNP comprising a reference CasX protein and reference gNA in a comparable assay system. In one embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is TTC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is ATC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is CTC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is GTC. In the foregoing embodiments, the increased editing efficiency and/or binding affinity for the one or more PAM sequences is at least 1.5-fold greater or more compared to the editing efficiency and/or binding affinity of an RNP of any one of the CasX proteins of SEQ ID NOS:1-3 and the gNA of Table 1 for the PAM sequences.

In some embodiments, a CasX protein can bind and/or modify (e.g., cleave, nick, methylate, demethylate, etc.) a target nucleic acid and/or a polypeptide associated with target nucleic acid (e.g., methylation or acetylation of a histone tail). In some embodiments, the CasX protein is catalytically dead (dCasX) but retains the ability to bind a target nucleic acid. An exemplary catalytically dead CasX protein comprises one or more mutations in the active site of the RuvC domain of the CasX protein. In some embodiments, a catalytically dead CasX protein comprises substitutions at residues 672, 769 and/or 935 of SEQ ID NO:1. In one embodiment, a catalytically dead CasX protein comprises substitutions of D672A, E769A and/or D935A in a reference CasX protein of SEQ ID NO:1. In other embodiments, a catalytically dead CasX protein comprises substitutions at amino acids 659, 756 and/or 922 in a reference CasX protein of SEQ ID NO:2. In some embodiments, a catalytically dead CasX protein comprises D659A, E756A and/or D922A substitutions in a reference CasX protein of SEQ ID NO:2. In further embodiments, a catalytically dead CasX protein comprises deletions of all or part of the RuvC domain of the CasX protein. It will be understood that the same foregoing substitutions can similarly be introduced into the CasX variants of the disclosure, resulting in a dCasX variant. In one embodiment, all or a portion of the RuvC domain is deleted from the CasX variant, resulting in a dCasX variant. Catalytically inactive dCasX variant proteins can, in some embodiments, be used for base editing or epigenetic modifications. With a higher affinity for DNA, in some embodiments, catalytically inactive dCasX variant proteins can, relative to catalytically active CasX, find their target nucleic acid faster, remain bound to target nucleic acid for longer periods of time, bind target nucleic acid in a more stable fashion, or a combination thereof, thereby improving these functions of the catalytically dead CasX variant protein compared to a CasX variant that retains its cleavage capability.

a. Non-Target Strand Binding Domain

The reference CasX proteins of the disclosure comprise a non-target strand binding domain (NTSBD). The NTSBD is a domain not previously found in any Cas proteins; for example this domain is not present in Cas proteins such as Cas9, Cas12a/Cpf1, Cas13, Cas14, CASCADE, CSM, or CSY. Without being bound to theory or mechanism, a NTSBD in a CasX allows for binding to the non-target DNA strand and may aid in unwinding of the non-target and target strands. The NTSBD is presumed to be responsible for the unwinding, or the capture, of a non-target DNA strand in the unwound state. The NTSBD is in direct contact with the non-target strand in CryoEM model structures derived to date and may contain a non-canonical zinc finger domain. The NTSBD may also play a role in stabilizing DNA during unwinding, guide RNA invasion and R-loop formation. In some embodiments, an exemplary NTSBD comprises amino acids 101-191 of SEQ ID NO:1 or amino acids 103-192 of SEQ ID NO:2. In some embodiments, the NTSBD of a reference CasX protein comprises a four-stranded beta sheet.

b. Target Strand Loading Domain

The reference CasX proteins of the disclosure comprise a Target Strand Loading (TSL) domain. The TSL domain is a domain not found in certain Cas proteins such as Cas9, CASCADE, CSM, or CSY. Without wishing to be bound by theory or mechanism, it is thought that the TSL domain is responsible for aiding the loading of the target DNA strand into the RuvC active site of a CasX protein. In some embodiments, the TSL acts to place or capture the target-strand in a folded state that places the scissile phosphate of the target strand DNA backbone in the RuvC active site. The TSL comprises a cys4 (CXXC, CXXC zinc finger/ribbon domain (SEQ ID NO: 48) that is separated by the bulk of the TSL. In some embodiments, an exemplary TSL comprises amino acids 825-934 of SEQ ID NO:1 or amino acids 813-921 of SEQ ID NO:2.

c. Helical I Domain

The reference CasX proteins of the disclosure comprise a helical I domain. Certain Cas proteins other than CasX have domains that may be named in a similar way. However, in some embodiments, the helical I domain of a CasX protein comprises one or more unique structural features, or comprises a unique sequence, or a combination thereof, compared to non-CasX proteins. For example, in some embodiments, the helical I domain of a CasX protein comprises one or more unique secondary structures compared to domains in other Cas proteins that may have a similar name. For example, in some embodiments the helical I domain in a CasX protein comprises one or more alpha helices of unique structure and sequence in arrangement, number and length compared to other CRISPR proteins. In certain embodiments, the helical I domain is responsible for interacting with the bound DNA and spacer of the guide RNA. Without wishing to be bound by theory, it is thought that in some cases the helical I domain may contribute to binding of the protospacer adjacent motif (PAM). In some embodiments, an exemplary helical I domain comprises amino acids 57-100 and 192-332 of SEQ ID NO:1, or amino acids 59-102 and 193-333 of SEQ ID NO:2. In some embodiments, the helical I domain of a reference CasX protein comprises one or more alpha helices.

d. Helical II Domain

The reference CasX proteins of the disclosure comprise a helical II domain. Certain Cas proteins other than CasX have domains that may be named in a similar way. However, in some embodiments, the helical II domain of a CasX protein comprises one or more unique structural features, or a unique sequence, or a combination thereof, compared to domains in other Cas proteins that may have a similar name. For example, in some embodiments, the helical II domain comprises one or more unique structural alpha helical bundles that align along the target DNA:guide RNA channel. In some embodiments, in a CasX comprising a helical II domain, the target strand and guide RNA interact with helical II (and the helical I domain, in some embodiments) to allow RuvC domain access to the target DNA. The helical II domain is responsible for binding to the guide RNA scaffold stem loop as well as the bound DNA. In some embodiments, an exemplary helical II domain comprises amino acids 333-509 of SEQ ID NO:1, or amino acids 334-501 of SEQ ID NO:2.

e. Oligonucleotide Binding Domain

The reference CasX proteins of the disclosure comprise an Oligonucleotide Binding Domain (OBD). Certain Cas proteins other than CasX have domains that may be named in a similar way. However, in some embodiments, the OBD comprises one or more unique functional features, or comprises a sequence unique to a CasX protein, or a combination thereof. For example, in some embodiments the bridged helix (BH), helical I domain, helical II domain, and Oligonucleotide Binding Domain (OBD) together are responsible for binding of a CasX protein to the guide RNA. Thus, for example, in some embodiments the OBD is unique to a CasX protein in that it interacts functionally with a helical I domain, or a helical II domain, or both, each of which may be unique to a CasX protein as described herein. Specifically, in CasX the OBD largely binds the RNA triplex of the guide RNA scaffold. The OBD may also be responsible for binding to the protospacer adjacent motif (PAM). An exemplary OBD domain comprises amino acids 1-56 and 510-660 of SEQ ID NO:1, or amino acids 1-58 and 502-647 of SEQ ID NO:2.

f. RuvC DNA Cleavage Domain

The reference CasX proteins of the disclosure comprise a RuvC domain, that includes 2 partial RuvC domains (RuvC-I and RuvC-II). The RuvC domain is the ancestral domain of all type 12 CRISPR proteins. The RuvC domain originates from a TNPB (transposase B) like transposase. Similar to other RuvC domains, the CasX RuvC domain has a DED catalytic triad that is responsible for coordinating a magnesium (Mg) ion and cleaving DNA. In some embodiments, the RuvC has a DED motif active site that is responsible for cleaving both strands of DNA (one by one, most likely the non-target strand first at 11-14 nucleotides (nt) into the targeted sequence and then the target strand next at 2-4 nucleotides after the target sequence). Specifically in CasX, the RuvC domain is unique in that it is also responsible for binding the guide RNA scaffold stem loop that is critical for CasX function. An exemplary RuvC domain comprises amino acids 661-824 and 935-986 of SEQ ID NO:1, or amino acids 648-812 and 922-978 of SEQ ID NO:2.

g. Reference CasX Proteins

The disclosure provides naturally-occurring CasX proteins (referred to herein as a “reference CasX protein”) that function as an endonuclease that catalyzes a double strand break at a specific sequence in a targeted double-stranded DNA (dsDNA). The sequence specificity is provided by the targeting sequence of the associated gNA to which it is complexed, which hybridizes to a target sequence within the target nucleic acid. For example, reference CasX proteins can be isolated from naturally occurring prokaryotes, such as Deltaproteobacteria, Planctomycetes, or Candidatus sungbacteria species. A reference CasX protein (sometimes referred to herein as a reference CasX protein) is a Type V CRISPR/Cas endonuclease belonging to the CasX (sometimes referred to as Cas12e) family of proteins that is capable of interacting with a guide NA to form a ribonucleoprotein (RNP) complex. In some embodiments, the RNP complex comprising the reference CasX protein can be targeted to a particular site in a target nucleic acid via base pairing between the targeting sequence (or spacer) of the gNA and a target sequence in the target nucleic acid. In some embodiments, the RNP comprising the reference CasX protein is capable of cleaving target DNA. In some embodiments, the RNP comprising the reference CasX protein is capable of nicking target DNA. In some embodiments, the RNP comprising the reference CasX protein is capable of editing target DNA, for example in those embodiments where the reference CasX protein is capable of cleaving or nicking DNA, followed by non-homologous end joining (NHEJ), homology-directed repair (HDR), homology-independent targeted integration (HITI), micro-homology mediated end joining (MMEJ), single strand annealing (SSA) or base excision repair (BER). In some embodiments, the RNP comprising the CasX protein is a catalytically dead (is catalytically inactive or has substantially no cleavage activity) CasX protein (dCasX), but retains the ability to bind the target DNA, described more fully, supra.

In some cases, a reference CasX protein is isolated or derived from Deltaproteobacter. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of:

(SEQ ID NO: 1)

| | |
|---|---|
| 1 | MEKRINKIRK KLSADNATKP VSRSGPMKTL LVRVMTDDLK |
| | KRLEKRRKKP EVMPQVISNN |
| 61 | AANNLRMLLD DYTKMKEAIL QVYWQEFKDD HVGLMCKFAQ |
| | PASKKIDQNK LKPEMDEKGN |
| 121 | LTTAGFACSQ TNYFGRCNVA EQVSEKGKAY TNYFGRCNVA |
| | EHEKLILLAQ LKPEKDSDEA |
| 181 | VTYSLGKFGQ RALDFYSIHV TKESTHPVKP LAQIAGNRYA |
| | SGPVGKALSD ACMGTIASFL |
| 241 | SKYQDIIIEH QKVVKGNQKR LESLRELAGK ENLEYPSVTL |
| | PPQPHTKEGV DAYNEVIARV |
| 301 | RMWVNLNLWQ KLKLSRDDAK PLLRLKGFPS FPVVERRENE |
| | VDWWNTINEV KKLIDAKRDM |
| 361 | GRVFWSGVTA EKRNTILEGY NYLPNENDHK KREGSLENPK |
| | KPAKRQFGDL LLYLEKKYAG |
| 421 | DWGKVFDEAW ERIDKKIAGL TSHIEREEAR NAEDAQSKAV |
| | LTDWLRAKAS FVLERLKEMD |
| 481 | EKEFYACEIQ LQKWYGDLRG NPFAVEAENR VVDISGFSIG |
| | SDGHSIQYRN LLAWKYLENG |
| 541 | KREFYLLMNY GKKGRIRFTD GTDIKKSGKW QGLLYGGGKA |
| | KVIDLTFDPD DEQLIILPLA |
| 601 | FGTRQGREFI WNDLLSLETG LIKLANGRVI EKTIYNKKIG |
| | RDEPALFVAL TFERREVVDP |
| 661 | SNIKPVNLIG VDRGENIPAV IALTDPEGCP LPEFKDSSGG |
| | PTDILRIGEG YKEKQRAIQA |
| 721 | AKEVEQRRAG GYSRKFASKS RNLADDMVRN SARDLFYHAV |
| | THDAVLVFEN LSRGFGRQGK |
| 781 | RTFMTERQYT KMEDWLTAKL AYEGLTSKTY LSKTLAQYTS |
| | KTCSNCGFTI TTADYDGMLV |
| 841 | RLKKTSDGWA TTLNNKELKA EGQITYYNRY KRQTVEKELS |
| | AELDRLSEES GNNDISKWTK |
| 901 | GRRDEALFLL KKRFSHRPVQ EQFVCLDCGH EVHADEQAAL |
| | NIARSWLFLN SNSTEFKSYK |
| 961 | SGKQPFVGAW QAFYKRRLKE VWKPNA. |

In some cases, a reference CasX protein is isolated or derived from Planctomycetes. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of:

(SEQ ID NO: 2)

| | |
|---|---|
| 1 | MQEIKRINKI RRRLVKDSNT KKAGKTGPMK TLLVRVMTPD |
| | LRERLENLRK KPENIPQPIS |
| 61 | NTSRANLNKL LTDYTEMKKA ILHVYWEEFQ KDPVGLMSRV |
| | AQPAPKNIDQ RKLIPVKDGN |
| 121 | ERLTSSGFAC SQCCQPLYVY KLEQVNDKGK PHTNYFGRCN |
| | VSEHERLILL SPHKPEANDE |
| 181 | LVTYSLGKFG QRALDFYSIH VTRESNHPVK PLEQIGGNSC |
| | ASGPVGKALS DACMGAVASF |
| 241 | LTKYQDIILE HQKVIKKNEK RLANLKDIAS ANGLAFPKIT |
| | LPPQPHTKEG IEAYNNVVAQ |
| 301 | IVIWVNLNLW QKLKIGRDEA KPLQRLKGFP SFPLVERQAN |
| | EVDWWDMVCN VKKLINEKKE |
| 361 | DGKVFWQNLA GYKRQEALLP YLSSEEDRKK GKKFARYQFG |
| | DLLLHLEKKH GEDWGKVYDE |
| 421 | AWERIDKKVE GLSKHIKLEE ERRSEDAQSK AALTDWLRAK |
| | ASFVIEGLKE ADKDEFCRCE |
| 481 | LKLQKWYGDL RGKPFAIEAE NSILDISGFS KQYNCAFIWQ |
| | KDGVKKLNLY LIINYFKGGK |
| 541 | LRFKKIKPEA FEANRFYTVI NKKSGEIVPM EVNFNFDDPN |
| | LIILPLAFGK RQGREFIWND |
| 601 | LLSLETGSLK LANGRVIEKT LYNRRTRQDE PALFVALTFE |
| | RREVLDSSNI KPMNLIGIDR |
| 661 | GENIPAVIAL TDPEGCPLSR FKDSLGNPTH ILRIGESYKE |
| | KQRTIQAAKE VEQRRAGGYS |
| 721 | RKYASKAKNL ADDMVRNTAR DLLYYAVTQD AMLIFENLSR |
| | GFGRQGKRTF MAERQYTRME |
| 781 | DWLTAKLAYE GLPSKTYLSK TLAQYTSKTC SNCGFTITSA |
| | DYDRVLEKLK KTATGWMTTI |
| 841 | NGKELKVEGQ ITYYNRYKRQ NVVKDLSVEL DRLSEESVNN |
| | DISSWTKGRS GEALSLLKKR |
| 901 | FSHRPVQEKF VCLNCGFETH ADEQAALNIA RSWLFLRSQE |
| | YKKYQTNKTT GNTDKRAFVE |
| 961 | TWQSFYRKKLKEVWKPAV. |

In some embodiments, the CasX protein comprises the sequence of SEQ ID NO: 2, or at least 60% similarity thereto. In some embodiments, the CasX protein comprises the sequence of SEQ ID NO: 2, or at least 80% similarity thereto. In some embodiments, the CasX protein comprises the sequence of SEQ ID NO: 2, or at least 90% similarity thereto. In some embodiments, the CasX protein comprises the sequence of SEQ ID NO: 2, or at least 95% similarity thereto. In some embodiments, the CasX protein consists of the sequence of SEQ ID NO: 2. In some embodiments, the CasX protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO: 2. These mutations can be insertions, deletions, amino acid substitutions, or any combinations thereof.

In some cases, a reference CasX protein is isolated or derived from Candidatus sungbacteria. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of

(SEQ ID NO: 3)

| | |
|---|---|
| 1 | MDNANKPSTK SLVNTTRISD HFGVTPGQVT RVFSFGIIPT |
| | KRQYAIIERW FAAVEAARER |
| 61 | LYGMLYAHFQ ENPPAYLKEK FSYETFFKGR PVLNGLRDID |
| | PTIMTSAVFT ALRHKAEGAM |
| 121 | AAFHTNHRRL FEEARKKMRE YAECLKANEA LLRGAADIDW |
| | DKIVNALRTR LNTCLAPEYD |
| 181 | AVIADFGALC AFRALIAETN ALKGAYNHAL NQMLPALVKV |
| | DEPEEAEESP RLRFFNGRIN |
| 241 | DLPKFPVAER ETPPDTETII RQLEDMARVI PDTAEILGYI |
| | HRIRHKAARR KPGSAVPLPQ |
| 301 | RVALYCAIRM ERNPEEDPST VAGHFLGEID RVCEKRRQGL |
| | VRTPFDSQIR ARYMDIISFR |
| 361 | ATLAHPDRWT EIQFLRSNAA SRRVRAETIS APFEGFSWTS |
| | NRTNPAPQYG MALAKDANAP |
| 421 | ADAPELCICL SPSSAAFSVR EKGGDLIYMR PTGGRRGKDN |
| | PGKEITWVPG SFDEYPASGV |
| 481 | ALKLRLYFGR SQARRMLTNK TWGLLSDNPR VFAANAELVG |
| | KKRNPQDRWK LFFHMVISGP |
| 541 | PPVEYLDFSS DVRSRARTVI GINRGEVNPL AYAVVSVEDG |
| | QVLEEGLLGK KEYIDQLIET |
| 601 | RRRISEYQSR EQTPPRDLRQ RVRHLQDTVL GSARAKIHSL |
| | IAFWKGILAI ERLDDQFHGR |
| 661 | EQKIIPKKTY LANKTGFMNA LSFSGAVRVD KKGNPWGGMI |
| | EIYPGGISRT CTQCGTVWLA |
| 721 | RRPKNPGHRD AMVVIPDIVD DAAATGFDNV DCDAGTVDYG |
| | ELFTLSREWV RLTPRYSRVM |
| 781 | RGTLGDLERA IRQGDDRKSR QMLELALEPQ PQWGQFFCHR |
| | CGFNGQSDVL AATNLARRAI |
| 841 | SLIRRLPDTD TPPTP. |

In some embodiments, the CasX protein comprises the sequence of SEQ ID NO: 3, or at least 60% similarity thereto. In some embodiments, the CasX protein comprises the sequence of SEQ ID NO: 3, or at least 80% similarity thereto. In some embodiments, the CasX protein comprises the sequence of SEQ ID NO: 3, or at least 90% similarity thereto. In some embodiments, the CasX protein comprises the sequence of SEQ ID NO: 3, or at least 95% similarity thereto. In some embodiments, the CasX protein consists of the sequence of SEQ ID NO: 3. In some embodiments, the CasX protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO: 3. These mutations can be insertions, deletions, amino acid substitutions, or any combinations thereof.

h. CasX Variant Proteins

The present disclosure provides variants of a reference CasX protein (interchangeably referred to herein as “CasX variant” or “CasX variant protein”), wherein the CasX variants comprise at least one modification in at least one domain of the reference CasX protein, including the sequences of SEQ ID NOS:1-3. In some embodiments, the CasX variant exhibits at least one improved characteristic compared to the reference CasX protein. All variants that improve one or more functions or characteristics of the CasX variant protein when compared to a reference CasX protein described herein are envisaged as being within the scope of the disclosure. In some embodiments, the modification is a mutation in one or more amino acids of the reference CasX. In other embodiments, the modification is a substitution of one or more domains of the reference CasX with one or more domains from a different CasX. In some embodiments, insertion includes the insertion of a part or all of a domain from a different CasX protein. Mutations can occur in any one or more domains of the reference CasX protein, and may include, for example, deletion of part or all of one or more domains, or one or more amino acid substitutions, deletions, or insertions in any domain of the reference CasX protein. The domains of CasX proteins include the non-target strand binding (NTSB) domain, the target strand loading (TSL) domain, the helical I domain, the helical II domain, the oligonucleotide binding domain (OBD), and the RuvC DNA cleavage domain. Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence.

In some embodiments, the CasX variant protein comprises at least one modification in at least each of two domains of the reference CasX protein, including the sequences of SEQ ID NOS:1-3. In some embodiments, the CasX variant protein comprises at least one modification in at least 2 domains, in at least 3 domains, at least 4 domains or at least 5 domains of the reference CasX protein. In some embodiments, the CasX variant protein comprises two or more modifications in at least one domain of the reference CasX protein. In some embodiments, the CasX variant protein comprises at least two modifications in at least one domain of the reference CasX protein, at least three modifications in at least one domain of the reference CasX protein or at least four modifications in at least one domain of the reference CasX protein. In some embodiments, wherein the CasX variant comprises two or more modifications compared to a reference CasX protein, each modification is made in a domain independently selected from the group consisting of a NTSBD, TSLD, Helical I domain, Helical II domain, OBD, and RuvC DNA cleavage domain.

In some embodiments, the at least one modification of the CasX variant protein comprises a deletion of at least a portion of one domain of the reference CasX protein, including the sequences of SEQ ID NOS:1-3. In some embodiments, the deletion is in the NTSBD, TSLD, Helical I domain, Helical II domain, OBD, or RuvC DNA cleavage domain.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping. In some embodiments, the CasX variants are designed, for example by selecting one or more desired mutations in a reference CasX. In certain embodiments, the activity of a reference CasX protein is used as a benchmark against which the activity of one or more CasX variants are compared, thereby measuring improvements in function of the CasX variants. Exemplary improvements of CasX variants include, but are not limited to, improved folding of the variant, improved binding affinity to the gNA, improved binding affinity to the target DNA, altered binding affinity to one or more PAM sequences, improved unwinding of the target DNA, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved protein stability, improved protein:gNA complex stability, improved protein solubility, improved protein:gNA complex solubility, improved protein yield, improved protein expression, and improved fusion characteristics, as described more fully, below.

In some embodiments of the CasX variants described herein, the at least one modification comprises: (a) a substitution of 1 to 100 consecutive or non-consecutive amino acids in the CasX variant compared to a reference CasX of SEQ ID NO:1, SEQ ID NO:2, or SEQ ID NO:3; (b) a deletion of 1 to 100 consecutive or non-consecutive amino acids in the CasX variant compared to a reference CasX; (c) an insertion of 1 to 100 consecutive or non-consecutive amino acids in the CasX compared to a reference CasX; or (d) any combination of (a)-(c). In some embodiments, the at least one modification comprises: (a) a substitution of 5-10 consecutive or non-consecutive amino acids in the CasX variant compared to a reference CasX of SEQ ID NO:1, SEQ ID NO:2, or SEQ ID NO:3; (b) a deletion of 1-5 consecutive or non-consecutive amino acids in the CasX variant compared to a reference CasX; (c) an insertion of 1-5 consecutive or non-consecutive amino acids in the CasX compared to a reference CasX; or (d) any combination of (a)-(c).

In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO:1, SEQ ID NO:2, or SEQ ID NO:3. These mutations can be insertions, deletions, amino acid substitutions, or any combinations thereof.

In some embodiments, the CasX variant protein comprises at least one amino acid substitution in at least one domain of a reference CasX protein. In some embodiments, the CasX variant protein comprises at least about 1-4 amino acid substitutions, 1-10 amino acid substitutions, 1-20 amino acid substitutions, 1-30 amino acid substitutions, 1-40 amino acid substitutions, 1-50 amino acid substitutions, 1-60 amino acid substitutions, 1-70 amino acid substitutions, 1-80 amino acid substitutions, 1-90 amino acid substitutions, 1-100 amino acid substitutions, 2-10 amino acid substitutions, 2-20 amino acid substitutions, 2-30 amino acid substitutions, 3-10 amino acid substitutions, 3-20 amino acid substitutions, 3-30 amino acid substitutions, 4-10 amino acid substitutions, 4-20 amino acid substitutions, 3-300 amino acid substitutions, 5-10 amino acid substitutions, 5-20 amino acid substitutions, 5-30 amino acid substitutions, 10-50 amino acid substitutions, or 20-50 amino acid substitutions, relative to a reference CasX protein, which can be consecutive or non-consecutive, or in different domains. As used herein “consecutive amino acids” refer to amino acids that are contiguous in the primary sequence of a polypeptide. In some embodiments, the CasX variant protein comprises at least about 100 or more amino acid substitutions relative to a reference CasX protein. In some embodiments, the amino acid substitutions are conservative substitutions. In other embodiments, the substitutions are non-conservative; e.g., a polar amino acid is substituted for a non-polar amino acid, or vice versa.

Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

In some embodiments, a CasX variant protein comprises at least one amino acid deletion relative to a reference CasX protein. In some embodiments, a CasX variant protein comprises a deletion of 1-4 amino acids, 1-10 amino acids, 1-20 amino acids, 1-30 amino acids, 1-40 amino acids, 1-50 amino acids, 1-60 amino acids, 1-70 amino acids, 1-80 amino acids, 1-90 amino acids, 1-100 amino acids, 2-10 amino acids, 2-20 amino acids, 2-30 amino acids, 3-10 amino acids, 3-20 amino acids, 3-30 amino acids, 4-10 amino acids, 4-20 amino acids, 3-300 amino acids, 5-10 amino acids, 5-20 amino acids, 5-30 amino acids, 10-50 amino acids or 20-50 amino acids relative to a reference CasX protein. In some embodiments, a CasX protein comprises a deletion of at least about 100 consecutive amino acids relative to a reference CasX protein. In some embodiments, a CasX variant protein comprises a deletion of at least 1, 2, 3, 4, 5, 6, 7, 8, 9, 10, 20, 30, 40, 50 or 100 consecutive amino acids relative to a reference CasX protein. In some embodiments, a CasX variant protein comprises a deletion of 1, 2, 3, 4, 5, 6, 7, 8, 9 or 10 consecutive amino acids.

In some embodiments, a CasX variant protein comprises two or more deletions relative to a reference CasX protein, and the two or more deletions are not consecutive amino acids. For example, a first deletion may be in a first domain of the reference CasX protein, and a second deletion may be in a second domain of the reference CasX protein. In some embodiments, a CasX variant protein comprises 2, 3, 4, 5, 6, 7, 8, 9, 10, 11, 12, 13, 14, 15, 16, 17, 18, 19 or 20 non-consecutive deletions relative to a reference CasX protein. In some embodiments, a CasX variant protein comprises at least 20 non-consecutive deletions relative to a reference CasX protein. Each non-consecutive deletion may be of any length of amino acids described herein, e.g., 1-4 amino acids, 1-10 amino acids, and the like.

In some embodiments, the CasX variant protein comprises one or more amino acid insertions relative to the sequence of SEQ ID NOS:1, 2, or 3. In some embodiments, a CasX variant protein comprises an insertion of 1 amino acid, an insertion of 2-3 consecutive or non-consecutive amino acids, 2-4 consecutive or non-consecutive amino acids, 2-5 consecutive or non-consecutive amino acids, 2-6 consecutive or non-consecutive amino acids, 2-7 consecutive or non-consecutive amino acids, 2-8 consecutive or non-consecutive amino acids, 2-9 consecutive or non-consecutive amino acids, 2-10 consecutive or non-consecutive amino acids, 2-20 consecutive or non-consecutive amino acids, 2-30 consecutive or non-consecutive amino acids, 2-40 consecutive or non-consecutive amino acids, 2-50 consecutive or non-consecutive amino acids, 2-60 consecutive or non-consecutive amino acids, 2-70 consecutive or non-consecutive amino acids, 2-80 consecutive or non-consecutive amino acids, 2-90 consecutive or non-consecutive amino acids, 2-100 consecutive or non-consecutive amino acids, 3-10 consecutive or non-consecutive amino acids, 3-20 consecutive or non-consecutive amino acids, 3-30 consecutive or non-consecutive amino acids, 4-10 consecutive or non-consecutive amino acids, 4-20 consecutive or non-consecutive amino acids, 3-300 consecutive or non-consecutive amino acids, 5-10 consecutive or non-consecutive amino acids, 5-20 consecutive or non-consecutive amino acids, 5-30 consecutive or non-consecutive amino acids, 10-50 consecutive or non-consecutive amino acids or 20-50 consecutive or non-consecutive amino acids relative to a reference CasX protein. In some embodiments, the CasX variant protein comprises an insertion of 2, 3, 4, 5, 6, 7, 8, 9, 10, 11, 12, 13, 14, 15, 16, 17, 18, 19 or 20 consecutive or non-consecutive amino acids. In some embodiments, a CasX variant protein comprises an insertion of at least about 100 consecutive or non-consecutive amino acids. Any amino acid, or combination of amino acids, can be inserted in the insertions described herein to generate a CasX variant protein.

Any permutation of the substitution, insertion and deletion embodiments described herein can be combined to generate a CasX variant protein of the disclosure. For example, a CasX variant protein can comprise at least one substitution and at least one deletion relative to a reference CasX protein sequence, at least one substitution and at least one insertion relative to a reference CasX protein sequence, at least one insertion and at least one deletion relative to a reference CasX protein sequence, or at least one substitution, one insertion and one deletion relative to a reference CasX protein sequence.

In some embodiments, the CasX variant protein has at least about 60% sequence similarity to SEQ ID NO:2 or a portion thereof. In some embodiments, the CasX variant protein comprises a substitution of Y789T of SEQ ID NO:2, a deletion of P793 of SEQ ID NO:2, a substitution of Y789D of SEQ ID NO:2, a substitution of T72S of SEQ ID NO:2, a substitution of I546V of SEQ ID NO:2, a substitution of E552A of SEQ ID NO:2, a substitution of A636D of SEQ ID NO:2, a substitution of F536S of SEQ ID NO:2, a substitution of A708K of SEQ ID NO:2, a substitution of Y797L of SEQ ID NO:2, a substitution of L792G SEQ ID NO:2, a substitution of A739V of SEQ ID NO:2, a substitution of G791M of SEQ ID NO:2, an insertion of A at position 661 of SEQ ID NO:2, a substitution of A788W of SEQ ID NO:2, a substitution of K390R of SEQ ID NO:2, a substitution of A751S of SEQ ID NO:2, a substitution of E385A of SEQ ID NO:2, an insertion of P at position 696 of SEQ ID NO:2, an insertion of M at position 773 of SEQ ID NO:2, a substitution of G695H of SEQ ID NO:2, an insertion of AS at position 793 of SEQ ID NO:2, an insertion of AS at position 795 of SEQ ID NO:2, a substitution of C477R of SEQ ID NO:2, a substitution of C477K of SEQ ID NO:2, a substitution of C479A of SEQ ID NO:2, a substitution of C479L of SEQ ID NO:2, a substitution of I55F of SEQ ID NO:2, a substitution of K210R of SEQ ID NO:2, a substitution of C233S of SEQ ID NO:2, a substitution of D231N of SEQ ID NO:2, a substitution of Q338E of SEQ ID NO:2, a substitution of Q338R of SEQ ID NO:2, a substitution of L379R of SEQ ID NO:2, a substitution of K390R of SEQ ID NO:2, a substitution of L481Q of SEQ ID NO:2, a substitution of F495S of SEQ ID NO:2, a substitution of D600N of SEQ ID NO:2, a substitution of T886K of SEQ ID NO:2, a substitution of A739V of SEQ ID NO:2, a substitution of K460N of SEQ ID NO:2, a substitution of I199F of SEQ ID NO:2, a substitution of G492P of SEQ ID NO:2, a substitution of T153I of SEQ ID NO:2, a substitution of R591I of SEQ ID NO:2, an insertion of AS at position 795 of SEQ ID NO:2, an insertion of AS at position 796 of SEQ ID NO:2, an insertion of L at position 889 of SEQ ID NO:2, a substitution of E121D of SEQ ID NO:2, a substitution of S270W of SEQ ID NO:2, a substitution of E712Q of SEQ ID NO:2, a substitution of K942Q of SEQ ID NO:2, a substitution of E552K of SEQ ID NO:2, a substitution of K25Q of SEQ ID NO:2, a substitution of N47D of SEQ ID NO:2, an insertion of T at position 696 of SEQ ID NO:2, a substitution of L685I of SEQ ID NO:2, a substitution of N880D of SEQ ID NO:2, a substitution of Q102R of SEQ ID NO:2, a substitution of M734K of SEQ ID NO:2, a substitution of A724S of SEQ ID NO:2, a substitution of T704K of SEQ ID NO:2, a substitution of P224K of SEQ ID NO:2, a substitution of K25R of SEQ ID NO:2, a substitution of M29E of SEQ ID NO:2, a substitution of H152D of SEQ ID NO:2, a substitution of S219R of SEQ ID NO:2, a substitution of E475K of SEQ ID NO:2, a substitution of G226R of SEQ ID NO:2, a substitution of A377K of SEQ ID NO:2, a substitution of E480K of SEQ ID NO:2, a substitution of K416E of SEQ ID NO:2, a substitution of H164R of SEQ ID NO:2, a substitution of K767R of SEQ ID NO:2, a substitution of I7F of SEQ ID NO:2, a substitution of M29R of SEQ ID NO:2, a substitution of H435R of SEQ ID NO:2, a substitution of E385Q of SEQ ID NO:2, a substitution of E385K of SEQ ID NO:2, a substitution of I279F of SEQ ID NO:2, a substitution of D489S of SEQ ID NO:2, a substitution of D732N of SEQ ID NO:2, a substitution of A739T of SEQ ID NO:2, a substitution of W885R of SEQ ID NO:2, a substitution of E53K of SEQ ID NO:2, a substitution of A238T of SEQ ID NO:2, a substitution of P283Q of SEQ ID NO:2, a substitution of E292K of SEQ ID NO:2, a substitution of Q628E of SEQ ID NO:2, a substitution of R388Q of SEQ ID NO:2, a substitution of G791M of SEQ ID NO:2, a substitution of L792K of SEQ ID NO:2, a substitution of L792E of SEQ ID NO:2, a substitution of M779N of SEQ ID NO:2, a substitution of G27D of SEQ ID NO:2, a substitution of K955R of SEQ ID NO:2, a substitution of S867R of SEQ ID NO:2, a substitution of R693I of SEQ ID NO:2, a substitution of F189Y of SEQ ID NO:2, a substitution of V635M of SEQ ID NO:2, a substitution of F399L of SEQ ID NO:2, a substitution of E498K of SEQ ID NO:2, a substitution of E386R of SEQ ID NO:2, a substitution of V254G of SEQ ID NO:2, a substitution of P793S of SEQ ID NO:2, a substitution of K188E of SEQ ID NO:2, a substitution of QT945KI of SEQ ID NO:2, a substitution of T620P of SEQ ID NO:2, a substitution of T946P of SEQ ID NO:2, a substitution of TT949PP of SEQ ID NO:2, a substitution of N952T of SEQ ID NO:2, a substitution of K682E of SEQ ID NO:2, a substitution of K975R of SEQ ID NO:2, a substitution of L212P of SEQ ID NO:2, a substitution of E292R of SEQ ID NO:2, a substitution of 1303K of SEQ ID NO:2, a substitution of C349E of SEQ ID NO:2, a substitution of E385P of SEQ ID NO:2, a substitution of E386N of SEQ ID NO:2, a substitution of D387K of SEQ ID NO:2, a substitution of L404K of SEQ ID NO:2, a substitution of E466H of SEQ ID NO:2, a substitution of C477Q of SEQ ID NO:2, a substitution of C477H of SEQ ID NO:2, a substitution of C479A of SEQ ID NO:2, a substitution of D659H of SEQ ID NO:2, a substitution of T806V of SEQ ID NO:2, a substitution of K808S of SEQ ID NO:2, an insertion of AS at position 797 of SEQ ID NO:2, a substitution of V959M of SEQ ID NO:2, a substitution of K975Q of SEQ ID NO:2, a substitution of W974G of SEQ ID NO:2, a substitution of A708Q of SEQ ID NO:2, a substitution of V711K of SEQ ID NO:2, a substitution of D733T of SEQ ID NO:2, a substitution of L742W of SEQ ID NO:2, a substitution of V747K of SEQ ID NO:2, a substitution of F755M of SEQ ID NO:2, a substitution of M771A of SEQ ID NO:2, a substitution of M771Q of SEQ ID NO:2, a substitution of W782Q of SEQ ID NO:2, a substitution of G791F, of SEQ ID NO:2 a substitution of L792D of SEQ ID NO:2, a substitution of L792K of SEQ ID NO:2, a substitution of P793Q of SEQ ID NO:2, a substitution of P793G of SEQ ID NO:2, a substitution of Q804A of SEQ ID NO:2, a substitution of Y966N of SEQ ID NO:2, a substitution of Y723N of SEQ ID NO:2, a substitution of Y857R of SEQ ID NO:2, a substitution of S890R of SEQ ID NO:2, a substitution of S932M of SEQ ID NO:2, a substitution of L897M of SEQ ID NO:2, a substitution of R624G of SEQ ID NO:2, a substitution of S603G of SEQ ID NO:2, a substitution of N737S of SEQ ID NO:2, a substitution of L307K of SEQ ID NO:2, a substitution of 1658V of SEQ ID NO:2, an insertion of PT at position 688 of SEQ ID NO:2, an insertion of SA at position 794 of SEQ ID NO:2, a substitution of S877R of SEQ ID NO:2, a substitution of N580T of SEQ ID NO:2, a substitution of V335G of SEQ ID NO:2, a substitution of T620S of SEQ ID NO:2, a substitution of W345G of SEQ ID NO:2, a substitution of T280S of SEQ ID NO:2, a substitution of L406P of SEQ ID NO:2, a substitution of A612D of SEQ ID NO:2, a substitution of A751S of SEQ ID NO:2, a substitution of E386R of SEQ ID NO:2, a substitution of V351M of SEQ ID NO:2, a substitution of K210N of SEQ ID NO:2, a substitution of D40A of SEQ ID NO:2, a substitution of E773G of SEQ ID NO:2, a substitution of H207L of SEQ ID NO:2, a substitution of T62A SEQ ID NO:2, a substitution of T287P of SEQ ID NO:2, a substitution of T832A of SEQ ID NO:2, a substitution of A893S of SEQ ID NO:2, an insertion of V at position 14 of SEQ ID NO:2, an insertion of AG at position 13 of SEQ ID NO:2, a substitution of RI V of SEQ ID NO:2, a substitution of R12N of SEQ ID NO:2, a substitution of R13H of SEQ ID NO:2, an insertion of Y at position 13 of SEQ ID NO:2, a substitution of R12L of SEQ ID NO:2, an insertion of Q at position 13 of SEQ ID NO:2, an substitution of V15S of SEQ ID NO:2, an insertion of D at position 17 of SEQ ID NO:2 or a combination thereof.

In some embodiments, the CasX variant comprises at least one modification in the NTSB domain.

In some embodiments, the CasX variant comprises at least one modification in the TSL domain. In some embodiments, the at least one modification in the TSL domain comprises an amino acid substitution of one or more of amino acids Y857, S890, or S932 of SEQ ID NO:2.

In some embodiments, the CasX variant comprises at least one modification in the helical I domain. In some embodiments, the at least one modification in the helical I domain comprises an amino acid substitution of one or more of amino acids S219, L249, E259, Q252, E292, L307, or D318 of SEQ ID NO:2.

In some embodiments, the CasX variant comprises at least one modification in the helical II domain. In some embodiments, the at least one modification in the helical II domain comprises an amino acid substitution of one or more of amino acids D361, L379, E385, E386, D387, F399, L404, R458, C477, or D489 of SEQ ID NO:2.

In some embodiments, the CasX variant comprises at least one modification in the OBD domain. In some embodiments, the at least one modification in the OBD comprises an amino acid substitution of one or more of amino acids F536, E552, T620, or 1658 of SEQ ID NO:2.

In some embodiments, the CasX variant comprises at least one modification in the RuvC DNA cleavage domain. In some embodiments, the at least one modification in the RuvC DNA cleavage domain comprises an amino acid substitution of one or more of amino acids K682, G695, A708, V711, D732, A739, D733, L742, V747, F755, M771, M779, W782, A788, G791, L792, P793, Y797, M799, Q804, 5819, or Y857 or a deletion of amino acid P793 of SEQ ID NO:2.

In some embodiments, the CasX variant comprises at least one modification compared to the reference CasX sequence of SEQ ID NO:2 is selected from one or more of: (a) an amino acid substitution of L379R; (b) an amino acid substitution of A708K; (c) an amino acid substitution of T620P; (d) an amino acid substitution of E385P; (e) an amino acid substitution of Y857R; (f) an amino acid substitution of I658V; (g) an amino acid substitution of F399L; (h) an amino acid substitution of Q252K; (i) an amino acid substitution of L404K; and (j) an amino acid deletion of P793.

In some embodiments, a CasX variant comprises at least two amino acid changes to the sequence of a reference CasX variant protein selected from the group consisting of: a substitution of Y789T of SEQ ID NO:2, a deletion of P793 of SEQ ID NO:2, a substitution of Y789D of SEQ ID NO:2, a substitution of T72S of SEQ ID NO:2, a substitution of I546V of SEQ ID NO:2, a substitution of E552A of SEQ ID NO:2, a substitution of A636D of SEQ ID NO:2, a substitution of F536S of SEQ ID NO:2, a substitution of A708K of SEQ ID NO:2, a substitution of Y797L of SEQ ID NO:2, a substitution of L792G SEQ ID NO:2, a substitution of A739V of SEQ ID NO:2, a substitution of G791M of SEQ ID NO:2, an insertion of A at position 661 of SEQ ID NO:2, a substitution of A788W of SEQ ID NO:2, a substitution of K390R of SEQ ID NO:2, a substitution of A751S of SEQ ID NO:2, a substitution of E385A of SEQ ID NO:2, an insertion of P at position 696 of SEQ ID NO:2, an insertion of M at position 773 of SEQ ID NO:2, a substitution of G695H of SEQ ID NO:2, an insertion of AS at position 793 of SEQ ID NO:2, an insertion of AS at position 795 of SEQ ID NO:2, a substitution of C477R of SEQ ID NO:2, a substitution of C477K of SEQ ID NO:2, a substitution of C479A of SEQ ID NO:2, a substitution of C479L of SEQ ID NO:2, a substitution of I55F of SEQ ID NO:2, a substitution of K210R of SEQ ID NO:2, a substitution of C233S of SEQ ID NO:2, a substitution of D231N of SEQ ID NO:2, a substitution of Q338E of SEQ ID NO:2, a substitution of Q338R of SEQ ID NO:2, a substitution of L379R of SEQ ID NO:2, a substitution of K390R of SEQ ID NO:2, a substitution of L481Q of SEQ ID NO:2, a substitution of F495S of SEQ ID NO:2, a substitution of D600N of SEQ ID NO:2, a substitution of T886K of SEQ ID NO:2, a substitution of A739V of SEQ ID NO:2, a substitution of K460N of SEQ ID NO:2, a substitution of I199F of SEQ ID NO:2, a substitution of G492P of SEQ ID NO:2, a substitution of T153I of SEQ ID NO:2, a substitution of R591I of SEQ ID NO:2, an insertion of AS at position 795 of SEQ ID NO:2, an insertion of AS at position 796 of SEQ ID NO:2, an insertion of L at position 889 of SEQ ID NO:2, a substitution of E121D of SEQ ID NO:2, a substitution of S270W of SEQ ID NO:2, a substitution of E712Q of SEQ ID NO:2, a substitution of K942Q of SEQ ID NO:2, a substitution of E552K of SEQ ID NO:2, a substitution of K25Q of SEQ ID NO:2, a substitution of N47D of SEQ ID NO:2, an insertion of T at position 696 of SEQ ID NO:2, a substitution of L685I of SEQ ID NO:2, a substitution of N880D of SEQ ID NO:2, a substitution of Q102R of SEQ ID NO:2, a substitution of M734K of SEQ ID NO:2, a substitution of A724S of SEQ ID NO:2, a substitution of T704K of SEQ ID NO:2, a substitution of P224K of SEQ ID NO:2, a substitution of K25R of SEQ ID NO:2, a substitution of M29E of SEQ ID NO:2, a substitution of H152D of SEQ ID NO:2, a substitution of S219R of SEQ ID NO:2, a substitution of E475K of SEQ ID NO:2, a substitution of G226R of SEQ ID NO:2, a substitution of A377K of SEQ ID NO:2, a substitution of E480K of SEQ ID NO:2, a substitution of K416E of SEQ ID NO:2, a substitution of H164R of SEQ ID NO:2, a substitution of K767R of SEQ ID NO:2, a substitution of I7F of SEQ ID NO:2, a substitution of M29R of SEQ ID NO:2, a substitution of H435R of SEQ ID NO:2, a substitution of E385Q of SEQ ID NO:2, a substitution of E385K of SEQ ID NO:2, a substitution of I279F of SEQ ID NO:2, a substitution of D489S of SEQ ID NO:2, a substitution of D732N of SEQ ID NO:2, a substitution of A739T of SEQ ID NO:2, a substitution of W885R of SEQ ID NO:2, a substitution of E53K of SEQ ID NO:2, a substitution of A238T of SEQ ID NO:2, a substitution of P283Q of SEQ ID NO:2, a substitution of E292K of SEQ ID NO:2, a substitution of Q628E of SEQ ID NO:2, a substitution of R388Q of SEQ ID NO:2, a substitution of G791M of SEQ ID NO:2, a substitution of L792K of SEQ ID NO:2, a substitution of L792E of SEQ ID NO:2, a substitution of M779N of SEQ ID NO:2, a substitution of G27D of SEQ ID NO:2, a substitution of K955R of SEQ ID NO:2, a substitution of S867R of SEQ ID NO:2, a substitution of R693I of SEQ ID NO:2, a substitution of F189Y of SEQ ID NO:2, a substitution of V635M of SEQ ID NO:2, a substitution of F399L of SEQ ID NO:2, a substitution of E498K of SEQ ID NO:2, a substitution of E386R of SEQ ID NO:2, a substitution of V254G of SEQ ID NO:2, a substitution of P793S of SEQ ID NO:2, a substitution of K188E of SEQ ID NO:2, a substitution of QT945KI of SEQ ID NO:2, a substitution of T620P of SEQ ID NO:2, a substitution of T946P of SEQ ID NO:2, a substitution of TT949PP of SEQ ID NO:2, a substitution of N952T of SEQ ID NO:2, a substitution of K682E of SEQ ID NO:2, a substitution of K975R of SEQ ID NO:2, a substitution of L212P of SEQ ID NO:2, a substitution of E292R of SEQ ID NO:2, a substitution of 1303K of SEQ ID NO:2, a substitution of C349E of SEQ ID NO:2, a substitution of E385P of SEQ ID NO:2, a substitution of E386N of SEQ ID NO:2, a substitution of D387K of SEQ ID NO:2, a substitution of L404K of SEQ ID NO:2, a substitution of E466H of SEQ ID NO:2, a substitution of C477Q of SEQ ID NO:2, a substitution of C477H of SEQ ID NO:2, a substitution of C479A of SEQ ID NO:2, a substitution of D659H of SEQ ID NO:2, a substitution of T806V of SEQ ID NO:2, a substitution of K808S of SEQ ID NO:2, an insertion of AS at position 797 of SEQ ID NO:2, a substitution of V959M of SEQ ID NO:2, a substitution of K975Q of SEQ ID NO:2, a substitution of W974G of SEQ ID NO:2, a substitution of A708Q of SEQ ID NO:2, a substitution of V711K of SEQ ID NO:2, a substitution of D733T of SEQ ID NO:2, a substitution of L742W of SEQ ID NO:2, a substitution of V747K of SEQ ID NO:2, a substitution of F755M of SEQ ID NO:2, a substitution of M771A of SEQ ID NO:2, a substitution of M771Q of SEQ ID NO:2, a substitution of W782Q of SEQ ID NO:2, a substitution of G791F, of SEQ ID NO:2 a substitution of L792D of SEQ ID NO:2, a substitution of L792K of SEQ ID NO:2, a substitution of P793Q of SEQ ID NO:2, a substitution of P793G of SEQ ID NO:2, a substitution of Q804A of SEQ ID NO:2, a substitution of Y966N of SEQ ID NO:2, a substitution of Y723N of SEQ ID NO:2, a substitution of Y857R of SEQ ID NO:2, a substitution of S890R of SEQ ID NO:2, a substitution of S932M of SEQ ID NO:2, a substitution of L897M of SEQ ID NO:2, a substitution of R624G of SEQ ID NO:2, a substitution of S603G of SEQ ID NO:2, a substitution of N737S of SEQ ID NO:2, a substitution of L307K of SEQ ID NO:2, a substitution of I658V of SEQ ID NO:2, an insertion of PT at position 688 of SEQ ID NO:2, an insertion of SA at position 794 of SEQ ID NO:2, a substitution of S877R of SEQ ID NO:2, a substitution of N580T of SEQ ID NO:2, a substitution of V335G of SEQ ID NO:2, a substitution of T620S of SEQ ID NO:2, a substitution of W345G of SEQ ID NO:2, a substitution of T280S of SEQ ID NO:2, a substitution of L406P of SEQ ID NO:2, a substitution of A612D of SEQ ID NO:2, a substitution of A751S of SEQ ID NO:2, a substitution of E386R of SEQ ID NO:2, a substitution of V351M of SEQ ID NO:2, a substitution of K210N of SEQ ID NO:2, a substitution of D40A of SEQ ID NO:2, a substitution of E773G of SEQ ID NO:2, a substitution of H207L of SEQ ID NO:2, a substitution of T62A SEQ ID NO:2, a substitution of T287P of SEQ ID NO:2, a substitution of T832A of SEQ ID NO:2, a substitution of A893S of SEQ ID NO:2, an insertion of V at position 14 of SEQ ID NO:2, an insertion of AG at position 13 of SEQ ID NO:2, a substitution of R11V of SEQ ID NO:2, a substitution of R12N of SEQ ID NO:2, a substitution of R13H of SEQ ID NO:2, an insertion of Y at position 13 of SEQ ID NO:2, a substitution of R12L of SEQ ID NO:2, an insertion of Q at position 13 of SEQ ID NO:2, an substitution of V15S of SEQ ID NO:2 and an insertion of D at position 17 of SEQ ID NO:2. In some embodiments, the at least two amino acid changes to a reference CasX protein are selected from the amino acid changes disclosed in the sequences of SEQ ID NOS: 49-150 as set forth in Table 4. In some embodiments, a CasX variant comprises any combination of the foregoing embodiments of this paragraph.

In some embodiments, a CasX variant protein comprises more than one substitution, insertion and/or deletion of a reference CasX protein amino acid sequence. In some embodiments, a CasX variant protein comprises a substitution of S794R and a substitution of Y797L of SEQ ID NO:2. In some embodiments, a CasX variant protein comprises a substitution of K416E and a substitution of A708K of SEQ ID NO:2. In some embodiments, a CasX variant protein comprises a substitution of A708K and a deletion of P793 of SEQ ID NO:2. In some embodiments, a CasX variant protein comprises a deletion of P793 and an insertion of AS at position 795 SEQ ID NO:2. In some embodiments, a CasX variant protein comprises a substitution of Q367K and a substitution of I425S of SEQ ID NO:2. In some embodiments, a CasX variant protein comprises a substitution of A708K, a deletion of P position 793 and a substitution A793V of SEQ ID NO:2. In some embodiments, a CasX variant protein comprises a substitution of Q338R and a substitution of A339E of SEQ ID NO:2. In some embodiments, a CasX variant protein comprises a substitution of Q338R and a substitution of A339K of SEQ ID NO:2. In some embodiments, a CasX variant protein comprises a substitution of S507G and a substitution of G508R of SEQ ID NO:2. In some embodiments, a CasX variant protein comprises a substitution of L379R, a substitution of A708K and a deletion of P at position 793 of SEQ ID NO:2. In some embodiments, a CasX variant protein comprises a substitution of C477K, a substitution of A708K and a deletion of P at position 793 of SEQ ID NO:2. In some embodiments, a CasX variant protein comprises a substitution of L379R, a substitution of C477K, a substitution of A708K and a deletion of P at position of 793 of SEQ ID NO:2. In some embodiments, a CasX variant protein comprises a substitution of L379R, a substitution of A708K, a deletion of P at position 793 and a substitution A739V of SEQ ID NO:2. In some embodiments, a CasX variant protein comprises a substitution of C477K, a substitution of A708K, a deletion of P at position 793 and a substitution of A739V of SEQ ID NO:2. In some embodiments, a CasX variant protein comprises a substitution of L379R, a substitution of C477K, a substitution of A708K, a deletion of P at position 793 and a substitution of A739V of SEQ ID NO:2. In some embodiments, a CasX variant protein comprises a substitution of L379R, a substitution of A708K, a deletion of P at position 793 and a substitution of M779N of SEQ ID NO:2. In some embodiments, a CasX variant protein comprises a substitution of L379R, a substitution of A708K, a deletion of P at position 793 and a substitution of M771N of SEQ ID NO:2. In some embodiments, a CasX variant protein comprises a substitution of L379R, a substitution of 708K, a deletion of P at position 793 and a substitution of D489S of SEQ ID NO:2. In some embodiments, a CasX variant protein comprises a substitution of L379R, a substitution of A708K, a deletion of P at position 793 and a substitution of A739T of SEQ ID NO:2. In some embodiments, a CasX variant protein comprises a substitution of L379R, a substitution of A708K, a deletion of P at position 793 and a substitution of D732N of SEQ ID NO:2. In some embodiments, a CasX variant protein comprises a substitution of L379R, a substitution of A708K, a deletion of P at position 793 and a substitution of G791M of SEQ ID NO:2. In some embodiments, a CasX variant protein comprises a substitution of L379R, a substitution of 708K, a deletion of P at position 793 and a substitution of Y797L of SEQ ID NO:2. In some embodiments, a CasX variant protein comprises a substitution of L379R, a substitution of C477K, a substitution of A708K, a deletion of P at position 793 and a substitution of M779N of SEQ ID NO:2. In some embodiments, a CasX variant protein comprises a substitution of L379R, a substitution of C477K, a substitution of A708K, a deletion of P at position 793 and a substitution of M771N of SEQ ID NO:2. In some embodiments, a CasX variant protein comprises a substitution of L379R, a substitution of C477K, a substitution of A708K, a deletion of P at position 793 and a substitution of D489S of SEQ ID NO:2. In some embodiments, a CasX variant protein comprises a substitution of L379R, a substitution of C477K, a substitution of A708K, a deletion of P at position 793 and a substitution of A739T of SEQ ID NO:2. In some embodiments, a CasX variant protein comprises a substitution of L379R, a substitution of C477K, a substitution of A708K, a deletion of P at position 793 and a substitution of D732N of SEQ ID NO:2. In some embodiments, a CasX variant protein comprises a substitution of L379R, a substitution of C477K, a substitution of A708K, a deletion of P at position 793 and a substitution of G791M of SEQ ID NO:2. In some embodiments, a CasX variant protein comprises a substitution of L379R, a substitution of C477K, a substitution of A708K, a deletion of P at position 793 and a substitution of Y797L of SEQ ID NO:2. In some embodiments, a CasX variant protein comprises a substitution of L379R, a substitution of C477K, a substitution of A708K, a deletion of P at position 793 and a substitution of T620P of SEQ ID NO:2. In some embodiments, a CasX variant protein comprises a substitution of A708K, a deletion of P at position 793 and a substitution of E386S of SEQ ID NO:2. In some embodiments, a CasX variant protein comprises a substitution of E386R, a substitution of F399L and a deletion of P at position 793 of SEQ ID NO:2. In some embodiments, a CasX variant protein comprises a substitution of R581I and A739V of SEQ ID NO:2. In some embodiments, a CasX variant comprises any combination of the foregoing embodiments of this paragraph.

In some embodiments, a CasX variant protein comprises more than one substitution, insertion and/or deletion of a reference CasX protein amino acid sequence. In some embodiments, a CasX variant protein comprises a substitution of A708K, a deletion of P at position 793 and a substitution of A739V of SEQ ID NO:2. In some embodiments, a CasX variant protein comprises a substitution of L379R, a substitution of A708K and a deletion of P at position 793 of SEQ ID NO:2. In some embodiments, a CasX variant protein comprises a substitution of C477K, a substitution of A708K and a deletion of P at position 793 of SEQ ID NO:2. In some embodiments, a CasX variant protein comprises a substitution of L379R, a substitution of C477K, a substitution of A708K and a deletion of P at position 793 of SEQ ID NO:2. In some embodiments, a CasX variant protein comprises a substitution of L379R, a substitution of A708K, a deletion of P at position 793 and a substitution of A739V of SEQ ID NO:2. In some embodiments, a CasX variant protein comprises a substitution of C477K, a substitution of A708K, a deletion of P at position 793 and a substitution of A739 of SEQ ID NO:2. In some embodiments, a CasX variant protein comprises a substitution of L379R, a substitution of C477K, a substitution of A708K, a deletion of P at position 793 and a substitution of A739V of SEQ ID NO:2. In some embodiments, a CasX variant protein comprises a substitution of L379R, a substitution of C477K, a substitution of A708K, a deletion of P at position 793 and a substitution of T620P of SEQ ID NO:2. In some embodiments, a CasX variant protein comprises a substitution of M771A of SEQ ID NO:2. In some embodiments, a CasX variant protein comprises a substitution of L379R, a substitution of A708K, a deletion of P at position 793 and a substitution of D732N of SEQ ID NO:2. In some embodiments, a CasX variant comprises any combination of the foregoing embodiments of this paragraph.

In some embodiments, a CasX variant protein comprises a substitution of W782Q of SEQ ID NO:2. In some embodiments, a CasX variant protein comprises a substitution of M771Q of SEQ ID NO:2. In some embodiments, a CasX variant protein comprises a substitution of R458I and a substitution of A739V of SEQ ID NO:2. In some embodiments, a CasX variant protein comprises a substitution of L379R, a substitution of A708K, a deletion of P at position 793 and a substitution of M771N of SEQ ID NO:2. In some embodiments, a CasX variant protein comprises a substitution of L379R, a substitution of A708K, a deletion of P at position 793 and a substitution of A739T of SEQ ID NO:2. In some embodiments, a CasX variant protein comprises a substitution of L379R, a substitution of C477K, a substitution of A708K, a deletion of P at position 793 and a substitution of D489S of SEQ ID NO:2. In some embodiments, a CasX variant protein comprises a substitution of L379R, a substitution of C477K, a substitution of A708K, a deletion of P at position 793 and a substitution of D732N of SEQ ID NO:2. In some embodiments, a CasX variant protein comprises a substitution of V711K of SEQ ID NO:2. In some embodiments, a CasX variant protein comprises a substitution of L379R, a substitution of C477K, a substitution of A708K, a deletion of P at position 793 and a substitution of Y797L of SEQ ID NO:2. In some embodiments, a CasX variant protein comprises a substitution of L379R, a substitution of A708K and a deletion of P at position 793 of SEQ ID NO:2. In some embodiments, a CasX variant protein comprises a substitution of L379R, a substitution of C477K, a substitution of A708K, a deletion of P at position 793 and a substitution of M771N of SEQ ID NO:2. In some embodiments, a CasX variant protein comprises a substitution of A708K, a substitution of P at position 793 and a substitution of E386S of SEQ ID NO:2. In some embodiments, a CasX variant protein comprises a substitution of L379R, a substitution of C477K, a substitution of A708K and a deletion of P at position 793 of SEQ ID NO:2. In some embodiments, a CasX variant protein comprises a substitution of L792D of SEQ ID NO:2. In some embodiments, a CasX variant protein comprises a substitution of G791F of SEQ ID NO:2. In some embodiments, a CasX variant protein comprises a substitution of A708K, a deletion of P at position 793 and a substitution of A739V of SEQ ID NO:2. In some embodiments, a CasX variant protein comprises a substitution of L379R, a substitution of A708K, a deletion of P at position 793 and a substitution of A739V of SEQ ID NO:2. In some embodiments, a CasX variant protein comprises a substitution of C477K, a substitution of A708K and a substitution of P at position 793 of SEQ ID NO:2. In some embodiments, a CasX variant protein comprises a substitution of L249I and a substitution of M771N of SEQ ID NO:2. In some embodiments, a CasX variant protein comprises a substitution of V747K of SEQ ID NO:2. In some embodiments, a CasX variant protein comprises a substitution of L379R, a substitution of C477, a substitution of A708K, a deletion of P at position 793 and a substitution of M779N of SEQ ID NO:2. In some embodiments, a CasX variant protein comprises a substitution of F755M. In some embodiments, a CasX variant comprises any combination of the foregoing embodiments of this paragraph.

In some embodiments, a CasX variant protein comprises at least one modification compared to the reference CasX sequence of SEQ ID NO:2, wherein the at least one modification is selected from one or more of: an amino acid substitution of L379R; an amino acid substitution of A708K; an amino acid substitution of T620P; an amino acid substitution of E385P; an amino acid substitution of Y857R; an amino acid substitution of I658V; an amino acid substitution of F399L; an amino acid substitution of Q252K; and an amino acid deletion of [P793]. In some embodiments, a CasX variant protein comprises at least one modification compared to the reference CasX sequence of SEQ ID NO:2, wherein the at least one modification is selected from one or more of: an amino acid substitution of L379R; an amino acid substitution of A708K; an amino acid substitution of T620P; an amino acid substitution of E385P; an amino acid substitution of Y857R; an amino acid substitution of I658V; an amino acid substitution of F399L; an amino acid substitution of Q252K; an amino acid substitution of L404K; and an amino acid deletion of [P793]. In other embodiments, a CasX variant protein comprises any combination of the foregoing substitutions or deletions compared to the reference CasX sequence of SEQ ID NO:2. In other embodiments, the CasX variant protein can, in addition to the foregoing substitutions or deletions, further comprise a substitution of an NTSB and/or a helical 1b domain from the reference CasX of SEQ ID NO:1.

In some embodiments, the CasX variant protein comprises between 400 and 2000 amino acids, between 500 and 1500 amino acids, between 700 and 1200 amino acids, between 800 and 1100 amino acids, or between 900 and 1000 amino acids.

In some embodiments, the CasX variant protein comprises one or more modifications in a region of non-contiguous residues that form a channel in which gNA:target DNA complexing occurs. In some embodiments, the CasX variant protein comprises one or more modifications comprising a region of non-contiguous residues that form an interface which binds with the gNA. For example, in some embodiments of a reference CasX protein, the helical I, helical II and OBD domains all contact or are in proximity to the gNA:target DNA complex, and one or more modifications to non-contiguous residues within any of these domains may improve function of the CasX variant protein.

In some embodiments, the CasX variant protein comprises one or more modifications in a region of non-contiguous residues that form a channel which binds with the non-target strand DNA. For example, a CasX variant protein can comprise one or more modifications to non-contiguous residues of the NTSBD. In some embodiments, the CasX variant protein comprises one or more modifications in a region of non-contiguous residues that form an interface which binds with the PAM. For example, a CasX variant protein can comprise one or more modifications to non-contiguous residues of the helical I domain or OBD. In some embodiments, the CasX variant protein comprises one or more modifications comprising a region of non-contiguous surface-exposed residues. As used herein, “surface-exposed residues” refers to amino acids on the surface of the CasX protein, or amino acids in which at least a portion of the amino acid, such as the backbone or a part of the side chain is on the surface of the protein. Surface exposed residues of cellular proteins such as CasX, which are exposed to an aqueous intracellular environment, are frequently selected from positively charged hydrophilic amino acids, for example arginine, asparagine, aspartate, glutamine, glutamate, histidine, lysine, serine, and threonine. Thus, for example, in some embodiments of the variants provided herein, a region of surface exposed residues comprises one or more insertions, deletions, or substitutions compared to a reference CasX protein. In some embodiments, one or more positively charged residues are substituted for one or more other positively charged residues, or negatively charged residues, or uncharged residues, or any combinations thereof. In some embodiments, one or more amino acids residues for substitution are near bound nucleic acid, for example residues in the RuvC domain or helical I domain that contact target DNA, or residues in the OBD or helical II domain that bind the gNA, can be substituted for one or more positively charged or polar amino acids.

In some embodiments, the CasX variant protein comprises one or more modifications in a region of non-contiguous residues that form a core through hydrophobic packing in a domain of the reference CasX protein. Without wishing to be bound by any theory, regions that form cores through hydrophobic packing are rich in hydrophobic amino acids such as valine, isoleucine, leucine, methionine, phenylalanine, tryptophan, and cysteine. For example, in some reference CasX proteins, RuvC domains comprise a hydrophobic pocket adjacent to the active site. In some embodiments, between 2 to 15 residues of the region are charged, polar, or base-stacking. Charged amino acids (sometimes referred to herein as residues) may include, for example, arginine, lysine, aspartic acid, and glutamic acid, and the side chains of these amino acids may form salt bridges provided a bridge partner is also present. Polar amino acids may include, for example, glutamine, asparagine, histidine, serine, threonine, tyrosine, and cysteine. Polar amino acids can, in some embodiments, form hydrogen bonds as proton donors or acceptors, depending on the identity of their side chains. As used herein, “base-stacking” includes the interaction of aromatic side chains of an amino acid residue (such as tryptophan, tyrosine, phenylalanine, or histidine) with stacked nucleotide bases in a nucleic acid. Any modification to a region of non-contiguous amino acids that are in close spatial proximity to form a functional part of the CasX variant protein is envisaged as within the scope of the disclosure.

i. CasX Variant Proteins with Domains from Multiple Source Proteins

In certain embodiments, the disclosure provides a chimeric CasX protein comprising protein domains from two or more different CasX proteins, such as two or more reference CasX proteins, or two or more CasX variant protein sequences as described herein. As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, Helical I, Helical II, OBD and RuvC domains. In some embodiments, the second domain is selected from the group consisting of the NTSB, TSL, Helical I, Helical II, OBD and RuvC domains with the second domain being different from the foregoing first domain. For example, a chimeric CasX protein may comprise an NTSB, TSL, Helical I, Helical II, OBD domains from a CasX protein of SEQ ID NO:2, and a RuvC domain from a CasX protein of SEQ ID NO:1, or vice versa. As a further example, a chimeric CasX protein may comprise an NTSB, TSL, Helical II, OBD and RuvC domain from CasX protein of SEQ ID NO:2, and a Helical I domain from a CasX protein of SEQ ID NO:1, or vice versa. Thus, in certain embodiments, a chimeric CasX protein may comprise an NTSB, TSL, Helical II, OBD and RuvC domain from a first CasX protein, and a Helical I domain from a second CasX protein. In some embodiments of the chimeric CasX proteins, the domains of the first CasX protein are derived from the sequences of SEQ ID NO:1, SEQ ID NO:2 or SEQ ID NO:3, and the domains of the second CasX protein are derived from the sequences of SEQ ID NO:1, SEQ ID NO:2 or SEQ ID NO:3, and the first and second CasX proteins are not the same. In some embodiments, domains of the first CasX protein comprise sequences derived from SEQ ID NO:1 and domains of the second CasX protein comprise sequences derived from SEQ ID NO:2. In some embodiments, domains of the first CasX protein comprise sequences derived from SEQ ID NO:1 and domains of the second CasX protein comprise sequences derived from SEQ ID NO:3. In some embodiments, domains of the first CasX protein comprise sequences derived from SEQ ID NO:2 and domains of the second CasX protein comprise sequences derived from SEQ ID NO:3. In some embodiments, the CasX variant comprises SEQ ID NOS: 130-138 or 141-144 the sequences of which are set forth in Table 4. In some embodiments, the CasX variant comprises a sequence of SEQ ID NOS: 72, 94, 113, 135, 138, 144, 239, 277, or 280. In some embodiments, the CasX variant comprises a sequence of SEQ ID NOS: 94, 72, 138, 144 or 280. In some embodiments, a CasX variant protein comprises at least one chimeric domain comprising a first part from a first CasX protein and a second part from a second, different CasX protein. As used herein, a “chimeric domain” refers to a domain containing at least two parts isolated or derived from different sources, such as two naturally occurring proteins or portions of domains from two reference CasX proteins. The at least one chimeric domain can be any of the NTSB, TSL, helical I, helical II, OBD or RuvC domains as described herein. In some embodiments, the first portion of a CasX domain comprises a sequence of SEQ ID NO:1 and the second portion of a CasX domain comprises a sequence of SEQ ID NO:2. In some embodiments, the first portion of the CasX domain comprises a sequence of SEQ ID NO:1 and the second portion of the CasX domain comprises a sequence of SEQ ID NO:3. In some embodiments, the first portion of the CasX domain comprises a sequence of SEQ ID NO:2 and the second portion of the CasX domain comprises a sequence of SEQ ID NO:3. In some embodiments, the at least one chimeric domain comprises a chimeric RuvC domain. As an example of the foregoing, the chimeric RuvC domain comprises amino acids 661 to 824 of SEQ ID NO:1 and amino acids 922 to 978 of SEQ ID NO:2. As an alternative example of the foregoing, a chimeric RuvC domain comprises amino acids 648 to 812 of SEQ ID NO:2 and amino acids 935 to 986 of SEQ ID NO:1. In some embodiments, a CasX protein comprises a first domain from a first CasX protein and a second domain from a second CasX protein, and at least one chimeric domain comprising at least two parts isolated from different CasX proteins using the approach of the embodiments described in this paragraph. In the foregoing embodiments, the chimeric CasX proteins having domains or portions of domains derived from SEQ ID NOS:1, 2 and 3, can further comprise amino acid insertions, deletions, or substitutions of any of the embodiments disclosed herein.

In some embodiments, a CasX variant protein comprises a sequence set forth in Tables 4, 6, 7, 8, or 10. In some embodiments, a CasX variant protein consists of a sequence set forth in Table 4. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence of SEQ ID NOS: 49-150, 233-235, 238-252, or 272-281 as set forth in Tables 4, 6, 7, 8, or 10. In other embodiments, a CasX variant protein comprises a sequence of SEQ ID NOS: 49-150 as set forth in Table 4, and further comprises one or more NLS disclosed herein at or near either the N-terminus, the C-terminus, or both. It will be understood that in some cases, the N-terminal methionine of the CasX variants of the Tables is removed from the expressed CasX variant during post-translational modification.

TABLE 4
CasX Variant Sequences

| Description* | Amino Acid Sequence |
|---|---|
| TSL, | MQEIKRINKIRRRLVKDSNTKKAGKTGPMKTLLVRVMTPDLRERLENLRKKPENI |
| Helical I, | PQPISNTSRANLNKLLTDYTEMKKAILHVYWEEFQKDPVGLMSRVAQPASKKIDQ |
| Helical II, | NKLKPEMDEKGNLTTAGFACSQCGQPLFVYKLEQVSEKGKAYTNYFGRCNVAEHE |
| OBD and | KLILLAQLKPEKDSDEAVTYSLGKFGQRALDFYSIHVTRESNHPVKPLEQIGGNS |
| RuvC | CASGPVGKALSDACMGAVASFLTKYQDIILEHQKVIKKNEKRLANLKDIASANGL |
| domains | AFPKITLPPQPHTKEGIEAYNNVVAQIVIWVNLNLWQKLKIGRDEAKPLQRLKGF |
| from SEQ | PSFPLVERQANEVDWWDMVCNVKKLINEKKEDGKVFWQNLAGYKRQEALRPYLSS |
| ID NO: 2 | EEDRKKGKKFARYQFGDLLLHLEKKHGEDWGKVYDEAWERIDKKVEGLSKHIKLE |
| and an | EERRSEDAQSKAALTDWLRAKASFVIEGLKEADKDEFCRCELKLQKWYGDLRGKP |
| NTSB | FAIEAENSILDISGFSKQYNCAFIWQKDGVKKLNLYLIINYFKGGKLRFKKIKPE |
| domain | AFEANRFYTVINKKSGEIVPMEVNFNFDDPNLIILPLAFGKRQGREFIWNDLLSL |
| from SEQ | ETGSLKLANGRVIEKTLYNRRTRQDEPALFVALTFERREVLDSSNIKPMNLIGID |
| ID NO: 1 | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMITINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 49) |
| NTSB, | MQEIKRINKIRRRLVKDSNTKKAGKTGPMKTLLVRVMTPDLRERLENLRKKPENI |
| Helical I, | PQPISNTSRANLNKLLTDYTEMKKAILHVYWEEFQKDPVGLMSRVAQPAPKNIDQ |
| Helical II, | RKLIPVKDGNERLTSSGFACSQCCQPLYVYKLEQVNDKGKPHTNYFGRCNVSEHE |
| OBD and | RLILLSPHKPEANDELVTYSLGKFGQRALDFYSIHVTRESNHPVKPLEQIGGNSC |
| RuvC | ASGPVGKALSDACMGAVASFLTKYQDIILEHQKVIKKNEKRLANLKDIASANGLA |
| domains | FPKITLPPQPHTKEGIEAYNNVVAQIVIWVNLNLWQKLKIGRDEAKPLQRLKGFP |
| from SEQ | SFPLVERQANEVDWWDMVCNVKKLINEKKEDGKVFWQNLAGYKRQEALRPYLSSE |
| ID NO: 2 | EDRKKGKKFARYQFGDLLLHLEKKHGEDWGKVYDEAWERIDKKVEGLSKHIKLEE |
| and a TSL | ERRSEDAQSKAALTDWLRAKASFVIEGLKEADKDEFCRCELKLQKWYGDLRGKPF |
| domain | AIEAENSILDISGFSKQYNCAFIWQKDGVKKLNLYLIINYFKGGKLRFKKIKPEA |
| from SEQ | FEANRFYTVINKKSGEIVPMEVNFNFDDPNLIILPLAFGKRQGREFIWNDLLSLE |
| ID NO: 1. | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITTADYDGML |
| | VRLKKTSDGWATTLNNKELKAEGQITYYNRYKRQTVEKELSAELDRLSEESGNND |
| | ISKWTKGRRDEALFLLKKRFSHRPVQEQFVCLDCGHEVHADEQAALNIARSWLFL |
| | RSQEYKKYQTNKTTGNTDKRAFVETWQSFYRKKLKEVWKPAV (SEQ ID NO: |
| | 50) |
| TSL, | MEKRINKIRKKLSADNATKPVSRSGPMKTLLVRVMTDDLKKRLEKRRKKPEVMPQ |
| Helical I, | VISNNAANNLRMLLDDYTKMKEAILQVYWQEFKDDHVGLMCKFAQPAPKNIDQRK |
| Helical II, | LIPVKDGNERLTSSGFACSQCCQPLYVYKLEQVNDKGKPHTNYFGRCNVSEHERL |
| OBD and | ILLSPHKPEANDELVTYSLGKFGQRALDFYSIHVTKESTHPVKPLAQIAGNRYAS |
| RuvC | GPVGKALSDACMGTIASFLSKYQDIIIEHQKVVKGNQKRLESLRELAGKENLEYP |
| domains | SVTLPPQPHTKEGVDAYNEVIARVRMWVNLNLWQKLKLSRDDAKPLLRLKGFPSF |
| from SEQ | PVVERRENEVDWWNTINEVKKLIDAKRDMGRVFWSGVTAEKRNTILEGYNYLPNE |
| ID NO: 1 | NDHKKREGSLENPKKPAKRQFGDLLLYLEKKYAGDWGKVFDEAWERIDKKIAGLT |
| and an | SHIEREEARNAEDAQSKAVLTDWLRAKASFVLERLKEMDEKEFYACEIQLQKWYG |
| NTSB | DLRGNPFAVEAENRVVDISGFSIGSDGHSIQYRNLLAWKYLENGKREFYLLMNYG |
| domain | KKGRIRFTDGTDIKKSGKWQGLLYGGGKAKVIDLTFDPDDEQLIILPLAFGTRQG |
| from SEQ | REFIWNDLLSLETGLIKLANGRVIEKTIYNKKIGRDEPALFVALTFERREVVDPS |
| ID NO: 2 | NIKPVNLIGVDRGENIPAVIALTDPEGCPLPEFKDSSGGPTDILRIGEGYKEKQR |
| | AIQAAKEVEQRRAGGYSRKFASKSRNLADDMVRNSARDLFYHAVTHDAVLVFENL |
| | SRGFGRQGKRTFMTERQYTKMEDWLTAKLAYEGLTSKTYLSKTLAQYTSKTCSNC |
| | GFTITTADYDGMLVRLKKTSDGWATTLNNKELKAEGQITYYNRYKRQTVEKELSA |
| | ELDRLSEESGNNDISKWIKGRRDEALFLLKKRFSHRPVQEQFVCLDCGHEVHADE |
| | QAALNIARSWLFLNSNSTEFKSYKSGKQPFVGAWQAFYKRRLKEVWKPNA (SEQ |
| | ID NO: 51) |
| NTSB, | MEKRINKIRKKLSADNATKPVSRSGPMKTLLVRVMTDDLKKRLEKRRKKPEVMPQ |
| Helical I, | VISNNAANNLRMLLDDYTKMKEAILQVYWQEFKDDHVGLMCKFAQPASKKIDQNK |
| Helical II, | LKPEMDEKGNLTTAGFACSQCGQPLFVYKLEQVSEKGKAYTNYFGRCNVAEHEKL |
| OBD and | ILLAQLKPEKDSDEAVTYSLGKFGQRALDFYSIHVTKESTHPVKPLAQIAGNRYA |
| RuvC | SGPVGKALSDACMGTIASFLSKYQDIIIEHQKVVKGNQKRLESLRELAGKENLEY |
| domains | PSVTLPPQPHTKEGVDAYNEVIARVRMWVNLNLWQKLKLSRDDAKPLLRLKGFPS |
| from SEQ | FPVVERRENEVDWWNTINEVKKLIDAKRDMGRVFWSGVTAEKRNTILEGYNYLPN |
| ID NO: 1 | ENDHKKREGSLENPKKPAKRQFGDLLLYLEKKYAGDWGKVFDEAWERIDKKIAGL |
| and an TSL | TSHIEREEARNAEDAQSKAVLTDWLRAKASFVLERLKEMDEKEFYACEIQLQKWY |
| domain | GDLRGNPFAVEAENRVVDISGFSIGSDGHSIQYRNLLAWKYLENGKREFYLLMNY |
| from SEQ | GKKGRIRFTDGTDIKKSGKWQGLLYGGGKAKVIDLTFDPDDEQLIILPLAFGTRQ |
| ID NO: 2. | GREFIWNDLLSLETGLIKLANGRVIEKTIYNKKIGRDEPALFVALTFERREVVDP |
| | SNIKPVNLIGVDRGENIPAVIALTDPEGCPLPEFKDSSGGPTDILRIGEGYKEKQ |
| | RAIQAAKEVEQRRAGGYSRKFASKSRNLADDMVRNSARDLFYHAVTHDAVLVFEN |
| | LSRGFGRQGKRTFMTERQYTKMEDWLTAKLAYEGLTSKTYLSKTLAQYTSKTCSN |
| | CGFTITSADYDRVLEKLKKTATGWMTTINGKELKVEGQITYYNRYKRQNVVKDLS |
| | VELDRLSEESVNNDISSWTKGRSGEALSLLKKRFSHRPVQEKFVCLNCGFETHAD |
| | EQAALNIARSWLFLNSNSTEFKSYKSGKQPFVGAWQAFYKRRLKEVWKPNA |
| | (SEQ ID NO: 52) |
| NTSB, | MQEIKRINKIRRRLVKDSNTKKAGKTGPMKTLLVRVMTPDLRERLENLRKKPENI |
| TSL, | PQPISNTSRANLNKLLTDYTEMKKAILHVYWEEFQKDPVGLMSRVAQPAPKNIDQ |
| Helical I, | RKLIPVKDGNERLTSSGFACSQCCQPLYVYKLEQVNDKGKPHTNYFGRCNVSEHE |
| Helical II | RLILLSPHKPEANDELVTYSLGKFGQRALDFYSIHVTRESNHPVKPLEQIGGNSC |
| and OBD | ASGPVGKALSDACMGAVASFLTKYQDIILEHQKVIKKNEKRLANLKDIASANGLA |
| domains | FPKITLPPQPHTKEGIEAYNNVVAQIVIWVNLNLWQKLKIGRDEAKPLQRLKGFP |
| SEQ ID | SFPLVERQANEVDWWDMVCNVKKLINEKKEDGKVFWQNLAGYKRQEALRPYLSSE |
| NO: 2 and | EDRKKGKKFARYQFGDLLLHLEKKHGEDWGKVYDEAWERIDKKVEGLSKHIKLEE |
| an | ERRSEDAQSKAALTDWLRAKASFVIEGLKEADKDEFCRCELKLQKWYGDLRGKPF |
| exogenous | AIEAENSILDISGFSKQYNCAFIWQKDGVKKLNLYLIINYFKGGKLRFKKIKPEA |
| RuvC | FEANRFYTVINKKSGEIVPMEVNFNFDDPNLIILPLAFGKRQGREFIWNDLLSLE |
| domain or a | TGSLKLANGRVIEKTLYNRRTRQDEPALFVALTFERREVLDSSNIKPVNLIGVDR |
| portion | GENIPAVIALTDPEGCPLPEFKDSSGGPTDILRIGEGYKEKQRAIQAAKEVEQRR |
| thereof | AGGYSRKFASKSRNLADDMVRNSARDLFYHAVTHDAVLVFENLSRGFGRQGKRTF |
| from a | MTERQYTKMEDWLTAKLAYEGLTSKTYLSKTLAQYTSKTCSNCGFTITSADYDRV |
| second | LEKLKKTATGWMTTINGKELKVEGQITYYNRYKRQNVVKDLSVELDRLSEESVNN |
| CasX | DISSWTKGRSGEALSLLKKRFSHRPVQEKFVCLNCGFETHA (SEQ ID NO: |
| protein. | 53) |
| | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHA (SEQ ID NO: |
| | 54) |
| NTSB, | MQEIKRINKIRRRLVKDSNTKKAGKTGPMKTLLVRVMTPDLRERLENLRKKPENI |
| TSL, | PQPISNNAANNLRMLLDDYTKMKEAILQVYWQEFKDDHVGLMCKFAQPAPKNIDQ |
| Helical II, | RKLIPVKDGNERLTSSGFACSQCCQPLYVYKLEQVNDKGKPHTNYFGRCNVSEHE |
| OBD and | RLILLSPHKPEANDELVTYSLGKFGQRALDFYSIHVTKESTHPVKPLAQIAGNRY |
| RuvC | ASGPVGKALSDACMGTIASFLSKYQDIIIEHQKVVKGNQKRLESLRELAGKENLE |
| domains | YPSVTLPPQPHTKEGVDAYNEVIARVRMWVNLNLWQKLKLSRDDAKPLLRLKGFP |
| from SEQ | SFPLVERQANEVDWWDMVCNVKKLINEKKEDGKVFWQNLAGYKRQEALRPYLSSE |
| ID NO: 2 | EDRKKGKKFARYQFGDLLLHLEKKHGEDWGKVYDEAWERIDKKVEGLSKHIKLEE |
| and a | ERRSEDAQSKAALTDWLRAKASFVIEGLKEADKDEFCRCELKLQKWYGDLRGKPF |
| Helical I | AIEAENSILDISGFSKQYNCAFIWQKDGVKKLNLYLIINYFKGGKLRFKKIKPEA |
| domain | FEANRFYTVINKKSGEIVPMEVNFNFDDPNLIILPLAFGKRQGREFIWNDLLSLE |
| from SEQ | TGSLKLANGRVIEKTLYNRRTRQDEPALFVALTFERREVLDSSNIKPMNLIGIDR |
| ID NO: 1 | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 55) |
| NTSB, | MQEIKRINKIRRRLVKDSNTKKAGKTGPMKTLLVRVMTPDLRERLENLRKKPENI |
| TSL, | PQPISNTSRANLNKLLTDYTEMKKAILHVYWEEFQKDPVGLMSRVAQPAPKNIDQ |
| Helical I, | RKLIPVKDGNERLTSSGFACSQCCQPLYVYKLEQVNDKGKPHTNYFGRCNVSEHE |
| OBD and | RLILLSPHKPEANDELVTYSLGKFGQRALDFYSIHVTRESNHPVKPLEQIGGNSC |
| RuvC | ASGPVGKALSDACMGAVASFLTKYQDIILEHQKVIKKNEKRLANLKDIASANGLA |
| domains | FPKITLPPQPHTKEGIEAYNNVVAQIVIWVNLNLWQKLKIGRDEAKPLQRLKGFP |
| from SEQ | SFPVVERRENEVDWWNTINEVKKLIDAKRDMGRVFWSGVTAEKRNTILEGYNYLP |
| ID NO: 2 | NENDHKKREGSLENPKKPAKRQFGDLLLYLEKKYAGDWGKVFDEAWERIDKKIAG |
| and a | LTSHIEREEARNAEDAQSKAVLTDWLRAKASFVLERLKEMDEKEFYACEIQLQKW |
| Helical II | YGDLRGNPFAVEAENSILDISGFSKQYNCAFIWQKDGVKKLNLYLIINYFKGGKL |
| domain | RFKKIKPEAFEANRFYTVINKKSGEIVPMEVNFNFDDPNLIILPLAFGKRQGREF |
| from SEQ | IWNDLLSLETGSLKLANGRVIEKTLYNRRTRQDEPALFVALTFERREVLDSSNIK |
| ID NO: 1 | PMNLIGIDRGENIPAVIALTDPEGCPLSRFKDSLGNPTHILRIGESYKEKQRTIQ |
| | AKKEVEQRRAGGYSRKYASKAKNLADDMVRNTARDLLYYAVTQDAMLIFENLSRG |
| | FGRQGKRTEMAERQYTRMEDWLTAKLAYEGLSKTYLSKTLAQYTSKTCSNCGFTI |
| | TSADYDRVLEKLKKTATGWMTTINGKELKVEGQITYYNRYKRQNVVKDLSVELDR |
| | LSEESVNNDISSWTKGRSGEALSLLKKRFSHRPVQEKFVCLNCGFETHADEQAAL |
| | NIARSWLFLRSQEYKKYQTNKTTGNTDKRAFVETWQSFYRKKLKEVWKPAV |
| | (SEQ ID NO: 56) |
| NTSB, | MISNTSRANLNKLLTDYTEMKKAILHVYWEEFQKDPVGLMSRVAQPAPKNIDQRK |
| TSL, | LIPVKDGNERLTSSGFACSQCCQPLYVYKLEQVNDKGKPHTNYFGRCNVSEHERL |
| Helical I, | ILLSPHKPEANDELVTYSLGKFGQRALDFYSIHVTRESNHPVKPLEQIGGNSCAS |
| Helical II | GPVGKALSDACMGAVASFLTKYQDIILEHQKVIKKNEKRLANLKDIASANGLAFP |
| and RuvC | KITLPPQPHTKEGIEAYNNVVAQIVIWVNLNLWQKLKIGRDEAKPLQRLKGFPSF |
| domains | PLVERQANEVDWWDMVCNVKKLINEKKEDGKVFWQNLAGYKRQEALRPYLSSEED |
| from a first | RKKGKKFARYQFGDLLLHLEKKHGEDWGKVYDEAWERIDKKVEGLSKHIKLEEER |
| CasX | RSEDAQSKAALTDWLRAKASFVIEGLKEADKDEFCRCELKLQKWYGDLRGKPFAI |
| protein and | EAENRVVDISGFSIGSDGHSIQYRNLLAWKYLENGKREFYLLMNYGKKGRIRFTD |
| an | GTDIKKSGKWQGLLYGGGKAKVIDLTFDPDDEQLIILPLAFGTRQGREFIWNDLL |
| exogenous | SLETGLIKLANGRVIEKTIYNKKIGRDEPALFVALTFERREVVDPSNIKPMNLIG |
| OBD or a | IDRGENIPAVIALTDPEGCPLSRFKDSLGNPTHILRIGESYKEKQRTIQAKKEVE |
| part thereof | QRRAGGYSRKYASKAKNLADDMVRNTARDLLYYAVTQDAMLIFENLSRGFGRQGK |
| from a | RTFMAERQYTRMEDWLTAKLAYEGLSKTYLSKTLAQYTSKTCSNCGFTITSADYD |
| second | RVLEKLKKTATGWMTTINGKELKVEGQITYYNRYKRQNVVKDLSVELDRLSEESV |
| CasX | NNDISSWTKGRSGEALSLLKKRFSHRPVQEKFVCLNCGFETHADEQAALNIARSW |
| protein | LFLRSQEYKKYQTNKTTGNTDKRAFVETWQSFYRKKLKEVWKPAV (SEQ ID |
| | NO: 57) |
| | MEKRINKIRKKLSADNATKPVSRSGPMKTLLVRVMTDDLKKRLEKRRKKPEVMPQ |
| | VISNTSRANLNKLLTDYTEMKKAILHVYWEEFQKDPVGLMSRVAQPAPKNIDQRK |
| | LIPVKDGNERLTSSGFACSQCCQPLYVYKLEQVNDKGKPHTNYFGRCNVSEHERL |
| | ILLSPHKPEANDELVTYSLGKFGQRALDFYSIHVTRESNHPVKPLEQIGGNSCAS |
| | GPVGKALSDACMGAVASFLTKYQDIILEHQKVIKKNEKRLANLKDIASANGLAFP |
| | KITLPPQPHTKEGIEAYNNVVAQIVIWVNLNLWQKLKIGRDEAKPLQRLKGFPSF |
| | PLVERQANEVDWWDMVCNVKKLINEKKEDGKVFWQNLAGYKRQEALRPYLSSEED |
| | RKKGKKFARYQFGDLLLHLEKKHGEDWGKVYDEAWERIDKKVEGLSKHIKLEEER |
| | RSEDAQSKAALTDWLRAKASFVIEGLKEADKDEFCRCELKLQKWYGDLRGKPFAI |
| | EAENSILDISGFSKQYNCAFIWQKDGVKKLNLYLIINYFKGGKLRFKKIKPEAFE |
| | ANRFYTVINKKSGEIVPMEVNFNFDDPNLIILPLAFGKRQGREFIWNDLLSLETG |
| | SLKLANGRVIEKTLYNRRTRQDEPALFVALTFERREVLDSSNIKPMNLIGIDRGE |
| | NIPAVIALTDPEGCPLSRFKDSLGNPTHILRIGESYKEKQRTIQAKKEVEQRRAG |
| | GYSRKYASKAKNLADDMVRNTARDLLYYAVTQDAMLIFENLSRGFGRQGKRTEMA |
| | ERQYTRMEDWLTAKLAYEGLSKTYLSKTLAQYTSKTCSNCGFTITSADYDRVLEK |
| | LKKTATGWMTTINGKELKVEGQITYYNRYKRQNVVKDLSVELDRLSEESVNNDIS |
| | SWTKGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 58) |
| | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENRVVDISGFSIGSDGHSIQYRNLLAWKYLENGKREFYLLMNYGKKGRIRF |
| | TDGTDIKKSGKWQGLLYGGGKAKVIDLTFDPDDEQLIILPLAFGTRQGREFIWND |
| | LLSLETGLIKLANGRVIEKTIYNKKIGRDEPALFVALTFERREVVDPSNIKPMNL |
| | IGIDRGENIPAVIALTDPEGCPLSRFKDSLGNPTHILRIGESYKEKQRTIQAKKE |
| | VEQRRAGGYSRKYASKAKNLADDMVRNTARDLLYYAVTQDAMLIFENLSRGFGRQ |
| | GKRTFMAERQYTRMEDWLTAKLAYEGLSKTYLSKTLAQYTSKTCSNCGFTITSAD |
| | YDRVLEKLKKTATGWMTTINGKELKVEGQITYYNRYKRQNVVKDLSVELDRLSEE |
| | SVNNDISSWTKGRSGEALSLLKKRFSHRPVQEKFVCLNCGFETHADEQAALNIAR |
| | SWLFLRSQEYKKYQTNKTTGNTDKRAFVETWQSFYRKKLKEVWKPAV (SEQ ID |
| | NO: 59) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGDLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKPLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of T620P | AGGYSRKYASKAKNLADDMVRNTARDLLYYAVTQDAMLIFENLSRGFGRQGKRTF |
| of SEQ ID | MAERQYTRMEDWLTAKLAYEGLSKTYLSKTLAQYTSKTCSNCGFTITSADYDRVL |
| NO: 2 | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 60) |
| substitution | MQEIKRINKIRRRLVKDSNTKKAGKTGPMKTLLVRVMTPDLRERLENLRKKPENI |
| of M771A | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | AAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPA (SEQ ID NO: |
| | 61) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of A708K, | ASGPVGKALSDACMGAVASFLTKYQDIILEHQKVIKKNEKRLANLKDIASANGLA |
| a deletion | FPKITLPPQPHTKEGIEAYNNVVAQIVIWVNLNLWQKLKIGRDEAKPLQRLKGFP |
| of P at | SFPLVERQANEVDWWDMVCNVKKLINEKKEDGKVFWQNLAGYKRQEALRPYLSSE |
| position | EDRKKGKKFARYQFGDLLLHLEKKHGEDWGKVYDEAWERIDKKVEGLSKHIKLEE |
| 793 and a | ERRSEDAQSKAALTDWLRAKASFVIEGLKEADKDEFCRCELKLQKWYGDLRGKPF |
| substitution | AIEAENSILDISGFSKQYNCAFIWQKDGVKKLNLYLIINYFKGGKLRFKKIKPEA |
| of D732N | FEANRFYTVINKKSGEIVPMEVNFNFDDPNLIILPLAFGKRQGREFIWNDLLSLE |
| of SEQ ID | TGSLKLANGRVIEKTLYNRRTRQDEPALFVALTFERREVLDSSNIKPMNLIGIDR |
| NO: 2. | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 62) |
| substitution | MQEIKRINKIRRRLVKDSNTKKAGKTGPMKTLLVRVMTPDLRERLENLRKKPENI |
| of W782Q | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | MAERQYTRMEDQLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 63) |
| substitution | MQEIKRINKIRRRLVKDSNTKKAGKTGPMKTLLVRVMTPDLRERLENLRKKPENI |
| of M771Q | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2 | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | QAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 64) |
| substitution | MQEIKRINKIRRRLVKDSNTKKAGKTGPMKTLLVRVMTPDLRERLENLRKKPENI |
| of R458I | PQPISNTSRANLNKLLTDYTEMKKAILHVYWEEFQKDPVGLMSRVAQPAPKNIDQ |
| and a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of A739V | ASGPVGKALSDACMGAVASFLTKYQDIILEHQKVIKKNEKRLANLKDIASANGLA |
| of SEQ ID | FPKITLPPQPHTKEGIEAYNNVVAQIVIWVNLNLWQKLKIGRDEAKPLQRLKGFP |
| NO: 2. | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLIAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTVRDLLYYAVTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 65) |
| L379R, a | MQEIKRINKIRRRLVKDSNTKKAGKTGPMKTLLVRVMTPDLRERLENLRKKPENI |
| substitution | PQPISNTSRANLNKLLTDYTEMKKAILHVYWEEFQKDPVGLMSRVAQPAPKNIDQ |
| of A708K, | RKLIPVKDGNERLTSSGFACSQCCQPLYVYKLEQVNDKGKPHTNYFGRCNVSEHE |
| a deletion | RLILLSPHKPEANDELVTYSLGKFGQRALDFYSIHVTRESNHPVKPLEQIGGNSC |
| of P at | ASGPVGKALSDACMGAVASFLTKYQDIILEHQKVIKKNEKRLANLKDIASANGLA |
| position | FPKITLPPQPHTKEGIEAYNNVVAQIVIWVNLNLWQKLKIGRDEAKPLQRLKGFP |
| 793 and a | SFPLVERQANEVDWWDMVCNVKKLINEKKEDGKVFWQNLAGYKRQEALRPYLSSE |
| substitution | EDRKKGKKFARYQFGDLLLHLEKKHGEDWGKVYDEAWERIDKKVEGLSKHIKLEE |
| of M771N | ERRSEDAQSKAALTDWLRAKASFVIEGLKEADKDEFCRCELKLQKWYGDLRGKPF |
| of SEQ ID | AIEAENSILDISGFSKQYNCAFIWQKDGVKKLNLYLIINYFKGGKLRFKKIKPEA |
| NO: 2 | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | NAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 66) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of A708K, | ASGPVGKALSDACMGAVASFLTKYQDIILEHQKVIKKNEKRLANLKDIASANGLA |
| a deletion | FPKITLPPQPHTKEGIEAYNNVVAQIVIWVNLNLWQKLKIGRDEAKPLQRLKGFP |
| of P at | SFPLVERQANEVDWWDMVCNVKKLINEKKEDGKVFWQNLAGYKRQEALRPYLSSE |
| position | EDRKKGKKFARYQFGDLLLHLEKKHGEDWGKVYDEAWERIDKKVEGLSKHIKLEE |
| 793 and a | ERRSEDAQSKAALTDWLRAKASFVIEGLKEADKDEFCRCELKLQKWYGDLRGKPF |
| substitution | AIEAENSILDISGFSKQYNCAFIWQKDGVKKLNLYLIINYFKGGKLRFKKIKPEA |
| of A739T | FEANRFYTVINKKSGEIVPMEVNFNFDDPNLIILPLAFGKRQGREFIWNDLLSLE |
| of SEQ ID | TGSLKLANGRVIEKTLYNRRTRQDEPALFVALTFERREVLDSSNIKPMNLIGIDR |
| NO: 2 | GENIPAVIALTDPEGCPLSRFKDSLGNPTHILRIGESYKEKQRTIQAKKEVEQRR |
| | AGGYSRKYASKAKNLADDMVRNTTRDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 67) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGSLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKTLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of D489S | AGGYSRKYASKAKNLADDMVRNTARDLLYYAVTQDAMLIFENLSRGFGRQGKRTF |
| of SEQ ID | MAERQYTRMEDWLTAKLAYEGLSKTYLSKTLAQYTSKTCSNCGFTITSADYDRVL |
| NO: 2. | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 68) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGDLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKTLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of D732N | AGGYSRKYASKAKNLANDMVRNTARDLLYYAVTQDAMLIFENLSRGFGRQGKRTF |
| of SEQ ID | MAERQYTRMEDWLTAKLAYEGLSKTYLSKTLAQYTSKTCSNCGFTITSADYDRVL |
| NO: 2. | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 69) |
| substitution | MQEIKRINKIRRRLVKDSNTKKAGKTGPMKTLLVRVMTPDLRERLENLRKKPENI |
| of V711K | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEKEQRR |
| | AGGYSRKYASKAKNLADDMVRNTARDLLYYAVTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 70) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGDLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKTLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of Y797L | AGGYSRKYASKAKNLADDMVRNTARDLLYYAVTQDAMLIFENLSRGFGRQGKRTF |
| of SEQ ID | MAERQYTRMEDWLTAKLAYEGLSKILLSKTLAQYTSKTCSNCGFTITSADYDRVL |
| NO: 2. | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 71) |
| 119: | MQEIKRINKIRRRLVKDSNTKKAGKTGPMKTLLVRVMTPDLRERLENLRKKPENI |
| substitution | PQPISNTSRANLNKLLTDYTEMKKAILHVYWEEFQKDPVGLMSRVAQPAPKNIDQ |
| of L379R, | RKLIPVKDGNERLTSSGFACSQCCQPLYVYKLEQVNDKGKPHTNYFGRCNVSEHE |
| a | RLILLSPHKPEANDELVTYSLGKFGQRALDFYSIHVTRESNHPVKPLEQIGGNSC |
| substitution | ASGPVGKALSDACMGAVASFLTKYQDIILEHQKVIKKNEKRLANLKDIASANGLA |
| of A708K | FPKITLPPQPHTKEGIEAYNNVVAQIVIWVNLNLWQKLKIGRDEAKPLQRLKGFP |
| and a | SFPLVERQANEVDWWDMVCNVKKLINEKKEDGKVFWQNLAGYKRQEALRPYLSSE |
| deletion of | EDRKKGKKFARYQFGDLLLHLEKKHGEDWGKVYDEAWERIDKKVEGLSKHIKLEE |
| P at | ERRSEDAQSKAALTDWLRAKASFVIEGLKEADKDEFCRCELKLQKWYGDLRGKPF |
| position | AIEAENSILDISGFSKQYNCAFIWQKDGVKKLNLYLIINYFKGGKLRFKKIKPEA |
| 793 of SEQ | FEANRFYTVINKKSGEIVPMEVNFNFDDPNLIILPLAFGKRQGREFIWNDLLSLE |
| ID NO: 2. | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 72) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGDLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKTLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of M771N | AGGYSRKYASKAKNLADDMVRNTARDLLYYAVTQDAMLIFENLSRGFGRQGKRTF |
| of SEQ ID | NAERQYTRMEDWLTAKLAYEGLSKTYLSKTLAQYTSKTCSNCGFTITSADYDRVL |
| NO: 2. | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 73) |
| substitution | MQEIKRINKIRRRLVKDSNTKKAGKTGPMKTLLVRVMTPDLRERLENLRKKPENI |
| of A708K, | PQPISNTSRANLNKLLTDYTEMKKAILHVYWEEFQKDPVGLMSRVAQPAPKNIDQ |
| a deletion | RKLIPVKDGNERLTSSGFACSQCCQPLYVYKLEQVNDKGKPHTNYFGRCNVSEHE |
| of P at | RLILLSPHKPEANDELVTYSLGKFGQRALDFYSIHVTRESNHPVKPLEQIGGNSC |
| position | ASGPVGKALSDACMGAVASFLTKYQDIILEHQKVIKKNEKRLANLKDIASANGLA |
| 793 and a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALLPYLSSE |
| of E386S | EDRKKGKKFARYQFGDLLLHLEKKHGEDWGKVYDEAWERIDKKVEGLSKHIKLEE |
| of SEQ ID | ERRSEDAQSKAALTDWLRAKASFVIEGLKEADKDEFCRCELKLQKWYGDLRGKPF |
| NO: 2. | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 74) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K | EDRKKGKKFARYQFGDLLLHLEKKHGEDWGKVYDEAWERIDKKVEGLSKHIKLEE |
| and a | ERRSEDAQSKAALTDWLRAKASFVIEGLKEADKDEFKRCELKLQKWYGDLRGKPF |
| deletion of | AIEAENSILDISGFSKQYNCAFIWQKDGVKKLNLYLIINYFKGGKLRFKKIKPEA |
| P at | FEANRFYTVINKKSGEIVPMEVNFNFDDPNLIILPLAFGKRQGREFIWNDLLSLE |
| position | TGSLKLANGRVIEKTLYNRRTRQDEPALFVALTFERREVLDSSNIKPMNLIGIDR |
| 793 of SEQ | GENIPAVIALTDPEGCPLSRFKDSLGNPTHILRIGESYKEKQRTIQAKKEVEQRR |
| ID NO: 2. | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 75) |
| substitution | MQEIKRINKIRRRLVKDSNTKKAGKTGPMKTLLVRVMTPDLRERLENLRKKPENI |
| of L792D | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | MAERQYTRMEDWLTAKLAYEGDPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 76) |
| substitution | MQEIKRINKIRRRLVKDSNTKKAGKTGPMKTLLVRVMTPDLRERLENLRKKPENI |
| of G791F | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | MAERQYTRMEDWLTAKLAYEFLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 77) |
| substitution | MQEIKRINKIRRRLVKDSNTKKAGKTGPMKTLLVRVMTPDLRERLENLRKKPENI |
| of A708K, | PQPISNTSRANLNKLLTDYTEMKKAILHVYWEEFQKDPVGLMSRVAQPAPKNIDQ |
| a deletion | RKLIPVKDGNERLTSSGFACSQCCQPLYVYKLEQVNDKGKPHTNYFGRCNVSEHE |
| of P at | RLILLSPHKPEANDELVTYSLGKFGQRALDFYSIHVTRESNHPVKPLEQIGGNSC |
| position | ASGPVGKALSDACMGAVASFLTKYQDIILEHQKVIKKNEKRLANLKDIASANGLA |
| 793 and a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALLPYLSSE |
| of A739V | EDRKKGKKFARYQFGDLLLHLEKKHGEDWGKVYDEAWERIDKKVEGLSKHIKLEE |
| of SEQ ID | ERRSEDAQSKAALTDWLRAKASFVIEGLKEADKDEFCRCELKLQKWYGDLRGKPF |
| NO: 2. | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTVRDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 78) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of A708K, | ASGPVGKALSDACMGAVASFLTKYQDIILEHQKVIKKNEKRLANLKDIASANGLA |
| a deletion | FPKITLPPQPHTKEGIEAYNNVVAQIVIWVNLNLWQKLKIGRDEAKPLQRLKGFP |
| of P at | SFPLVERQANEVDWWDMVCNVKKLINEKKEDGKVFWQNLAGYKRQEALRPYLSSE |
| position | EDRKKGKKFARYQFGDLLLHLEKKHGEDWGKVYDEAWERIDKKVEGLSKHIKLEE |
| 793 and a | ERRSEDAQSKAALTDWLRAKASFVIEGLKEADKDEFCRCELKLQKWYGDLRGKPF |
| substitution | AIEAENSILDISGFSKQYNCAFIWQKDGVKKLNLYLIINYFKGGKLRFKKIKPEA |
| of A739V | FEANRFYTVINKKSGEIVPMEVNFNFDDPNLIILPLAFGKRQGREFIWNDLLSLE |
| of SEQ ID | TGSLKLANGRVIEKTLYNRRTRQDEPALFVALTFERREVLDSSNIKPMNLIGIDR |
| NO: 2. | GENIPAVIALTDPEGCPLSRFKDSLGNPTHILRIGESYKEKQRTIQAKKEVEQRR |
| | AGGYSRKYASKAKNLADDMVRNTVRDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 79) |
| substitution | MQEIKRINKIRRRLVKDSNTKKAGKTGPMKTLLVRVMTPDLRERLENLRKKPENI |
| of C477K, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of A708K | ASGPVGKALSDACMGAVASFLTKYQDIILEHQKVIKKNEKRLANLKDIASANGLA |
| and a | FPKITLPPQPHTKEGIEAYNNVVAQIVIWVNLNLWQKLKIGRDEAKPLQRLKGFP |
| deletion of | SFPLVERQANEVDWWDMVCNVKKLINEKKEDGKVFWQNLAGYKRQEALLPYLSSE |
| P at | EDRKKGKKFARYQFGDLLLHLEKKHGEDWGKVYDEAWERIDKKVEGLSKHIKLEE |
| position | ERRSEDAQSKAALTDWLRAKASFVIEGLKEADKDEFKRCELKLQKWYGDLRGKPF |
| 793 of SEQ | AIEAENSILDISGFSKQYNCAFIWQKDGVKKLNLYLIINYFKGGKLRFKKIKPEA |
| ID NO: 2. | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 80) |
| substitution | MQEIKRINKIRRRLVKDSNTKKAGKTGPMKTLLVRVMTPDLRERLENLRKKPENI |
| of L249I | PQPISNTSRANLNKLLTDYTEMKKAILHVYWEEFQKDPVGLMSRVAQPAPKNIDQ |
| and a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of M771N | ASGPVGKALSDACMGAVASFLTKYQDIIIEHQKVIKKNEKRLANLKDIASANGLA |
| of SEQ ID | FPKITLPPQPHTKEGIEAYNNVVAQIVIWVNLNLWQKLKIGRDEAKPLQRLKGFP |
| NO: 2. | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGESKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | NAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 81) |
| substitution | MQEIKRINKIRRRLVKDSNTKKAGKTGPMKTLLVRVMTPDLRERLENLRKKPENI |
| of V747K | PQPISNTSRANLNKLLTDYTEMKKAILHVYWEEFQKDPVGLMSRVAQPAPKNIDQ |
| of SEQ ID | RKLIPVKDGNERLTSSGFACSQCCQPLYVYKLEQVNDKGKPHTNYFGRCNVSEHE |
| NO: 2. | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAKTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 82) |
| substitution | MQEIKRINKIRRRLVKDSNTKKAGKTGPMKTLLVRVMTPDLRERLENLRKKPENI |
| of L379R, | PQPISNTSRANLNKLLTDYTEMKKAILHVYWEEFQKDPVGLMSRVAQPAPKNIDQ |
| a | RKLIPVKDGNERLTSSGFACSQCCQPLYVYKLEQVNDKGKPHTNYFGRCNVSEHE |
| substitution | RLILLSPHKPEANDELVTYSLGKFGQRALDFYSIHVTRESNHPVKPLEQIGGNSC |
| of C477K, | ASGPVGKALSDACMGAVASFLTKYQDIILEHQKVIKKNEKRLANLKDIASANGLA |
| a | FPKITLPPQPHTKEGIEAYNNVVAQIVIWVNLNLWQKLKIGRDEAKPLQRLKGFP |
| substitution | SFPLVERQANEVDWWDMVCNVKKLINEKKEDGKVFWQNLAGYKRQEALRPYLSSE |
| of A708K, | EDRKKGKKFARYQFGDLLLHLEKKHGEDWGKVYDEAWERIDKKVEGLSKHIKLEE |
| a deletion | ERRSEDAQSKAALTDWLRAKASFVIEGLKEADKDEFKRCELKLQKWYGDLRGKPF |
| of P at | AIEAENSILDISGFSKQYNCAFIWQKDGVKKLNLYLIINYFKGGKLRFKKIKPEA |
| position | FEANRFYTVINKKSGEIVPMEVNFNFDDPNLIILPLAFGKRQGREFIWNDLLSLE |
| 793 and a | TGSLKLANGRVIEKTLYNRRTRQDEPALFVALTFERREVLDSSNIKPMNLIGIDR |
| substitution | GENIPAVIALTDPEGCPLSRFKDSLGNPTHILRIGESYKEKQRTIQAKKEVEQRR |
| of M779N | AGGYSRKYASKAKNLADDMVRNTARDLLYYAVTQDAMLIFENLSRGFGRQGKRTF |
| of SEQ ID | MAERQYTRNEDWLTAKLAYEGLSKTYLSKTLAQYTSKTCSNCGFTITSADYDRVL |
| NO: 2. | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 83) |
| L379R, | MQEIKRINKIRRRLVKDSNTKKAGKTGPMKTLLVRVMTPDLRERLENLRKKPENI |
| F755M | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIMENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 84) |
| 429: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 85) |
| 430: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 86) |
| 431: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| E386N | SFPLVERQANEVDWWDMVCNVKKLINEKKEDGKVFWQNLAGYKRQEALRPYLSSE |
| | NDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 87) |
| 432: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| L404K | SFPLVERQANEVDWWDMVCNVKKLINEKKEDGKVFWQNLAGYKRQEALRPYLSSE |
| | EDRKKGKKFARYQFGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 88) |
| 433: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQVRALDFYSIHVTRESNHPVKPLEQIGGNS |
| Y857R, | CASGPVGKALSDACMGAVASFLTKYQDIILEHQKVIKKNEKRLANLKDIASANGL |
| I658V, | AFPKITLPPQPHTKEGIEAYNNVVAQIVIWVNLNLWQKLKIGRDEAKPLQRLKGF |
| ?V192 | PSFPLVERQANEVDWWDMVCNVKKLINEKKEDGKVFWQNLAGYKRQEALRPYLSS |
| | EEDRKKGKKFARYQFGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMTTINGKELKVEGQITYYNRRKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 89) |
| 434: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| L404K, | SFPLVERQANEVDWWDMVCNVKKLINEKKEDGKVFWQNLAGYKRQEALRPYLSSE |
| E386N | NDRKKGKKFARYQFGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 90) |
| 435: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 91) |
| 436: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| E386N | NDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 92) |
| 437: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| C477S | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFSRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 93) |
| 438: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| L404K | EDRKKGKKFARYQLGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 94) |
| 439: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| E386N, | NDRKKGKKFARYQLGDLLKHLEKKHGEDWGKVYDEAWERIDKKVEGLSKHIKLEE |
| C477S, | ERRSEDAQSKAALTDWLRAKASFVIEGLKEADKDEFSRCELKLQKWYGDLRGKPF |
| L404K | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 95) |
| 440: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| Y797L | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGESKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKILLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 96) |
| 441: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| Y797L, | NDRKKGKKFARYQLGDLLLHLEKKHGEDWGKVYDEAWERIDKKVEGLSKHIKLEE |
| E386N | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTLLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 97) |
| 442: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| F399L, | SFPLVERQANEVDWWDMVCNVKKLINEKKEDGKVFWQNLAGYKRQEALRPYLSSE |
| Y797L, | NDRKKGKKFARYQLGDLLKHLEKKHGEDWGKVYDEAWERIDKKVEGLSKHIKLEE |
| E386N, | ERRSEDAQSKAALTDWLRAKASFVIEGLKEADKDEFSRCELKLQKWYGDLRGKPF |
| C477S, | AIEAENSILDISGFSKQYNCAFIWQKDGVKKLNLYLIINYFKGGKLRFKKIKPEA |
| L404K | FEANRFYTVINKKSGEIVPMEVNENEDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKILLSKTLAQYTSKTCSNCGFTITSADYDRVL |
| | EKLKKTATGWMITINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 98) |
| 443: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| Y797L | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKILLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 99) |
| 444: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| Y797L, | SFPLVERQANEVDWWDMVCNVKKLINEKKEDGKVFWQNLAGYKRQEALRPYLSSE |
| L404K | EDRKKGKKFARYQFGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTLLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 100) |
| 445: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| Y797L, | SFPLVERQANEVDWWDMVCNVKKLINEKKEDGKVFWQNLAGYKRQEALRPYLSSE |
| E386N | NDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKILLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 101) |
| 446: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| I658V, | FPKITLPPQPHTKEGIEAYNNVVAQIVIWVNLNLWQKLKIGRDEAKPLQRLKGFP |
| Y797L, | SFPLVERQANEVDWWDMVCNVKKLINEKKEDGKVFWQNLAGYKRQEALRPYLSSE |
| E386N, | NDRKKGKKFARYQFGDLLKHLEKKHGEDWGKVYDEAWERIDKKVEGLSKHIKLEE |
| C477S, | ERRSEDAQSKAALTDWLRAKASFVIEGLKEADKDEFSRCELKLQKWYGDLRGKPF |
| L404K | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKILLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 102) |
| 447: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| E386N | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | NDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 103) |
| 448: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| Y857R, | ASGPVGKALSDACMGAVASFLTKYQDIILEHQKVIKKNEKRLANLKDIASANGLA |
| E386N, | FPKITLPPQPHTKEGIEAYNNVVAQIVIWVNLNLWQKLKIGRDEAKPLQRLKGFP |
| L404K | SFPLVERQANEVDWWDMVCNVKKLINEKKEDGKVFWQNLAGYKRQEALRPYLSSE |
| | NDRKKGKKFARYQFGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 104) |
| 449: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| D732N, | ASGPVGKALSDACMGAVASFLTKYQDIILEHQKVIKKNEKRLANLKDIASANGLA |
| E385P, | FPKITLPPQPHTKEGIEAYNNVVAQIVIWVNLNLWQKLKIGRDEAKPLQRLKGFP |
| Y857R | SFPLVERQANEVDWWDMVCNVKKLINEKKEDGKVFWQNLAGYKRQEALRPYLSSP |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 105) |
| 450: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| D732N, | ASGPVGKALSDACMGAVASFLTKYQDIILEHQKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 106) |
| 451: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| D732N, | ASGPVGKALSDACMGAVASFLTKYQDIILEHQKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | EDRKKGKKFARYQLGDLLLHLEKKHGEDWGKVYDEAWERIDKKVEGLSKHIKLEE |
| F399L | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 107) |
| 452: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| D732N, | ASGPVGKALSDACMGAVASFLTKYQDIILEHQKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | NDRKKGKKFARYQFGDLLLHLEKKHGEDWGKVYDEAWERIDKKVEGLSKHIKLEE |
| E386N | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 108) |
| 453: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| D732N, | ASGPVGKALSDACMGAVASFLTKYQDIILEHQKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | EDRKKGKKFARYQFGDLLKHLEKKHGEDWGKVYDEAWERIDKKVEGLSKHIKLEE |
| L404K | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLANDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 109) |
| 454: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| Q252K | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 110) |
| 455: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | EDRKKGKKFARYQFGDLLLHLEKKHGEDWGKVYDEAWERIDKKVEGLSKHIKLEE |
| Q252K | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 111) |
| 456: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | NDRKKGKKFARYQFGDLLLHLEKKHGEDWGKVYDEAWERIDKKVEGLSKHIKLEE |
| E386N, | ERRSEDAQSKAALTDWLRAKASFVIEGLKEADKDEFCRCELKLQKWYGDLRGKPF |
| Q252K | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 112) |
| 457: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | EDRKKGKKFARYQLGDLLLHLEKKHGEDWGKVYDEAWERIDKKVEGLSKHIKLEE |
| F399L, | ERRSEDAQSKAALTDWLRAKASFVIEGLKEADKDEFCRCELKLQKWYGDLRGKPF |
| Q252K | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 113) |
| 458: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Y857R, | SFPLVERQANEVDWWDMVCNVKKLINEKKEDGKVFWQNLAGYKRQEALRPYLSSP |
| I658V, | EDRKKGKKFARYQFGDLLKHLEKKHGEDWGKVYDEAWERIDKKVEGLSKHIKLEE |
| L404K, | ERRSEDAQSKAALTDWLRAKASFVIEGLKEADKDEFCRCELKLQKWYGDLRGKPF |
| Q252K | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 114) |
| 459: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHQKVIKKNEKRLANLKDIASANGLA |
| Y857R, | FPKITLPPQPHTKEGIEAYNNVVAQIVIWVNLNLWQKLKIGRDEAKPLQRLKGFP |
| I658V, | SFPLVERQANEVDWWDMVCNVKKLINEKKEDGKVFWQNLAGYKRQEALRPYLSSE |
| E386N | NDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 115) |
| 460: | MQEIKRINKIRRRLVKDSNTKKAGKTGPMKTLLVRVMTPDLRERLENLRKKPENI |
| L379R, | PQPISNTSRANLNKLLTDYTEMKKAILHVYWEEFQKDPVGLMSRVAQPAPKNIDQ |
| A708K, | RKLIPVKDGNERLTSSGFACSQCCQPLYVYKLEQVNDKGKPHTNYFGRCNVSEHE |
| P793_, | RLILLSPHKPEANDELVTYSLGKFGQRALDFYSIHVTRESNHPVKPLEQIGGNSC |
| T620P, | ASGPVGKALSDACMGAVASFLTKYQDIILEHKKVIKKNEKRLANLKDIASANGLA |
| E385P, | FPKITLPPQPHTKEGIEAYNNVVAQIVIWVNLNLWQKLKIGRDEAKPLQRLKGFP |
| Q252K | SFPLVERQANEVDWWDMVCNVKKLINEKKEDGKVFWQNLAGYKRQEALRPYLSSP |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKPLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 116) |
| 278 | QEIKRINKIRRRLVKDSNTKKAGKTGPMKTLLVRVMTPDLRERLENLRKKPENIP |
| | QPISNTSRANLNKLLTDYTEMKKAILHVYWEEFQKDPVGLMSRVAQPAPKNIDQR |
| | KLIPVKDGNERLTSSGFACSQCCQPLYVYKLEQVNDKGKPHTNYFGRCNVSEHER |
| | LILLSPHKPEANDELVTYSLGKFGQRALDFYSIHVTRESNHPVKPLEQIGGNSCA |
| | SGPVGKALSDACMGAVASFLTKYQDIILEHQKVIKKNEKRLANLKDIASANGLAF |
| | PKITLPPQPHTKEGIEAYNNVVAQIVIWVNLNLWQKLKIGRDEAKPLQRLKGFPS |
| | FPLVERQANEVDWWDMVCNVKKLINEKKEDGKVFWQNLAGYKRQEALRPYLSSEE |
| | DRKKGKKFARYQFGDLLLHLEKKHGEDWGKVYDEAWERIDKKVEGLSKHIKLEEE |
| | RRSEDAQSKAALTDWLRAKASFVIEGLKEADKDEFCRCELKLQKWYGDLRGKPFA |
| | IEAENSILDISGFSKQYNCAFIWQKDGVKKLNLYLIINYFKGGKLRFKKIKPEAF |
| | EANRFYTVINKKSGEIVPMEVNFNFDDPNLIILPLAFGKRQGREFIWNDLLSLET |
| | GSLKLANGRVIEKTLYNRRTRQDEPALFVALTFERREVLDSSNIKPMNLIGIDRG |
| | ENIPAVIALTDPEGCPLSRFKDSLGNPTHILRIGESYKEKQRTIQAKKEVEQRRA |
| | GGYSRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDRVLE |
| | KLKKTATGWMITINGKELKVEGQITYYNRYKRQNVVKDLSVELDRLSEESVNNDI |
| | SSWTKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID NO: |
| | 117) |
| 279 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 118) |
| 280 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGESKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 119) |
| 285 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 120) |
| 286 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 121) |
| 287 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 122) |
| 288 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTMSSGFACSQCCQPLYVYKLEQVNDKGKPHTNYFGRCNVSEH |
| | ERLILLSPHKPEANDELVTYSLGKFGQRALDFYSIHVTRESNHPVKPLEQIGGNS |
| | CASGPVGKALSDACMGAVASFLTKYQDIILEHQKVIKKNEKRLANLKDIASANGL |
| | AFPKITLPPQPHTKEGIEAYNNVVAQIVIWVNLNLWQKLKIGRDEAKPLQRLKGF |
| | PSFPLVERQANEVDWWDMVCNVKKLINEKKEDGKVFWQNLAGYKRQEALRPYLSS |
| | EEDRKKGKKFARYQFGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGID |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 123) |
| 290 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 124) |
| 291 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 125) |
| 293 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 126) |
| 300 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 127) |
| 492 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 128) |
| 493 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 129) |
| 387: | QEIKRINKIRRRLVKDSNTKKAGKTGPMKTLLVRVMTPDLRERLENLRKKPENIP |
| NTSB | QPISNTSRANLNKLLTDYTEMKKAILHVYWEEFQKDPVGLMSRVAQPASKKIDQN |
| swap from | KLKPEMDEKGNLTTAGFACSQCGQPLFVYKLEQVSEKGKAYTNYFGRCNVAEHEK |
| SEQ ID | LILLAQLKPEKDSDEAVTYSLGKFGQRALDFYSIHVTRESNHPVKPLEQIGGNSC |
| NO: 1 | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 130) |
| 395: | QEIKRINKIRRRLVKDSNTKKAGKTGPMKTLLVRVMTPDLRERLENLRKKPENIP |
| Helical 1B | QPISNTSRANLNKLLTDYTEMKKAILHVYWEEFQKDPVGLMSRVAQPAPKNIDQR |
| swap from | KLIPVKDGNERLTSSGFACSQCCQPLYVYKLEQVNDKGKPHTNYFGRCNVSEHER |
| SEQ ID | LILLSPHKPEANDELVTYSLGKFGQRALDFYSIHVTKESTHPVKPLAQIAGNRYA |
| NO: 1 | SGPVGKALSDACMGTIASFLSKYQDIIIEHQKVVKGNQKRLESLRELAGKENLEY |
| | PSVTLPPQPHTKEGVDAYNEVIARVRMWVNLNLWQKLKLSRDDAKPLLRLKGFPS |
| | FPLVERQANEVDWWDMVCNVKKLINEKKEDGKVFWQNLAGYKRQEALRPYLSSEE |
| | DRKKGKKFARYQFGDLLLHLEKKHGEDWGKVYDEAWERIDKKVEGLSKHIKLEEE |
| | RRSEDAQSKAALTDWLRAKASFVIEGLKEADKDEFCRCELKLQKWYGDLRGKPFA |
| | IEAENSILDISGESKQYNCAFIWQKDGVKKLNLYLIINYFKGGKLRFKKIKPEAF |
| | EANRFYTVINKKSGEIVPMEVNFNFDDPNLIILPLAFGKRQGREFIWNDLLSLET |
| | GSLKLANGRVIEKTLYNRRTRQDEPALFVALTFERREVLDSSNIKPMNLIGIDRG |
| | ENIPAVIALTDPEGCPLSRFKDSLGNPTHILRIGESYKEKQRTIQAKKEVEQRRA |
| | GGYSRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDRVLE |
| | KLKKTATGWMTTINGKELKVEGQITYYNRYKRQNVVKDLSVELDRLSEESVNNDI |
| | SSWTKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID NO: |
| | 131) |
| 485: | QEIKRINKIRRRLVKDSNTKKAGKTGPMKTLLVRVMTPDLRERLENLRKKPENIP |
| Helical 1B | QPISNTSRANLNKLLTDYTEMKKAILHVYWEEFQKDPVGLMSRVAQPAPKNIDQR |
| swap from | KLIPVKDGNERLTSSGFACSQCCQPLYVYKLEQVNDKGKPHTNYFGRCNVSEHER |
| SEQ ID | LILLSPHKPEANDELVTYSLGKFGQRALDFYSIHVTKESTHPVKPLAQIAGNRYA |
| NO: 1 | SGPVGKALSDACMGTIASFLSKYQDIIIEHQKVVKGNQKRLESLRELAGKENLEY |
| | PSVTLPPQPHTKEGVDAYNEVIARVRMWVNLNLWQKLKLSRDDAKPLLRLKGFPS |
| | FPLVERQANEVDWWDMVCNVKKLINEKKEDGKVFWQNLAGYKRQEALRPYLSSEE |
| | DRKKGKKFARYQLGDLLLHLEKKHGEDWGKVYDEAWERIDKKVEGLSKHIKLEEE |
| | RRSEDAQSKAALTDWLRAKASFVIEGLKEADKDEFCRCELKLQKWYGDLRGKPFA |
| | IEAENSILDISGFSKQYNCAFIWQKDGVKKLNLYLIINYFKGGKLRFKKIKPEAF |
| | EANRFYTVINKKSGEIVPMEVNFNFDDPNLIILPLAFGKRQGREFIWNDLLSLET |
| | GSLKLANGRVIEKTLYNRRTRQDEPALFVALTFERREVLDSSNIKPMNLIGVDRG |
| | ENIPAVIALTDPEGCPLSRFKDSLGNPTHILRIGESYKEKQRTIQAKKEVEQRRA |
| | GGYSRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDRVLE |
| | KLKKTATGWMTTINGKELKVEGQITYYNRRKRQNVVKDLSVELDRLSEESVNNDI |
| | SSWTKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID NO: |
| | 132) |
| 486: | QEIKRINKIRRRLVKDSNTKKAGKTGPMKTLLVRVMTPDLRERLENLRKKPENIP |
| Helical 1B | QPISNTSRANLNKLLTDYTEMKKAILHVYWEEFQKDPVGLMSRVAQPAPKNIDQR |
| swap from | KLIPVKDGNERLTSSGFACSQCCQPLYVYKLEQVNDKGKPHTNYFGRCNVSEHER |
| SEQ ID | LILLSPHKPEANDELVTYSLGKFGQRALDFYSIHVTKESTHPVKPLAQIAGNRYA |
| NO: 1 | SGPVGKALSDACMGTIASFLSKYQDIIIEHQKVVKGNQKRLESLRELAGKENLEY |
| | PSVTLPPQPHTKEGVDAYNEVIARVRMWVNLNLWQKLKLSRDDAKPLLRLKGFPS |
| | FPLVERQANEVDWWDMVCNVKKLINEKKEDGKVFWQNLAGYKRQEALRPYLSSEE |
| | DRKKGKKFARYQLGDLLKHLEKKHGEDWGKVYDEAWERIDKKVEGLSKHIKLEEE |
| | RRSEDAQSKAALTDWLRAKASFVIEGLKEADKDEFCRCELKLQKWYGDLRGKPFA |
| | IEAENSILDISGFSKQYNCAFIWQKDGVKKLNLYLIINYFKGGKLRFKKIKPEAF |
| | EANRFYTVINKKSGEIVPMEVNFNFDDPNLIILPLAFGKRQGREFIWNDLLSLET |
| | GSLKLANGRVIEKTLYNRRTRQDEPALFVALTFERREVLDSSNIKPMNLIGVDRG |
| | ENIPAVIALTDPEGCPLSRFKDSLGNPTHILRIGESYKEKQRTIQAKKEVEQRRA |
| | GGYSRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDRVLE |
| | KLKKTATGWMTTINGKELKVEGQITYYNRRKRQNVVKDLSVELDRLSEESVNNDI |
| | SSWTKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID NO: |
| | 133) |
| 487: | QEIKRINKIRRRLVKDSNTKKAGKTGPMKTLLVRVMTPDLRERLENLRKKPENIP |
| Helical 1B | QPISNTSRANLNKLLTDYTEMKKAILHVYWEEFQKDPVGLMSRVAQPAPKNIDQR |
| swap from | KLIPVKDGNERLTSSGFACSQCCQPLYVYKLEQVNDKGKPHTNYFGRCNVSEHER |
| SEQ ID | LILLSPHKPEANDELVTYSLGKFGQRALDFYSIHVTKESTHPVKPLAQIAGNRYA |
| NO: 1 | SGPVGKALSDACMGTIASFLSKYQDIIIEHQKVVKGNQKRLESLRELAGKENLEY |
| | PSVTLPPQPHTKEGVDAYNEVIARVRMWVNLNLWQKLKLSRDDAKPLLRLKGFPS |
| | FPLVERQANEVDWWDMVCNVKKLINEKKEDGKVFWQNLAGYKRQEALRPYLSSEE |
| | DRKKGKKFARYQLGDLLLHLEKKHGEDWGKVYDEAWERIDKKVEGLSKHIKLEEE |
| | RRSEDAQSKAALTDWLRAKASFVIEGLKEADKDEFCRCELKLQKWYGDLRGKPFA |
| | IEAENSILDISGFSKQYNCAFIWQKDGVKKLNLYLIINYFKGGKLRFKKIKPEAF |
| | EANRFYTVINKKSGEIVPMEVNENEDDPNLIILPLAFGKRQGREFIWNDLLSLET |
| | GSLKLANGRVIEKTLYNRRTRQDEPALFVALTFERREVLDSSNIKPMNLIGVDRG |
| | ENIPAVIALTDPEGCPLSRFKDSLGNPTHILRIGESYKEKQRTIQAKKEVEQRRA |
| | GGYSRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDRVLE |
| | KLKKTATGWMTTINGKELKVEGQITYYNRYKRQNVVKDLSVELDRLSEESVNNDI |
| | SSWTKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID NO: |
| | 134) |
| 488: | QEIKRINKIRRRLVKDSNTKKAGKTGPMKTLLVRVMTPDLRERLENLRKKPENIP |
| NTSB and | QPISNTSRANLNKLLTDYTEMKKAILHVYWEEFQKDPVGLMSRVAQPASKKIDQN |
| Helical 1B | KLKPEMDEKGNLTTAGFACSQCGQPLFVYKLEQVSEKGKAYTNYFGRCNVAEHEK |
| swap from | LILLAQLKPEKDSDEAVTYSLGKFGQRALDFYSIHVTKESTHPVKPLAQIAGNRY |
| SEQ ID | ASGPVGKALSDACMGTIASFLSKYQDIIIEHQKVVKGNQKRLESLRELAGKENLE |
| NO: 1 | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 135) |
| 489: | QEIKRINKIRRRLVKDSNTKKAGKTGPMKTLLVRVMTPDLRERLENLRKKPENIP |
| NTSB and | QPISNTSRANLNKLLTDYTEMKKAILHVYWEEFQKDPVGLMSRVAQPASKKIDQN |
| Helical 1B | KLKPEMDEKGNLTTAGFACSQCGQPLFVYKLEQVSEKGKAYTNYFGRCNVAEHEK |
| swap from | LILLAQLKPEKDSDEAVTYSLGKFGQRALDFYSIHVTKESTHPVKPLAQIAGNRY |
| SEQ ID | ASGPVGKALSDACMGTIASFLSKYQDIIIEHQKVVKGNQKRLESLRELAGKENLE |
| NO: 1 | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 136) |
| 490: | QEIKRINKIRRRLVKDSNTKKAGKTGPMKTLLVRVMTPDLRERLENLRKKPENIP |
| NTSB and | QPISNTSRANLNKLLTDYTEMKKAILHVYWEEFQKDPVGLMSRVAQPASKKIDQN |
| Helical 1B | KLKPEMDEKGNLTTAGFACSQCGQPLFVYKLEQVSEKGKAYTNYFGRCNVAEHEK |
| swap from | LILLAQLKPEKDSDEAVTYSLGKFGQRALDFYSIHVTKESTHPVKPLAQIAGNRY |
| SEQ ID | ASGPVGKALSDACMGTIASFLSKYQDIIIEHQKVVKGNQKRLESLRELAGKENLE |
| NO: 1 | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLKHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRRKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 137) |
| 491: | QEIKRINKIRRRLVKDSNTKKAGKTGPMKTLLVRVMTPDLRERLENLRKKPENIP |
| NTSB and | QPISNTSRANLNKLLTDYTEMKKAILHVYWEEFQKDPVGLMSRVAQPASKKIDQN |
| Helical 1B | KLKPEMDEKGNLTTAGFACSQCGQPLFVYKLEQVSEKGKAYTNYFGRCNVAEHEK |
| swap from | LILLAQLKPEKDSDEAVTYSLGKFGQRALDFYSIHVTKESTHPVKPLAQIAGNRY |
| SEQ ID | ASGPVGKALSDACMGTIASFLSKYQDIIIEHQKVVKGNQKRLESLRELAGKENLE |
| NO: 1 | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 138) |
| 494: | QEIKRINKIRRRLVKDSNTKKAGKTGPMKTLLVRVMTPDLRERLENLRKKPENIP |
| NTSB | QPISNTSRANLNKLLTDYTEMKKAILHVYWEEFQKDPVGLMSRVAQPASKKIDQN |
| swap from | KLKPEMDEKGNLTTAGFACSQCGQPLFVYKLEQVSEKGKAYTNYFGRCNVAEHEK |
| SEQ ID | LILLAQLKPEKDSDEAVTYSLGKFGQRALDFYSIHVTRESNHPVKPLEQIGGNSC |
| NO: 1 | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMITINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID NO: |
| | 139) |
| 328: | MQEIKRINKIRRRLVKDSNTKKAGKTGPMKTLLVRVMTPDLRERLENLRKKPENI |
| S867G | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALLPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAAKEVEQRR |
| | AGGYSRKYASKAKNLADDMVRNTARDLLYYAVTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLGVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 140) |
| 388: | MQEIKRINKIRRRLVKDSNTKKAGKTGPMKTLLVRVMTPDLRERLENLRKKPENI |
| L379R + | PQPISNTSRANLNKLLTDYTEMKKAILHVYWEEFQKDPVGLMSRVAQPAPKNIDQ |
| A708K + | RKLIPVKDGNERLTSSGFACSQCCQPLYVYKLEQVNDKGKPHTNYFGRCNVSEHE |
| [P793] + | RLILLSPHKPEANDELVTYSLGKFGQRALDFYSIHVTRESNHPVKPLEQIGGNSC |
| X1 | ASGPVGKALSDACMGAVASFLTKYQDIILEHQKVIKKNEKRLANLKDIASANGLA |
| Helical2 | FPKITLPPQPHTKEGIEAYNNVVAQIVIWVNLNLWQKLKIGRDEAKPLQRLKGFP |
| swap | SFPVVERRENEVDWWNTINEVKKLIDAKRDMGRVFWSGVTAEKRNTILEGYNYLP |
| | NENDHKKREGSLENPKKPAKRQFGDLLLYLEKKYAGDWGKVEDEAWERIDKKIAG |
| | LTSHIEREEARNAEDAQSKAVLTDWLRAKASFVLERLKEMDEKEFYACEIQLQKW |
| | YGDLRGNPFAVEAENSILDISGFSKQYNCAFIWQKDGVKKLNLYLIINYFKGGKL |
| | RFKKIKPEAFEANRFYTVINKKSGEIVPMEVNFNEDDPNLIILPLAFGKRQGREF |
| | IWNDLLSLETGSLKLANGRVIEKTLYNRRTRQDEPALFVALTFERREVLDSSNIK |
| | PMNLIGIDRGENIPAVIALTDPEGCPLSRFKDSLGNPTHILRIGESYKEKQRTIQ |
| | AKKEVEQRRAGGYSRKYASKAKNLADDMVRNTARDLLYYAVTQDAMLIFENLSRG |
| | FGRQGKRTFMAERQYTRMEDWLTAKLAYEGLSKTYLSKTLAQYTSKTCSNCGFTI |
| | TSADYDRVLEKLKKTATGWMTTINGKELKVEGQITYYNRYKRQNVVKDLSVELDR |
| | LSEESVNNDISSWTKGRSGEALSLLKKRFSHRPVQEKFVCLNCGFETHADEQAAL |
| | NIARSWLFLRSQEYKKYQTNKTTGNTDKRAFVETWQSFYRKKLKEVWKPAV |
| | (SEQ ID NO: 141) |
| 389: | MQEIKRINKIRRRLVKDSNTKKAGKTGPMKTLLVRVMTPDLRERLENLRKKPENI |
| L379R + | PQPISNTSRANLNKLLTDYTEMKKAILHVYWEEFQKDPVGLMSRVAQPAPKNIDQ |
| A708K + | RKLIPVKDGNERLTSSGFACSQCCQPLYVYKLEQVNDKGKPHTNYFGRCNVSEHE |
| [P793] + | RLILLSPHKPEANDELVTYSLGKFGQRALDFYSIHVTRESNHPVKPLEQIGGNSC |
| X1 RuvC1 | ASGPVGKALSDACMGAVASFLTKYQDIILEHQKVIKKNEKRLANLKDIASANGLA |
| swap | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPVNLIGVDR |
| | GENIPAVIALTDPEGCPLPEFKDSSGGPTDILRIGEGYKEKQRAIQAAKEVEQRR |
| | AGGYSRKFASKSRNLADDMVRNSARDLFYHAVTHDAVLVFENLSRGFGRQGKRTF |
| | MTERQYTKMEDWLTAKLAYEGLTSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 142) |
| 390: | MQEIKRINKIRRRLVKDSNTKKAGKTGPMKTLLVRVMTPDLRERLENLRKKPENI |
| L379R + | PQPISNTSRANLNKLLTDYTEMKKAILHVYWEEFQKDPVGLMSRVAQPAPKNIDQ |
| A708K + | RKLIPVKDGNERLTSSGFACSQCCQPLYVYKLEQVNDKGKPHTNYFGRCNVSEHE |
| [P793] + | RLILLSPHKPEANDELVTYSLGKFGQRALDFYSIHVTRESNHPVKPLEQIGGNSC |
| X1 RuvC2 | ASGPVGKALSDACMGAVASFLTKYQDIILEHQKVIKKNEKRLANLKDIASANGLA |
| swap | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | NSNSTEFKSYKSGKQPFVGAWQAFYKRRLKEVWKPNA (SEQ ID NO: 143) |
| 527: | QEIKRINKIRRRLVKDSNTKKAGKTRGPMKTLLVRVMTPDLRERLENLRKKPENI |
| ^R26 in | PQPISNTSRANLNKLLTDYTEMKKAILHVYWEEFQKDPVGLMSRVAQPASKKIDQ |
| 491 | NKLKPEMDEKGNLTTAGFACSQCGQPLFVYKLEQVSEKGKAYTNYFGRCNVAEHE |
| | KLILLAQLKPEKDSDEAVTYSLGKFGQRALDFYSIHVTKESTHPVKPLAQIAGNR |
| | YASGPVGKALSDACMGTIASFLSKYQDIIIEHQKVVKGNQKRLESLRELAGKENL |
| | EYPSVTLPPQPHTKEGVDAYNEVIARVRMWVNLNLWQKLKLSRDDAKPLLRLKGF |
| | PSFPLVERQANEVDWWDMVCNVKKLINEKKEDGKVFWQNLAGYKRQEALRPYLSS |
| | EEDRKKGKKFARYQLGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID |
| | NO: 144) |
| 528: | QEIKRINKIRRRLVKDSNTKKAGKTGPMKTLLVRVMTPDLRERLENLRKKPENIP |
| G223 Y in | QPISNTSRANLNKLLTDYTEMKKAILHVYWEEFQKDPVGLMSRVAQPASKKIDQN |
| 515 | KLKPEMDEKGNLTTAGFACSQCGQPLFVYKLEQVSEKGKAYTNYFGRCNVAEHEK |
| | LILLAQLKPEKDSDEAVTYSLGKFGQRALDFYSIHVTKESTHPVKPLAQIAGNRY |
| | ASYPVGKALSDACMGTIASFLSKYQDIIIEHQKVVKGNQKRLESLRELAGKENLE |
| | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID NO: |
| | 145) |
| 529: | QEIKRINKIRRRLVKDSNTKKAGKTGPMKTLLVRVMTPDLRERLENLRKKPENIP |
| G223N in | QPISNTSRANLNKLLTDYTEMKKAILHVYWEEFQKDPVGLMSRVAQPASKKIDQN |
| 515 | KLKPEMDEKGNLTTAGFACSQCGQPLFVYKLEQVSEKGKAYTNYFGRCNVAEHEK |
| | LILLAQLKPEKDSDEAVTYSLGKFGQRALDFYSIHVTKESTHPVKPLAQIAGNRY |
| | ASNPVGKALSDACMGTIASFLSKYQDIIIEHQKVVKGNQKRLESLRELAGKENLE |
| | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGVDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLPSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID |
| | NO: 146) |
| 530: | QEIKRINKIRRRLVKDSNTKKAGKTGPMKTLLVRVMTPDLRERLENLRKKPENIP |
| ^W539 in | QPISNTSRANLNKLLTDYTEMKKAILHVYWEEFQKDPVGLMSRVAQPASKKIDQN |
| 515 | KLKPEMDEKGNLTTAGFACSQCGQPLFVYKLEQVSEKGKAYTNYFGRCNVAEHEK |
| | LILLAQLKPEKDSDEAVTYSLGKFGQRALDFYSIHVTKESTHPVKPLAQIAGNRY |
| | ASGPVGKALSDACMGTIASFLSKYQDIIIEHQKVVKGNQKRLESLRELAGKENLE |
| | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGWGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLPSKTYLSKTLAQYTSKTCSNCGFTITSADYDR |
| | VLEKLKKTATGWMTTINGKELKVEGQITYYNRYKRQNVVKDLSVELDRLSEESVN |
| | NDISSWTKGRSGEALSLLKKRFSHRPVQEKFVCLNCGFETHADEQAALNIARSWL |
| | FLRSQEYKKYQTNKTTGNTDKRAFVETWQSFYRKKLKEVWKPAV (SEQ ID |
| | NO: 147) |
| 531: | QEIKRINKIRRRLVKDSNTKKAGKTGPMKTLLVRVMTPDLRERLENLRKKPENIP |
| ^Y539 in | QPISNTSRANLNKLLTDYTEMKKAILHVYWEEFQKDPVGLMSRVAQPASKKIDQN |
| 515 | KLKPEMDEKGNLTTAGFACSQCGQPLFVYKLEQVSEKGKAYTNYFGRCNVAEHEK |
| | LILLAQLKPEKDSDEAVTYSLGKFGQRALDFYSIHVTKESTHPVKPLAQIAGNRY |
| | ASGPVGKALSDACMGTIASFLSKYQDIIIEHQKVVKGNQKRLESLRELAGKENLE |
| | YPSVTLPPQPHTKEGVDAYNEVIARVRMWVNLNLWQKLKLSRDDAKPLLRLKGFP |
| | SFPLVERQANEVDWWDMVCNVKKLINEKKEDGKVFWQNLAGYKRQEALRPYLSSE |
| | EDRKKGKKFARYQLGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGYGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLPSKTYLSKTLAQYTSKTCSNCGFTITSADYDR |
| | VLEKLKKTATGWMTTINGKELKVEGQITYYNRYKRQNVVKDLSVELDRLSEESVN |
| | NDISSWTKGRSGEALSLLKKRFSHRPVQEKFVCLNCGFETHADEQAALNIARSWL |
| | FLRSQEYKKYQTNKTTGNTDKRAFVETWQSFYRKKLKEVWKPAV (SEQ ID |
| | NO: 147) |
| 532: | QEIKRINKIRRRLVKDSNTKKAGKTRGPMKTLLVRVMTPDLRERLENLRKKPENI |
| ^R26 in | PQPISNTSRANLNKLLTDYTEMKKAILHVYWEEFQKDPVGLMSRVAQPASKKIDQ |
| 515 | NKLKPEMDEKGNLTTAGFACSQCGQPLFVYKLEQVSEKGKAYTNYFGRCNVAEHE |
| | KLILLAQLKPEKDSDEAVTYSLGKFGQRALDFYSIHVTKESTHPVKPLAQIAGNR |
| | YASGPVGKALSDACMGTIASFLSKYQDIIIEHQKVVKGNQKRLESLRELAGKENL |
| | EYPSVTLPPQPHTKEGVDAYNEVIARVRMWVNLNLWQKLKLSRDDAKPLLRLKGF |
| | PSFPLVERQANEVDWWDMVCNVKKLINEKKEDGKVFWQNLAGYKRQEALRPYLSS |
| | EEDRKKGKKFARYQLGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLPSKTYLSKTLAQYTSKTCSNCGFTITSADYDR |
| | VLEKLKKTATGWMTTINGKELKVEGQITYYNRYKRQNVVKDLSVELDRLSEESVN |
| | NDISSWTKGRSGEALSLLKKRFSHRPVQEKFVCLNCGFETHADEQAALNIARSWL |
| | FLRSQEYKKYQTNKTTGNTDKRAFVETWQSFYRKKLKEVWKPAV (SEQ ID |
| | NO: 149) |
| 533: | QEIKRINKIRRRLVKDSNTKKAGKTRGPMKTLLVRVMTPDLRERLENLRKKPENI |
| ^R26 and | PQPISNTSRANLNKLLTDYTEMKKAILHVYWEEFQKDPVGLMSRVAQPASKKIDQ |
| G223 Yin | NKLKPEMDEKGNLTTAGFACSQCGQPLFVYKLEQVSEKGKAYTNYFGRCNVAEHE |
| 515 | KLILLAQLKPEKDSDEAVTYSLGKFGQRALDFYSIHVTKESTHPVKPLAQIAGNR |
| | YASYPVGKALSDACMGTIASFLSKYQDIIIEHQKVVKGNQKRLESLRELAGKENL |
| | EYPSVTLPPQPHTKEGVDAYNEVIARVRMWVNLNLWQKLKLSRDDAKPLLRLKGF |
| | PSFPLVERQANEVDWWDMVCNVKKLINEKKEDGKVFWQNLAGYKRQEALRPYLSS |
| | EEDRKKGKKFARYQLGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGVD |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLPSKTYLSKTLAQYTSKTCSNCGFTITSADYDR |
| | VLEKLKKTATGWMTTINGKELKVEGQITYYNRYKRQNVVKDLSVELDRLSEESVN |
| | NDISSWTKGRSGEALSLLKKRFSHRPVQEKFVCLNCGFETHADEQAALNIARSWL |
| | FLRSQEYKKYQTNKTTGNTDKRAFVETWQSFYRKKLKEVWKPAV (SEQ ID |
| | NO: 150) |

In some embodiments, the CasX variant protein comprises a sequence selected from the group consisting of SEQ ID NOs: 49-150, 233-235, 238-252, 272-281.

In some embodiments, the CasX variant protein has one or more improved characteristic of the CasX protein when compared to a reference CasX protein, for example a reference protein of SEQ ID NO:1, SEQ ID NO:2 or SEQ ID NO:3. In some embodiments, the at least one improved characteristic of the CasX variant is at least about 1.1 to about 100,000-fold improved relative to the reference protein. In some embodiments, the at least one improved characteristic of the CasX variant is at least about 1.1 to about 10,000-fold improved, at least about 1.1 to about 1,000-fold improved, at least about 1.1 to about 500-fold improved, at least about 1.1 to about 400-fold improved, at least about 1.1 to about 300-fold improved, at least about 1.1 to about 200-fold improved, at least about 1.1 to about 100-fold improved, at least about 1.1 to about 50-fold improved, at least about 1.1 to about 40-fold improved, at least about 1.1 to about 30-fold improved, at least about 1.1 to about 20-fold improved, at least about 1.1 to about 10-fold improved, at least about 1.1 to about 9-fold improved, at least about 1.1 to about 8-fold improved, at least about 1.1 to about 7-fold improved, at least about 1.1 to about 6-fold improved, at least about 1.1 to about 5-fold improved, at least about 1.1 to about 4-fold improved, at least about 1.1 to about 3-fold improved, at least about 1.1 to about 2-fold improved, at least about 1.1 to about 1.5-fold improved, at least about 1.5 to about 3-fold improved, at least about 1.5 to about 4-fold improved, at least about 1.5 to about 5-fold improved, at least about 1.5 to about 10-fold improved, at least about 5 to about 10-fold improved, at least about 10 to about 20-fold improved, at least 10 to about 30-fold improved, at least 10 to about 50-fold improved or at least 10 to about 100-fold improved than the reference CasX protein. In some embodiments, the at least one improved characteristic of the CasX variant is at least about 10 to about 1000-fold improved relative to the reference CasX protein.

In some embodiments, the one or more improved characteristics of the CasX variant protein is at least about 1.1, at least about 5, at least about 10, at least about 20, at least about 30, at least about 40, at least about 50, at least about 60, at least about 70, at least about 80, at least about 90, at least about 100, at least about 250, at least about 500, or at least about 1000, at least about 5,000, at least about 10,000, or at least about 100,000-fold improved relative to a reference CasX protein of SEQ ID NO:1, SEQ ID NO:2 or SEQ ID NO:3. In other cases, the one or more improved characteristics of the CasX variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the reference CasX of SEQ ID NO:1, SEQ ID NO:2 or SEQ ID NO:3.

Exemplary characteristics that can be improved in CasX variant proteins relative to the same characteristics in reference CasX proteins include, but are not limited to, improved folding of the variant, improved binding affinity to the gNA, improved binding affinity for a wider spectrum of PAM sequences, improved unwinding of the target DNA, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved protein stability, improved protein:gNA complex stability, improved protein solubility, improved protein:gNA complex solubility, improved protein yield, improved protein expression, and improved fusion characteristics. In some embodiments, the variant comprises at least one improved characteristic. In other embodiments, the variant comprises at least two improved characteristics. In further embodiments, the variant comprises at least three improved characteristics. In some embodiments, the variant comprises at least four improved characteristics. In still further embodiments, the variant comprises at least five, at least six, at least seven, at least eight, at least nine, at least ten, at least eleven, at least twelve, at least thirteen, or more improved characteristics. These improved characteristics are described in more detail below.

j. Protein Stability

In some embodiments, the disclosure provides a CasX variant protein with improved stability relative to a reference CasX protein. In some embodiments, improved stability of the CasX variant protein results in expression of a higher steady state of protein, which improves editing efficiency. In some embodiments, improved stability of the CasX variant protein results in a larger fraction of CasX protein that remains folded in a functional conformation and improves editing efficiency or improves purifiability for manufacturing purposes. As used herein, a “functional conformation” refers to a CasX protein that is in a conformation where the protein is capable of binding a gNA and target DNA. In embodiments wherein the CasX variant does not carry one or more mutations rendering it catalytically dead, the CasX variant is capable of cleaving, nicking, or otherwise modifying the target DNA. For example, a functional CasX variant can, in some embodiments, be used for gene-editing, and a functional conformation refers to an “editing-competent” conformation. In some exemplary embodiments, including those embodiments where the CasX variant protein results in a larger fraction of CasX protein that remains folded in a functional conformation, a lower concentration of CasX variant is needed for applications such as gene editing compared to a reference CasX protein. Thus, in some embodiments, the CasX variant with improved stability has improved efficiency compared to a reference CasX in one or more gene editing contexts.

In some embodiments, the disclosure provides a CasX variant protein having improved thermostability relative to a reference CasX protein. In some embodiments, the CasX variant protein has improved thermostability of the CasX variant protein at a particular temperature range. Without wishing to be bound by any theory, some reference CasX proteins natively function in organisms with niches in groundwater and sediment; thus, some reference CasX proteins may have evolved to exhibit optimal function at lower or higher temperatures that may be desirable for certain applications. For example, one application of CasX variant proteins is gene editing of mammalian cells, which is typically carried out at about 37° C. In some embodiments, a CasX variant protein as described herein has improved thermostability compared to a reference CasX protein at a temperature of at least 16° C., at least 18° C., at least 20° C., at least 22° C., at least 24° C., at least 26° C., at least 28° C., at least 30° C., at least 32° C., at least 34° C., at least 35° C., at least 36° C., at least 37° C., at least 38° C., at least 39° C., at least 40° C., at least 41° C., at least 42° C., at least 44° C., at least 46° C., at least 48° C., at least 50° C., at least 52° C., or greater. In some embodiments, a CasX variant protein has improved thermostability and functionality compared to a reference CasX protein that results in improved gene editing functionality, such as mammalian gene editing applications, which may include human gene editing applications. Improved thermostability of the nuclease may be evaluated through a variety of methods known to one of skill in the art.

In some embodiments, the disclosure provides a CasX variant protein having improved stability of the CasX variant protein:gNA complex relative to the reference CasX protein:gNA complex such that the RNP remains in a functional form. Stability improvements can include increased thermostability, resistance to proteolytic degradation, enhanced pharmacokinetic properties, stability across a range of pH conditions, salt conditions, and tonicity. Improved stability of the complex may, in some embodiments, lead to improved editing efficiency. In some embodiments, the RNP of the CasX variant and gNA variant has at least a 5%, at least a 10%, at least a 15%, or at least a 20%, or at least a 5-20% higher percentage of cleavage-competent RNP compared to an RNP of the reference CasX of SEQ ID NOS: 1-3 and the gNA of any one of SEQ ID NOS:4-16 of Table 1. Exemplary data of increased cleavage-competent RNP are provided in the Examples.

In some embodiments, the disclosure provides a CasX variant protein having improved thermostability of the CasX variant protein:gNA complex relative to the reference CasX protein:gNA complex. In some embodiments, a CasX variant protein has improved thermostability relative to a reference CasX protein. In some embodiments, the CasX variant protein:gNA complex has improved thermostability relative to a complex comprising a reference CasX protein at temperatures of at least 16° C., at least 18° C., at least 20° C., at least 22° C., at least 24° C., at least 26° C., at least 28° C., at least 30° C., at least 32° C., at least 34° C., at least 35° C., at least 36° C., at least 37° C., at least 38° C., at least 39° C., at least 40° C., at least 41° C., at least 42° C., at least 44° C., at least 46° C., at least 48° C., at least 50° C., at least 52° C., or greater. In some embodiments, a CasX variant protein has improved thermostability of the CasX variant protein:gNA complex compared to a reference CasX protein:gNA complex, which results in improved function for gene editing applications, such as mammalian gene editing applications, which may include human gene editing applications. Improved thermostability of the RNP may be evaluated through a variety of methods known to one of skill in the art.

In some embodiments, the improved stability and/or thermostability of the CasX variant protein comprises faster folding kinetics of the CasX variant protein relative to a reference CasX protein, slower unfolding kinetics of the CasX variant protein relative to a reference CasX protein, a larger free energy release upon folding of the CasX variant protein relative to a reference CasX protein, a higher temperature at which 50% of the CasX variant protein is unfolded (Tm) relative to a reference CasX protein, or any combination thereof. These characteristics may be improved by a wide range of values; for example, at least 1.1, at least 1.5, at least 10, at least 50, at least 100, at least 500, at least 1,000, at least 5,000, or at least a 10,000-fold improved, as compared to a reference CasX protein. In some embodiments, improved thermostability of the CasX variant protein comprises a higher Tm of the CasX variant protein relative to a reference CasX protein. In some embodiments, the Tm of the CasX variant protein is between about 20° C. to about 30° C., between about 30° C. to about 40° C., between about 40° C. to about 50° C., between about 50° C. to about 60° C., between about 60° C. to about 70° C., between about 70° C. to about 80° C., between about 80° C. to about 90° C. or between about 90° C. to about 100° C. Thermal stability is determined by measuring the “melting temperature” (Tₘ), which is defined as the temperature at which half of the molecules are denatured. Methods of measuring characteristics of protein stability such as Tm and the free energy of unfolding are known to persons of ordinary skill in the art, and can be measured using standard biochemical techniques in vitro. For example, Tm may be measured using Differential Scanning Calorimetry, a thermo-analytical technique in which the difference in the amount of heat required to increase the temperature of a sample and a reference is measured as a function of temperature (Chen et al (2003) Pharm Res 20:1952-60; Ghirlando et al (1999) Immunol Lett 68:47-52). Alternatively, or in addition, CasX variant protein Tm may be measured using commercially available methods such as the ThermoFisher Protein Thermal Shift system. Alternatively, or in addition, circular dichroism may be used to measure the kinetics of folding and unfolding, as well as the Tm (Murray et al. (2002) J. Chromatogr Sci 40:343-9). Circular dichroism (CD) relies on the unequal absorption of left-handed and right-handed circularly polarized light by asymmetric molecules such as proteins. Certain structures of proteins, for example alpha-helices and beta-sheets, have characteristic CD spectra. Accordingly, in some embodiments, CD may be used to determine the secondary structure of a CasX variant protein.

In some embodiments, improved stability and/or thermostability of the CasX variant protein comprises improved folding kinetics of the CasX variant protein relative to a reference CasX protein. In some embodiments, folding kinetics of the CasX variant protein are improved relative to a reference CasX protein by at least about 5, at least about 10, at least about 50, at least about 100, at least about 500, at least about 1,000, at least about 2,000, at least about 3,000, at least about 4,000, at least about 5,000, or at least about a 10,000-fold improvement. In some embodiments, folding kinetics of the CasX variant protein are improved relative to a reference CasX protein by at least about 1 kJ/mol, at least about 5 kJ/mol, at least about 10 kJ/mol, at least about 20 kJ/mol, at least about 30 kJ/mol, at least about 40 kJ/mol, at least about 50 kJ/mol, at least about 60 kJ/mol, at least about 70 kJ/mol, at least about 80 kJ/mol, at least about 90 kJ/mol, at least about 100 kJ/mol, at least about 150 kJ/mol, at least about 200 kJ/mol, at least about 250 kJ/mol, at least about 300 kJ/mol, at least about 350 kJ/mol, at least about 400 kJ/mol, at least about 450 kJ/mol, or at least about 500 kJ/mol.

Exemplary amino acid changes that can increase the stability of a CasX variant protein relative to a reference CasX protein may include, but are not limited to, amino acid changes that increase the number of hydrogen bonds within the CasX variant protein, increase the number of disulfide bridges within the CasX variant protein, increase the number of salt bridges within the CasX variant protein, strengthen interactions between parts of the CasX variant protein, increase the buried hydrophobic surface area of the CasX variant protein, or any combinations thereof.

k. Protein Yield

In some embodiments, the disclosure provides a CasX variant protein having improved yield during expression and purification relative to a reference CasX protein. In some embodiments, the yield of CasX variant proteins purified from bacterial or eukaryotic host cells is improved relative to a reference CasX protein. In some embodiments, the bacterial host cells are Escherichia coli cells. In some embodiments, the eukaryotic cells are yeast, plant (e.g. tobacco), insect (e.g. Spodoptera frugiperda sf9 cells), mouse, rat, hamster, guinea pig, monkey, or human cells. In some embodiments, the eukaryotic host cells are mammalian cells, including, but not limited to human embryonic kidney 293 (HEK293) cells, HEK292T cells, baby hamster kidney (BHK) cells, NS0 cells, SP2/0 cells, YO myeloma cells, P3X63 mouse myeloma cells, PER cells, PER.C6 cells, hybridoma cells, NIH3T3 cells, COS, HeLa, or Chinese hamster ovary (CHO) cells.

In some embodiments, improved yield of the CasX variant protein is achieved through codon optimization. Cells use 64 different codons, 61 of which encode the 20 standard amino acids, while another 3 function as stop codons. In some cases, a single amino acid is encoded by more than one codon. Different organisms exhibit bias towards use of different codons for the same naturally occurring amino acid. Therefore, the choice of codons in a protein coding sequence, and matching codon choice to the organism in which the protein will be expressed, can, in some cases, significantly affect protein translation and therefore protein expression levels. In some embodiments, the CasX variant protein is encoded by a nucleic acid that has been codon optimized. In some embodiments, the nucleic acid encoding the CasX variant protein has been codon optimized for expression in a bacterial cell, a yeast cell, an insect cell, a plant cell, or a mammalian cell. In some embodiments, the mammal cell is a mouse, a rat, a hamster, a guinea pig, a monkey, or a human. In some embodiments, the CasX variant protein is encoded by a nucleic acid that has been codon optimized for expression in a human cell. In some embodiments, the CasX variant protein is encoded by a nucleic acid from which nucleotide sequences that reduce translation rates in prokaryotes and eukaryotes have been removed. For example, runs of greater than three thymine residues in a row can reduce translation rates in certain organisms or internal polyadenylation signals can reduce translation.

In some embodiments, improvements in solubility and stability, as described herein, result in improved yield of the CasX variant protein relative to a reference CasX protein.

Improved protein yield during expression and purification can be evaluated by methods known in the art. For example, the amount of CasX variant protein can be determined by running the protein on an SDS-page gel, and comparing the CasX variant protein to a either a control whose amount or concentration is known in advance to determine an absolute level of protein. Alternatively, or in addition, a purified CasX variant protein can be run on an SDS-page gel next to a reference CasX protein undergoing the same purification process to determine relative improvements in CasX variant protein yield. Alternatively, or in addition, levels of protein can be measured using immunohistochemical methods such as Western blot or ELISA with an antibody to CasX, or by HPLC. For proteins in solution, concentration can be determined by measuring of the protein's intrinsic UV absorbance, or by methods which use protein-dependent color changes such as the Lowry assay, the Smith copper/bicinchoninic assay or the Bradford dye assay. Such methods can be used to calculate the total protein (such as, for example, total soluble protein) yield obtained by expression under certain conditions. This can be compared, for example, to the protein yield of a reference CasX protein under similar expression conditions.

### 1. Protein Solubility

In some embodiments, a CasX variant protein has improved solubility relative to a reference CasX protein. In some embodiments, a CasX variant protein has improved solubility of the CasX:gNA ribonucleoprotein complex variant relative to a ribonucleoprotein complex comprising a reference CasX protein.

In some embodiments, an improvement in protein solubility leads to higher yield of protein from protein purification techniques such as purification from E. coli. Improved solubility of CasX variant proteins may, in some embodiments, enable more efficient activity in cells, as a more soluble protein may be less likely to aggregate in cells. Protein aggregates can in certain embodiments be toxic or burdensome on cells, and, without wishing to be bound by any theory, increased solubility of a CasX variant protein may ameliorate this result of protein aggregation. Further, improved solubility of CasX variant proteins may allow for enhanced formulations permitting the delivery of a higher effective dose of functional protein, for example in a desired gene editing application. In some embodiments, improved solubility of a CasX variant protein relative to a reference CasX protein results in improved yield of the CasX variant protein during purification of at least about 5, at least about 10, at least about 20, at least about 30, at least about 40, at least about 50, at least about 60, at least about 70, at least about 80, at least about 90, at least about 100, at least about 250, at least about 500, or at least about 1000-fold greater yield. In some embodiments, improved solubility of a CasX variant protein relative to a reference CasX protein improves activity of the CasX variant protein in cells by at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.5, at least about 4, at least about 4.5, at least about 5, at least about 5.5, at least about 6, at least about 6.5, at least about 7.0, at least about 7.5, at least about 8, at least about 8.5, at least about 9, at least about 9.5, at least about 10, at least about 11, at least about 12, at least about 13, at least about 14, or at least about 15-fold greater activity. Improved solubility of the nuclease may be evaluated through a variety of methods known to one of skill in the art, including by taking densitometry readings on a gel of the soluble fraction of lysed E. coli. Alternatively, or addition, improvements in CasX variant protein solubility can be measured by measuring the maintenance of soluble protein product through the course of a full protein purification. For example, soluble protein product can be measured at one or more steps of gel affinity purification, tag cleavage, cation exchange purification, running the protein on a sizing column. In some embodiments, the densitometry of every band of protein on a gel is read after each step in the purification process. CasX variant proteins with improved solubility may, in some embodiments, maintain a higher concentration at one or more steps in the protein purification process when compared to the reference CasX protein, while an insoluble protein variant may be lost at one or more steps due to buffer exchanges, filtration steps, interactions with a purification column, and the like.

In some embodiments, improving the solubility of CasX variant proteins results in a higher yield in terms of mg/L of protein during protein purification when compared to a reference CasX protein.

In some embodiments, improving the solubility of CasX variant proteins enables a greater amount of editing events compared to a less soluble protein when assessed in editing assays such as the EGFP disruption assays described herein.

m. Protein Affinity for the gNA

In some embodiments, a CasX variant protein has improved affinity for the gNA relative to a reference CasX protein, leading to the formation of the ribonucleoprotein complex. Increased affinity of the CasX variant protein for the gNA may, for example, result in a lower K_{d} for the generation of a RNP complex, which can, in some cases, result in a more stable ribonucleoprotein complex formation. In some embodiments, increased affinity of the CasX variant protein for the gNA results in increased stability of the ribonucleoprotein complex when delivered to human cells. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity, for example in vivo or in vitro gene editing.

In some embodiments, a higher affinity (tighter binding) of a CasX variant protein to a gNA allows for a greater amount of editing events when both the CasX variant protein and the gNA remain in an RNP complex. Increased editing events can be assessed using editing assays such as the EGFP disruption assay described herein.

In some embodiments, the K_{d} of a CasX variant protein for a gNA is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the CasX variant has about 1.1 to about 10-fold increased binding affinity to the gNA compared to the reference CasX protein of SEQ ID NO: 2.

Without wishing to be bound by theory, in some embodiments amino acid changes in the Helical I domain can increase the binding affinity of the CasX variant protein with the gNA targeting sequence, while changes in the Helical II domain can increase the binding affinity of the CasX variant protein with the gNA scaffold stem loop, and changes in the oligonucleotide binding domain (OBD) increase the binding affinity of the CasX variant protein with the gRNA triplex.

Methods of measuring CasX protein binding affinity for a CasX gNA include in vitro methods using purified CasX protein and gNA. The binding affinity for reference CasX and variant proteins can be measured by fluorescence polarization if the gNA or CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the reference CasX and variant proteins of the disclosure for specific gNAs such as reference gNAs and variants thereof include, but are not limited to, isothermal calorimetry (ITC), and surface plasmon resonance (SPR), as well as the methods of the Examples.

n. Affinity for Target Nucleic Acid

In some embodiments, a CasX variant protein has improved binding affinity for a target nucleic acid sequence relative to the affinity of a reference CasX protein for a target nucleic acid sequence. In some embodiments, the improved affinity for the target nucleic acid sequence comprises improved affinity for the target nucleic acid sequence, improved binding affinity to a wider spectrum of PAM sequences, an improved ability to search DNA for the target nucleic acid sequence, or any combinations thereof. Without wishing to be bound by theory, it is thought that CRISPR/Cas system proteins such as CasX may find their target nucleic acid sequences by one-dimension diffusion along a DNA molecule. The process is thought to include (1) binding of the ribonucleoprotein to the DNA molecule followed by (2) stalling at the target nucleic acid sequence, either of which may be, in some embodiments, affected by improved affinity of CasX proteins for a target nucleic acid sequence, thereby improving function of the CasX variant protein compared to a reference CasX protein.

In some embodiments, a CasX variant protein with improved target nucleic acid sequence affinity has increased overall affinity for DNA. In some embodiments, a CasX variant protein with improved target nucleic acid affinity has increased affinity for specific PAM sequences other than the canonical TTC PAM recognized by the reference CasX proteins of SEQ ID NOS: 1 or 2, including binding affinity for PAM sequences selected from the group consisting of TTC, ATC, GTC, and CTC. Without wishing to be bound by theory, it is possible that these protein variants will interact more strongly with DNA overall and will have an increased ability to access and edit sequences within the target DNA due to the ability to bind additional PAM sequences beyond those of wild-type Cas X, thereby allowing for a more efficient search process of the CasX protein for the target sequence. A higher overall affinity for DNA also, in some embodiments, can increase the frequency at which a CasX protein can effectively start and finish a binding and unwinding step, thereby facilitating target strand invasion and R-loop formation, and ultimately the cleavage of a target nucleic acid sequence.

Without wishing to be bound by theory, it is possible that amino acid changes in the NTSBD that increase the efficiency of unwinding, or capture, of a non-target DNA strand in the unwound state, can increase the affinity of CasX variant proteins for target DNA. Alternatively, or in addition, amino acid changes in the NTSBD that increase the ability of the NTSBD to stabilize DNA during unwinding can increase the affinity of CasX variant proteins for target DNA. Alternatively, or in addition, amino acid changes in the OBD may increase the affinity of CasX variant protein binding to the protospacer adjacent motif (PAM), thereby increasing affinity of the CasX variant protein for the target nucleic acid sequence. Alternatively, or in addition, amino acid changes in the Helical I and/or II, RuvC and TSL domains that increase the affinity of the CasX variant protein for the target nucleic acid strand can increase the affinity of the CasX variant protein for the target nucleic acid sequence.

In some embodiments, the CasX variant protein has increased binding affinity to the target nucleic acid sequence compared to the reference protein of SEQ ID NO: 1, SEQ ID NO: 2, or SEQ ID NO: 3. In some embodiments, affinity of a CasX variant protein of the disclosure for a target nucleic acid molecule is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

In some embodiments, a CasX variant protein has improved binding affinity for the non-target strand of the target nucleic acid. As used herein, the term “non-target strand” refers to the strand of the DNA target nucleic acid sequence that does not form Watson and Crick base pairs with the targeting sequence in the gNA, and is complementary to the target strand.

Methods of measuring CasX protein (such as reference or variant) affinity for a target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include in vitro biochemical assays that measure DNA cleavage events over time.

CasX variant proteins with higher affinity for their target nucleic acid sequence may, in some embodiments, cleave the target nucleic acid sequence more rapidly than a reference CasX protein that does not have increased affinity for the target nucleic acid sequence.

In some embodiments, the CasX variant protein is catalytically dead (dCasX). In some embodiments, the disclosure provides RNP comprising a catalytically-dead CasX protein that retains the ability to bind target DNA. An exemplary catalytically-dead CasX variant protein comprises one or more mutations in the active site of the RuvC domain of the CasX protein. In some embodiments, a catalytically-dead CasX variant protein comprises substitutions at residues 672, 769 and/or 935 of SEQ ID NO: 1. In some embodiments, a catalytically-dead CasX variant protein comprises substitutions of D672A, E769A and/or D935A in the reference CasX protein of SEQ ID NO: 1. In some embodiments, a catalytically-dead CasX protein comprises substitutions at amino acids 659, 765 and/or 922 of SEQ ID NO: 2. In some embodiments, a catalytically-dead CasX protein comprises D659A, E756A and/or D922A substitutions in a reference CasX protein of SEQ ID NO: 2. In further embodiments, a catalytically-dead reference CasX protein comprises deletions of all or part of the RuvC domain of the reference CasX protein.

In some embodiments, improved affinity for DNA of a CasX variant protein also improves the function of catalytically inactive versions of the CasX variant protein. In some embodiments, the catalytically inactive version of the CasX variant protein comprises one or mutations in the DED motif in the RuvC. Catalytically dead CasX variant proteins can, in some embodiments, be used for base editing or epigenetic modifications. With a higher affinity for DNA, in some embodiments, catalytically-dead CasX variant proteins can, relative to catalytically active CasX, find their target DNA faster, remain bound to target DNA for longer periods of time, bind target DNA in a more stable fashion, or a combination thereof, thereby improving the function of the catalytically-dead CasX variant protein.

o. Improved Specificity for a Target Site

In some embodiments, a CasX variant protein has improved specificity for a target nucleic acid sequence relative to a reference CasX protein. As used herein, “specificity,” interchangeably referred to as “target specificity,” refers to the degree to which a CRISPR/Cas system ribonucleoprotein complex cleaves off-target sequences that are similar, but not identical to the target nucleic acid sequence; e.g., a CasX variant RNP with a higher degree of specificity would exhibit reduced off-target cleavage of sequences relative to a reference CasX protein. The specificity, and the reduction of potentially deleterious off-target effects, of CRISPR/Cas system proteins can be vitally important in order to achieve an acceptable therapeutic index for use in mammalian subjects.

In some embodiments, a CasX variant protein has improved specificity for a target site within the target nucleic acid sequence that is complementary to the targeting sequence of the gNA.

Without wishing to be bound by theory, it is possible that amino acid changes in the Helical I and II domains that increase the specificity of the CasX variant protein for the target nucleic acid strand can increase the specificity of the CasX variant protein for the target nucleic acid sequence overall. In some embodiments, amino acid changes that increase specificity of CasX variant proteins for target nucleic acid sequence may also result in decreased affinity of CasX variant proteins for DNA.

Methods of testing CasX protein (such as variant or reference) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and sgRNA is PCR amplified, denatured and rehybridized to form hetero-duplex DNA, containing one wild type strand and one strand with an indel. Mismatches are recognized and cleaved by mismatch detection nucleases, such as Surveyor nuclease or T7 endonuclease I.

p. Unwinding of DNA

In some embodiments, a CasX variant protein has improved ability of unwinding DNA relative to a reference CasX protein. Poor dsDNA unwinding has been shown previously to impair or prevent the ability of CRISPR/Cas system proteins AnaCas9 or Cas14s to cleave DNA. Therefore, without wishing to be bound by any theory, it is likely that increased DNA cleavage activity by some CasX variant proteins of the disclosure is due, at least in part, to an increased ability to find and unwind the dsDNA at a target site.

Without wishing to be bound by theory, it is thought that amino acid changes in the NTSB domain may produce CasX variant proteins with increased DNA unwinding characteristics. Alternatively, or in addition, amino acid changes in the OBD or the helical domain regions that interact with the PAM may also produce CasX variant proteins with increased DNA unwinding characteristics.

Methods of measuring the ability of CasX proteins (such as variant or reference) to unwind DNA include, but are not limited to, in vitro assays that observe increased on rates of dsDNA targets in fluorescence polarization or biolayer interferometry.

q. Catalytic Activity

The ribonucleoprotein complex of the CasX:gNA systems disclosed herein comprise a reference CasX protein or variant thereof that bind to a target nucleic acid sequence and cleaves the target nucleic acid sequence. In some embodiments, a CasX variant protein has improved catalytic activity relative to a reference CasX protein. Without wishing to be bound by theory, it is thought that in some cases cleavage of the target strand can be a limiting factor for Cas12-like molecules in creating a dsDNA break. In some embodiments, CasX variant proteins improve bending of the target strand of DNA and cleavage of this strand, resulting in an improvement in the overall efficiency of dsDNA cleavage by the CasX ribonucleoprotein complex.

In some embodiments, a CasX variant protein has increased nuclease activity compared to a reference CasX protein. Variants with increased nuclease activity can be generated, for example, through amino acid changes in the RuvC nuclease domain. In some embodiments, the CasX variant comprises a nuclease domain having nickase activity. In the foregoing, the CasX nickase of a CasX:gNA system generates a single-stranded break within 10-18 nucleotides 3′ of a PAM site in the non-target strand. In other embodiments, the CasX variant comprises a nuclease domain having double-stranded cleavage activity. In the foregoing, the CasX of the CasX:gNA system generates a double-stranded break within 18-26 nucleotides 5′ of a PAM site on the target strand and 10-18 nucleotides 3′ on the non-target strand. Nuclease activity can be assayed by a variety of methods, including those of the Examples. In some embodiments, a CasX variant has a K_{cleave} constant that is at least 2-fold, or at least 3-fold, or at least 4-fold, or at least 5-fold, or at least 6-fold, or at least 7-fold, or at least 8-fold, or at least 9-fold, or at least 10-fold greater compared to a reference CasX.

In some embodiments, a CasX variant protein has increased target strand loading for double strand cleavage compared to a reference CasX. Variants with increased target strand loading activity can be generated, for example, through amino acid changes in the TLS domain.

Without wishing to be bound by theory, amino acid changes in the TSL domain may result in CasX variant proteins with improved catalytic activity. Alternatively, or in addition, amino acid changes around the binding channel for the RNA:DNA duplex may also improve catalytic activity of the CasX variant protein.

In some embodiments, a CasX variant protein has increased collateral cleavage activity compared to a reference CasX protein. As used herein, “collateral cleavage activity” refers to additional, non-targeted cleavage of nucleic acids following recognition and cleavage of a target nucleic acid sequence. In some embodiments, a CasX variant protein has decreased collateral cleavage activity compared to a reference CasX protein.

In some embodiments, for example those embodiments encompassing applications where cleavage of the target nucleic acid sequence is not a desired outcome, improving the catalytic activity of a CasX variant protein comprises altering, reducing, or abolishing the catalytic activity of the CasX variant protein. In some embodiments, a ribonucleoprotein complex comprising a dCasX variant protein binds to a target nucleic acid sequence and does not cleave the target nucleic acid.

In some embodiments, the CasX ribonucleoprotein complex comprising a CasX variant protein binds a target DNA but generates a single stranded nick in the target DNA. In some embodiments, particularly those embodiments wherein the CasX protein is a nickase, a CasX variant protein has decreased target strand loading for single strand nicking. Variants with decreased target strand loading may be generated, for example, through amino acid changes in the TSL domain.

Exemplary methods for characterizing the catalytic activity of CasX proteins may include, but are not limited to, in vitro cleavage assays, including those of the Examples, below. In some embodiments, electrophoresis of DNA products on agarose gels can interrogate the kinetics of strand cleavage.

r. Affinity for C9orf72 Target DNA and RNA

In some embodiments, a ribonucleoprotein complex comprising a reference CasX protein or a CasX variant protein binds to a target C9orf72 DNA and cleaves the target nucleic acid sequence. In some embodiments, the ribonucleoprotein complex creates a double-stranded break in the target nucleic acid. In other embodiments, the ribonucleoprotein complex creates a single-stranded break in the target nucleic acid. In some embodiments, variants of a reference CasX protein increase the specificity of the CasX variant protein for a target C9orf72 RNA, and increase the activity of the CasX variant protein with respect to a target RNA when compared to the reference CasX protein. For example, CasX variant proteins can display increased binding affinity for target RNAs, or increased cleavage of target RNAs, when compared to reference CasX proteins. In some embodiments, a ribonucleoprotein complex comprising a CasX variant protein binds to a target RNA and/or cleaves the target RNA. In some embodiments, a CasX variant has at least about two-fold to about 10-fold increased binding affinity to the C9orf72 target RNA compared to the reference protein of SEQ ID NO: 1, SEQ ID NO: 2, or SEQ ID NO: 3.

s. Combinations of Mutations

The present disclosure provides Cas X variants that are a combination of mutations from separate CasX variant proteins. In some embodiments, any variant to any domain described herein can be combined with other variants described herein. In some embodiments, any variant within any domain described herein can be combined with other variants described herein, in the same domain. Combinations of different amino acid changes may in some embodiments produce new optimized variants whose function is further improved by the combination of amino acid changes. In some embodiments, the effect of combining amino acid changes on CasX protein function is linear. As used herein, a combination that is linear refers to a combination whose effect on function is equal to the sum of the effects of each individual amino acid change when assayed in isolation. In some embodiments, the effect of combining amino acid changes on CasX protein function is synergistic. As used herein, a combination of variants that is synergistic refers to a combination whose effect on function is greater than the sum of the effects of each individual amino acid change when assayed in isolation. In some embodiments, combining amino acid changes produces CasX variant proteins in which more than one function of the CasX protein has been improved relative to the reference CasX protein.

t. CasX Fusion Proteins

In some embodiments, the disclosure provides CasX proteins comprising a heterologous protein fused to the CasX. In some cases, the CasX is a reference CasX protein. In other cases, the CasX is a CasX variant of any of the embodiments described herein.

In some embodiments, the CasX variant protein is fused to one or more proteins or domains thereof that has a different activity of interest, resulting in a fusion protein. For example, in some embodiments, the CasX variant protein is fused to a protein (or domain thereof) that inhibits transcription, modifies a target nucleic acid sequence, or modifies a polypeptide associated with a nucleic acid (e.g., histone modification).

In some embodiments, a heterologous polypeptide (or heterologous amino acid such as a cysteine residue or a non-natural amino acid) can be inserted at one or more positions within a CasX protein to generate a CasX fusion protein. In other embodiments, a cysteine residue can be inserted at one or more positions within a CasX protein followed by conjugation of a heterologous polypeptide described below. In some alternative embodiments, a heterologous polypeptide or heterologous amino acid can be added at the N- or C-terminus of the reference or CasX variant protein. In other embodiments, a heterologous polypeptide or heterologous amino acid can be inserted internally within the sequence of the CasX protein.

In some embodiments, the reference CasX or variant fusion protein retains RNA-guided sequence specific target nucleic acid binding and cleavage activity. In some cases, the reference CasX or variant fusion protein has (retains) 50% or more of the activity (e.g., cleavage and/or binding activity) of the corresponding reference CasX or variant protein that does not have the insertion of the heterologous protein. In some cases, the reference CasX fusion or CasX variant fusion protein retains at least about 60%, or at least about 70% or more, at least about 80%, or at least about 90%, or at least about 92%, or at least about 95%, or at least about 98%, or at least about 100% of the activity (e.g., cleavage and/or binding activity) of the corresponding CasX protein that does not have the insertion of the heterologous protein.

In some cases, the reference CasX or variant fusion protein retains (has) target nucleic acid binding activity relative to the activity of the CasX protein without the inserted heterologous amino acid or heterologous polypeptide. In some cases, the reference CasX or variant fusion protein retains at least about 60%, or at least about 70% or more, at least about 80%, or at least about 90%, or at least about 92%, or at least about 95%, or at least about 98%, or at least about 100% of the binding activity of the corresponding CasX protein that does not have the insertion of the heterologous protein.

In some cases, the reference CasX or variant fusion protein retains (has) target nucleic acid binding and/or cleavage activity relative to the activity of the parent CasX protein without the inserted heterologous amino acid or heterologous polypeptide. For example, in some cases, the reference CasX or variant fusion protein has (retains) 50% or more of the binding and/or cleavage activity of the corresponding parent CasX protein (the CasX protein that does not have the insertion). For example, in some cases, the reference CasX or variant fusion protein has (retains) 60% or more (70% or more, 80% or more, 90% or more, 92% or more, 95% or more, 98% or more, or 100%) of the binding and/or cleavage activity of the corresponding CasX parent protein (the CasX protein that does not have the insertion). Methods of measuring cleaving and/or binding activity of a CasX protein and/or a CasX fusion protein will be known to one of ordinary skill in the art and any convenient method can be used.

A variety of heterologous polypeptides are suitable for inclusion in a reference CasX or CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

In some cases, a fusion partner has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET domain containing 1A, histone lysine methyltransferase (SET1A), SET domain containing 1B, histone lysine methyltransferase (SET1B), lysine methyltransferase 2A (MLL1 to 5, ASCL1 (ASH1) achaete-scute family bHLH transcription factor 1 (ASH1), SET and MYND domain containing 2 (SYMD2), nuclear receptor binding SET domain protein 1 (NSD1), and the like; histone lysine demethylases such as lysine demethylase 3A (JHDM2a)/Lysine-specific demethylase 3B (JHDM2b), lysine demethylase 6A (UTX), lysine demethylase 6B (JMJD3), and the like; histone acetyltransferases such as lysine acetyltransferase 2A (GCN5), lysine acetyltransferase 2B (PCAF), CREB binding protein (CBP), ElA binding protein p300 (p300), TATA-box binding protein associated factor 1 (TAF1), lysine acetyltransferase 5 (TIP60/PLIP), lysine acetyltransferase 6A (MOZ/MYST3), lysine acetyltransferase 6B (MORF/MYST4), SRC proto-oncogene, non-receptor tyrosine kinase (SRC1), nuclear receptor coactivator 3 (ACTR), MYB binding protein 1a (P160), clock circadian regulator (CLOCK), and the like; and DNA demethylases such as Ten-Eleven Translocation (TET) dioxygenase 1 (TET1CD), tet methylcytosine dioxygenase 1 (TET1), demeter (DME), demeter-like 1 (DML1), demeter-like 2 (DML2), protein ROS1 (ROS1), and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as PR/SET domain containing protein (Pr-SET7/8), lysine methyltransferase 5B (SUV4-20H1), PR/SET domain 2 (RIZ1), and the like; histone lysine demethylases such as lysine demethylase 4A (JMJD2A/JHDM3A), lysine demethylase 4B (JMJD2B), lysine demethylase 4C (JMJD2C/GASC1), lysine demethylase 4D (JMJD2D), lysine demethylase 5A (JARID1A/RBP2), lysine demethylase 5B (JARID1B/PLU-1), lysine demethylase 5C (JARID 1C/SMCX), lysine demethylase 5D (JARID1D/SMCY), and the like; histone lysine deacetylases such as histone deacetylase 1 (HDAC1), HDAC2, HDAC3, HDAC8, HDAC4, HDAC5, HDAC7, HDAC9, sirtuin 1 (SIRT1), SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), methyltransferase 1 (MET1), S-adenosyl-L-methionine-dependent methyltransferases superfamily protein (DRM3) (plants), DNA cytosine methyltransferase MET2a (ZMET2), chromomethylase 1 (CMT1), chromomethylase 2 (CMT2) (plants), and the like; and periphery recruitment elements such as Lamin A, Lamin B, and the like.

In some cases the fusion partner has enzymatic activity that modifies the target nucleic acid sequence (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat APOBECl), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a reference CasX or CasX variant protein of the present disclosure is fused to a polypeptide selected from a domain for increasing transcription (e.g., a VP16 domain, a VP64 domain), a domain for decreasing transcription (e.g., a KRAB domain, e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fokl nuclease), or a base editor (e.g., cytidine deaminase such as APOBEC1).

In some cases, the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB 1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARIDlC/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of suitable fusion partners are (i) a dihydrofolate reductase (DHFR) destabilization domain (e.g., to generate a chemically controllable subject RNA-guided polypeptide or a conditionally active RNA-guided polypeptide), and (ii) a chloroplast transit peptide.

Suitable chloroplast transit peptides include, but are not limited to:

(SEQ ID NO: 151)
MASMISSSAVTTVSRASRGQSAAMAPFGGLKSMTGFPVRKVNTDITSIT
SNGGRVKCMQVWPPIGKKKFETLSYLPPLTRDSRA;
(SEQ ID NO: 152)
MASMISSSAVTTVSRASRGQSAAMAPFGGLKSMTGFPVRKVNTDITSIT
SNGGRVKS;
(SEQ ID NO: 153)
MASSMLSSATMVASPAQATMVAPENGLKSSAAFPATRKANNDITSITSN
GGRVNCMQVWPPIEKKKFETLSYLPDLTDSGGRVNC;
(SEQ ID NO: 154)
MAQVSRICNGVQNPSLISNLSKSSQRKSPLSVSLKTQQHPRAYPISSSW
GLKKSGMTLIGSELRPLKVMSSVSTAC;
(SEQ ID NO: 155)
MAQVSRICNGVWNPSLISNLSKSSQRKSPLSVSLKTQQHPRAYPISSSW
GLKKSGMTLIGSELRPLKVMSSVSTAC;
(SEQ ID NO: 156)
MAQINNMAQGIQTLNPNSNFHKPQVPKSSSFLVFGSKKLKNSANSMLVL
KKDSIFMQLFCSFRISASVAAC;
(SEQ ID NO: 157)
MAALVTSQLATSGTVLSVTDRFRRPGFQGLRPRNPADAALGMRTVGASA
APKQSRKPHRFDRRCLSMVV;
(SEQ ID NO: 158)
MAALTTSQLATSATGFGIADRSAPSSLLRHGFQGLKPRSPAGGDATSLS
VTTSARATPKQQRSVQRGSRRFPSVVVC;
(SEQ ID NO: 159)
MASSVLSSAAVATRSNVAQANMVAPFTGLKSAASFPVSRKQNLDITSIA
SNGGRVQC;
(SEQ ID NO: 160)
MESLAATSVFAPSRVAVPAARALVRAGTVVPTRRTSSTSGTSGVKCSAA
VTPQASPVISRSAAAA;
and
(SEQ ID NO: 161)
MGAAATSMQSLKFSNRLVPPSRRLSPVPNNVTCNNLPKSAAPVRTVKCC
ASSWNSTINGAAATTNGASAASS.

In some cases, a reference CasX or variant polypeptide of the present disclosure can include an endosomal escape peptide. In some cases, an endosomal escape polypeptide comprises the amino acid sequence GLFXALLXLLXSLWXLLLXA (SEQ ID NO: 162), wherein each X is independently selected from lysine, histidine, and arginine. In some cases, an endosomal escape polypeptide comprises the amino acid sequence GLFHALLHLLHSLWHLLLHA (SEQ ID NO: 163), or HHHHHHHHHH (SEQ ID NO: 164).

Non-limiting examples of fusion partners for use when targeting ssRNA target nucleic acid sequences include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

A fusion partner can be any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; Endonucleases (for example RNase III, the CRR22 DYW domain, Dicer, and PIN (PilT N-terminus) domains from proteins such as SMG5 and SMG6); proteins and protein domains responsible for stimulating RNA cleavage (for example CPSF, CstF, CFIm and CFIIm); Exonucleases (for example XRN-1 or Exonuclease T); Deadenylases (for example HNT3); proteins and protein domains responsible for nonsense mediated RNA decay (for example UPF1, UPF2, UPF3, UPF3b, RNP SI, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI Dl and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP Al); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising Endonucleases; proteins and protein domains capable of stimulating RNA cleavage; Exonucleases; Deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as a fusion partner have modular organization, with separate sequence-specific RNA binding modules and splicing effector domains. For example, members of the serine/arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived post mitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cc-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pill/Abyl, etc.).

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion polypeptide does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases a reference or CasX variant polypeptide includes (is fused to) a nuclear localization signal (NLS) (e.g., in some cases 2 or more, 3 or more, 4 or more, or 5 or more 6 or more, 7 or more, 8 or more NLSs). Thus, in some cases, a reference or CasX variant polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases a reference or CasX variant polypeptide includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases a reference or CasX variant polypeptide includes (is fused to) between 2 and 5 NLSs (e.g., 2-4, or 2-3 NLSs).

Non-limiting examples of NLSs include sequences derived from: the NLS of the SV40 virus large T-antigen, having the amino acid sequence PKKKRKV (SEQ ID NO: 165); the NLS from nucleoplasmin (e.g., the nucleoplasmin bipartite NLS with the sequence KRPAATKKAGQAKKKK (SEQ ID NO: 166); the c-myc NLS having the amino acid sequence PAAKRVKLD (SEQ ID NO: 167) or RQRRNELKRSP (SEQ ID NO: 168); the hRNPAl M9 NLS having the sequence NQSSNFGPMKGGNFGGRSSGPYGGGGQYFAKPRNQGGY (SEQ ID NO: 169); the sequence RMRIZFKNKGKDTAELRRRRVEVSVELRKAKKDEQILKRRNV (SEQ ID NO: 170) of the IBB domain from importin-alpha; the sequences VSRKRPRP (SEQ ID NO: 171) and PPKKARED (SEQ ID NO: 172) of the myoma T protein; the sequence PQPKKKPL (SEQ ID NO: 173) of human p53; the sequence SALIKKKKKMAP (SEQ ID NO: 174) of mouse c-abl IV; the sequences DRLRR (SEQ ID NO: 175) and PKQKKRK (SEQ ID NO: 176) of the influenza virus NS1; the sequence RKLKKKIKKL (SEQ ID NO: 177) of the Hepatitis virus delta antigen; the sequence REKKKFLKRR (SEQ ID NO: 178) of the mouse Mxl protein; the sequence KRKGDEVDGVDEVAKKKSKK (SEQ ID NO: 179) of the human poly(ADP-ribose) polymerase; the sequence RKCLQAGMNLEARKTKK (SEQ ID NO: 180) of the steroid hormone receptors (human) glucocorticoid; the sequence PRPRKIPR (SEQ ID NO: 181) of Borna disease virus P protein (BDV-P1); the sequence PPRKKRTVV (SEQ ID NO: 182) of hepatitis C virus nonstructural protein (HCV-NS5A); the sequence NLSKKKKRKREK (SEQ ID NO: 183) of LEF1; the sequence RRPSRPFRKP (SEQ ID NO: 184) of ORF57 simirae; the sequence KRPRSPSS (SEQ ID NO: 185) of EBV LANA; the sequence KRGINDRNFWRGENERKTR (SEQ ID NO: 186) of Influenza A protein; the sequence PRPPKMARYDN (SEQ ID NO: 187) of human RNA helicase A (RHA); the sequence KRSFSKAF (SEQ ID NO: 188) of nucleolar RNA helicase II; the sequence KLKIKRPVK (SEQ ID NO: 189) of TUS-protein; the sequence PKKKRKVPPPPAAKRVKLD (SEQ ID NO: 190) associated with importin-alpha; the sequence PKTRRRPRRSQRKRPPT (SEQ ID NO: 191) from the Rex protein in HTLV-1; the sequence MSRRRKANPTKLSENAKKLAKEVEN (SEQ ID NO: 192) from the EGL-13 protein of Caenorhabditis elegans; and the sequences KTRRRPRRSQRKRPPT (SEQ ID NO: 193), RRKKRRPRRKKRR (SEQ ID NO: 194), PKKKSRKPKKKSRK (SEQ ID NO: 195), HKKKHPDASVNFSEFSK (SEQ ID NO: 196), QRPGPYDRPQRPGPYDRP (SEQ ID NO: 197), LSPSLSPLLSPSLSPL (SEQ ID NO: 198), RGKGGKGLGKGGAKRHRK (SEQ ID NO: 199), PKRGRGRPKRGRGR (SEQ ID NO: 200), PKKKRKVPPPPAAKRVKLD (SEQ ID NO: 190), PKKKRKVPPPPKKKRKV (SEQ ID NO: 201), the sequence PAKRARRGYKC (SEQ ID NO: 202) from CPV, the sequence KLGPRKATGRW (SEQ ID NO: 203) from B19, and the sequence PRRKREE (SEQ ID NO: 204) from hBOV. In general, NLS (or multiple NLSs) are of sufficient strength to drive accumulation of a reference or CasX variant fusion protein in the nucleus of a eukaryotic cell. Detection of accumulation in the nucleus may be performed by any suitable technique. For example, a detectable marker may be fused to a reference or CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined.

In some cases, a reference or CasX variant fusion protein includes a “Protein Transduction Domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a reference or CasX variant fusion protein. In some embodiments, a PTD is covalently linked to the carboxyl terminus of a reference or CasX variant fusion protein. In some cases, the PTD is inserted internally in the sequence of a reference or CasX variant fusion protein at a suitable insertion site. In some cases, a reference or CasX variant fusion protein includes (is conjugated to, is fused to) one or more PTDs (e.g., two or more, three or more, four or more PTDs). In some cases, a PTD includes one or more nuclear localization signals (NLS). Examples of PTDs include but are not limited to peptide transduction domain of HIV TAT comprising YGRKKRRQRRR (SEQ ID NO: 205), RKKRRQRR (SEQ ID NO: 206); YARAAARQARA (SEQ ID NO: 207); THRLPRRRRRR (SEQ ID NO: 208); and GGRRARRRRRR (SEQ ID NO: 209); a polyarginine sequence comprising a number of arginines sufficient to direct entry into a cell (e.g., 3, 4, 5, 6, 7, 8, 9, 10, or 10-50 arginines (SEQ ID NO: 210)); a VP22 domain (Zender et al. (2002) Cancer Gene Ther. 9(6):489-96); an Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97: 13003-13008); RRQRRTSKLMKR (SEQ ID NO: 211); Transportan GWTLNSAGYLLGKINLKALAALAKKIL (SEQ ID NO: 212); KALAWEAKLAKALAKALAKHLAKALAKALKCEA (SEQ ID NO: 213); and RQIKIWFQNRRMKWKK (SEQ ID NO: 214). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a reference or CasX variant fusion protein can include a CasX protein that is linked to an internally inserted heterologous amino acid or heterologous polypeptide (a heterologous amino acid sequence) via a linker polypeptide (e.g., one or more linker polypeptides). In some embodiments, a reference or CasX variant fusion protein can be linked at the C-terminal and/or N-terminal end to a heterologous polypeptide (fusion partner) via a linker polypeptide (e.g., one or more linker polypeptides) The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)n, glycine-serine polymer (including, for example, (GS)n, GSGGSn (SEQ ID NO: 215), GGSGGSn (SEQ ID NO: 216), and GGGSn (SEQ ID NO: 217), where n is an integer of at least one), glycine-alanine polymers, alanine-serine polymers, glycine-proline polymers, proline polymers and proline-alanine polymers. Example linkers can comprise amino acid sequences including, but not limited to, GGSG (SEQ ID NO: 218), GGSGG (SEQ ID NO: 219), GSGSG (SEQ ID NO: 220), GSGGG (SEQ ID NO: 221), GGGSG (SEQ ID NO: 222), GSSSG (SEQ ID NO: 223), GPGP (SEQ ID NO: 224), GGP, PPP, PPAPPA (SEQ ID NO: 225), PPPGPPP (SEQ ID NO: 226) and the like. The ordinarily skilled artisan will recognize that design of a peptide conjugated to any elements described above can include linkers that are all or partially flexible, such that the linker can include a flexible linker as well as one or more portions that confer less flexible structure.

### V. CasX:gNA Systems and Methods for Modification of C9orf72 Genes

The CasX proteins, guide nucleic acids, and variants thereof provided herein are useful for various applications, including as therapeutics, diagnostics, and for research. To effect the methods of the disclosure for gene editing, provided herein are programmable CasX:gNA systems. The programmable nature of the CasX:gNA system provided herein allows for the precise targeting to achieve the desired effect (nicking, cleaving, etc.) at one or more regions of predetermined interest in the target nucleic acid sequence encoding the C9orf72 protein, C9orf72 regulatory element, a non-coding region of the C9orf72 gene, or both. In some embodiments, the CasX:gNA systems provided herein comprise a CasX variant of any one of SEQ ID NOS: 49-150, 233-235, 238-252 or 272-281 as set forth in Tables 4, 6, 7, 8, or 10 or a variant sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gNA scaffold comprises a sequence of any one of SEQ ID NOS: 2101-2294 as set forth in Table 2 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gNA comprises a targeting sequence of any one of SEQ ID NOs: 309-343, 363-2100 or 2295-21835 or a sequence at least 65% identical, at least 70% identical, at least 75% identical, at least 80% identical, at least 85% identical, at least 90% identical, or at least 95% identical thereto and having between 15 and 30 nucleotides. In some embodiments, the targeting sequence of the gNA hybridizes to a target nucleic acid sequence that encodes one or more mutations of the C9orf72 protein of SEQ ID NO: 227 or 228, or one or more mutations that disrupt the function or expression of the C9orf72 protein. In another embodiment, the targeting sequence of the gNA hybridizes to a target nucleic acid sequence comprising a sequence that is 5′ or 3′ to the hexanucleotide repeat GGGGCC or its complement. In other embodiment, the targeting sequence of the gNA hybridizes to a target nucleic acid sequence comprising a regulatory element of the C9orf72 gene. In some embodiments, the targeting sequence of the gNA has a sequence that hybridizes with a C9orf72 exon sequence. In some embodiments, the targeting sequence of the gNA has a sequence that hybridizes with a C9orf72 intron sequence. In some embodiments, the targeting sequence of the gNA has a sequence that hybridizes with the intron 1 of the C9orf72 gene. In some embodiments, the targeting sequences of the plurality of gNAs have sequences that hybridize with a C9orf72 intron-exon junction sequence, a C9orf72 regulatory element, a C9orf72 coding region, a C9orf72 non-coding region, or combinations thereof. In some embodiments of the method, the gNA is chemically modified. In other embodiments, the disclosure provides one or more polynucleotides encoding the foregoing CasX variant proteins and gNAs. In some cases, the CasX:gNA system further comprises a donor template nucleic acid, wherein the donor template can be inserted by HDR or HITI repair mechanisms of the host cell to knock-down or knock-out the C9orf72 gene or, in other cases, to correct the mutation(s); e.g. by deletion of the mutant HRS repeat and insertion of an HRS having between 10 and 30 repeats of the GGGGCC sequence.

In some embodiments, the CasX:gNA systems provided herein comprise a CasX protein and a gNA, or one or more polynucleotides encoding a CasX protein and a gNA, wherein the targeting sequence of the gNA is complementary to, and therefore is capable of hybridizing with, a target nucleic acid sequence encoding the C9orf72 protein, C9orf72 regulatory element, a non-coding region of the C9orf72 gene (e.g., intron 1), sequences that abridge these regions, or is capable or hybridizing with a sequence complementary thereto. In particular embodiments, the targeting sequence of the gNA is complementary to, and therefore is capable of hybridizing with, a sequence within the HRS or a region that is 5′ or 3′ to the HRS. In another particular embodiment, the targeting sequence of the gNA is complementary to, and therefore is capable of hybridizing with a sequence within the promoter of the C9orf72. Exemplary, but non-limiting targeting sequences that can be used to target the C9orf72 HRS include SEQ ID NOS: 309-343 as set forth in Table 15. In some embodiments, the targeting sequence comprises a sequence of SEQ ID NOS: 309-343. In some embodiments, the CasX:gNA system comprises two targeting sequences selected from SEQ ID NOS: 309-343, and the two targeting sequences are not the same. In some embodiments, the CasX:gNA system comprises two targeting sequences, wherein the first targeting sequence comprises SEQ ID NO: 310 and the second targeting sequence is selected from the group consisting of SEQ ID NOS: 321-324. In some embodiments, the CasX:gNA system comprises two targeting sequences, wherein the first targeting sequence comprises SEQ ID NO: 319 and the second targeting sequence is selected from the group consisting of SEQ ID NOS: 321-325. In some embodiments, the CasX:gNA system comprises two targeting sequences, wherein the first targeting sequence comprises SEQ ID NO: 320 and the second targeting sequence is selected from the group consisting of SEQ ID NOS: 321-325. In some embodiments, the two targeting sequences comprise SEQ ID NOS: 310 and 321, SEQ ID NOS: 310 and 322, SEQ ID NOS: 310 and 323, SEQ ID NOS: 310 and 324, SEQ ID NOS: 319 and 321, SEQ ID NOS: 319 and 322, SEQ ID NOS: 319 and 323, SEQ ID NOS: 319 and 324, SEQ ID NOS: 319 and 325, SEQ ID NOS: 320 and 321, SEQ ID NOS: 320 and 322, SEQ ID NOS: 320 and 323, SEQ ID NOS: 320 and 324, or SEQ ID NOS: 320 and 325.

Introducing recombinant expression vectors comprising sequences encoding the CasX:gNA systems (and, optionally, the donor template sequences) of the disclosure into cells under in vitro conditions can occur in any suitable culture media and under any suitable culture conditions that promote the survival of the cells and production of the CasX:gNA. Introducing recombinant expression vectors into a target cell can be carried out in vivo, in vitro or ex vivo. In some embodiments of the method, vectors may be provided directly to a target host cell. For example, cells may be contacted with vectors having nucleic acids encoding the CasX and gNA of any of the embodiments described herein and, optionally, having a donor template sequence such that the vectors are taken up by the cells. Methods for contacting cells with nucleic acid vectors that are plasmids include electroporation, calcium chloride transfection, microinjection, transduction and lipofection are well known in the art. For viral vector delivery, cells can be contacted with viral particles comprising the subject viral expression vectors and the nucleic acid encoding the CasX and gNA and, optionally, the donor template. In some embodiments, the vector is an Adeno-Associated Viral (AAV) vector, wherein the AAV is selected from AAV1, AAV2, AAV3, AAV4, AAV5, AAV6, AAV7, AAV8, AAV9, AAV10, AAV11, AAV12, AAV 44.9, AAV-Rh74, or AAVRh10. Embodiments of AAV vectors are described more fully, below. In other embodiments, the vector is a lentiviral vector. Retroviruses, for example, lentiviruses, may be suitable for use in methods of the present disclosure. Commonly used retroviral vectors are “defective”, e.g., are unable to produce viral proteins required for productive infection, and are commonly referred to as virus-like particles. Rather, replication of the vector requires growth in a packaging cell line. Embodiments of retroviral vectors are described more fully, below.

In other embodiments, the disclosure provides methods of modifying target nucleic acid sequences using the CasX:gNA systems of any of the embodiments described herein, and the methods further comprise contacting the target nucleic acid sequence with an additional CRISPR protein, or a polynucleotide encoding the additional CRISPR protein. In some embodiments, the additional CRISPR protein is a CasX protein having a sequence different from the CasX of the CasX:gNA system. In some embodiments, the additional CRISPR protein is not a CasX protein; e.g., the additional CRISPR protein can be Cpf1, Cas9, Cas12a, or Cas13a.

In some embodiments, it may be desirable to knock-down or knock-out expression of the C9orf72 gene in the subject comprising mutations or duplications, for example dominant mutations or duplications leading to amyotrophic lateral sclerosis (ALS) and frontotemporal dementia (FTD). The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant or heterologous sequence. The term “knock-down” as used herein refers to reduction in the expression of a gene or its gene product(s). As a result of a gene knock-down, the protein activity or function may be attenuated or the protein levels may be reduced or eliminated. In such embodiments, gNAs having targeting sequences specific for a portion of the gene encoding the C9orf72 protein or the C9orf72 regulatory elements may be used. Depending on the CasX protein and gNA used, the event may be a cleavage event, allowing for knock-down/knock-out of expression. In some embodiments, C9orf72 gene expression may be disrupted or eliminated by introducing random insertions or deletions (indels), for example by utilizing the imprecise non-homologous DNA end joining (NHEJ) repair pathway. In such embodiments, the targeted region of the C9orf72 includes coding sequences (exons) of the C9orf72 gene, as inserting or deleting nucleotides within coding sequences can generate a frame shift mutation. This approach can also be used in non-coding regions such as introns, or regulatory elements to disturb expression of the C9orf72 gene. Thus, in some embodiments, the disclosure provides a CasX:gNA system utilized in methods of altering one or more target nucleic acid sequences of a cell, the methods comprising contacting said cell with the CasX:gNA system comprising a CasX protein and a gNA of the embodiments described herein, wherein the gRNA comprises a targeting sequence to a genomic target complementary to, and therefore capable of hybridizing with a sequence encoding C9orf72 protein, a sequence that is 5′ or 3′ to the HRS, the C9orf72 regulatory elements, or the complement of these sequences. In other embodiments, the disclosure provides methods of altering a target nucleic acid sequence of a cell, comprising contacting said cell with a nucleic acid encoding a CasX:gNA system comprising a CasX protein and a gNA of the embodiments described herein, wherein the gRNA comprises a targeting sequence to a genomic target complementary to, and therefore capable of hybridizing with a sequence encoding the C9orf72 protein, a sequence that is 5′ or 3′ to the HRS, a C9orf72 regulatory element, or the complement of these sequences. In other embodiments, the disclosure provides methods of altering a target nucleic acid sequence of a cell, comprising contacting said cell with a vector of the embodiments described herein comprising a nucleic acid encoding a CasX:gNA system comprising a CasX protein and a gNA, wherein the gNA comprises a targeting sequence complementary to, and therefore capable of hybridizing with, a sequence encoding C9orf72 protein, a sequence that is 5′ or 3′ to the HRS, a C9orf72 regulatory element, or the complement of these sequences. In some embodiments, the disclosure provides methods and CasX:gNA systems to knock-down or knock-out cellular expression of both C9orf72 alleles. In some embodiments, the disclosure provides methods and CasX:gNA systems to knock-down or knock-out cellular expression of a single C9orf72 allele. In other embodiments of the methods, the CasX:gNA system further comprises a donor template nucleic acid corresponding to all or at least a portion of a C9orf72 gene, wherein said donor template nucleic acid comprises a heterologous sequence, or a deletion, insertion, or mutation of one or more nucleotides in comparison to a genomic nucleic acid sequence encoding said portion of C9orf72, wherein the contacting results in the knock-down or knock-out of C9orf72. In the foregoing, the cell has been modified such that expression of the HRS is reduced by at least about 10%, at least about 20%, at least about 30%, at least about 40%, at least about 50%, at least about 60%, at least about 70%, at least about 80%, or at least about 90% in comparison to a cell that has not been modified. In other embodiments of the methods, the cell has been modified such that the cell does not express a detectable level of the HRS RNA or the DPR protein. In still other embodiments of the method, the donor template nucleic acid comprises a corrective sequence that upon insertion in the target nucleic acid by the CasX:gNA system, functional C9orf72 protein or physiologically-normal levels of C9orf72 can be expressed.

Thus, the CasX:gNA systems and methods described herein can be used, in combination with conventional molecular biology methods, to modify populations of cells (examples of which are described more fully, below) to produce cells having the capacity to produce a functional C9orf72 protein. This approach, therefore, could be used to generate populations of cells that can be administered to a subject with a disease such as ALS or FTD. In other embodiments, the CasX:gNA systems and methods described herein can be used to treat a subject by administration of components of the system or vectors encoding the CasX:gNA components to modify the C9orf72 gene of the target cells of the subject.

### VI. Polynucleotides and Vectors

In other embodiments, the present disclosure provides polynucleotides encoding the Type V nuclease proteins and the polynucleotides of the gNAs described herein. In some embodiments, the disclosure provides polynucleotides encoding the CasX proteins and the polynucleotides of the gNAs (e.g., the gDNAs and gRNAs) of any of the CasX:gNA system embodiments described herein, as well as sequences complementary to polynucleotides encoding the CasX proteins and the gNAs embodiments. In additional embodiments, the disclosure provides donor template polynucleotides encoding portions or all of a C9orf72 gene. In some cases, the C9orf72 gene of the donor template comprises a mutation or a heterologous sequence for knocking down or knocking out the C9orf72 gene in the target nucleic acid. In other cases, the donor template comprises a corrective sequence for knocking in a functional C9orf72 gene or portion thereof. In yet further embodiments, the disclosure relates to vectors comprising polynucleotides encoding the CasX proteins and the CasX gNAs described herein. In yet further embodiments, the disclosure relates to vectors comprising polynucleotides comprising the donor templates described herein.

In some embodiments, the disclosure provides polynucleotide sequences encoding the reference CasX of SEQ ID NOS: 1-3. In other embodiments, the disclosure provides polynucleotide sequences encoding the CasX variants of any of the embodiments described herein, including the CasX protein variants of SEQ ID NOS: 49-150, 233-235, 238-252, 272-281 as set forth in Tables 4 6-8 and 10, or sequences having at least about 50%, at least about 60%, at least about 70%, at least about 80%, at least about 90%, at least about 95%, at least about 96%, at least about 97%, at least about 98%, or at least about 99% sequence identity to a sequence of Table 4, or the complement of the polynucleotide sequences encoding the variants. In some embodiments, the disclosure provides an isolated polynucleotide sequence encoding a gNA sequence of any of the embodiments described herein, including scaffold the sequences of Table 1 and 2, or a sequence having at least about 50%, at least about 60%, at least about 70%, at least about 80%, at least about 90%, at least about 95%, at least about 96%, at least about 97%, at least about 98%, at least about 99% sequence identity thereto. In some embodiments, the polynucleotide encodes a gNA scaffold sequence selected from the group consisting of SEQ ID NOS:2101-2294, or a sequence having at least about 50%, at least about 60%, at least about 70%, at least about 80%, at least about 90%, at least about 95%, at least about 96%, at least about 97%, at least about 98%, at least about 99% sequence identity thereto.

In some embodiments, the disclosure provides polynucleotides encoding gNA scaffolds and further comprising a targeting sequence polynucleotide linked 3′ to the scaffold having a sequence of SEQ ID NOs: 309-343, 363-2100 or 2295-21835, or a sequence with at least about 65%, at least about 75%, at least about 85%, or at least about 95% identity to a sequence thereto that is complementary to, and therefore hybridizes with, the C9orf72 gene. In other embodiments, the disclosure provides a targeting polynucleotide having 15 nucleotides, 16 nucleotides, 17, nucleotides, 18 nucleotides, 19 nucleotides, 20 nucleotides, or 21 nucleotides. In some cases, the polynucleotide sequence encodes a gNA comprising a targeting sequence that hybridizes with a C9orf72 exon. In other cases, the polynucleotide sequence encodes a gNA comprising a targeting sequence that hybridizes with a C9orf72 intron. In other cases, the polynucleotide sequence encodes a gNA comprising a targeting sequence that hybridizes with a C9orf72 intron-exon junction. In other cases, the polynucleotide sequence encodes a gNA comprising a targeting sequence that hybridizes with an intergenic region of the C9orf72 gene. In other cases, the polynucleotide sequence encodes a gNA comprising a targeting sequence that hybridizes with a sequence located 5′ to the HRS. In other cases, the polynucleotide sequence encodes a gNA comprising a targeting sequence that hybridizes with a sequence located 3′ to the HRS. In other embodiments, the disclosure provides polynucleotide sequences encoding two or more gNAs, each having a scaffold and targeting sequence, that collectively hybridize with a sequence located 5′ and a sequence located 3′ to the HRS. In other embodiments, the polynucleotide sequence encodes a gNA comprising a targeting sequence that hybridizes with a C9orf72 regulatory element. In some cases, the C9orf72 regulatory element is a C9orf72 promoter or enhancer. In some cases, the C9orf72 regulatory element is located 5′ of the C9orf72 transcription start site, 3′ of the C9orf72 transcription start, or in a C9orf72 intron. In some cases, the C9orf72 regulatory element is in an intron of the C9orf72 gene. In other cases, the C9orf72 regulatory element comprises the 5′ UTR of the C9orf72 gene. In still other cases, the C9orf72 regulatory element comprises the 3′UTR of the C9orf72 gene.

In other embodiments, the disclosure provides donor template nucleic acids, wherein the donor template comprises a nucleotide sequence having homology to a C9orf72 target nucleic acid sequence but not complete identity to the target sequence of the target nucleic acid for which gene editing is intended. In some embodiments, the C9orf72 donor template is intended for gene editing and comprises all or at least a portion of a C9orf72 gene. In some embodiments, the C9orf72 donor template comprises a sequence that hybridizes with the C9orf72 gene. In other embodiments, the C9orf72 donor sequence comprises a sequence that encodes at least a portion of a C9orf72 exon. In other embodiments, the C9orf72 donor template has a sequence that encodes at least a portion of a C9orf72 intron. In other embodiments, the C9orf72 donor template has a sequence that encodes at least a portion of a C9orf72 intron-exon junction. In other embodiments, the C9orf72 donor template has a sequence that encodes at least a portion of an intergenic region of the C9orf72 gene. In other embodiments, the C9orf72 donor template has a sequence that encodes at least a portion of a C9orf72 regulatory element. In some cases, the C9orf72 donor template is a wild-type sequence that encodes all or a portion of SEQ ID NO: 227 or 228. In other cases, the C9orf72 donor template sequence comprises one or more mutations relative to a wild-type C9orf72 gene and may contain one or more single base changes, insertions, deletions, inversions or rearrangements with respect to the genomic sequence, provided that there is sufficient homology with the target sequence to support homology-directed repair, or the donor template has homologous arms, whereupon insertion can result in splicing out of regions comprising, for example, the hexanucleotide repeat such that a functional C9orf72 protein can be expressed. In a particular embodiment, the C9orf72 donor template sequence comprises from 10 to about 30 copies of the hexanucleotide repeat GGGGCC. In the foregoing embodiments, the donor template can range in size from 10-10,000 nucleotides. In some embodiments, the donor template is a single-stranded DNA template. In other embodiments, the donor template is a single stranded RNA template. In other embodiments, the donor template is a double-stranded DNA template.

In some embodiments, the disclosure relates to methods to produce polynucleotide sequences encoding the reference CasX, the CasX variants, or the gNA of any of the embodiments described herein, including variants thereof, as well as methods to express the proteins expressed or RNA transcribed by the polynucleotide sequences. In general, the methods include producing a polynucleotide sequence coding for the reference CasX, the CasX variants, or the gNA of any of the embodiments described herein and incorporating the encoding gene into an expression vector appropriate for a host cell. For production of the encoded reference CasX, the CasX variants, or the gNA of any of the embodiments described herein, the method includes transforming an appropriate host cell with an expression vector comprising the encoding polynucleotide, and culturing the host cell under conditions causing or permitting the resulting reference CasX, the CasX variants, or the gNA of any of the embodiments described herein to be expressed or transcribed in the transformed host cell, thereby producing the reference CasX, the CasX variants, or the gNA, which is recovered by methods described herein or by standard purification methods known in the art, including the methods of the Examples. Standard recombinant techniques in molecular biology are used to make the polynucleotides and expression vectors of the present disclosure.

In accordance with the disclosure, polynucleotide sequences that encode the reference CasX, the CasX variants, or the gNA of any of the embodiments described herein are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the reference CasX, the CasX variants, or the gNA that is used to transform a host cell for expression of the composition.

In one approach, a construct is first prepared containing the DNA sequence encoding a reference CasX, a CasX variant, or a gNA. Exemplary methods for the preparation of such constructs are described in the Examples. The construct is then used to create an expression vector suitable for transforming a host cell, such as a prokaryotic or eukaryotic host cell for the expression and recovery of the polypeptide construct. Where desired, the host cell is an E. coli cell. In other embodiments, the host cell is selected from BHK cells, HEK293 cells, HEK293T cells, Lenti-X HEK293 cells, NS0 cells, SP2/0 cells, YO myeloma cells, P3X63 mouse myeloma cells, PER cells, PER.C6 cells, hybridoma cells, NIH3T3 cells, COS, HeLa, CHO, or yeast cells. Exemplary methods for the creation of expression vectors, the transformation of host cells and the expression and recovery of reference CasX, the CasX variants, or the gNA are described in the Examples.

The gene or genes encoding for the reference CasX, the CasX variants, or the gNA constructs can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., CasX and gNA) genes of a desired sequence. Genes encoding polypeptide compositions are assembled from oligonucleotides using standard techniques of gene synthesis.

In some embodiments, the nucleotide sequence encoding a CasX protein is codon optimized. This type of optimization can entail a mutation of an encoding nucleotide sequence to mimic the codon preferences of the intended host organism or cell while encoding the same CasX protein. Thus, the codons can be changed, but the encoded protein remains unchanged. For example, if the intended target cell of the CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. As another non-limiting example, if the intended host cell were a plant cell, then a plant codon-optimized CasX protein variant-encoding nucleotide sequence could be generated. As another non-limiting example, if the intended host cell were an insect cell, then an insect codon-optimized CasX protein-encoding nucleotide sequence could be generated. The gene design can be performed using algorithms that optimize codon usage and amino acid composition appropriate for the host cell utilized in the production of the reference CasX, the CasX variants, or the gNA. In one method of the disclosure, a library of polynucleotides encoding the components of the constructs is created and then assembled, as described above. The resulting genes are then assembled and the resulting genes used to transform a host cell and produce and recover the reference CasX, the CasX variants, or the gNA compositions for evaluation of its properties, as described herein.

In some embodiments, a nucleotide sequence encoding a gNA is operably linked to a control element, e.g., a transcriptional control element, such as a promoter. In some embodiments, a nucleotide sequence encoding a CasX protein is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., the cell is selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

Non-limiting examples of eukaryotic promoters (promoters functional in a eukaryotic cell) include EF1alpha, EF1alpha core promoter, those from cytomegalovirus (CMV) immediate early, herpes simplex virus (HSV) thymidine kinase, early and late SV40, long terminal repeats (LTRs) from retrovirus, and mouse metallothionein-I. Further non-limiting examples of eukaryotic promoters include the CMV promoter full-length promoter, the minimal CMV promoter, the chicken β-actin promoter, the hPGK promoter, the HSV TK promoter, the Mini-TK promoter, the human synapsin I promoter which confers neuron-specific expression, the Mecp2 promoter for selective expression in neurons, the minimal IL-2 promoter, the Rous sarcoma virus enhancer/promoter (single), the spleen focus-forming virus long terminal repeat (LTR) promoter, the SV40 promoter, the SV40 enhancer and early promoter, the TBG promoter: promoter from the human thyroxine-binding globulin gene (Liver specific), the PGK promoter, the human ubiquitin C promoter, the UCOE promoter (Promoter of HNRPA2B1-CBX3), the Histone H2 promoter, the Histone H3 promoter, the U1a1 small nuclear RNA promoter (226 nt), the U1b2 small nuclear RNA promoter (246 nt) 26, the TTR minimal enhancer/promoter, the b-kinesin promoter, the human eIF4A1 promoter, the ROSA26 promoter and the glyceraldehyde 3-phosphate dehydrogenase (GAPDH) promoter.

Selection of the appropriate vector and promoter is well within the level of ordinary skill in the art, as it related to controlling expression, e.g., for modifying a protein involved in antigen processing, antigen presentation, antigen recognition, and/or antigen response and/or its regulatory element. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, FLAG tag, fluorescent protein, etc.) that can be fused to the CasX protein, thus resulting in a chimeric CasX protein that are used for purification or detection.

In some embodiments, a nucleotide sequence encoding each of a gNA variant or a CasX protein is operably linked to an inducible promoter, a constitutively active promoter, a spatially restricted promoter (i.e., transcriptional control element, enhancer, tissue specific promoter, cell type specific promoter, etc.), or a temporally restricted promoter. In other embodiments, individual nucleotide sequences encoding the gNA or the CasX are linked to one of the foregoing categories of promoters, which are then introduced into the cells to be modified by conventional methods, described below.

In certain embodiments, suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), a POL1 promoter, a 7SK promoter, tRNA promoters and the like.

In some embodiments, one or more nucleotide sequences encoding a CasX and gNA and, optionally, comprising a donor template, are each operably linked to (under the control of) a promoter operable in a eukaryotic cell. Examples of inducible promoters may include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore, in some embodiments, be regulated by molecules including, but not limited to, doxycycline; estrogen and/or an estrogen analog; IPTG; etc.

In certain embodiments, inducible promoters suitable for use may include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR, etc.), tetracycline regulated promoters, (e.g., promoter systems including Tet Activators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Recombinant expression vectors of the disclosure can also comprise elements that facilitate robust expression of CasX proteins and the gNAs of the disclosure. For example, recombinant expression vectors can include one or more of a polyadenylation signal (PolyA), an intronic sequence or a post-transcriptional regulatory element such as a woodchuck hepatitis post-transcriptional regulatory element (WPRE). Exemplary polyA sequences include hGH poly(A) signal (short), HSV TK poly(A) signal, synthetic polyadenylation signals, SV40 poly(A) signal, β-globin poly(A) signal and the like. A person of ordinary skill in the art will be able to select suitable elements to include in the recombinant expression vectors described herein.

The polynucleotides encoding the reference CasX, the CasX variants, and the gNA sequences can then be individually cloned into one or more expression vectors. In some embodiments, the present disclosure provides vectors comprising the polynucleotides selected from the group consisting of a retroviral vector, a lentiviral vector, an adenoviral vector, an adeno-associated viral (AAV) vector, a virus-like particle (VLP), a herpes simplex virus (HSV) vector, a plasmid, a minicircle, a nanoplasmid, a DNA vector, and an RNA vector. In some embodiments, the vector is a recombinant expression vector that comprises a nucleotide sequence encoding a CasX protein. In other embodiments, the disclosure provides a recombinant expression vector comprising a nucleotide sequence encoding a CasX protein and a nucleotide sequence encoding a gNA. In some cases, the nucleotide sequence encoding the CasX protein variant and/or the nucleotide sequence encoding the gNA are operably linked to a promoter that is operable in a cell type of choice. In other embodiments, the nucleotide sequence encoding the CasX protein variant and the nucleotide sequence encoding the gNA are provided in separate vectors operably linked to a promoter.

In some embodiments, provided herein are one or more recombinant expression vectors comprising one or more of: (i) a nucleotide sequence of a donor template nucleic acid where the donor template comprises a nucleotide sequence having homology to a target sequence of a target nucleic acid (e.g., a target genome); (ii) a nucleotide sequence that encodes a gNA that hybridizes to a target sequence of the locus of the targeted genome (e.g., configured as a single or dual guide RNA) operably linked to a promoter that is operable in a target cell such as a eukaryotic cell; and (iii) a nucleotide sequence encoding a CasX protein operably linked to a promoter that is operable in a target cell such as a eukaryotic cell. In some embodiments, the sequences encoding the donor template, the gNA and the CasX protein are in different recombinant expression vectors, and in other embodiments one or more polynucleotide sequences (for the donor template, CasX, and the gNA) are in the same recombinant expression vector. In other cases, the CasX and gNA are delivered to the target cell as an RNP (e.g., by electroporation or chemical means) and the donor template is delivered by a vector.

The polynucleotide sequence(s) are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the protein involved in antigen processing, antigen presentation, antigen recognition, and/or antigen response can be determined using any nucleic acid or protein assay known in the art. For example, the presence of transcribed mRNA of reference CasX or the CasX variants can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of the polynucleotide.

The disclosure provides for the use of plasmid expression vectors containing replication and control sequences that are compatible with and recognized by the host cell and are operably linked to the gene encoding the polypeptide for controlled expression of the polypeptide or transcription of the RNA. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired. The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell.

The polynucleotides and recombinant expression vectors can be delivered to the target host cells by a variety of methods. Such methods include, but are not limited to, viral infection, transfection, lipofection, electroporation, calcium phosphate precipitation, polyethyleneimine (PEI)-mediated transfection, DEAE-dextran mediated transfection, microinjection, liposome-mediated transfection, particle gun technology, nucleofection, direct addition by cell penetrating CasX proteins that are fused to or recruit donor DNA, cell squeezing, calcium phosphate precipitation, direct microinjection, nanoparticle-mediated nucleic acid delivery, and using the commercially available TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like.

In accordance with the disclosure, nucleic acid sequences that encode the reference CasX, the CasX variants, or the gNA of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the reference CasX, the CasX variants, or the gNA that is used to transform a host cell for expression of the composition.

In one approach, a construct is first prepared containing the DNA sequence encoding a reference CasX, a CasX variant, or a gNA. Exemplary methods for the preparation of such constructs are described in the Examples. The construct is then used to create an expression vector suitable for transforming a host cell, such as a prokaryotic or eukaryotic host cell for the expression and recovery of the polypeptide construct. Where desired, the host cell is an E. coli cell. In other embodiments, the host cell is selected from BHK cells, HEK293 cells, HEK293T cells, Lenti-X HEK293 cells, NS0 cells, SP2/0 cells, YO myeloma cells, P3X63 mouse myeloma cells, PER cells, PER.C6 cells, hybridoma cells, NIH3T3 cells, COS, HeLa, CHO, or yeast cells. Exemplary methods for the creation of expression vectors, the transformation of host cells and the expression and recovery of reference CasX, the CasX variants, or the gNA are described in the Examples.

The gene or genes encoding for the reference CasX, the CasX variants, or the gNA constructs can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., CasX and gNA) genes of a desired sequence. Genes encoding polypeptide compositions are assembled from oligonucleotides using standard techniques of gene synthesis.

In some embodiments, the nucleotide sequence encoding a CasX protein is codon optimized. This type of optimization can entail a mutation of an encoding nucleotide sequence to mimic the codon preferences of the intended host organism or cell while encoding the same CasX protein. Thus, the codons can be changed, but the encoded protein remains unchanged. For example, if the intended target cell of the CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. As another non-limiting example, if the intended host cell were a plant cell, then a plant codon-optimized CasX protein variant-encoding nucleotide sequence could be generated. As another non-limiting example, if the intended host cell were an insect cell, then an insect codon-optimized CasX protein-encoding nucleotide sequence could be generated. The gene design can be performed using algorithms that optimize codon usage and amino acid composition appropriate for the host cell utilized in the production of the reference CasX, the CasX variants, or the gNA. In one method of the disclosure, a library of polynucleotides encoding the components of the constructs is created and then assembled, as described above. The resulting genes are then assembled and the resulting genes used to transform a host cell and produce and recover the reference CasX, the CasX variants, or the gNA compositions for evaluation of its properties, as described herein.

In some embodiments, a nucleotide sequence encoding a gNA is operably linked to a control element, e.g., a transcriptional control element, such as a promoter. In some embodiments, a nucleotide sequence encoding a CasX protein is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., the cell is selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

Non-limiting examples of eukaryotic promoters (promoters functional in a eukaryotic cell) include EF1alpha, EF1alpha core promoter, those from cytomegalovirus (CMV) immediate early, herpes simplex virus (HSV) thymidine kinase, early and late SV40, long terminal repeats (LTRs) from retrovirus, and mouse metallothionein-I. Further non-limiting examples of eukaryotic promoters include the CMV promoter full-length promoter, the minimal CMV promoter, the chicken 3-actin promoter, the RSV promoter, the HIV-Ltr promoter, the hPGK promoter, the HSV TK promoter, the Mini-TK promoter, the human synapsin I promoter which confers neuron-specific expression, the Mecp2 promoter for selective expression in neurons, the minimal IL-2 promoter, the Rous sarcoma virus enhancer/promoter (single), the spleen focus-forming virus long terminal repeat (LTR) promoter, the SV40 promoter, the SV40 enhancer and early promoter, the TBG promoter: promoter from the human thyroxine-binding globulin gene (Liver specific), the PGK promoter, the human ubiquitin C promoter, the UCOE promoter (Promoter of HNRPA2B1-CBX3), the Histone H2 promoter, the Histone H3 promoter, the U1a1 small nuclear RNA promoter (226 nt), the U1b2 small nuclear RNA promoter (246 nt) 26, the TTR minimal enhancer/promoter, the b-kinesin promoter, the human eIF4A1 promoter, the ROSA26 promoter and the glyceraldehyde 3-phosphate dehydrogenase (GAPDH) promoter. In some embodiments, the promoter used in the gNA construct is U6 (Kunkel, G R et al. U6 small nuclear RNA is transcribed by RNA polymerase III. Proc Natl Acad Sci USA. 83(22):8575 (1986)).

Selection of the appropriate vector and promoter is well within the level of ordinary skill in the art, as it related to controlling expression, e.g., for modifying a C9orf72 gene. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

In some embodiments, a nucleotide sequence encoding each of a gNA variant or a CasX protein is operably linked to an inducible promoter, a constitutively active promoter, a spatially restricted promoter (i.e., transcriptional control element, enhancer, tissue specific promoter, cell type specific promoter, etc.), or a temporally restricted promoter. In other embodiments, individual nucleotide sequences encoding the gNA or the CasX are linked to one of the foregoing categories of promoters, which are then introduced into the cells to be modified by conventional methods, described below.

In certain embodiments, suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), a POL1 promoter, a 7SK promoter, tRNA promoters and the like.

In some embodiments, one or more nucleotide sequences encoding a CasX and gNA and, optionally, comprising a donor template, are each operably linked to (under the control of) a promoter operable in a eukaryotic cell. Examples of inducible promoters may include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore, in some embodiments, be regulated by molecules including, but not limited to, doxycycline; estrogen and/or an estrogen analog; IPTG; etc.

In certain embodiments, inducible promoters suitable for use may include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR, etc.), tetracycline regulated promoters, (e.g., promoter systems including Tet Activators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Recombinant expression vectors of the disclosure can also comprise elements that facilitate robust expression of CasX proteins and the gNAs of the disclosure. For example, recombinant expression vectors can include one or more of a polyadenylation signal (PolyA), an intronic sequence or a post-transcriptional regulatory element such as a woodchuck hepatitis post-transcriptional regulatory element (WPRE). Exemplary polyA sequences include hGH poly(A) signal (short), HSV TK poly(A) signal, synthetic polyadenylation signals, SV40 poly(A) signal, β-globin poly(A) signal and the like. A person of ordinary skill in the art will be able to select suitable elements to include in the recombinant expression vectors described herein.

The polynucleotides encoding the reference CasX, the CasX variants, and the gNA sequences can then be individually cloned into one or more expression vectors. In some embodiments, the present disclosure provides vectors comprising the polynucleotides selected from the group consisting of a retroviral vector, a lentiviral vector, an adenoviral vector, an adeno-associated viral (AAV) vector, a virus-like particle (VLP), a herpes simplex virus (HSV) vector, a plasmid, a minicircle, a nanoplasmid, a DNA vector, and an RNA vector. In some embodiments, the vector is a recombinant expression vector that comprises a nucleotide sequence encoding a CasX protein. In other embodiments, the disclosure provides a recombinant expression vector comprising a nucleotide sequence encoding a CasX protein and a nucleotide sequence encoding a gNA. In some cases, the nucleotide sequence encoding the CasX protein variant and/or the nucleotide sequence encoding the gNA are operably linked to a promoter that is operable in a cell type of choice. In other embodiments, the nucleotide sequence encoding the CasX protein variant and the nucleotide sequence encoding the gNA are provided in separate vectors operably linked to a promoter.

In some embodiments, provided herein are one or more recombinant expression vectors comprising one or more of: (i) a nucleotide sequence of a donor template nucleic acid where the donor template comprises a nucleotide sequence having homology to a target sequence of a target nucleic acid (e.g., a target genome); (ii) a nucleotide sequence that encodes a gNA that hybridizes to a target sequence of the locus of the targeted genome (e.g., configured as a single or dual guide RNA) operably linked to a promoter that is operable in a target cell such as a eukaryotic cell; and (iii) a nucleotide sequence encoding a CasX protein operably linked to a promoter that is operable in a target cell such as a eukaryotic cell. In some embodiments, the sequences encoding the donor template, the gNA and the CasX protein are in different recombinant expression vectors, and in other embodiments one or more polynucleotide sequences (for the donor template, CasX, and the gNA) are in the same recombinant expression vector. In other cases, the CasX and gNA are delivered to the target cell as an RNP (e.g., by electroporation or chemical means) and the donor template is delivered by a vector.

The polynucleotide sequence(s) are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the protein involved in antigen processing, antigen presentation, antigen recognition, and/or antigen response can be determined using any nucleic acid or protein assay known in the art. For example, the presence of transcribed mRNA of reference CasX or the CasX variants can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of the polynucleotide.

The disclosure provides for the use of plasmid expression vectors containing replication and control sequences that are compatible with and recognized by the host cell and are operably linked to the gene encoding the polypeptide for controlled expression of the polypeptide or transcription of the RNA. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired. The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell.

The polynucleotides and recombinant expression vectors can be delivered to the target host cells by a variety of methods. Such methods include, but are not limited to, viral infection, transfection, lipofection, electroporation, calcium phosphate precipitation, polyethyleneimine (PEI)-mediated transfection, DEAE-dextran mediated transfection, microinjection, liposome-mediated transfection, particle gun technology, nucleofection, direct addition by cell penetrating CasX proteins that are fused to or recruit donor DNA, cell squeezing, calcium phosphate precipitation, direct microinjection, nanoparticle-mediated nucleic acid delivery, and using the commercially available TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like.

A recombinant expression vector sequence can be packaged into a virus or virus-like particle (also referred to herein as a “VLP” or “virion”) for subsequent infection and transformation of a cell, ex vivo, in vitro or in vivo. Such VLP or virions will typically include proteins that encapsidate or package the vector genome. Suitable expression vectors may include viral expression vectors based on vaccinia virus; poliovirus; adenovirus; a retroviral vector (e.g., Murine Leukemia Virus), spleen necrosis virus, and vectors derived from retroviruses such as Rous Sarcoma Virus, Harvey Sarcoma Virus, avian leukosis virus, retrovirus, a lentivirus, human immunodeficiency virus, myeloproliferative sarcoma virus, and mammary tumor virus; and the like.

In some embodiments, a recombinant expression vector of the present disclosure is a recombinant adeno-associated virus (AAV) vector. In a particular embodiment, a recombinant expression vector of the present disclosure is a recombinant retrovirus vector. In another particular embodiment, a recombinant expression vector of the present disclosure is a recombinant lentivirus vector.

AAV is a small (20 nm), nonpathogenic virus that is useful in treating human diseases in situations that employ a viral vector for delivery to a cell such as a eukaryotic cell, either in vivo or ex vivo for cells to be prepared for administration to a subject. A construct is generated, for example, encoding any of the CasX proteins and gNA embodiments as described herein, and optionally a donor template, and can be flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV 10, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences.

An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide comprising a CasX protein and/or sgNA and, optionally, a donor template of any of the embodiments described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2^{nd} Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein.)

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome.

By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, AAV capsids utilized for delivery of the CasX, gNA, and, optionally, donor template nucleotides, to a host cell can be derived from any of several AAV serotypes, including without limitation, AAV1, AAV2, AAV3, AAV4, AAV5, AAV6, AAV7, AAV8, AAV9, AAV10, AAV-Rh74 (Rhesus macaque-derived AAV), and AAVRh10, and the AAV ITRs are derived from AAV serotype 2. In a particular embodiment, AAV1, AAV7, AAV6, AAV8, or AAV9 are utilized for delivery of the CasX, gNA, and, optionally, donor template nucleotides, to a host muscle cell.

In order to produce rAAV viral particles, an AAV expression vector is introduced into a suitable host cell using known techniques, such as by transfection. Packaging cells are typically used to form virus particles; such cells include HEK293 or HEK293T cells (and other cells described herein or known in the art), which package adenovirus. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector. In some embodiments, the disclosure provides host cells comprising the AAV vectors of the embodiments disclosed herein.

In other embodiments, suitable vectors may include virus-like particles (VLP). Virus-like particles (VLPs) are particles that closely resemble viruses, but do not contain viral genetic material and are therefore non-infectious. In some embodiments, VLPs comprise a polynucleotide encoding a transgene of interest, for example any of the CasX protein and/or a gNA embodiments, and, optionally, donor template polynucleotides described herein, packaged with one or more viral structural proteins.

In other embodiments, the disclosure provides VLPs produced in vitro that comprise a CasX:gNA RNP complex and, optionally, a donor template. Combinations of structural proteins from different viruses can be used to create VLPs, including components from virus families including Parvoviridae (e.g., adeno-associated virus), Retroviridae (e.g., alpharetrovirus, a betaretrovirus, a gammaretrovirus, a deltaretrovirus, a epsilonretrovirus, or a lentivirus), Flaviviridae (e.g., Hepatitis C virus), Paramyxoviridae (e.g., Nipah) and bacteriophages (e.g., Qβ, AP205). In some embodiments, the disclosure provides VLP systems designed using components of retrovirus, including lentiviruses (such as HIV) and alpharetrovirus, betaretrovirus, gammaretrovirus, deltaretrovirus, epsilonretrovirus, in which individual plasmids comprising polynucleotides encoding the various components are introduced into a packaging cell that, in turn, produce the VLP. In some embodiments, the disclosure provides VLP comprising one or more components of i) protease, ii) a protease cleavage site, iii) one or more components of a gag polyprotein selected from a matrix protein (MA), a nucleocapsid protein (NC), a capsid protein (CA), a p1 peptide, a p6 peptide, a P2A peptide, a P2B peptide, a P10 peptide, a p12 peptide, a PP21/24 peptide, a P12/P3/P8 peptide, and a P20 peptide; v) CasX; vi) gNA, and vi) targeting glycoproteins or antibody fragments wherein the resulting VLP particle encapsidates a CasX:gNA RNP. The targeting glycoproteins or antibody fragments on the surface that provides tropism of the VLP to the target cell, wherein upon administration and entry into the target cell, the RNP molecule is free to be transported into the nucleus of the cell. In other embodiments, the disclosure provides VLP of the foregoing and further comprises one or more components of a pol polyprotein (e.g. a protease), and, optionally, a second CasX or a donor template. The foregoing offers advantages over other vectors in the art in that viral transduction to dividing and non-dividing cells is efficient and that the VLP delivers potent and short-lived RNP that escape a subject's immune surveillance mechanisms that would otherwise detect a foreign protein.

In some embodiments, the disclosure provides host cells comprising polynucleotides or vectors encoding one or more components selected from i) one or more components of a gag polyprotein (the components of which are listed, supra); ii) a CasX protein of any of the embodiments described herein; iii) a protease cleavage site; iv) a protease; v) a guide RNA of any of the embodiments described herein; vi) a pol polyprotein or portions thereof (e.g., a protease); vii) a pseudotyping glycoprotein or antibody fragment that provides for binding and fusion of the VLP to a target cell; and viii) a donor template. The disclosure contemplates multiple configurations of the arrangement of the encoded components, including duplicates of some of the encoded components. The envelope glycoprotein can be derived from any enveloped viruses known in the art to confer tropism to VLP, including but not limited to the group consisting of Argentine hemorrhagic fever virus, Australian bat virus, Autographa californica multiple nucleopolyhedrovirus, Avian leukosis virus, baboon endogenous virus, Bolivian hemorrhagic fever virus, Borna disease virus, Breda virus, Bunyamwera virus, Chandipura virus, Chikungunya virus, Crimean-Congo hemorrhagic fever virus, Dengue fever virus, Duvenhage virus, Eastern equine encephalitis virus, Ebola hemorrhagic fever virus, Ebola Zaire virus, enteric adenovirus, Ephemerovirus, Epstein-Bar virus (EBV), European bat virus 1, European bat virus 2, Fug Synthetic gP Fusion, Gibbon ape leukemia virus, Hantavirus, Hendra virus, hepatitis A virus, hepatitis B virus, hepatitis C virus, hepatitis D virus, hepatitis E virus, hepatitis G Virus (GB virus C), herpes simplex virus type 1, herpes simplex virus type 2, human cytomegalovirus (HHV5), human foamy virus, human herpesvirus (HHV), human Herpesvirus 7, human herpesvirus type 6, human herpesvirus type 8, human immunodeficiency virus 1 (HIV-1), human metapneumovirus, human T-lymphotro pic virus 1, influenza A, influenza B, influenza C virus, Japanese encephalitis virus, Kaposi's sarcoma-associated herpesvirus (HHV8), Kaysanur Forest disease virus, La Crosse virus, Lagos bat virus, Lassa fever virus, lymphocytic choriomeningitis virus (LCMV), Machupo virus, Marburg hemorrhagic fever virus, measles virus, Middle eastern respiratory syndrome-related coronavirus, Mokola virus, Moloney murine leukemia virus, monkey pox, mouse mammary tumor virus, mumps virus, murine gammaherpesvirus, Newcastle disease virus, Nipah virus, Nipah virus, Norwalk virus, Omsk hemorrhagic fever virus, papilloma virus, parvovirus, pseudorabies virus, Quaranfil virus, rabies virus, RD114 Endogenous Feline Retrovirus, respiratory syncytial virus (RSV), Rift Valley fever virus, Ross River virus, rRotavirus, Rous sarcoma virus, rubella virus, Sabia-associated hemorrhagic fever virus, SARS-associated coronavirus (SARS-CoV), Sendai virus, Tacaribe virus, Thogotovirus, tick-borne encephalitis causing virus, varicella zoster virus (HHV3), varicella zoster virus (HHV3), variola major virus, variola minor virus, Venezuelan equine encephalitis virus, Venezuelan hemorrhagic fever virus, vesicular stomatitis virus (VSV), VSV-G, Vesiculovirus, West Nile virus, western equine encephalitis virus, and Zika Virus. In some embodiments, the packaging cell used for the production of VLP is selected from the group consisting of HEK293 cells, Lenti-X HEK293T cells, BHK cells, HepG2 cells, Saos-2 cells, HuH7 cells, NS0 cells, SP2/0 cells, YO myeloma cells, A549 cells, P3X63 mouse myeloma cells, PER cells, PER.C6 cells, hybridoma cells, VERO cells, NIH3T3 cells, COS cells, WI38 cells, MRC5 cells, A549 cells, HeLa cells, CHO cells, or HT1080 cells.

Upon production and recovery of the VLP comprising the CasX:gNA RNP of any of the embodiments described herein, the VLP can be used in methods to edit target cells of subjects by the administering of such VLP, as described more fully, below.

### VII. Cells

In still further aspects, provided herein are cells comprising a C9orf72 gene modified by any of the CasX:gNA systems embodiments described herein. In some cases, cells that have been genetically modified in this way may be administered to a subject for purposes such as gene therapy, e.g., to treat a disease associated with a defect in the C9orf72 gene. In other cases, the cells are modified in vivo in a subject with a C9orf72-related disease. In some embodiments, the present disclosure provides a population of cells that has been modified to excise the hexanucleotide repeat expansion region of the C9orf72 gene such that a functional C9orf72 protein is expressed. In some cases of the foregoing, the cell to be modified comprises one or more mutations in the C9orf72 gene that disrupt the function or expression of the C9orf72 protein. In other cases of the foregoing, the cell to be modified comprises a HRS expansion in the C9orf72 gene such that excess RNA or DPR protein is produced and incorporated into the cell. In other cases of the foregoing, the cell to be modified comprises one or more mutations or truncations of the C9orf72 protein of SEQ ID NO: 227 or 228.

In some embodiments, the population of cells are modified by a Type V Cas nuclease and one or more guides targeted to sequences proximal to the associated with the hexanucleotide repeat expansion region of the C9orf72 target nucleic acid. In some embodiments, the disclosure provides methods and populations of cells modified by introducing into each cell of the population: i) a CasX:gNA system comprising a CasX and a gNA of any one of the embodiments described herein; ii) a CasX:gNA system comprising a CasX, a gNA, and a donor template of any one of the embodiments described herein; iii) a nucleic acid encoding the CasX and the gNA, and optionally comprising the donor template; iv) a vector selected from the group consisting of a retroviral vector, a lentiviral vector, an adenoviral vector, an adeno-associated viral (AAV) vector, and a herpes simplex virus (HSV) vector and comprising the nucleic acid of (iii), above; v) a VLP comprising the CasX:gNA system of any one of the embodiments described herein; or vi) combinations of two or more of (i) to (v), wherein the target nucleic acid sequence of the cells targeted by the gNA is modified by the CasX protein and, optionally, the donor template. In the foregoing, the donor template comprises at least a portion of a C9orf72 gene, wherein the C9orf72 gene portion is selected from a C9orf72 exon, a C9orf72 intron, a C9orf72 intron-exon junction, a C9orf72 regulatory element (e.g., a promoter), a C9orf72 coding region, a C9orf72 non-coding region, or combinations thereof or the entirety of the C9orf72 gene, and the modification of the cell results in the correction of the mutation to a wild-type sequence, the replacement of all or a portion of the hexanucleotide repeat expansion region or the knocking-down or knocking-out of the C9orf72 gene. In some cases, the donor template can comprise a nucleic acid encoding all or a portion of the sequence of SEQ ID NO: 227 or 228 or comprises a polynucleotide sequence that spans all or a portion of chr9: 27,546,546 to 27,573,866 of the human genome (GRCh37/hg19) (the notation refers to the chromosome 4 (chr4), starting at the 27,546,546 bp of that chromosome, and extending to the 27,573,866 bp of that chromosome) or a portion thereof. In other cases, the donor template can comprise a heterologous sequence compared to a wild-type C9orf72 gene in order to knock down or knock out the gene. In still other cases, the donor template comprises a hexanucleotide repeat of a GGGGCC sequence wherein the number of repeats ranges from 10 to about 30 repeats. In the foregoing, the donor template would be used to replace the defective sequence of the cell having hundreds to thousands of the hexanucleotide repeats. The donor template would further comprise homologous arms that are 5′ and 3′ to the cleavage sites introduced by the nuclease to facilitate its insertion by HDR. The donor template can range in size from 10-30,000 nucleotides, or 20-10,000 nucleotides, or 100-1000 nucleotides. In some cases, the donor template is a single-stranded DNA template or a single stranded RNA template. In other cases, the donor template is a double-stranded DNA template. In some cases, the cell is contacted with a CasX and at least a first gNA wherein the gNA is a guide RNA (gRNA). In some cases, the cell is contacted with a CasX and at least a first and a second gNA wherein the gNA is a guide RNA (gRNA). In other cases, the cell is contacted with a CasX and a gNA wherein the gNA is a guide DNA (gDNA). In other cases, the cell is contacted with a CasX and a gNA wherein the gNA is a chimera comprising DNA and RNA. As described herein, in embodiments of any of the combinations, each of said gNA molecules (a combination of the scaffold and targeting sequence, which can be configured as a sgRNA or a dgRNA) can be provided as an RNP with a CasX embodiment described herein for incorporation into the cells of the embodiments. In some embodiments, the cells of the population are contacted with an RNP of a CasX comprising a sequence of SEQ ID NOS: 49-150, 233-235, 238-252, or 272-281 as set forth in Tables 4, 6-8 and 10, or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gNA scaffold comprises a sequence of SEQ ID NOS: 2101-2294 as set forth in Table 2 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gNA comprises a targeting sequence of SEQ ID NOs: 309-343, 363-2100 and 2295-21835 or a sequence at least 65% identical, at least 70% identical, at least 75% identical, at least 80% identical, at least 85% identical, at least 90% identical, or at least 95% identical thereto and having between 15 and 21 amino acids.

In some embodiments, the modified C9orf72 gene of the modified cell comprises a single-stranded break, resulting in a mutation, an insertion, or a deletion by the repair mechanisms of the cell. In other embodiments, the modified C9orf72 gene of the cell comprises a double-stranded break, resulting in a mutation, an insertion, or a deletion by the repair mechanisms of the cell. For example, the CasX:gNA system can introduce into the cell an indel, e.g., a frameshift mutation, at or near the initiation point of the C9orf72 gene. In some embodiments, the cell is modified by contact with a CasX and a first gNA targeting the target nucleic acid 5′ to the hexanucleotide repeat expansion region and a second gNA targeting the target nucleic acid 3′ to the hexanucleotide repeat expansion region wherein the hexanucleotide repeat expansion region is excised from the C9orf72 gene, wherein the modification results in the cells ability to produce a wild-type or a functional C9orf72 protein. In some embodiments, the population of cells have been modified such that expression of the hexanucleotide transcript RNA or the DPR is reduced by at least about 10%, at least about 20%, at least about 30%, at least about 40%, at least about 50%, at least about 60%, at least about 70%, at least about 80%, at least about 90%, or at least about 95% in comparison to a cell that has not been modified. In other embodiments, at least 30%, at least 40%, at least 50%, at least 60%, at least 05%, at least 75%, at least 80%, at least 85%, at least 90%, or at least 95% of the modified cells do not express a detectable level of the hexanucleotide transcript RNA or the DPR. In some embodiments, the first gNA targeting sequence is selected from the group consisting of SEQ ID NOS: 310 and 319-320, and the second gNA targeting sequence is selected from the group consisting of SEQ ID NOS: 321-325. Reduced or eliminated expression of a hexanucleotide transcript RNA or the DPR can be measured by ELISA or electrochemiluminescence assays, sense G4C2-repeat transcripts can be analyzed by RNA fluorescence in situ hybridization (FISH) assay (Batra, R and Lee, C. Mouse Models of C9orf72 Hexanucleotide Repeat Expansion in Amyotrophic Lateral Sclerosis/Frontotemporal Dementia. Frontiers Cell. Neurosci. 11:196 (2017)) or other methods know in the art, or as described in the Examples. In some embodiments, the disclosure provides a population of cells modified such that expression of a functional C9orf72 protein is increased by at least about 10%, at least about 20%, at least about 30%, at least about 40%, at least about 50%, at least about 60%, at least about 70%, at least about 80%, or at least about 90% in comparison to a cell that has not been modified.

In some cases, the modification of a cell's C9orf72 gene that comprises one or more mutations or duplications occurs in vitro. In such cases, a population of the modified cell can then be administered to a subject. An RNP can be introduced into the cells to be modified via any suitable method, including via electroporation, injection, nucleofection, delivery via liposomes, delivery by nanoparticles, or using a protein transduction domain (PTD) conjugated to one or more components of the CasX:gNA. In other cases, the CasX and the one or more gNA are introduced into the population of cells as encoding polynucleotides using a vector; embodiments of which are described herein. Additional methods of modification of the cells using the CasX:gNA system components include viral infection, transfection, conjugation, protoplast fusion, particle gun technology, calcium phosphate precipitation, direct microinjection, and the like. The choice of method is generally dependent on the type of cell being transformed and the circumstances under which the transformation is taking place; e.g., in vitro, ex vivo, or in vivo. A general discussion of these methods can be found in Ausubel, et al, Short Protocols in Molecular Biology, 3rd ed., Wiley & Sons, 1995.

In other cases, the modification of a cell's C9orf72 gene that comprises one or more mutations or duplications occurs in vivo. In such cases, the CasX and gNA, and, optionally, the donor template are administered to the subject. In other cases, the CasX and gNA, and, optionally, the donor template are administered to a subject within a vector that encodes the CasX and one or more gNA and, optionally, contains the donor template. In still other cases, the CasX and gNA, and, optionally, the donor template are administered to a subject within a vector such as a VLP that encapsidates the RNP and, optionally, contains the donor template. In the foregoing, the modification corrects the one or more mutations or, in the alternative, the modification is the inhibition or suppression of the expression of the hexanucleotide transcript RNA or the DPR, the expression of a functional C9orf72 protein, or the expression of the wild-type or a functional C9orf72 protein.

A cell that can serve as a recipient for a CasX protein and/or gNA of the present disclosure and/or a nucleic acid comprising a nucleotide sequence encoding a CasX protein and/or a CasX gNA variant and, optionally, a donor template, can be any of a variety of cells, including, e.g., in vitro cells; in vivo cells; ex vivo cells; primary cells; cancer cells; animal cells; etc. A cell can be a recipient of a CasX RNP of the present disclosure. A cell can be a recipient of a single component of a CasX system of the present disclosure. In certain embodiments, as provided herein, a cell can be an in vitro cell (e.g., established cultured cell line including, but not limited to HEK293 cells, HEK293-F cells, BHK cells, NS0 cells, SP2/0 cells, YO myeloma cells, P3X63 mouse myeloma cells, PER cells, PER.C6 cells, hybridoma cells, NIH3T3 cells, COS, HeLa, or CHO cells). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be an animal cell or derived from an animal cell. A cell can be a vertebrate cell or derived from a vertebrate cell. A cell can be a mammalian cell or derived from a mammalian cell. A cell can be a rodent cell or derived from a rodent cell. A cell can be a non-human primate cell or derived from a non-human primate cell. A cell can be a human cell or derived from a human cell.

In some embodiments, the modified cell is a eukaryotic cell, wherein the eukaryotic cell is selected from the group consisting of a rodent cell, a mouse cell, a rat cell, a primate cell, a non-human primate cell, and a human cell. In some embodiments, the modified cell is a human cell. In other embodiments, the cells are autologous with respect to a subject to be administered the cell. In other embodiments, the cells are allogeneic with respect to a subject to be administered the cell. In some embodiments, the modified cell is a cell of the central nervous system (CNS). In some embodiments, the modified cell is selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes. In the foregoing, the populations of cells have utility in the treatment of a C9orf72-related disease, wherein the population of cells are administered to a subject having a C9orf72-related disease. In some cases of the foregoing, the cell to be modified comprises one or more mutations in the C9orf72 gene that disrupt the function or expression of the C9orf72 protein. In other cases of the foregoing, the cell to be modified comprises a HRS expansion in the C9orf72 gene such that excess RNA or DPR protein is produced and incorporated into the cell. In other cases of the foregoing, the cell to be modified comprises one or more mutations or truncations of the C9orf72 protein of SEQ ID NO: 227 or 228.

In other embodiments, the disclosure provides populations of modified cells for use in in a subject with a C9orf72-related disease. In some embodiments, the disclosure provides a method of treating a subject having a C9orf72-related disease, the method comprising administering to the subject an effective amount of a plurality of modified cells of any one of the embodiments described herein wherein the modified cells express physiologically-normal levels of C9orf72. In some embodiments, the C9orf72-related disease is selected from the group consisting of amyotrophic lateral sclerosis (ALS) and frontotemporal dementia (FTD).

### VIII. Applications

The CasX:gNA systems comprising CasX proteins, guides, and variants thereof provided herein are useful in methods for modifying the C9orf72 target nucleic acid sequence in various applications, including therapeutics, diagnostics, and research.

In the methods of modifying a C9orf72 target nucleic acid sequence in a cell described herein, the methods utilize any of the embodiments of the CasX:gNA system described herein, and optionally include a donor template described herein. In some cases, the methods knock-down the expression of the mutant C9orf72. In other cases, the methods knock-out the expression of the mutant C9orf72. In still other cases, the methods result in the expression of functional C9orf72 protein.

In some embodiments, the methods comprise contacting the target nucleic acid sequence with a CasX protein and a guide nucleic acid (gNA) comprising a targeting sequence, wherein said contacting results in modification of the target nucleic acid sequence by the CasX protein. In some embodiments, the methods comprise introducing into a cell the CasX protein or a nucleic acid encoding the CasX protein and the gNA or the nucleic acid encoding the gNA, wherein the target nucleic acid sequence comprises a C9orf72 gene and wherein the targeting sequence comprises a sequence complementary to a portion of the C9orf72 gene encoding the C9orf72 protein, a C9orf72 regulatory element, or both the C9orf72 encoding sequence and a C9orf72 regulatory element, wherein the contacting results in the modification of the C9orf72 gene. In some embodiments, the targeting sequence of the gNA comprises a sequence of SEQ ID NOS: 309-343, 363-2100 and 2295-21835, or a sequence having at least about 65%, at least about 75%, at least about 85%, or at least about 95% identity thereto. In some embodiments, the scaffold of the gNA comprises a sequence of SEQ ID NOS: 4, 5 or 2101-2294, or a sequence having at least about 50%, at least about 60%, at least about 70%, at least about 80%, at least about 90%, at least about 95%, at least about 95%, at least about 96%, at least about 97%, at least about 98%, at least about 99%, or 100% sequence identity thereto. In some embodiments, the CasX protein is a CasX variant protein of any of the embodiments described herein, or a reference CasX protein SEQ ID NO: 1, SEQ ID NO: 2, or SEQ ID NO: 3.

In some embodiments, the modified C9orf72 gene of the modified cell comprises a single-stranded break, resulting in a mutation, an insertion, or a deletion by the repair mechanisms of the cell. In other embodiments, the modified C9orf72 gene of the modified cell comprises a double-stranded break, resulting in a mutation, an insertion, or a deletion by the repair mechanisms of the cell. For example, the CasX:gNA system can introduce into the cell an indel, e.g., a frameshift mutation, at or near the initiation point of the C9orf72 gene. In other embodiments, the modified C9orf72 gene of the cell has been modified by the insertion of the donor template wherein the C9orf72 gene has been knocked down or knocked out. In the foregoing, the cells have been modified such that expression of the HRS or the DPR protein is reduced by at least about 10%, at least about 20%, at least about 30%, at least about 40%, at least about 50%, at least about 60%, at least about 70%, at least about 80%, or at least about 90% in comparison to a cell that has not been modified. In other embodiments, at least 70%, at least 75%, at least 80%, at least 85%, at least 90%, or at least 95% of the modified cells do not express a detectable level of HRS RNA or DPR. Reduced or eliminated expression of a HRS RNA or DPR protein can be measured by ELISA or electrochemiluminescence assays (Mcdonald, D., et al. Quantification Assays for Total and Polyglutamine-Expanded Huntingtin Proteins. PLoS ONE 9(5): e96854 (2014)) or other methods know in the art, or as described in the Examples.

In some embodiments of the methods of modifying a C9orf72 target nucleic acid sequence, the target nucleic acid sequence comprises a C9orf72 gene having one or more mutations or duplications, and the targeting sequence of the gNA has a sequence that is complementary to, and therefore can hybridize with the C9orf72 gene. In some cases, the C9orf72 gene has a wild-type nucleic acid sequence. In other embodiments, the method comprises contacting the target nucleic acid sequence with a plurality (e.g., two or more) of gNAs targeted to different or overlapping regions of the C9orf72 gene with one or more mutations or duplications. In some embodiments of the method, the target nucleic acid is a DNA. In some embodiments of the method, the target nucleic acid is an RNA. In some embodiments, the gNA is a guide RNA (gRNA). In some embodiments, the gNA is a guide DNA (gDNA). In some embodiments, the gNA is a single-molecule gNA (sgNA). In other embodiments, the gNA is a dual-molecule gNA (dgNA). In some embodiments, the gNA is a chimeric gRNA-gDNA. In some embodiments, the method comprises contacting the target nucleic acid sequence with a pre-complexed CasX protein-gNA (i.e., an RNP). In some embodiments, the C9orf72 gene comprises a mutation or duplication and the modifying comprises introducing a single-stranded break in the target nucleic acid. In other embodiments, the C9orf72 gene comprises a mutation or duplication and the modifying comprises introducing a double-stranded break in the target nucleic acid. In the foregoing, the resulting modification can be an insertion, deletion, substitution, duplication, or inversion of one or more nucleotides as compared to the wild-type sequence. In some embodiments, the modification corrects a gain of function mutation. In other embodiments, the modification corrects a loss of function mutation. The mutations to be modified can comprise one or more mutations or duplications that disrupt the function or expression of the C9orf72 protein.

In some embodiments, the methods of modifying a target nucleic acid sequence comprise contacting a C9orf72 gene with a CasX protein and gNA pair and a donor template comprising a corrective sequence that can be inserted or knocked-in at the break site introduced by the CasX. For example, an exogenous donor template which may comprise a corrective sequence (or a deletion or insertion to knock-out the defective sequence) to be integrated is flanked by an upstream sequence and a downstream sequence (e.g., homologous arms) with homology to the target nucleic acid sequence to facilitate its introduction into a cell. In some embodiments, the donor template ranges in size from 10-10,000 nucleotides. In other embodiments, the donor template ranges in size from 100-1,000 nucleotides. In some embodiments, the donor template is a single-stranded DNA template or a single stranded RNA template. In other embodiments, the donor template is a double-stranded DNA template.

In some embodiments of the methods, the CasX is a catalytically inactive CasX (dCasX) protein that retains the ability to bind to the gNA and the target nucleic acid sequence comprising the mutation, thereby interfering with transcription of mutant C9orf72. In some embodiments, the methods comprise contacting a C9orf72 gene with a CasX protein and gNA and does not comprise contacting the target nucleic acid sequence with a donor template polynucleotide, and the target nucleic acid sequence is cleaved by the CasX nuclease and is modified such that nucleotides within the target nucleic acid sequence are deleted or inserted according to the cells own repair pathways. In some embodiments, the editing occurs in vivo inside of a cell, for example in a cell in an organism or subject. In some embodiments, the cell is a eukaryotic cell. Exemplary eukaryotic cells may include cells selected from the group consisting of a rodent cell, a mouse cell, a rat cell, a primate cell, a non-human primate cell, and a human cell. In some embodiments, the cell is a human cell. In some embodiments, the cell is a non-human primate cell. In some embodiments of the method, the cell is a selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

Methods of introducing a nucleic acid (e.g., a nucleic acid comprising a donor polynucleotide sequence, one or more nucleic acids encoding a CasX protein and/or gNA) into a cell are known in the art, and any convenient method can be used to introduce a nucleic acid (e.g., an expression construct) into a cell. Suitable methods include; e.g., viral infection or contact with a virus-like particle (VLP) with tropism for the target cell. Retroviruses, for example, lentiviruses, may be suitable for use in methods of the present disclosure. Commonly used retroviral vectors are “defective”, e.g., are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, and this envelope protein determines the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Nucleic acids can also be introduced by direct micro-injection (e.g., injection of RNA).

In other embodiments, the disclosure relates to methods to produce CasX proteins and nucleic acids encoding the CasX compositions of any of the embodiments described herein, or sequences complementary to the polynucleotide sequences, including homologous variants thereof, as well as methods to express the CasX proteins expressed by the polynucleotide sequences. A CasX protein of the present disclosure may be produced in vitro by eukaryotic cells or by prokaryotic cells. For production by host cells, in general, the methods include producing a polynucleotide sequence coding for the CasX proteins of any of the embodiments described herein and incorporating the encoding gene into an expression vector appropriate for a host cell. For production of the encoded CasX proteins of any of the embodiments described herein, the method includes transforming an appropriate host cell with an expression vector, and culturing the host cell under conditions causing or permitting the resulting CasX protein to be expressed in the transformed host cell, thereby producing the CasX protein, which is recovered by methods described herein or by standard protein purification methods known in the art. Standard recombinant techniques in molecular biology are used to make the polynucleotides and expression vectors of the present disclosure.

In some embodiments of the methods of altering a C9orf72 target nucleic acid sequence of a cell, or to induce cleavage of a target nucleic acid sequence, the CasX gNA and/or the CasX protein of the present disclosure and/or the donor template sequence, whether they be introduced as nucleic acids or polypeptides, are provided to the cells by a vector or particle of the embodiments described herein. Providing the vector or particle to the cells may be repeated with a frequency of about every day to about every 4 days, e.g., every 1.5 days, every 2 days, every 3 days, or any other frequency from about every day to about every four days, or weekly, or monthly. The agent(s) may be provided to the subject cells one or more times, e.g., one time, twice, three times, or more than three times.

In embodiments in which two or more different targeting complexes are provided to the cell (e.g., two CasX gNAs with different targeting sequences), the complexes may be provided simultaneously (e.g., as two polypeptides and/or nucleic acids), or delivered simultaneously. Alternatively, they may be provided consecutively, e.g., the targeting complex being provided first, followed by the second targeting complex, etc. or vice versa.

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also, as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are referred to as polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

### IX. Therapeutic Methods

The present disclosure provides methods of treating a C9orf72-related disease in a subject in need thereof, including but not limited to amyotrophic lateral sclerosis (ALS) and frontotemporal dementia (FTD). In some embodiments, the methods of the disclosure can prevent, treat and/or ameliorate a C9orf72-related disease of a subject by the administering to the subject of a composition of the disclosure. A number of therapeutic strategies have been used to design the compositions for use in the methods of treatment of a subject with a C9orf72-related disease. Additionally, the methods can be used to treat a subject in advance of any symptom of a C9orf72-related disease. Accordingly, the prophylactic administration of a modified cell population or a therapeutically effective amount of the CasX:gNA system composition(s) or the polynucleic acids encoding the CasX:gNA systems of the embodiments can serve to prevent a C9orf72-related disease. In some embodiments, the composition administered to the subject further comprises pharmaceutically acceptable carrier, diluent or excipient.

In some cases, one of the alleles of the C9orf72 gene of the subject comprises an HRS. In some cases, one or both alleles of the C9orf72 gene of the subject comprises a mutation. In other cases, one or both alleles of the C9orf72 gene of the subject comprises a duplication of at least a portion of the C9orf72 gene. In other cases, one or both alleles of the C9orf72 gene of the subject comprises a duplication of the C9orf72 gene. In other cases, the C9orf72 gene encodes a mutation that alters the function or expression of the C9orf72 protein such as, but not limited to, substitutions, deletions or insertions of one or more nucleotides as compared to the wild-type sequence.

In some embodiments, the disclosure provides methods of treating a C9orf72 or related disease in a subject in need thereof comprising modifying a C9orf72 gene in a cell of the subject, the modifying comprising contacting said cells with a therapeutically effective dose of i) a composition comprising a CasX and a gNA of any of the embodiments described herein; ii) a composition comprising a CasX, a gNA, and a donor template of any of the embodiments described herein; iii) one or more nucleic acids encoding or comprising the compositions of (i) or (ii); iv) a vector selected from the group consisting of a retroviral vector, a lentiviral vector, an adenoviral vector, an adeno-associated viral (AAV) vector, a herpes simplex virus (HSV) vector and comprising the nucleic acids of (iii); v) a VLP comprising the composition of (i) or (ii); or vi) combinations of two or more of (i)-(v), wherein the C9orf72 gene of the cells is modified by the CasX protein and, optionally, the donor template such that a wild-type or a functional C9orf72 protein is expressed. In some embodiments of the method of treating a C9orf72-related disease in a subject, a second gNA is utilized, wherein the second gNA has a targeting sequence complementary to a different or overlapping portion of the target nucleic acid compared to the first gNA (e.g., 5′ and 3′ to the hexanucleotide repeat expansion), resulting in an additional break in the C9orf72 target nucleic acid of the cells of the subject. In the foregoing, the gene can be modified by the NHEJ host repair mechanisms, or utilized in conjunction with a donor template that is inserted by HDR or HITI mechanisms to either excise, correct, or compensate for the mutation, such that expression of a wild-type or functional C9orf72 protein in modified cells is increased by at least about 50%, at least about 60%, at least about 70%, at least about 80%, at least about 90%, or at least about 95% in comparison to a cell that has not been modified. In some embodiments, the method of treatment by administration of the modalities of (i)-(v), above, results in a knocking-down or knocking out of the C9orf72 gene such that expression of the HRS RNA and/or the DPR in modified cells is reduced by at least about 10%, at least about 20%, at least about 30%, at least about 40%, at least about 50%, at least about 60%, at least about 70%, at least about 80%, or at least about 90% in comparison to cells that have not been modified. In the embodiments of method of treatment, the C9orf72-related disease includes all diseases occurring due to expression of the HRS RNA and/or the DPR, the mutation of C9orf72, the duplication of the C9orf72 gene, or the abnormal expression of C9orf72 in the subject. The embodiments of the paragraph are more fully detailed, below.

In some embodiments, the method comprises administration of the vector comprising or encoding a CasX and a plurality of gNAs targeted to different locations in the C9orf72 gene, wherein the contacting of the cells of the subject with the CasX:gNA complexes results in modification of the target nucleic acid of the cells.

In some embodiments, the vector of the embodiments are administered to the subject at a therapeutically effective dose. In one particular embodiment, the vector is an AAV of the embodiments described herein, encoding components of the CasX:gNA system and, optionally, the donor template. In the foregoing, the AAV vector is selected from AAV1, AAV2, AAV3, AAV4, AAV5, AAV6, AAV7, AAV8, AAV9, AAV10, AAV11, AAV12, AAV 44.9, AAV-Rh74, or AAVRh10. In some embodiments, the AAV vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In some embodiments, the AAV vector is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg. In other embodiments, the method comprises administering a therapeutically effective dose of a VLP of the embodiments described herein to the subject, comprising components of the CasX:gNA system and, optionally, the donor template. In some embodiments, the VLP is administered to the subject at a dose of at least about 1×10⁵ particles/kg, at least about 1×10⁶ particles/kg, at least about 1×10⁷ particles/kg at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg. In some embodiments, the VLP is administered to the subject at a dose of at least about 1×10⁵ particles/kg to about 1×10¹⁶ particles/kg, or at least about 1×10⁶ particles/kg to about 1×10¹⁵ particles/kg, or at least about 1×10⁷ particles/kg to about 1×10¹⁴ particles/kg. The vector or VLP can be administered according to any of the treatment regimens disclosed herein, below.

In some embodiments, administering the C9orf72-targeting vector compositions of the disclosure to a subject delivers the CasX:gNA compositions to a cell of the subject, resulting in the editing of the C9orf72 target nucleic acid in said cell. The modified cell of the treated subject can be a eukaryotic cell selected from the group consisting of a rodent cell, a mouse cell, a rat cell, a primate cell, a non-human primate cell, and a human cell. In some embodiments, the eukaryotic cell of the treated subject is a human cell. In some embodiments, the cell is a cell selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes. In some embodiments, the cell comprises at least one modified allele of a C9orf72 gene in a cell wherein the modification is used to correct or compensate for a mutation or a duplication of a portion of the C9orf72 gene in the subject; e.g., the HRS. In other embodiments, the cell comprises at least one modified allele of a C9orf72 gene in a cell wherein the modification is used to knock-down or knock out the C9orf72 gene in the subject.

In other embodiments of the methods of treatment, the methods comprises further administering an additional CRISPR protein, or a polynucleotide encoding the additional CRISPR protein to the subject. In the foregoing embodiment, the additional CRISPR protein has a sequence different from the first CasX protein of the method. In some embodiments, the additional CRISPR protein is not a CasX protein; i.e., is a Cpf1, Cas9, Cas10, Cas12a, or Cas13a. In some cases, the gNA used in the method of treatment is a single-molecule gNA (sgNA). In other cases, the gNA is a dual-molecule gNA (dgNA). In still other cases, the method comprises contacting the target nucleic acid sequence with a plurality of gNAs targeted to different or overlapping sequences of the C9orf72 gene.

In some embodiments, the method of treatment comprises administering to the subject the CasX:gNA compositions or vectors via an administration route selected from the group consisting of subcutaneous, intradermal, intraneural, intranodal, intramedullary, intramuscular, intralumbar, intrathecal, subarachnoid, intraventricular, intracapsular, intravenous, intralymphatic, or intraperitoneal routes, wherein the administering method comprises injection, transfusion, or implantation. In some embodiments of the methods of treating a C9orf72-related disease in a subject, the subject is selected from the group consisting of mouse, rat, pig, non-human primate, and human. In particular embodiments, the subject is a human. In some embodiments, the cell of the subject to be modified by the methods of disclosure is a cell is selected from the group consisting of a Purkinje cell, frontal cortex neuron, motor cortex neuron, hippocampus neuron, cerebellum neuron, upper motor neuron, spinal cord neuron, spinal cord motor neuron, glial cell, and astrocytes.

A number of therapeutic strategies have been used to design the compositions for use in the methods of treatment of a subject with a C9orf72-related disease. In some embodiments, the invention provides a method of treatment of a subject having a C9orf72-related disease, the method comprising administering to the subject a CasX:gNA composition or a vector of any of the embodiments disclosed herein according to a treatment regimen comprising one or more consecutive doses using a therapeutically effective dose. In some embodiments of the treatment regimen, the therapeutically effective dose of the composition or vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of subcutaneous, intradermal, intraneural, intranodal, intramedullary, intramuscular, intralumbar, intrathecal, subarachnoid, intraventricular, intracapsular, intravenous, intralymphatic, or intraperitoneal routes, wherein the administering method is injection, transfusion, or implantation.

In some embodiments, the administering of the therapeutically effective amount of a CasX:gNA modality or a vector comprising a polynucleotide encoding a CasX protein and a guide nucleic acid disclosed herein, to knock down or knock out expression of C9orf72 in a subject with a C9orf72-related disease, wherein the modification leads to the prevention or amelioration of the underlying C9orf72-related disease such that an improvement is observed in the subject, notwithstanding that the subject may still be afflicted with the underlying disease. In other embodiments, the administering to the subject with a C9orf72-related disease of the therapeutically effective amount of a CasX:gNA modality or a vector comprising a polynucleotide encoding a CasX protein and a guide nucleic acid disclosed herein, corrects or compensates for the mutations such that expression of a wild-type or functional C9orf72 protein leads to the prevention or amelioration of the underlying C9orf72-related disease such that an improvement is observed in the subject, notwithstanding that the subject may still be afflicted with the underlying disease. In some embodiments, the administration of the therapeutically effective amount of the CasX-gNA modality leads to an improvement in at least one clinically-relevant parameter for a C9orf72-related disease, including, but not limited to neuronal cell death, neuroinflammation, TDP-43 related pathology, axonal and neuromuscular junction (NMJ) abnormalities, dendritic spine density changes at prefrontal cortex, electrophysiological deficits in neonatal cortical neurons, change from baseline in percent of predicted slow vital capacity (SVC), change from baseline in muscle strength, change from baseline in bulbar strength, ALS Functional Rating Scale (ALSFRS-(R)), combined assessment of function and survival, duration of response, time to death, time to tracheostomy, time to persistent assisted ventilation (DTP), forced vital capacity (% FVC); manual muscle test, maximum voluntary isometric contraction, duration of response, progression-free survival, time to progression of disease, and time-to-treatment failure. In some embodiments, the administration of the therapeutically effective amount of the CasX-gNA modality leads to an improvement in at least two clinically-relevant parameters for treatment of a C9orf72-related disease. The C9orf72-related disease can be FTD, ALS, or both. In some embodiments of the methods of treatment, the subject is selected from mouse, rat, pig, dog, non-human primate, and human.

In some embodiments, the method of treatment comprises administering a therapeutically-effective dose of a population of cells modified to correct or compensate for the mutation of the C9orf72 gene. Methods for modification of such populations of cells are described herein, supra. By the method of treatment, the administration of the modified cells results in the expression of wild-type or a functional C9orf72 protein in the subject. In some embodiments of the method of treatment, the dose of total cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. In one embodiment, the cells are autologous with respect to the subject to be administered the cells. In another embodiment, the cells are allogeneic with respect to the subject to be administered the cells.

In some embodiments, the methods of treatment further comprise administering a chemotherapeutic agent wherein the agent is effective in improving the signs or symptoms associated with a C9orf72-related disease, including but not limited to riluzole, ranolazine, radicava, and dextromethorphan HBr in combination with quinidine sulfate.

Methods of obtaining samples from treated subjects for analysis to determine the effectiveness of the treatment, such as body fluids or tissues, and methods of preparation of the samples to allow for analysis are well known to those skilled in the art. Methods for analysis of RNA and protein levels are discussed above and are well known to those skilled in the art. The effects of treatment can also be assessed by measuring biomarkers associated with the target gene expression in the aforementioned fluids, tissues or organs, collected from an animal contacted with one or more compounds of the invention, by routine clinical methods known in the art. Biomarkers of C9orf72 diseases include, but are not limited to, C9orf72 levels, C9orf72 RNA, GGGGCC repeat-containing RNA species (as well as the antisense GGCCCC RNA), polyadenylated C9orf72 RNA species retaining the hexanucleotide repeat-containing intron, DPR levels, and DPR RNA levels.

Several mouse models of C9orf72 hexanucleotide repeat expansion exist, and are suitable for evaluating the methods of treatment of the embodiments (Batra R, and Lee C W. Mouse Models of C9orf72 Hexanucleotide Repeat Expansion in Amyotrophic Lateral Sclerosis/Frontotemporal Dementia. Front Cell Neurosci. 2017; 11:196 (2017)).

### X. Kits and Articles of Manufacture

In other embodiments, provided herein are kits comprising a CasX protein, one or a plurality of CasX gNA of any of the embodiments of the disclosure comprising a targeting sequence specific for a C9orf72 gene, and a suitable container (for example a tube, vial or plate).

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

In some embodiments, the kit comprises a vector comprising a sequence encoding a CasX protein of the disclosure, a CasX gNA of the disclosure, optionally a donor template, or a combination thereof, and the kit further comprises a pharmaceutically acceptable carrier, diluent or excipient.

The present description sets forth numerous exemplary configurations, methods, parameters, and the like. It should be recognized, however, that such description is not intended as a limitation on the scope of the present disclosure, but is instead provided as a description of exemplary embodiments.

### Illustrative Embodiments

The invention can be understood with reference to the following illustrated, enumerated embodiments:

1. A CasX:gNA system comprising a CasX protein and a guide nucleic acid (gNA), wherein the gNA comprises a targeting sequence complementary to a target nucleic acid sequence comprising a chromosome 9 open reading frame 72 (C9orf72) gene.

2. The CasX:gNA system of embodiment 1, wherein the C9orf72 gene comprises one or more mutations.

3. The CasX:gNA system of embodiment 1, wherein the C9orf72 gene mutation comprises more than 30, more than 100, more than 500, more than 700, more than 1000, or more than 1600 copies of a hexanucleotide repeat sequence (HRS) GGGGCC.

4. The CasX:gNA system of embodiment 2 or embodiment 3, wherein the mutation is a loss-of-function mutation.

5. The CasX:gNA system of embodiment 2 or embodiment 3, wherein the mutation is a gain-of-function mutation.

6. The CasX:gNA system of any one of the preceding embodiments, wherein the gNA is a guide RNA (gRNA).

7. The CasX:gNA system of any one of embodiments 1-5, wherein the gNA is a guide DNA (gDNA).

8. The CasX:gNA system of any one of embodiments 1-5, wherein the gNA is a chimera comprising DNA and RNA.

9. The CasX:gNA system of any one of embodiments 1-8, wherein the gNA is a single-molecule gNA (sgNA).

10. The CasX:gNA system of any one of embodiments 1-8, wherein the gNA is a dual-molecule gNA (dgNA).

11. The CasX:gNA system of any one of embodiments 1-10, wherein the targeting sequence of the gNA is complementary to a sequence comprising one or more single nucleotide polymorphisms (SNPs) of the C9orf72 gene.

12. The CasX:gNA system of any one of embodiments 1-10, wherein the targeting sequence of the gNA comprises a sequence selected from the group consisting of sequences set forth in Table 3.

13. The CasX:gNA system of any one of embodiments 1-10, wherein the targeting sequence of the gNA comprises a sequence of Table 3 with a single nucleotide removed from the 3′ end of the sequence.

14. The CasX:gNA system of any one of embodiments 1-10, wherein the targeting sequence of the gNA comprises a sequence of Table 3 with two nucleotides removed from the 3′ end of the sequence.

15. The CasX:gNA system of any one of embodiments 1-10, wherein the targeting sequence of the gNA comprises a sequence of Table 3 with three nucleotides removed from the 3′ end of the sequence.

16. The CasX:gNA system of any one of embodiments 1-10, wherein the targeting sequence of the gNA comprises a sequence of Table 3 with four nucleotides removed from the 3′ end of the sequence.

17. The CasX:gNA system of any one of embodiments 1-10, wherein the targeting sequence of the gNA comprises a sequence of Table 3 with five nucleotides removed from the 3′ end of the sequence.

18. The CasX:gNA system of any one of embodiments 1-10, wherein the targeting sequence of the gNA comprises a sequence having at least about 65%, at least about 75%, at least about 85%, or at least about 95% identity to a sequence selected from the group consisting of sequences set forth in Table 3.

19. The CasX:gNA system of any one of embodiments 1-10, wherein the targeting sequence of the gNA comprises a sequence having one or more single nucleotide polymorphisms (SNP) relative to a sequence provided in Table 3.

20. The CasX:gNA system of any one of embodiments 1-19, wherein the targeting sequence of the gNA is complementary to a non-coding region of the C9orf72 gene.

21. The CasX:gNA system of any one of embodiments 1-19, wherein the targeting sequence of the gNA is complementary to a coding region of the C9orf72 gene.

22. The CasX:gNA system of any one of embodiments 1-19, wherein the targeting sequence of the gNA is complementary to a sequence of a C9orf72 exon.

23. The CasX:gNA system of any one of embodiments 1-19, wherein the targeting sequence of the gNA is complementary to a sequence of a C9orf72 intron.

24. The CasX:gNA system of any one of embodiments 1-19, wherein the targeting sequence of the gNA is complementary to a sequence of a C9orf72 intron-exon junction.

25. The CasX:gNA system of any one of embodiments 1-19, wherein the targeting sequence of the gNA is complementary to a sequence of a C9orf72 regulatory element.

26. The CasX:gNA system of any one of embodiments 1-19, wherein the targeting sequence of the gNA is complementary to a sequence of an intergenic region of the C9orf72 gene.

27. The CasX:gNA system of any one of embodiments 1-19, wherein the targeting sequence of the gNA is complementary to a sequence that is 5′ to the HRS.

28. The CasX:gNA system of embodiment 27, wherein the targeting sequence of the gNA is complementary to a sequence of intron 1 or a promoter of the C9orf72 gene.

29. The CasX:gNA system of any one of embodiments 1-28, further comprising a second gNA, wherein the second gNA has a targeting sequence complementary to a different or overlapping portion of the target nucleic acid sequence compared to the targeting sequence of the gNA of any one of the preceding embodiments.

30. The CasX:gNA system of embodiment 28, wherein the targeting sequence of the second gNA gNA is complementary to a sequence that is 5′ or 3′ to the HRS.

31. The CasX:gNA system of embodiment 30, wherein the targeting sequence of the gNA is complementary to a sequence of intron 1 of the C9orf72 gene.

32. The CasX:gNA system of any one of embodiments 1-31, wherein the gNA has a scaffold comprising a sequence selected from the group consisting of sequences set forth in Table 1 and Table 2, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto.

33. The CasX:gNA system of any one of embodiments 1-31, wherein the gNA has a scaffold comprising a sequence having at least one modification relative to a reference gNA sequence selected from the group consisting of the sequences of SEQ ID NOS: 4-16.

34. The CasX:gNA system of embodiment 33, wherein the at least one modification of the reference gNA comprises at least one substitution, deletion, or insertion of a nucleotide of the gNA sequence.

35. The CasX:gNA system of any one of embodiments 1-34, wherein the gNA is chemically modified.

36. The CasX:gNA system of any one of embodiments 1-35, wherein the CasX protein comprises a reference CasX protein having a sequence of any one of SEQ ID NOS: 1-3, a CasX variant protein having a sequence of Table 4, or a sequence having at least about 50%, at least about 60%, at least about 70%, at least about 80%, at least about 90%, at least about 95%, at least about 96%, at least about 97%, at least about 98%, or at least about 99% sequence identity thereto.

37. The CasX:gNA system of embodiment 36, wherein the CasX variant protein comprises at least one modification relative to a reference CasX protein having a sequence selected from SEQ ID NOS:1-3.

38. The CasX:gNA system of embodiment 37, wherein the at least one modification comprises at least one amino acid substitution, deletion, or insertion in a domain of the CasX variant protein relative to the reference CasX protein.

39. The CasX:gNA system of embodiment 38, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

40. The CasX:gNA system of any one of embodiments 36-39, wherein the CasX protein further comprises one or more nuclear localization signals (NLS).

41. The CasX:gNA system of embodiment 40, wherein the one or more NLS are selected from the group of sequences consisting of PKKKRKV (SEQ ID NO: 165), KRPAATKKAGQAKKKK (SEQ ID NO: 166), PAAKRVKLD (SEQ ID NO: 167), RQRRNELKRSP (SEQ ID NO: 168), NQSSNFGPMKGGNFGGRSSGPYGGGGQYFAKPRNQGGY (SEQ ID NO: 169), RMRIZFKNKGKDTAELRRRRVEVSVELRKAKKDEQILKRRNV (SEQ ID NO: 170), VSRKRPRP (SEQ ID NO: 171), PPKKARED (SEQ ID NO: 172), PQPKKKPL (SEQ ID NO: 173), SALIKKKKKMAP (SEQ ID NO: 174), DRLRR (SEQ ID NO:175), PKQKKRK (SEQ ID NO: 176), RKLKKKIKKL (SEQ ID NO: 177), REKKKFLKRR (SEQ ID NO: 178), KRKGDEVDGVDEVAKKKSKK (SEQ ID NO: 179), RKCLQAGMNLEARKTKK (SEQ ID NO: 180), PRPRKIPR (SEQ ID NO: 181), PPRKKRTVV (SEQ ID NO: 182), NLSKKKKRKREK (SEQ ID NO: 183), RRPSRPFRKP (SEQ ID NO: 184), KRPRSPSS (SEQ ID NO: 185), KRGINDRNFWRGENERKTR (SEQID NO: 186), PRPPKMARYDN (SEQ ID NO: 187), KRSFSKAF (SEQ ID NO: 188), KLKIKRPVK (SEQ ID NO: 189), PKTRRRPRRSQRKRPPT (SEQ ID NO: 191), RRKKRRPRRKKRR (SEQ ID NO: 194), PKKKSRKPKKKSRK (SEQ ID NO: 195), HKKKHPDASVNFSEFSK (SEQ ID NO: 196), QRPGPYDRPQRPGPYDRP (SEQ ID NO: 197), LSPSLSPLLSPSLSPL (SEQ ID NO: 198), RGKGGKGLGKGGAKRHRK (SEQ ID NO: 199), PKRGRGRPKRGRGR (SEQ ID NO: 200), MSRRRKANPTKLSENAKKLAKEVEN (SEQ ID NO: 192), PKKKRKVPPPPAAKRVKLD (SEQ ID NO: 190), and PKKKRKVPPPPKKKRKV (SEQ ID NO: 201).

42. The CasX:gNA system of embodiment 40 or embodiment 41, wherein the one or more NLS are at the C-terminus of the CasX protein.

43. The CasX:gNA system of embodiment 40 or embodiment 41, wherein the one or more NLS are at the N-terminus of the CasX protein.

44. The CasX:gNA system of embodiment 40 or embodiment 41, wherein the one or more NLS are at the N-terminus and C-terminus of the CasX protein.

45. The CasX:gNA system of any one of embodiments 36-44, wherein the CasX variant protein and the gNA exhibit at least one or more improved characteristics as compared to a reference CasX protein and the gNA of Table 1.

46. The CasX:gNA system of embodiment 45, wherein the improved characteristic is selected from the group consisting of improved folding of the CasX protein, improved binding affinity of the CasX protein to the gNA, improved ribonuclear protein complex (RNP) formation, higher percentage of cleavage-competent RNP, improved binding affinity to the target nucleic acid sequence, improved binding affinity for a PAM sequence, improved unwinding of the target nucleic acid sequence, increased activity, increased target nucleic acid sequence cleavage rate, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved CasX protein stability, improved protein:guide RNA complex stability, improved protein solubility, improved protein:gNA complex solubility, improved protein yield, improved protein expression, and improved fusion characteristics.

47. The CasX:gNA system of embodiment 45 or embodiment 46, wherein the improved characteristic of the CasX variant protein is at least about 1.1 to about 100,000-fold improved relative to the reference CasX protein of SEQ ID NO: 1, SEQ ID NO: 2, or SEQ ID NO: 3.

48. The CasX:gNA system of embodiment 45 or embodiment 46, wherein the improved characteristic of the CasX variant protein is at least about 10-fold, at least about 100-fold, at least about 1,000-fold, or at least about 10,000-fold improved relative to the reference CasX protein of SEQ ID NO: 1, SEQ ID NO: 2, or SEQ ID NO: 3.

49. The CasX:gNA system of any one of embodiments 46-48, wherein the improved characteristic is improved binding affinity to the target nucleic acid sequence.

50. The CasX:gNA system of any one of embodiments 46-48, wherein the improved characteristic is increased target nucleic acid sequence cleavage rate.

51. The CasX:gNA system of any one of embodiments 46-48, wherein the improved characteristic is increased binding affinity to one or more PAM sequences wherein the one or more PAM sequences are selected from the group consisting of TTC, ATC, GTC, and CTC.

52. The CasX:gNA system of embodiment 51, wherein the increased binding affinity for the one or more PAM sequences is at least 1.5-fold greater compared to the binding affinity of any one of the CasX proteins of SEQ ID NOS: 1-3 for the PAM sequences.

53. The CasX:gNA system of any one of the preceding embodiments, wherein the CasX variant protein and the gNA are associated together in an RNP.

54. The CasX:gNA system of embodiment 52, wherein the RNP has at least a 5%, at least a 10%, at least a 15%, or at least a 20% higher percentage of cleavage-competent RNP compared to an RNP of a reference CasX and the gNA of Table 1.

55. The CasX:gNA system of any one of embodiments 39-54, wherein the CasX variant protein comprises a nuclease domain having nickase activity.

56. The CasX:gNA system of embodiment 55, wherein the CasX variant can cleave only one strand of a double-stranded target nucleic acid molecule.

57. The CasX:gNA system of any one of embodiments 1-54, wherein the CasX variant protein comprises a nuclease domain having double-stranded cleavage activity.

58. The CasX:gNA system of any one of embodiments 1-44, wherein the CasX protein is a catalytically inactive CasX (dCasX) protein, and wherein the dCasX and the gNA retain the ability to bind to the target nucleic acid sequence.

59. The CasX:gNA system of embodiment 58, wherein the dCasX comprises a mutation at residues:
a. D672, E769, and/or D935 corresponding to the reference CasX protein of SEQ ID NO:1; or
b. D659, E756 and/or D922 corresponding to the reference CasX protein of SEQ ID NO: 2.

60. The CasX:gNA system of embodiment 59, wherein the mutation is a substitution of alanine for the residue.

61. The CasX:gNA system of any one of embodiments 1-57, further comprising a donor template nucleic acid.

62. The CasX:gNA system of embodiment 61, wherein the donor template comprises a nucleic acid comprising at least a portion of the C9orf72 gene, wherein the C9orf72 gene portion is selected from the group consisting of a C9orf72 exon, a C9orf72 intron, a C9orf72 intron-exon junction, a C9orf72 regulatory element, or a combination thereof.

63. The CasX:gNA system of embodiment 61 or embodiment 62, wherein the donor template comprises homologous arms complementary to sequences flanking a cleavage site in the target nucleic acid.

64. The CasX:gNA system of embodiment 61-63, wherein the donor template ranges in size from 10-15,000 nucleotides.

65. The CasX:gNA system of any one of embodiments 61-64, wherein the donor template is a single-stranded DNA template or a single stranded RNA template.

66. The CasX:gNA system of any one of embodiments 61-64, wherein the donor template is a double-stranded DNA template.

67. The CasX:gNA system of any one of embodiments 61-66, wherein the donor template comprises one or more mutations compared to a wild-type C9orf72 gene.

68. The CasX:gNA system of any one of embodiments 61-66, wherein the donor template comprises a heterologous sequence compared to a wild-type C9orf72 gene.

69. The CasX:gNA system of any one of embodiments 61-66, wherein the donor template comprises all or a portion of a wild-type C9orf72 gene.

70. A nucleic acid comprising a sequence that encodes the CasX:gNA system of any one of embodiments 1-60.

71. The nucleic acid of embodiment 70, wherein the sequences encoding the CasX protein and gNA are codon optimized for expression in a eukaryotic cell.

72. A vector comprising the nucleic acid of embodiment 70 or embodiment 71.

73. The vector of embodiment 72, wherein the vector further comprises a promoter.

74. A vector comprising a donor template, wherein the donor template comprises a nucleic acid comprising at least a portion of a C9orf72 gene, wherein the C9orf72 gene portion is selected from the group consisting of a C9orf72 exon, a C9orf72 intron, a C9orf72 intron-exon junction, and a C9orf72 regulatory element.

75. The vector of embodiment 74, wherein the donor template comprises one or more mutations compared to a wild-type C9orf72 gene or comprises a heterologous sequence flanked by two homologous arms complementary to sequences that are 5′ and 3′ to a cleavage site in the C9orf72 target nucleic acid.

76. The vector of embodiment 74 or embodiment 75, further comprising the nucleic acid of embodiment 70 or embodiment 71.

77. The vector of any one of embodiments 72-76, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, and an RNA vector.

78. The vector of embodiment 77, wherein the vector is an AAV vector.

79. The vector of embodiment 78, wherein the AAV vector is selected from AAV1, AAV2, AAV3, AAV4, AAV5, AAV6, AAV7, AAV8, AAV9, AAV10, AAV-Rh74, or AAVRh10.

80. The vector of embodiment 77, wherein the vector is a retroviral vector.

81. The vector of embodiment 77, wherein the vector encoding the VLP comprises one or more nucleic acids encoding a gag polyprotein, the CasX protein of any one of embodiments 36-60, and the gNA of any one of embodiments 1-35.

82. A virus-like particle (VLP) comprising the CasX protein of any one of embodiments 36-60, and the gNA of any one of embodiments 1-35.

83. The VLP of embodiment 82, wherein the CasX protein and the gNA are associated together in an RNP.

84. The VLP of embodiment 82 or embodiment 83, further comprising a pseudotyping viral envelope glycoprotein or antibody fragment that provides for binding and fusion of the VLP to a target cell.

85. A method of modifying a C9orf72 target nucleic acid sequence, the method comprising contacting the target nucleic acid sequence with a CasX protein and a guide nucleic acid (gNA) comprising a targeting sequence wherein said contacting comprises introducing into a cell:
a. the CasX:gNA system of any one of embodiments 1-69;
b. the nucleic acid of embodiment 70 or embodiment 71;
c. the vector as in any one of embodiments 72-81;
d. the VLP of any one of embodiments 82-84; or
e. combinations thereof,
wherein said contacting results in modification of the C9orf72 target nucleic acid sequence by the CasX protein.

86. The method of embodiment 85, wherein the CasX protein and the gNA are associated together in a ribonuclear protein complex (RNP).

87. The method of embodiment 85 or embodiment 86, further comprising a second gNA or a nucleic acid encoding the second gNA, wherein the second gNA has a targeting sequence complementary to a different portion of the target nucleic acid sequence or its complement compared to the guide of embodiment 85.

88. The method any one of embodiments 85-87, wherein the C9orf72 gene comprises a mutation.

89. The method of embodiment 88, wherein the mutation is a gain-of-function mutation.

90. The method of embodiment 88, wherein the mutation is a loss-of-function mutation.

91. The method of embodiment 88, wherein the C9orf72 gene mutation comprises more than 30, more than 100, more than 500, more than 700, more than 1000, or more than 1600 copies of a hexanucleotide repeat sequence GGGGCC.

92. The method of any one of embodiments 85-90, wherein the modifying comprises introducing a single-stranded break in the target nucleic acid sequence.

93. The method of any one of embodiments 85-90, wherein the modifying comprises introducing a double-stranded break in the target nucleic acid sequence.

94. The method of any one of embodiments 85-93, wherein the modifying comprises introducing an insertion, deletion, substitution, duplication, or inversion of one or more nucleotides in the target nucleic acid sequence.

95. The method of any one of embodiments 85-94, wherein the modifying of the target nucleic acid sequence occurs in vitro or ex vivo.

96. The method of any one of embodiments 85-95, wherein the modifying of the target nucleic acid sequence occurs inside of a cell.

97. The method of any one of embodiments 85-95, wherein the modifying of the target nucleic acid sequence occurs in vivo.

98. The method of any one of embodiments 85-97, wherein the cell is a eukaryotic cell.

99. The method of embodiment 98, wherein the eukaryotic cell is selected from the group consisting of a rodent cell, a mouse cell, a rat cell, a pig cell, a primate cell, and a non-human primate cell.

100. The method of embodiment 98, wherein the eukaryotic cell is a human cell.

101. The method of any one of embodiments 85-100, wherein the cell is selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

102. The method of any one of embodiments 85-101, wherein the method further comprises contacting the target nucleic acid sequence with a donor template comprising homologous arms complementary to sequences flanking a cleavage site in the target nucleic acid targeted by the CasX:gNA system of any one of embodiments 1-57.

103. The method of embodiment 102, wherein the donor template comprises one or more mutations compared to the wild-type C9orf72 gene sequence, and wherein the insertion results in a knock-down or knock-out of the C9orf72 gene.

104. The method of embodiment 102, wherein insertion of the donor template replaces some or all of the HRS of the C9orf72 gene.

105. The method of embodiment 102, wherein the donor template comprises all or a portion of a wild-type C9orf72 gene sequence, wherein the insertion corrects one or more mutation(s) of the C9orf72 gene.

106. The method of any one of embodiments 102-104, wherein the donor template ranges in size from 10-15,000 nucleotides.

107. The method of any one of embodiments 102-104, wherein the donor template ranges in size from 100-1,000 nucleotides.

108. The method of any one of embodiments 102-107, wherein the donor template is a single-stranded DNA template or a single stranded RNA template.

109. The method of any one of embodiments 102-107, wherein the donor template is a double-stranded DNA template.

110. The method of any one of embodiments 102-109, wherein the donor template is inserted by homology directed repair (HDR).

111. The method of any one of embodiments 85-110, wherein the target nucleic acid has been modified such that expression of the HRS or the DPR is reduced by at least about 10%, at least about 20%, at least about 30%, at least about 40%, at least about 50%, at least about 60%, at least about 70%, at least about 80%, or at least about 90% in comparison to target nucleic acid that has not been modified.

112. The method of any one of embodiments 85-111, wherein the vector is administered to a subject at a therapeutically effective dose.

113. The method of embodiment 112, wherein the subject is selected from the group consisting of mouse, rat, pig, and non-human primate.

114. The method of embodiment 112, wherein the subject is a human.

115. The method of any one of embodiments 85-114, wherein the vector is administered at a dose of at least about 1×108 vector genomes (vg), at least about 1×10⁹ vg, at least about 1×10¹⁰ vg at least about 1×10¹¹ vg, or at least about 1×10¹² vg, or at least about 1×10¹³ vg, or at least about 1×10¹⁴ vg, or at least about 1×10¹⁵ vg, or at least about 1×10¹⁶ vg.

116. The method of any one of embodiments 111-115, wherein the vector is administered by a route of administration selected from the group consisting of subcutaneous, intradermal, intraneural, intranodal, intramedullary, intramuscular, intralumbar, intrathecal, subarachnoid, intraventricular, intracapsular, intravenous, intralymphatic, or intraperitoneal routes, wherein the administering method is injection, transfusion, or implantation.

117. The method of any one of embodiments 85-116, comprising further contacting the target nucleic acid sequence with an additional CRISPR nuclease, or a polynucleotide encoding the additional CRISPR nuclease.

118. The method of embodiment 117, wherein the additional CRISPR nuclease is a CasX protein having a sequence different from the CasX protein of any of the preceding embodiments.

119. The method of embodiment 117, wherein the additional CRISPR nuclease is not a CasX protein.

120. A method of altering a C9orf72 target nucleic acid sequence of a cell, comprising contacting said cell with:
a) the CasX:gNA system of any one of embodiments 1-69;
b) the nucleic acid of embodiment 70 or embodiment 71;
c) the vector of any one of embodiments 72-81;
d) the VLP of any one of embodiments 82-84; or
e) combinations thereof,
wherein said contacting results in modification of the C9orf72 target nucleic acid sequence by the CasX protein.

121. The method of embodiment 120, wherein the cell has been modified such that expression of the HRS and/or the DPR is reduced by at least about 10%, at least about 20%, at least about 30%, at least about 40%, at least about 50%, at least about 60%, at least about 70%, at least about 80%, or at least about 90% in comparison to a cell that has not been modified.

122. The method of embodiment 120 or embodiment 121, wherein the cell has been modified such that the cell does not express a detectable level of a dipeptide repeat protein (DPR).

123. A population of cells modified by the method of embodiment 120 or embodiment 121, wherein the cells have been modified such that at least 10%, at least about 20%, at least about 30%, at least about 40%, at least about 50%, at least about 60%, at least about 70%, at least about 80%, or at least about 90% of the modified cells do not express a detectable level of DPR.

124. The population of cells of embodiment 123, wherein the cell is a non-primate mammalian cell, a non-human primate cell, or a human cell.

125. The population of cells of embodiment 123 or embodiment 124, wherein the cells are selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

126. A method of treating a C9orf72-related disorder in a subject in need thereof, comprising modifying a C9orf72 gene in a cell of the subject, the modifying comprising contacting said cell with;
a. CasX:gNA system of any one of embodiments 1-69;
b. the nucleic acid of embodiment 70 or embodiment 71;
c. the vector as in any one of embodiments 72-81;
d. the VLP of any one of embodiments 82-84; or
e. combinations thereof,
wherein said contacting results in modification of the C9orf72 target nucleic acid sequence by the CasX protein.

127. The method of embodiment 126, wherein the C9orf72-related disorder is amyotrophic lateral sclerosis (ALS) or frontotemporal dementia (FTD).

128. The CasX:gNA system of embodiment 126, wherein the targeting sequence of the gNA is complementary to a sequence that is 5′ to the HRS of the C9orf72 gene.

129. The method of any one of embodiments 126-128, further comprising a second gNA or a nucleic acid encoding the second gNA, wherein the second gNA has a targeting sequence complementary to a different or overlapping portion of the target nucleic acid sequence compared to the gNA of embodiment 126.

130. The CasX:gNA system of embodiment 129, wherein the targeting sequence of the second gNA is complementary to a sequence in intron 1 and is 3′ to the HRS of the C9orf72 gene.

131. The method of any one of embodiments 126-130, wherein the modifying introduces one or more mutations in the C9orf72 gene, or wherein expression of the HRS and/or the DPR is reduced by at least about 10%, at least about 20%, at least about 30%, at least about 40%, at least about 50%, at least about 60%, at least about 70%, at least about 80%, or at least about 90% in comparison to a cell that has not been modified.

132. The method of any one of embodiments 126-130, wherein the method comprises contacting the cell with the donor template of any one of embodiments 61-69.

133. The method of any one of embodiments 126-132, wherein the cell is selected from the group consisting of a Purkinje cell, frontal cortex neuron, motor cortex neuron, hippocampus neuron, cerebellum neuron, upper motor neuron, spinal cord neuron, spinal cord motor neuron, glial cell, and astrocytes.

134. The method of any one of embodiments 126-133, wherein the subject is selected from the group consisting of mouse, rat, pig, non-human primate, and human.

135. The method of embodiment 134, wherein the subject is a human.

136. The method of any one of embodiments 126-135, wherein the vector is administered to a subject at a therapeutically effective dose.

137. The method of any one of embodiments 126-136, wherein the vector is administered to the subject at a dose of at least about 1×10¹⁰ vector genomes (vg), or at least about 1×10¹¹ vg, or at least about 1×10¹² vg, or at least about 1×10¹³ vg, or at least about 1×10¹⁴ vg, or at least about 1×10¹⁵ vg, or at least about 1×10¹⁶ vg.

138. The method of any one of embodiments 126-136, wherein the vector is administered by a route of administration selected from the group consisting of subcutaneous, intradermal, intraneural, intranodal, intramedullary, intramuscular, intralumbar, intrathecal, subarachnoid, intraventricular, intracapsular, intravenous, intralymphatic, or intraperitoneal routes, wherein the administering method is injection, transfusion, or implantation.

139. The method of any one of embodiments 126-138, comprising further contacting the target nucleic acid sequence with an additional CRISPR nuclease, or a polynucleotide encoding the additional CRISPR protein.

140. The method of embodiment 139, wherein the additional CRISPR nuclease is a CasX protein having a sequence different from the CasX of any of the preceding embodiments.

141. The method of embodiment 140, wherein the additional CRISPR nuclease is not a CasX protein.

142. The method of any one of embodiments 126-141, wherein the method further comprises administering a chemotherapeutic agent.

143. The method of any one of embodiments 126-142, wherein the method results in improvement in at least one clinically-relevant parameter selected from the group consisting of neuronal cell death, neuroinflammation, TDP-43 related pathology, axonal and neuromuscular junction (NMJ) abnormalities, dendritic spine density changes at prefrontal cortex, electrophysiological deficits in neonatal cortical neurons, change from baseline in percent of predicted slow vital capacity (SVC), change from baseline in muscle strength, change from baseline in bulbar strength, ALS Functional Rating Scale (ALSFRS-(R)), combined assessment of function and survival, duration of response, time to death, time to tracheostomy, time to persistent assisted ventilation (DTP), forced vital capacity (% FVC); manual muscle test, maximum voluntary isometric contraction, duration of response, progression-free survival, time to progression of disease, and time-to-treatment failure.

144. The method of any one of embodiments 126-142, wherein the method results in improvement in at least two clinically-relevant parameters selected from the group consisting of neuronal cell death, neuroinflammation, TDP-43 related pathology, axonal and neuromuscular junction (NMJ) abnormalities, dendritic spine density changes at prefrontal cortex, electrophysiological deficits in neonatal cortical neurons, change from baseline in percent of predicted slow vital capacity (SVC), change from baseline in muscle strength, change from baseline in bulbar strength, ALS Functional Rating Scale (ALSFRS-(R)), combined assessment of function and survival, duration of response, time to death, time to tracheostomy, time to persistent assisted ventilation (DTP), forced vital capacity (% FVC); manual muscle test, maximum voluntary isometric contraction, duration of response, progression-free survival, time to progression of disease, and time-to-treatment failure.

### EXAMPLES

### Example 1: Creation, Expression and Purification of CasX Stx2

### 1. Growth and Expression

An expression construct for CasX Stx2 (also referred to herein as CasX2), derived from Planctomycetes (having the CasX amino acid sequence of SEQ ID NO: 2 and encoded by the sequence in Table 5, below), was constructed from gene fragments (Twist Biosciences) that were codon optimized for E. coli. The assembled construct contains a TEV-cleavable, C-terminal, TwinStrep tag and was cloned into a pBR322-derivative plasmid backbone containing an ampicillin resistance gene. The expression construct was transformed into chemically competent BL21* (DE3) E. coli and a starter culture was grown overnight in LB broth supplemented with carbenicillin at 37° C., 200 RPM, in UltraYield Flasks (Thomson Instrument Company). The following day, this culture was used to seed expression cultures at a 1:100 ratio (starter culture:expression culture). Expression cultures were Terrific Broth (Novagen) supplemented with carbenicillin and grown in UltraYield flasks at 37° C., 200 RPM. Once the cultures reached an optical density (OD) of 2, they were chilled to 16° C. and IPTG (isopropyl β-D-1-thiogalactopyranoside) was added to a final concentration of 1 mM, from a 1 M stock. The cultures were induced at 16° C., 200 RPM for 20 hours before being harvested by centrifugation at 4,000×g for 15 minutes, 4° C. The cell paste was weighed and resuspended in lysis buffer (50 mM HEPES-NaOH, 250 mM NaCl, 5 mM MgCl₂, 1 mM TCEP, 1 mM benzamidine-HCL, 1 mM PMSF, 0.5% CHAPS, 10% glycerol, pH 8) at a ratio of 5 mL of lysis buffer per gram of cell paste. Once resuspended, the sample was frozen at −80° C. until purification.

TABLE 5
DNA sequence of CasX Stx2 construct

| Construct | DNA Sequence |
|---|---|
| SV40 NLS-CasX- | ATGGCTCCGAAGAAGAAGCGAAAGGTCAGCCAGGAAATTAAACGCATCA |
| SV40 NLS-TEV | ACAAGATCCGCCGTCGTCTGGTAAAAGACAGCAATACGAAAAAAGCCGG |
| cleavage site- | AAAAACCGGTCCGATGAAAACGCTGCTGGTGCGCGTGATGACGCCGGAT |
| TwinStrep tag | CTCCGCGAACGTCTTGAGAATTTGCGTAAGAAACCTGAAAATATTCCGC |
| | AACCGATTTCTAACACCTCGCGCGCCAATCTGAATAAACTGCTGACCGA |
| | TTACACCGAAATGAAGAAAGCGATTCTGCACGTTTACTGGGAAGAGTTC |
| | CAGAAAGACCCGGTCGGTCTGATGAGCCGCGTTGCGCAACCTGCGCCGA |
| | AAAATATCGATCAGCGCAAGTTAATCCCGGTTAAAGATGGTAATGAACG |
| | TTTAACCTCCAGCGGCTTTGCCTGCAGTCAGTGCTGCCAGCCACTTTAT |
| | GTTTATAAACTTGAACAGGTTAACGATAAAGGGAAACCCCATACCAATT |
| | ATTTCGGCCGCTGCAATGTCAGCGAACATGAACGCCTGATTTTGTTAAG |
| | CCCGCATAAACCGGAAGCGAATGACGAACTGGTGACCTATTCCCTGGGT |
| | AAATTTGGTCAGCGGGCGCTGGATTTTTACAGCATTCATGTGACGCGGG |
| | AAAGTAACCATCCGGTAAAGCCACTGGAACAAATCGGCGGTAACAGCTG |
| | CGCCTCTGGCCCGGTTGGCAAAGCGCTTAGCGATGCCTGTATGGGCGCG |
| | GTGGCGAGCTTTCTGACAAAATACCAGGATATTATCCTGGAGCATCAGA |
| | AGGTGATCAAAAAGAACGAGAAACGTCTGGCAAATTTAAAGGATATTGC |
| | CTCCGCTAACGGCCTGGCGTTCCCGAAGATTACCTTACCGCCGCAGCCG |
| | CACACCAAAGAAGGTATCGAAGCGTATAACAACGTTGTTGCCCAGATCG |
| | TCATCTGGGTGAATCTCAACCTGTGGCAAAAACTGAAAATTGGTCGTGA |
| | TGAAGCAAAACCGTTGCAGCGACTGAAAGGATTCCCGTCGTTTCCGCTG |
| | GTTGAACGACAGGCGAACGAAGTGGATTGGTGGGATATGGTTTGTAACG |
| | TCAAAAAATTGATCAACGAAAAAAAGGAAGATGGCAAAGTTTTCTGGCA |
| | AAATCTGGCGGGTTACAAACGTCAGGAGGCGTTGCTTCCGTATCTCTCT |
| | TCAGAAGAAGATCGCAAAAAAGGCAAGAAGTTTGCTCGCTATCAGTTTG |
| | GCGATTTATTACTGCATCTGGAAAAAAAACACGGCGAAGACTGGGGCAA |
| | AGTGTACGATGAAGCCTGGGAGCGTATCGACAAAAAAGTGGAAGGTTTG |
| | TCGAAACATATTAAACTCGAAGAAGAGCGCCGCAGTGAAGATGCGCAGT |
| | CAAAAGCAGCGCTGACGGACTGGTTACGTGCGAAAGCCAGTTTTGTGAT |
| | TGAAGGATTAAAAGAAGCTGATAAAGATGAATTTTGCCGTTGCGAACTG |
| | AAACTGCAAAAATGGTATGGCGACCTGCGCGGCAAACCGTTCGCCATTG |
| | AGGCAGAAAATAGCATCCTTGATATCTCCGGTTTCAGCAAACAATATAA |
| | CTGCGCGTTTATTTGGCAGAAAGACGGCGTGAAAAAGCTTAACCTGTAT |
| | CTGATCATTAACTATTTTAAAGGCGGGAAACTGCGTTTCAAGAAAATCA |
| | AGCCGGAAGCATTTGAAGCCAATCGTTTTTATACCGTTATTAATAAAAA |
| | AAGCGGTGAAATCGTGCCGATGGAAGTTAATTTTAACTTTGATGATCCG |
| | AACTTGATTATTCTGCCGCTGGCATTCGGTAAACGGCAGGGCCGTGAGT |
| | TTATCTGGAACGACCTGTTATCGCTGGAAACGGGCAGCCTGAAATTAGC |
| | CAACGGTCGCGTCATTGAAAAAACGCTCTACAACCGCCGCACCCGCCAG |
| | GATGAGCCGGCACTGTTTGTCGCGCTGACCTTTGAACGGCGTGAAGTCC |
| | TCGATAGCAGCAACATCAAACCAATGAACCTTATCGGTATTGATCGTGG |
| | TGAAAACATTCCTGCCGTTATCGCCCTGACTGATCCAGAAGGCTGCCCG |
| | CTTTCTCGCTTCAAAGATTCACTGGGCAACCCGACCCATATCCTCCGTA |
| | TTGGCGAGAGCTACAAAGAGAAACAGCGTACCATTCAGGCAGCCAAAGA |
| | AGTGGAGCAGCGTCGCGCGGGCGGCTATAGCCGTAAATATGCCAGCAAA |
| | GCTAAAAACCTGGCGGATGACATGGTGCGTAACACGGCGCGCGATTTGC |
| | TGTACTACGCCGTCACCCAGGACGCGATGCTGATTTTTGAGAACCTCTC |
| | CCGCGGTTTTGGGCGTCAGGGTAAACGCACGTTTATGGCGGAACGCCAG |
| | TATACGCGTATGGAGGACTGGCTGACCGCGAAGCTGGCCTATGAAGGCT |
| | TGCCGTCTAAAACTTACCTGAGCAAGACCCTGGCTCAGTACACCAGTAA |
| | AACCTGTAGTAATTGCGGCTTTACCATCACCAGCGCCGATTATGACCGC |
| | GTGCTGGAAAAGCTGAAGAAAACCGCCACCGGCTGGATGACCACCATCA |
| | ATGGTAAAGAGCTTAAAGTCGAAGGGCAGATTACTTATTACAACCGTTA |
| | TAAGCGGCAAAACGTGGTGAAAGATCTGTCGGTTGAGCTGGACCGTTTG |
| | TCTGAAGAAAGCGTGAACAATGATATCAGCTCCTGGACCAAAGGTCGTT |
| | CCGGCGAAGCGTTAAGTCTGTTGAAAAAGCGCTTTAGCCATCGCCCGGT |
| | GCAGGAAAAATTCGTTTGCCTGAACTGTGGCTTCGAAACCCACGCCGAC |
| | GAGCAAGCGGCGCTCAATATTGCGCGTAGCTGGCTGTTCCTGCGCAGCC |
| | AGGAATATAAAAAATATCAAACCAACAAAACAACTGGCAATACCGACAA |
| | GCGTGCCTTTGTTGAAACCTGGCAGAGCTTCTATCGCAAAAAACTGAAA |
| | GAGGTCTGGAAACCGGCGGTAGCGCCAAAGAAAAAACGCAAAGTGAGCG |
| | AAAATCTTTATTTTCAAGGTAGCGCATGGAGTCATCCTCAATTCGAGAA |
| | AGGTGGAGGTTCTGGCGGTGGATCGGGAGGTTCAGCGTGGAGCCACCCG |
| | CAGTTCGAAAAAGGAAGGGGATCCGGCTGCTAA (SEQ ID NO: |
| | 229) |

### 2. Purification

Frozen samples were thawed overnight at 4° C. with magnetic stirring. The viscosity of the resulting lysate was reduced by sonication and lysis was completed by homogenization in three passes at 17 k PSI using an Emulsiflex C3 (Avestin). Lysate was clarified by centrifugation at 50,000×g, 4° C., for 30 minutes and the supernatant was collected. The clarified supernatant was applied to a Heparin 6 Fast Flow column (GE Life Sciences) by gravity flow. The column was washed with 5 CV of Heparin Buffer A (50 mM HEPES-NaOH, 250 mM NaCl, 5 mM MgCl₂, 1 mM TCEP, 10% glycerol, pH 8), then with 5 CV of Heparin Buffer B (Buffer A with the NaCl concentration adjusted to 500 mM). Protein was eluted with 5 CV of Heparin Buffer C (Buffer A with the NaCl concentration adjusted to 1 M), collected in fractions. Fractions were assayed for protein by Bradford Assay and protein-containing fractions were pooled. The pooled heparin eluate was applied to a Strep-Tactin XT Superflow column (IBA Life Sciences) by gravity flow. The column was washed with 5 CV of Strep Buffer (50 mM HEPES-NaOH, 500 mM NaCl, 5 mM MgCl₂, 1 mM TCEP, 10% glycerol, pH 8). Protein was eluted from the column using 5 CV of Strep Buffer with 50 mM D-Biotin added and collected in fractions. CasX-containing fractions were pooled, concentrated at 4° C. using a 30 kDa cut-off spin concentrator, and purified by size exclusion chromatography on a Superdex 200 pg column (GE Life Sciences). The column was equilibrated with SEC Buffer (25 mM sodium phosphate, 300 mM NaCl, 1 mM TCEP, 10% glycerol, pH 7.25) operated by an AKTA Pure FPLC system (GE Life Sciences). CasX-containing fractions that eluted at the appropriate molecular weight were pooled, concentrated at 4° C. using a 30 kDa cut-off spin concentrator, aliquoted, and snap-frozen in liquid nitrogen before being stored at −80° C.

### 3. Results

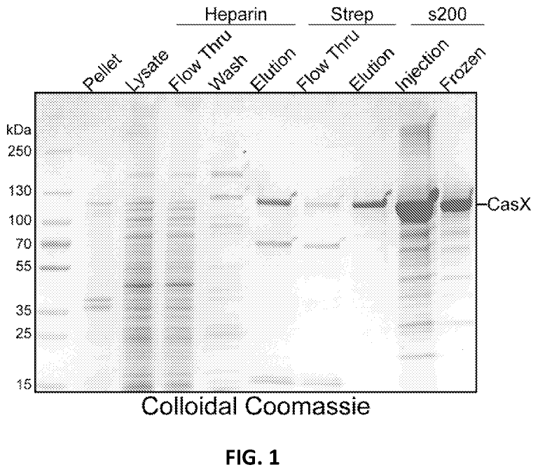

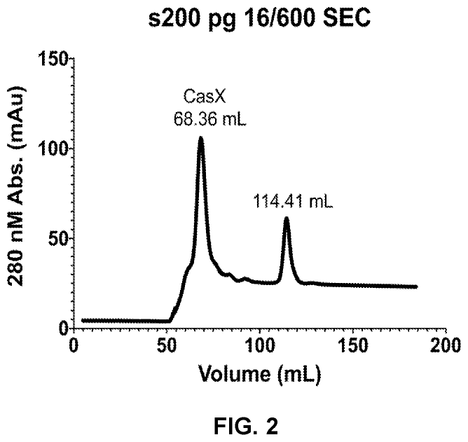

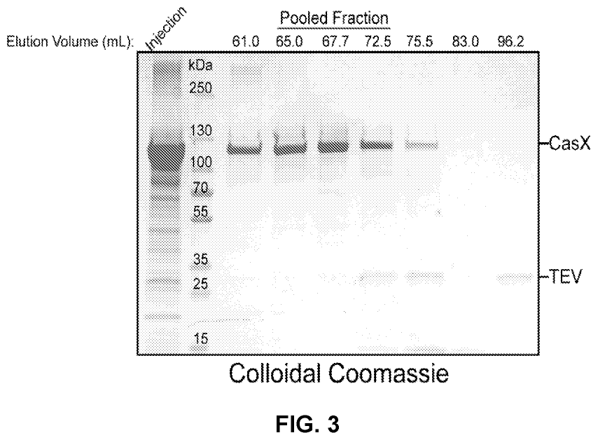

Samples from throughout the purification were resolved by SDS-PAGE and visualized by colloidal Coomassie staining, as shown in FIG. 1 and FIG. 3. In FIG. 1, the lanes, from left to right, are: molecular weight standards, Pellet: insoluble portion following cell lysis, Lysate: soluble portion following cell lysis, Flow Thru: protein that did not bind the Heparin column, Wash: protein that eluted from the column in wash buffer, Elution: protein eluted from the heparin column with elution buffer, Flow Thru: Protein that did not bind the StrepTactinXT column, Elution: protein eluted from the StrepTactin XT column with elution buffer, Injection: concentrated protein injected onto the s200 gel filtration column, Frozen: pooled fractions from the s200 elution that have been concentrated and frozen. In FIG. 3, the lanes from right to left, are the injection (sample of protein injected onto the gel filtration column) molecular weight markers, lanes 3-9 are samples from the indicated elution volumes. Results from the gel filtration are shown in FIG. 2. The 68.36 mL peak corresponds to the apparent molecular weight of CasX and contained the majority of CasX protein. The average yield was 0.75 mg of purified CasX protein per liter of culture, with 75% purity, as evaluated by colloidal Coomassie staining.

### Example 2: CasX Construct 119, 438 and 457

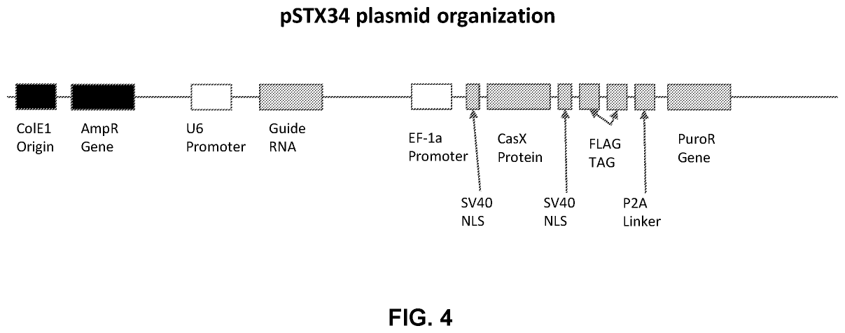

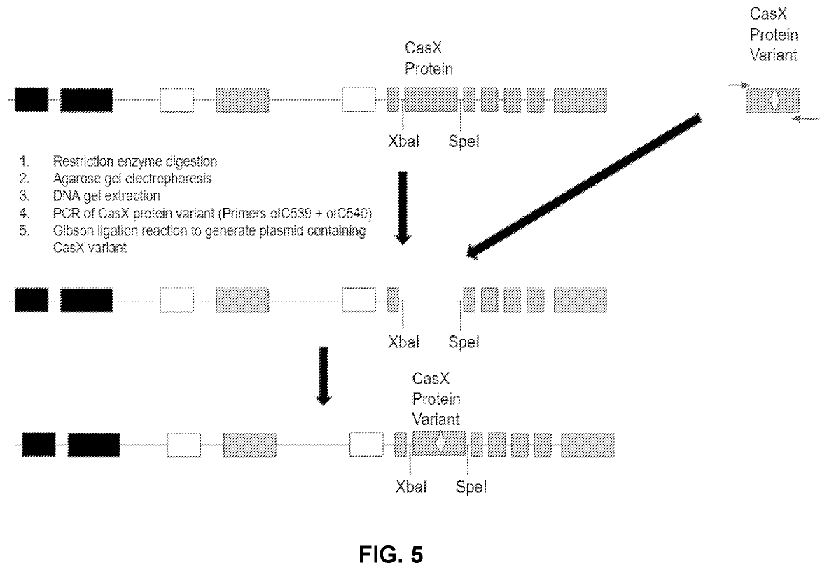

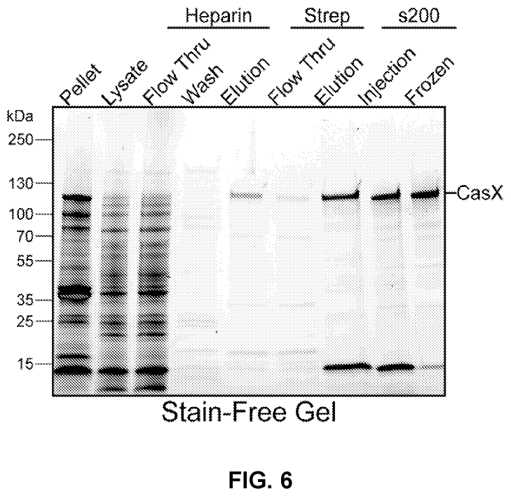

In order to generate the CasX 119, 438, and 457 constructs (sequences in Table 6), the codon-optimized CasX 37 construct (based on the CasX Stx2 construct of Example 1, encoding Planctomycetes CasX SEQ ID NO:2, with a A708K substitution and a [P793] deletion with fused NLS, and linked guide and non-targeting sequences) was cloned into a mammalian expression plasmid (pStX; see FIG. 4) using standard cloning methods. To build CasX 119, the CasX 37 construct DNA was PCR amplified in two reactions using Q5 DNA polymerase (New England BioLabs Cat #M0491L) according to the manufacturer's protocol, using primers oIC539 and oIC88 as well as oIC87 and oIC540 respectively (see FIG. 5). To build CasX 457, the CasX 365 construct DNA was PCR amplified in four reactions using Q5 DNA polymerase using primers oIC539 and oIC212, oIC211 and oIC376, oIC375 and oIC551, and oIC550 and oIC540 respectively. To build CasX 438, the CasX 119 construct DNA was PCR amplified in four reactions using Q5 DNA polymerase using primers oIC539 and oIC689, oIC688 and oIC376, oIC375 and oIC551, and oIC550 and oIC540 respectively. The resulting PCR amplification products were then purified using Zymoclean DNA clean and concentrator (Zymo Research Cat #4014) according to the manufacturer's protocol. The pStX backbone was digested using XbaI and SpeI in order to remove the 2931 base pair fragment of DNA between the two sites in plasmid pStx34. The digested backbone fragment was purified by gel extraction from a 1% agarose gel (Gold Bio Cat #A-201-500) using Zymoclean Gel DNA Recovery Kit (Zymo Research Cat #D4002) according to the manufacturer's protocol. The three fragments were then pieced together using Gibson assembly (New England BioLabs Cat #E2621S) following the manufacturer's protocol. Assembled products in the pStx34 were transformed into chemically-competent or electro-competent Turbo Competent E. coli bacterial cells, plated on LB-Agar plates (LB: Teknova Cat #L9315, Agar: Quartzy Cat #214510) containing carbenicillin. Individual colonies were picked and miniprepped using Qiagen Qiaprep spin Miniprep Kit (Qiagen Cat #27104) following the manufacturer's protocol. The resultant plasmids were sequenced using Sanger sequencing to ensure correct assembly. pStX34 includes an EF-1α promoter for the protein as well as a selection marker for both puromycin and carbenicillin. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase (New England BioLabs Cat #M0202L) and an appropriate restriction enzyme for the plasmid. Golden Gate products were transformed into chemically or electro-competent cells such as NEB Turbo competent E. coli (NEB Cat #C2984I), plated on LB-Agar plates containing carbenicillin. Individual colonies were picked and miniprepped using Qiagen Qiaprep spin Miniprep Kit (Qiagen Cat #27104) and following the manufacturer's protocol. The resultant plasmids were sequenced using Sanger sequencing to ensure correct ligation. SaCas9 and SpyCas9 control plasmids were prepared similarly to pStX plasmids described above, with the protein and guide regions of pStX exchanged for the respective protein and guide. Targeting sequences for SaCas9 and SpyCas9 were either obtained from the literature or were rationally designed according to established methods. The expression and recovery of the CasX 119 and 457 proteins was performed using the general methodologies of Example 1 (however the DNA sequences were codon optimized for expression in E. coli). The results of analytical assays for CasX 119 are shown in FIGS. 6-8. The average yield of the CasX 119 was 1.56 mg of purified CasX protein per liter of culture at 75% purity, as evaluated by colloidal Coomassie staining. FIG. 6 shows an SDS-PAGE gel of purification samples, visualized on a Bio-Rad Stain-Free™ gel, as described. The lanes, from left to right, are: Pellet: insoluble portion following cell lysis, Lysate: soluble portion following cell lysis, Flow Thru: protein that did not bind the Heparin column, Wash: protein that eluted from the column in wash buffer, Elution: protein eluted from the heparin column with elution buffer, Flow Thru: Protein that did not bind the StrepTactinXT column, Elution: protein eluted from the StrepTactin XT column with elution buffer, Injection: concentrated protein injected onto the s200 gel filtration column, Frozen: pooled fractions from the s200 elution that have been concentrated and frozen.

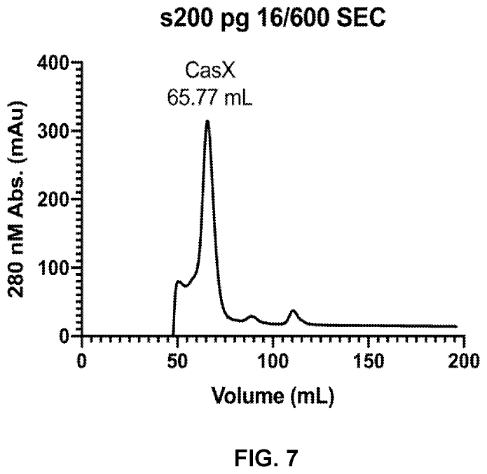

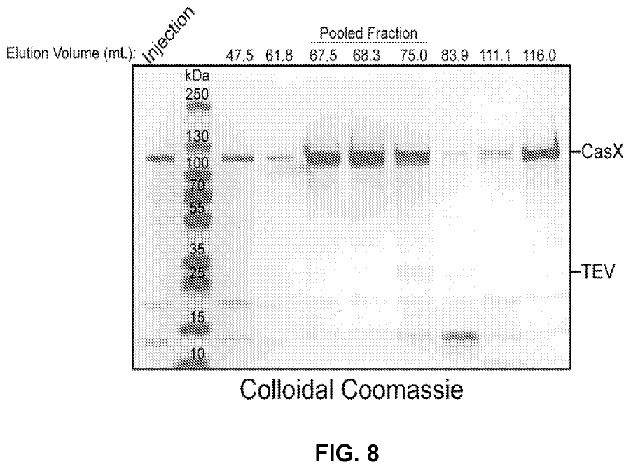

FIG. 7 shows the chromatogram of Superdex 200 16/600 pg Gel Filtration, as described. Gel filtration run of CasX variant 119 protein plotted as 280 nm absorbance against elution volume. The 65.77 mL peak corresponds to the apparent molecular weight of CasX variant 119 and contained the majority of CasX variant 119 protein. FIG. 8 shows an SDS-PAGE gel of gel filtration samples, stained with colloidal Coomassie, as described. Samples from the indicated fractions were resolved by SDS-PAGE and stained with colloidal Coomassie. From right to left, Injection: sample of protein injected onto the gel filtration column, molecular weight markers, lanes 3-10: samples from the indicated elution volumes.

TABLE 6
Sequences of CasX 119, 438 and 457

| | DNA | |
|---|---|---|
| Construct | Sequence | Amino Acid Sequence |
| CasX | (SEQ ID | QEIKRINKIRRRLVKDSNTKKAGKTGPMKTLLVRVMTPDLRERLEN |
| 119 | NO: 230) | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHQKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSEEDRKKGKKFARYQFGDLLLHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNENFDDPNLIILPLAFGKRQGREFIWN |
| | | DLLSLETGSLKLANGRVIEKTLYNRRTRQDEPALFVALTFERREVL |
| | | DSSNIKPMNLIGIDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTEMAERQYTRMEDWL |
| | | TAKLAYEGLSKTYLSKTLAQYTSKTCSNCGFTITSADYDRVLEKLK |
| | | KTATGWMTTINGKELKVEGQITYYNRYKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 233) |
| CasX | (SEQ ID | QEIKRINKIRRRLVKDSNTKKAGKTGPMKTLLVRVMTPDLRERLEN |
| 457 | NO: 231) | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHKKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSPEDRKKGKKFARYQLGDLLLHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNENFDDPNLIILPLAFGKRQGREFIWN |
| | | DLLSLETGSLKLANGRVIEKPLYNRRTRQDEPALFVALTFERREVL |
| | | DSSNIKPMNLIGVDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTFMAERQYTRMEDWL |
| | | TAKLAYEGLSKTYLSKTLAQYTSKTCSNCGFTITSADYDRVLEKLK |
| | | KTATGWMTTINGKELKVEGQITYYNRRKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 234) |
| CasX | (SEQ ID | QEIKRINKIRRRLVKDSNTKKAGKTGPMKTLLVRVMTPDLRERLEN |
| 438 | NO: 232) | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHQKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSEEDRKKGKKFARYQLGDLLKHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNENFDDPNLIILPLAFGKRQGREFIWN |
| | | DLLSLETGSLKLANGRVIEKTLYNRRTRQDEPALFVALTFERREVL |
| | | DSSNIKPMNLIGVDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTEMAERQYTRMEDWL |
| | | TAKLAYEGLSKTYLSKTLAQYTSKTCSNCGFTITSADYDRVLEKLK |
| | | KTATGWMTTINGKELKVEGQITYYNRRKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 235) |

### Example 3: CasX Construct 488 and 491

In order to generate the CasX 488 construct (sequences in Table 7), the codon-optimized CasX 119 construct (based on the CasX Stx2 construct of Example 1, encoding Planctomycetes CasX SEQ ID NO:2, with a A708K substitution, a L379R substitution, and a [P793] deletion with fused NLS, and linked guide and non-targeting sequences) was cloned into a mammalian expression plasmid (pStX; see FIG. 4) using standard cloning methods. Construct CasX 1 (based on the CasX Stx1 construct of Example 1, encoding CasX SEQ TD NO: 1) was cloned into a destination vector using standard cloning methods. To build CasX 488, the CasX 119 construct DNA was PCR amplified using Q5 DNA polymerase using primers oIC765 and oIC762 (see FIG. 5). The CasX 1 construct was PCR amplified using Q5 DNA polymerase using primers oIC766 and oIC784. The PCR products were purified by gel extraction from a 1% agarose gel using Zymoclean Gel DNA Recovery Kit. The two fragments were then pieced together using Gibson assembly. Assembled products in pStx1 were transformed into chemically-competent Turbo Competent E. coli bacterial cells, plated on LB-Agar plates containing kanamycin. Individual colonies were picked and miniprepped using Qiagen Qiaprep spin Miniprep Kit. The resultant plasmids were sequenced using Sanger sequencing to ensure correct assembly. The correct clones were then subcloned into the mammalian expression vector pStx34 using restriction enzyme cloning. The pStx34 backbone and the CasX 488 clone in pStx1 were digested with XbaI and BamHI respectively. The digested backbone and insert fragments were purified by gel extraction from a 1% agarose gel using Zymoclean Gel DNA Recovery Kit. The clean backbone and insert were then ligated together using T4 Ligase (New England Biolabs Cat #M0202L) according to the manufacturer's protocol. The ligated product was transformed into chemically-competent Turbo Competent E. coli bacterial cells, plated on LB-Agar plates containing carbenicillin. Individual colonies were picked and miniprepped using Qiagen Qiaprep spin Miniprep Kit. The resultant plasmids were sequenced using Sanger sequencing to ensure correct assembly.

In order to generate CasX 491 (sequences in Table 7), the CasX 484 construct DNA was PCR amplified using Q5 DNA polymerase using primers oIC765 and oIC762 (see FIG. 5). The CasX 1 construct was PCR amplified using Q5 DNA polymerase using primers oIC766 and oIC784. The PCR products were purified by gel extraction from a 1% agarose gel using Zymoclean Gel DNA Recovery Kit. The two fragments were then pieced together using Gibson assembly. Assembled products in pStx1 were transformed into chemically-competent Turbo Competent E. coli bacterial cells, plated on LB-Agar plates containing kanamycin. Individual colonies were picked and miniprepped using Qiagen Qiaprep spin Miniprep Kit. The resultant plasmids were sequenced using Sanger sequencing to ensure correct assembly. The correct clones were then subcloned into the mammalian expression vector pStx34 using restriction enzyme cloning. The pStx34 backbone and the CasX 491 clone in pStx1 were digested with XbaI and BamHI respectively. The digested backbone and insert fragments were purified by gel extraction from a 1% agarose gel using Zymoclean Gel DNA Recovery Kit. The clean backbone and insert were then ligated together using T4 Ligase (New England Biolabs Cat #M0202L) according to the manufacturer's protocol. The ligated product was transformed into chemically-competent Turbo Competent E. coli bacterial cells, plated on LB-Agar plates containing carbenicillin. Individual colonies were picked and miniprepped using Qiagen Qiaprep spin Miniprep Kit. The resultant plasmids were sequenced using Sanger sequencing to ensure correct assembly. pStX34 includes an EF-1α promoter for the protein as well as a selection marker for both puromycin and carbenicillin. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli, plated on LB-Agar plates containing carbenicillin. Individual colonies were picked and miniprepped using Qiagen Qiaprep spin Miniprep Kit. The resultant plasmids were sequenced using Sanger sequencing to ensure correct ligation. SaCas9 and SpyCas9 control plasmids were prepared similarly to pStX plasmids described above, with the protein and guide regions of pStX exchanged for the respective protein and guide. Targeting sequences for SaCas9 and SpyCas9 were either obtained from the literature or were rationally designed according to established methods. The expression and recovery of the CasX constructs was performed using the general methodologies of Example 1 and Example 2, with similar results obtained.

TABLE 7
Sequences of CasX 488 and 491

| | DNA | |
|---|---|---|
| Construct | Sequence | Amino Acid Sequence |
| CasX 488 | (SEQ ID | QEIKRINKIRRRLVKDSNTKKAGKTGPMKTLLVRVMTPDLRERLE |
| | NO: 236) | NLRKKPENIPQPISNTSRANLNKLLTDYTEMKKAILHVYWEEFQK |
| | | DPVGLMSRVAQPASKKIDQNKLKPEMDEKGNLTTAGFACSQCGQP |
| | | LFVYKLEQVSEKGKAYTNYFGRCNVAEHEKLILLAQLKPEKDSDE |
| | | AVTYSLGKFGQRALDFYSIHVTKESTHPVKPLAQIAGNRYASGPV |
| | | GKALSDACMGTIASFLSKYQDIIIEHQKVVKGNQKRLESLRELAG |
| | | KENLEYPSVTLPPQPHTKEGVDAYNEVIARVRMWVNLNLWQKLKL |
| | | SRDDAKPLLRLKGFPSFPLVERQANEVDWWDMVCNVKKLINEKKE |
| | | DGKVFWQNLAGYKRQEALRPYLSSEEDRKKGKKFARYQFGDLLLH |
| | | LEKKHGEDWGKVYDEAWERIDKKVEGLSKHIKLEEERRSEDAQSK |
| | | AALTDWLRAKASFVIEGLKEADKDEFCRCELKLQKWYGDLRGKPF |
| | | AIEAENSILDISGFSKQYNCAFIWQKDGVKKLNLYLIINYFKGGK |
| | | LRFKKIKPEAFEANRFYTVINKKSGEIVPMEVNENFDDPNLIILP |
| | | LAFGKRQGREFIWNDLLSLETGSLKLANGRVIEKTLYNRRTRQDE |
| | | PALFVALTFERREVLDSSNIKPMNLIGIDRGENIPAVIALTDPEG |
| | | CPLSRFKDSLGNPTHILRIGESYKEKQRTIQAKKEVEQRRAGGYS |
| | | RKYASKAKNLADDMVRNTARDLLYYAVTQDAMLIFENLSRGFGRQ |
| | | GKRTFMAERQYTRMEDWLTAKLAYEGLSKTYLSKTLAQYTSKTCS |
| | | NCGFTITSADYDRVLEKLKKTATGWMTTINGKELKVEGQITYYNR |
| | | YKRQNVVKDLSVELDRLSEESVNNDISSWTKGRSGEALSLLKKRF |
| | | SHRPVQEKFVCLNCGFETHADEQAALNIARSWLFLRSQEYKKYQT |
| | | NKTTGNTDKRAFVETWQSFYRKKLKEVWKPAV (SEQ ID NO: |
| | | 238) |
| CasX 491 | (SEQ ID | QEIKRINKIRRRLVKDSNTKKAGKTGPMKTLLVRVMTPDLRERLE |
| | NO: 237) | NLRKKPENIPQPISNTSRANLNKLLTDYTEMKKAILHVYWEEFQK |
| | | DPVGLMSRVAQPASKKIDQNKLKPEMDEKGNLTTAGFACSQCGQP |
| | | LFVYKLEQVSEKGKAYTNYFGRCNVAEHEKLILLAQLKPEKDSDE |
| | | AVTYSLGKFGQRALDFYSIHVTKESTHPVKPLAQIAGNRYASGPV |
| | | GKALSDACMGTIASFLSKYQDIIIEHQKVVKGNQKRLESLRELAG |
| | | KENLEYPSVTLPPQPHTKEGVDAYNEVIARVRMWVNLNLWQKLKL |
| | | SRDDAKPLLRLKGFPSFPLVERQANEVDWWDMVCNVKKLINEKKE |
| | | DGKVFWQNLAGYKRQEALRPYLSSEEDRKKGKKFARYQLGDLLLH |
| | | LEKKHGEDWGKVYDEAWERIDKKVEGLSKHIKLEEERRSEDAQSK |
| | | AALTDWLRAKASFVIEGLKEADKDEFCRCELKLQKWYGDLRGKPF |
| | | AIEAENSILDISGESKQYNCAFIWQKDGVKKLNLYLIINYFKGGK |
| | | LRFKKIKPEAFEANRFYTVINKKSGEIVPMEVNENFDDPNLIILP |
| | | LAFGKRQGREFIWNDLLSLETGSLKLANGRVIEKTLYNRRTRQDE |
| | | PALFVALTFERREVLDSSNIKPMNLIGVDRGENIPAVIALTDPEG |
| | | CPLSRFKDSLGNPTHILRIGESYKEKQRTIQAKKEVEQRRAGGYS |
| | | RKYASKAKNLADDMVRNTARDLLYYAVTQDAMLIFENLSRGFGRQ |
| | | GKRTEMAERQYTRMEDWLTAKLAYEGLSKTYLSKTLAQYTSKTCS |
| | | NCGFTITSADYDRVLEKLKKTATGWMITINGKELKVEGQITYYNR |
| | | YKRQNVVKDLSVELDRLSEESVNNDISSWTKGRSGEALSLLKKRF |
| | | SHRPVQEKFVCLNCGFETHADEQAALNIARSWLELRSQEYKKYQT |
| | | NKTTGNTDKRAFVETWQSFYRKKLKEVWKPAV (SEQ ID NO: |
| | | 239) |

### Example 4: Design and Generation of CasX Constructs 278-280, 285-288, 290, 291, 293, 300, 492, and 493

In order to generate the CasX 278-280, 285-288, 290, 291, 293, 300, 492, and 493 constructs (sequences in Table 8), the N- and C-termini of the codon-optimized CasX 119 construct (based on the CasX Stx37 construct of Example 2, encoding Planctomycetes CasX SEQ TD NO:2, with a A708K substitution and a [P793] deletion with fused NLS, and linked guide and non-targeting sequences) in a mammalian expression vector were manipulated to delete or add NLS sequences (sequences in Table 9). Constructs 278, 279, and 280 were manipulations of the N- and C-termini using only an SV40 NLS sequence. Construct 280 had no NLS on the N-terminus and added two SV40 NLS' on the C-terminus with a triple proline linker in between the two SV40 NLS sequences. Constructs 278, 279, and 280 were made by amplifying pStx34.119.174.NT with Q5 DNA polymerase using primers oIC527 and oIC528, oIC730 and oIC522, and oIC730 and oIC530 for the first fragments each and using oIC529 and oIC520, oIC519 and oIC731, and oIC529 and oIC731 to create the second fragments each. These fragments were purified by gel extraction from a 1% agarose gel using Zymoclean Gel DNA Recovery Kit. The respective fragments were cloned together using Gibson assembly. Assembled products in the pStx34 were transformed into chemically-competent Turbo Competent E. coli bacterial cells, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct assembly. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically- or electro-competent cells such as NEB Turbo competent E. coli, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation.

In order to generate constructs 285-288, 290, 291, 293, and 300, a nested PCR method was used for cloning. The backbone vector and PCR template used was construct pStx34 279.119.174.NT, having the CasX 119, guide 174, and non-targeting spacer. Construct 278 has the configuration SV40NLS-CasX119. Construct 279 has the configuration CasX119-SV40NLS. Construct 280 has the configuration CasX119-SV40NLS-PPP linker-SV40NLS. Construct 285 has the configuration CasX119-SV40NLS-PPP linker-SynthNLS3. Construct 286 has the configuration CasX119-SV40NLS-PPP linker-SynthNLS4. Construct 287 has the configuration CasX119-SV40NLS-PPP linker-SynthNLS5. Construct 288 has the configuration CasX119-SV40NLS-PPP linker-SynthNLS6. Constrict 290 has the configuration CasX119-SV40NLS-PPP linker-EGL-13 NLS. Construct 291 has the configuration CasX119-SV40NLS-PPP linker-c-Myc NLS. Construct 293 has the configuration CasX119-SV40NLS-PPP linker-Nucleolar RNA Helicase II NLS. Construct 300 has the configuration CasX119-SV40NLS-PPP linker-Influenza A protein NLS. Construct 492 has the configuration SV40NLS-CasX119-SV40NLS-PPP linker-SV40NLS. Construct 493 has the configuration SV40NLS-CasX119-SV40NLS-PPP linker-c-Myc NLS. Each variant had a set of three PCRs; two of which were nested, were purified by gel extraction, digested, and then ligated into the digested and purified backbone. Assembled products in the pStx34 were transformed into chemically-competent Turbo Competent E. coli bacterial cells, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct assembly. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into the resulting pStX individually or in bulk by Golden Gate assembly using T4 DNA Ligase and an appropriate restriction enzyme for the plasmid. Golden Gate products were transformed into chemically- or electro-competent cells such as NEB Turbo competent E. coli, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation.

In order to generate constructs 492 and 493, constructs 280 and 291 were digested using XbaI and BamHI (NEB #R0145S and NEB #R3136S) according to the manufacturer's protocol. Next, they were purified by gel extraction from a 1% agarose gel using Zymoclean Gel DNA Recovery Kit. Finally, they were ligated using T4 DNA ligase (NEB #M0202S) according to the manufacturer's protocol into the digested and purified pStx34.119.174.NT using XbaI and BamHI and Zymoclean Gel DNA Recovery Kit. Assembled products in the pStx34 were transformed into chemically-competent Turbo Competent E. coli bacterial cells, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct assembly. Sequences encoding the targeting spacer sequences that target the gene of interest were designed based on CasX PAM locations. Targeting sequence DNA was ordered as single-stranded DNA (ssDNA) oligos (Integrated DNA Technologies) consisting of the targeting spacer sequence and the reverse complement of this sequence. These two oligos were annealed together and cloned into each pStX individually or in bulk by Golden Gate assembly using T4 DNA Ligase and an appropriate restriction enzyme for the respective plasmids. Golden Gate products were transformed into chemically- or electro-competent cells such as NEB Turbo competent E. coli plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation. The plasmids were used to produce and recover CasX protein utilizing the general methodologies of Examples 1 and 2.

TABLE 8
CasX 278-280, 285-288, 290, 291, 293, 300, 492, and 493 sequences

| Construct | Amino Acid Sequence |
|---|---|
| 278 | MAPKKKRKVSRQEIKRINKIRRRLVKDSNTKKAGKTGPMKTLLVRVMTPDLRERL |
| | ENLRKKPENIPQPISNTSRANLNKLLTDYTEMKKAILHVYWEEFQKDPVGLMSRV |
| | AQPAPKNIDQRKLIPVKDGNERLTSSGFACSQCCQPLYVYKLEQVNDKGKPHTNY |
| | FGRCNVSEHERLILLSPHKPEANDELVTYSLGKFGQRALDFYSIHVTRESNHPVK |
| | PLEQIGGNSCASGPVGKALSDACMGAVASFLTKYQDIILEHQKVIKKNEKRLANL |
| | KDIASANGLAFPKITLPPQPHTKEGIEAYNNVVAQIVIWVNLNLWQKLKIGRDEA |
| | KPLQRLKGFPSFPLVERQANEVDWWDMVCNVKKLINEKKEDGKVFWQNLAGYKRQ |
| | EALRPYLSSEEDRKKGKKFARYQFGDLLLHLEKKHGEDWGKVYDEAWERIDKKVE |
| | GLSKHIKLEEERRSEDAQSKAALTDWLRAKASFVIEGLKEADKDEFCRCELKLQK |
| | WYGDLRGKPFAIEAENSILDISGFSKQYNCAFIWQKDGVKKLNLYLIINYFKGGK |
| | LRFKKIKPEAFEANRFYTVINKKSGEIVPMEVNFNEDDPNLIILPLAFGKRQGRE |
| | FIWNDLLSLETGSLKLANGRVIEKTLYNRRTRQDEPALFVALTFERREVLDSSNI |
| | KPMNLIGIDRGENIPAVIALTDPEGCPLSRFKDSLGNPTHILRIGESYKEKQRTI |
| | QAKKEVEQRRAGGYSRKYASKAKNLADDMVRNTARDLLYYAVTQDAMLIFENLSR |
| | GFGRQGKRTFMAERQYTRMEDWLTAKLAYEGLSKTYLSKTLAQYTSKTCSNCGFT |
| | ITSADYDRVLEKLKKTATGWMTTINGKELKVEGQITYYNRYKRQNVVKDLSVELD |
| | RLSEESVNNDISSWTKGRSGEALSLLKKRFSHRPVQEKFVCLNCGFETHADEQAA |
| | LNIARSWLFLRSQEYKKYQTNKTTGNTDKRAFVETWQSFYRKKLKEVWKPAV |
| | (SEQ ID NO: 240) |
| 279 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKV |
| | (SEQ ID NO: 241) |
| 280 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVEWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPP |
| | KKKRKV (SEQ ID NO: 242) |
| 285 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPH |
| | KKKHPDASVNFSEFSK (SEQ ID NO: 243) |
| 286 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPQ |
| | RPGPYDRPORPGPYDRP (SEQ ID NO: 244) |
| 287 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPL |
| | SPSLSPLLSPSLSPL (SEQ ID NO: 245) |
| 288 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTMSSGFACSQCCQPLYVYKLEQVNDKGKPHTNYFGRCNVSEH |
| | ERLILLSPHKPEANDELVTYSLGKFGQRALDFYSIHVTRESNHPVKPLEQIGGNS |
| | CASGPVGKALSDACMGAVASFLTKYQDIILEHQKVIKKNEKRLANLKDIASANGL |
| | AFPKITLPPQPHTKEGIEAYNNVVAQIVIWVNLNLWQKLKIGRDEAKPLQRLKGF |
| | PSFPLVERQANEVDWWDMVCNVKKLINEKKEDGKVFWQNLAGYKRQEALRPYLSS |
| | EEDRKKGKKFARYQFGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNENFDDPNLIILPLAFGKRQGREFIWNDLLSL |
| | ETGSLKLANGRVIEKTLYNRRTRQDEPALFVALTFERREVLDSSNIKPMNLIGID |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARSWLE |
| | LRSQEYKKYQTNKTTGNTDKRAFVETWQSFYRKKLKEVWKPAVTSPKKKRKVPPP |
| | RGKGGKGLGKGGAKRHRK (SEQ ID NO: 246) |
| 290 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPS |
| | RRRKANPTKLSENAKKLAKEVEN (SEQ ID NO: 247) |
| 291 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPP |
| | AAKRVKLD (SEQ ID NO: 248) |
| 293 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPK |
| | RSFSKAF (SEQ ID NO: 249) |
| 300 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNENFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLEL |
| | RSQEYKKYQTNKTTGNTDKRAFVETWQSFYRKKLKEVWKPAVTSPKKKRKVPPPK |
| | RGINDRNFWRGENERKTR (SEQ ID NO: 250) |
| 492 | MAPKKKRKVSRMQEIKRINKIRRRLVKDSNTKKAGKTGPMKTLLVRVMTPDLRER |
| | LENLRKKPENIPQPISNTSRANLNKLLTDYTEMKKAILHVYWEEFQKDPVGLMSR |
| | VAQPAPKNIDQRKLIPVKDGNERLTSSGFACSQCCQPLYVYKLEQVNDKGKPHTN |
| | YFGRCNVSEHERLILLSPHKPEANDELVTYSLGKFGQRALDFYSIHVTRESNHPV |
| | KPLEQIGGNSCASGPVGKALSDACMGAVASFLTKYQDIILEHQKVIKKNEKRLAN |
| | LKDIASANGLAFPKITLPPQPHTKEGIEAYNNVVAQIVIWVNLNLWQKLKIGRDE |
| | AKPLQRLKGFPSFPLVERQANEVDWWDMVCNVKKLINEKKEDGKVFWQNLAGYKR |
| | QEALRPYLSSEEDRKKGKKFARYQFGDLLLHLEKKHGEDWGKVYDEAWERIDKKV |
| | EGLSKHIKLEEERRSEDAQSKAALTDWLRAKASFVIEGLKEADKDEFCRCELKLQ |
| | KWYGDLRGKPFAIEAENSILDISGFSKQYNCAFIWQKDGVKKLNLYLIINYFKGG |
| | KLRFKKIKPEAFEANRFYTVINKKSGEIVPMEVNENFDDPNLIILPLAFGKRQGR |
| | EFIWNDLLSLETGSLKLANGRVIEKTLYNRRTRQDEPALFVALTFERREVLDSSN |
| | IKPMNLIGIDRGENIPAVIALTDPEGCPLSRFKDSLGNPTHILRIGESYKEKQRT |
| | IQAKKEVEQRRAGGYSRKYASKAKNLADDMVRNTARDLLYYAVTQDAMLIFENLS |
| | RGFGRQGKRTFMAERQYTRMEDWLTAKLAYEGLSKTYLSKTLAQYTSKTCSNCGF |
| | TITSADYDRVLEKLKKTATGWMTTINGKELKVEGQITYYNRYKRQNVVKDLSVEL |
| | DRLSEESVNNDISSWTKGRSGEALSLLKKRFSHRPVQEKFVCLNCGFETHADEQA |
| | ALNIARSWLFLRSQEYKKYQTNKTTGNTDKRAFVETWQSFYRKKLKEVWKPAVTS |
| | PKKKRKVPPPPKKKRKV (SEQ ID NO: 251) |
| 493 | MAPKKKRKVSRMQEIKRINKIRRRLVKDSNTKKAGKTGPMKTLLVRVMTPDLRER |
| | LENLRKKPENIPQPISNTSRANLNKLLTDYTEMKKAILHVYWEEFQKDPVGLMSR |
| | VAQPAPKNIDQRKLIPVKDGNERLTSSGFACSQCCQPLYVYKLEQVNDKGKPHTN |
| | YFGRCNVSEHERLILLSPHKPEANDELVTYSLGKFGQRALDFYSIHVTRESNHPV |
| | KPLEQIGGNSCASGPVGKALSDACMGAVASFLTKYQDIILEHQKVIKKNEKRLAN |
| | LKDIASANGLAFPKITLPPQPHTKEGIEAYNNVVAQIVIWVNLNLWQKLKIGRDE |
| | AKPLQRLKGFPSFPLVERQANEVDWWDMVCNVKKLINEKKEDGKVFWQNLAGYKR |
| | QEALRPYLSSEEDRKKGKKFARYQFGDLLLHLEKKHGEDWGKVYDEAWERIDKKV |
| | EGLSKHIKLEEERRSEDAQSKAALTDWLRAKASFVIEGLKEADKDEFCRCELKLQ |
| | KWYGDLRGKPFAIEAENSILDISGFSKQYNCAFIWQKDGVKKLNLYLIINYFKGG |
| | KLRFKKIKPEAFEANRFYTVINKKSGEIVPMEVNENFDDPNLIILPLAFGKRQGR |
| | EFIWNDLLSLETGSLKLANGRVIEKTLYNRRTRQDEPALFVALTFERREVLDSSN |
| | IKPMNLIGIDRGENIPAVIALTDPEGCPLSRFKDSLGNPTHILRIGESYKEKQRT |
| | IQAKKEVEQRRAGGYSRKYASKAKNLADDMVRNTARDLLYYAVTQDAMLIFENLS |
| | RGFGRQGKRTEMAERQYTRMEDWLTAKLAYEGLSKTYLSKTLAQYTSKTCSNCGF |
| | TITSADYDRVLEKLKKTATGWMTTINGKELKVEGQITYYNRYKRQNVVKDLSVEL |
| | DRLSEESVNNDISSWTKGRSGEALSLLKKRFSHRPVQEKFVCLNCGFETHADEQA |
| | ALNIARSWLFLRSQEYKKYQTNKTTGNTDKRAFVETWQSFYRKKLKEVWKPAVTS |
| | PKKKRKVPPPPAAKRVKLD (SEQ ID NO: 252) |

TABLE 9
Nuclear localization sequence list

| CasX | NLS | DNA Sequence | Amino Acid Sequence |
|---|---|---|---|
| 278, 279, | SV40 | CCAAAGAAGAAGCGGAAGGTC | PKKKRKV (SEQ ID |
| 280, 492, | | (SEQ ID NO: 253) | NO: 165) |
| 493 | | | |
| 285 | SynthNLS3 | CACAAGAAGAAACATCCAGACGC | HKKKHPDASVNFSEF |
| | | ATCAGTCAACTTTAGCGAGTTCA | SK (SEQ ID NO: |
| | | GTAAA (SEQ ID NO: 254) | 196) |
| 286 | SynthNLS4 | CAGCGCCCTGGGCCTTACGATAG | QRPGPYDRPQRPGPY |
| | | GCCGCAAAGACCCGGACCGTATG | DRP (SEQ ID NO: |
| | | ATCGCCCT (SEQ ID NO: | 197) |
| | | 255) | |
| 287 | SynthNLS5 | CTCAGCCCGAGTCTTAGTCCACT | LSPSLSPLLSPSLSP |
| | | GCTTTCCCCGTCCCTGTCTCCAC | L (SEQ ID NO: |
| | | TG (SEQ ID NO: 256) | 198) |
| 288 | SynthNLS6 | CGGGGCAAGGGTGGCAAGGGGCT | RGKGGKGLGKGGAKR |
| | | TGGCAAGGGGGGGGCAAAGAGGC | HRK (SEQ ID NO: |
| | | ACAGGAAG (SEQ ID NO: | 199) |
| | | 257) | |
| 290 | EGL-13 | AGCCGCCGCAGAAAAGCCAATCC | SRRRKANPTKLSENA |
| | | TACAAAACTGTCAGAAAATGCGA | KKLAKEVEN (SEQ |
| | | AAAAACTTGCTAAGGAGGTGGAA | ID NO: 192) |
| | | AAC (SEQ ID NO: 258) | |
| 291 | c-Myc | CCTGCCGCAAAGCGAGTGAAATT | PAAKRVKLD (SEQ |
| | | GGAC (SEQ ID NO: 259) | ID NO: 167) |
| 293 | Nucleolar RNA | AAGCGGTCCTTCAGTAAGGCCTT | KRSESKAF (SEQ |
| | Helicase II | T (SEQ ID NO: 260) | ID NO: 188) |
| 300 | Influenza A | AAACGGGGAATAAACGACCGGAA | KRGINDRNFWRGENE |
| | protein | CTTCTGGCGCGGGGAAAACGAGC | RKTR (SEQ ID |
| | | GCAAAACCCGA (SEQ ID NO: | NO: 186) |
| | | 261) | |

### Example 5: Design and Generation of CasX Constructs 387, 395, 485-491, and 494

In order to generate CasX 395, CasX 485, CasX 486, CasX 487, the codon optimized CasX 119 (based on the CasX 37 construct of Example 2, encoding Planctomycetes CasX SEQ ID NO: 2, with a A708K substitution and a [P793] deletion with fused NLS, and linked guide and non-targeting sequences), CasX 435, CasX 438, and CasX 484 (each based on CasX 119 construct of Example 2 encoding Planctomycetes CasX SEQ ID NO: 2, with a L379R substitution, a A708K substitution, and a [P793] deletion with fused NLS, and linked guide and non-targeting sequences) were cloned respectively into a 4 kb staging vector comprising a KanR marker, colE1 ori, and CasX with fused NLS (pStx1) using standard cloning methods. Gibson primers were designed to amplify the CasX SEQ ID NO: 1 Helical I domain from amino acid 192-331 in its own vector to replace this corresponding region (aa 193-332) on CasX 119, CasX 435, CasX 438, and CasX 484 in pStx1 respectively. The Helical I domain from CasX SEQ ID NO: 1 was amplified with primers oIC768 and oIC784 using Q5 DNA polymerase according to the manufacturer's protocol. The destination vector containing the desired CasX variant was amplified with primers oIC765 and oIC764 using Q5 DNA polymerase according to the manufacturer's protocol. The two fragments were purified by gel extraction from a 1% agarose gel using Zymoclean Gel DNA Recovery Kit. The insert and backbone fragments were then pieced together using Gibson assembly. Assembled products in the pStx1 staging vector were transformed into chemically-competent Turbo Competent E. coli bacterial cells, plated on LB-Agar plates containing kanamycin and incubated at 37° C. Individual colonies were picked and miniprepped using Qiagen Qiaprep spin Miniprep Kit. The resultant plasmids were sequenced using Sanger sequencing to ensure correct assembly. Correct clones were then cut and pasted into a mammalian expression plasmid (see FIG. 5) using standard cloning methods. The resultant plasmids were sequenced using Sanger sequencing to ensure correct assembly. Sequences encoding the targeting spacer sequences that target the gene of interest were designed based on CasX PAM locations. Targeting spacer sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation.

In order to generate CasX 488, CasX 489, CasX 490, and CasX 491 (sequences in Table 10), the codon optimized CasX 119 (based on the CasX 37 construct of Example 2, encoding Planctomycetes CasX SEQ ID NO: 2, with a A708K substitution and a [P793] deletion with fused NLS, and linked guide and non-targeting sequences), CasX 435, CasX 438, and CasX 484 (each based on CasX119 construct of Example 2 encoding Planctomycetes CasX SEQ ID NO: 2, with a L379R substitution, a A708K substitution, and a [P793] deletion with fused NLS, and linked guide and non-targeting sequences) were cloned respectively into a 4 kb staging vector that was made up of a KanR marker, colE1 ori, and STX with fused NLS (pStx1) using standard cloning methods. Gibson primers were designed to amplify the CasX Stx1 NTSB domain from amino acid 101-191 and Helical I domain from amino acid 192-331 in its own vector to replace this similar region (aa 103-332) on CasX 119, CasX 435, CasX 438, and CasX 484 in pStx1 respectively. The NTSB and Helical I domain from CasX SEQ ID NO: 1 were amplified with primers oIC766 and oIC784 using Q5 DNA polymerase according to the manufacturer's protocol. The destination vector containing the desired CasX variant was amplified with primers oIC762 and oIC765 using Q5 DNA polymerase according to the manufacturer's protocol. The two fragments were purified by gel extraction from a 1% agarose gel using Zymoclean Gel DNA Recovery Kit. The insert and backbone fragments were then pieced together using Gibson assembly. Assembled products in the pStx1 staging vector were transformed into chemically-competent Turbo Competent E. coli bacterial cells, plated on LB-Agar plates containing kanamycin and incubated at 37° C. Individual colonies were picked and miniprepped using Qiagen Qiaprep spin Miniprep Kit. The resultant plasmids were sequenced using Sanger sequencing to ensure correct assembly. Correct clones were then cut and pasted into a mammalian expression plasmid (see FIG. 5) using standard cloning methods. The resultant plasmids were sequenced using Sanger sequencing to ensure correct assembly. Sequences encoding the targeting spacer sequences that target the gene of interest were designed based on CasX PAM locations. Targeting spacer sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation.

In order to generate CasX 387 and CasX 494 (sequences in Table 10), the codon optimized CasX 119 (based on the CasX 37 construct of Example 2, encoding Planctomycetes CasX SEQ ID NO:2, with a A708K substitution and a [P793] deletion with fused NLS, and linked guide and non-targeting sequences) and CasX 484 (based on CasX119 construct of Example 2 encoding Planctomycetes CasX SEQ ID NO: 2, with a L379R substitution, a A708K substitution, and a [P793] deletion with fused NLS, and linked guide and non-targeting sequences) were cloned respectively into a 4 kb staging vector that was made up of a KanR marker, colE1 ori, and STX with fused NLS (pStx1) using standard cloning methods. Gibson primers were designed to amplify the CasX Stx1 NTSB domain from amino acid 101-191 in its own vector to replace this similar region (aa 103-192) on CasX 119 and CasX 484 in pStx1 respectively. The NTSB domain from CasX Stx1 was amplified with primers oIC766 and oIC767 using Q5 DNA polymerase according to the manufacturer's protocol. The destination vector containing the desired CasX variant was amplified with primers oIC763 and oIC762 using Q5 DNA polymerase according to the manufacturer's protocol. The two fragments were purified by gel extraction from a 1% agarose gel using Zymoclean Gel DNA Recovery Kit. The insert and backbone fragments were then pieced together using Gibson assembly. Assembled products in the pStx1 staging vector were transformed into chemically-competent Turbo Competent E. coli bacterial cells, plated on LB-Agar plates containing kanamycin and incubated at 37° C. Individual colonies were picked and miniprepped using Qiagen Qiaprep spin Miniprep Kit. The resultant plasmids were sequenced using Sanger sequencing to ensure correct assembly. Correct clones were then cut and pasted into a mammalian expression plasmid (see FIG. 5) using standard cloning methods. The resultant plasmids were sequenced using Sanger sequencing to ensure correct assembly. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase and an appropriate restriction enzyme for the plasmid. Golden Gate products were transformed into chemically or electro-competent cells such as NEB Turbo competent E. coli, plated on LB-Agar plates containing carbenicillin and incubated at 37° C. Individual colonies were picked and miniprepped using Qiagen Qiaprep spin Miniprep Kit. The resultant plasmids were sequenced using Sanger sequencing to ensure correct ligation. Sequences of the resulting constructs are listed in Table 10.

TABLE 10
Sequences of CasX 395 and 485-491

| | DNA | |
|---|---|---|
| Construct | Sequence | Amino Acid Sequence |
| CasX 387 | (SEQ | MAPKKKRKVSRQEIKRINKIRRRLVKDSNTKKAGKTGPMKTLLVRV |
| | ID NO: 262) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGE |
| | | ACSQCGQPLFVYKLEQVSEKGKAYTNYFGRCNVAEHEKLILLAQLK |
| | | PEKDSDEAVTYSLGKFGQRALDFYSIHVTRESNHPVKPLEQIGGNS |
| | | CASGPVGKALSDACMGAVASFLTKYQDIILEHQKVIKKNEKRLANL |
| | | KDIASANGLAFPKITLPPQPHTKEGIEAYNNVVAQIVIWVNLNLWQ |
| | | KLKIGRDEAKPLQRLKGFPSFPLVERQANEVDWWDMVCNVKKLINE |
| | | KKEDGKVFWQNLAGYKRQEALRPYLSSEEDRKKGKKFARYQFGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGIDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGETIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKE |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 272) |
| CasX 395 | (SEQ ID | MAPKKKRKVSRQEIKRINKIRRRLVKDSNTKKAGKTGPMKTLLVRV |
| | NO: 263) | MTPDLRERLENLRKKPENIPQPISNTSRANLNKLLTDYTEMKKAIL |
| | | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQFGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGIDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTEMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRYKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKVTSPKKKRKV (SEQ |
| | | ID NO: 273) |
| CasX 485 | (SEQ | MAPKKKRKVSRQEIKRINKIRRRLVKDSNTKKAGKTGPMKTLLVRV |
| | ID NO: 264) | MTPDLRERLENLRKKPENIPQPISNTSRANLNKLLTDYTEMKKAIL |
| | | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTEMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRRKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKV (SEQ ID NO: |
| | | 274) |
| CasX 486 | (SEQ ID | MAPKKKRKVSRQEIKRINKIRRRLVKDSNTKKAGKTGPMKTLLVRV |
| | NO: 265) | MTPDLRERLENLRKKPENIPQPISNTSRANLNKLLTDYTEMKKAIL |
| | | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLK |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTEMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRRKRQNVVKD |
| | | LSVELDRLSEESVNNDISSWTKGRSGEALSLLKKRFSHRPVQEKFV |
| | | CLNCGFETHADEQAALNIARSWLELRSQEYKKYQTNKTTGNTDKRA |
| | | FVETWQSFYRKKLKEVWKPAVTSPKKKRKV (SEQ ID NO: |
| | | 275) |
| CasX 487 | (SEQ | MAPKKKRKVSRQEIKRINKIRRRLVKDSNTKKAGKTGPMKTLLVRV |
| | ID NO: 266) | MTPDLRERLENLRKKPENIPQPISNTSRANLNKLLTDYTEMKKAIL |
| | | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTEMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRYKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKV (SEQ ID NO: |
| | | 276) |
| CasX 488 | (SEQ ID | MAPKKKRKVSRQEIKRINKIRRRLVKDSNTKKAGKTGPMKTLLVRV |
| | NO: 267) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQFGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGIDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 277) |
| CasX 489 | (SEQ ID | MAPKKKRKVSRQEIKRINKIRRRLVKDSNTKKAGKTGPMKTLLVRV |
| | NO: 268) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGETIT |
| | | SADYDRVLEKLKKTATGWMTTINGKELKVEGQITYYNRRKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 278) |
| CasX 490 | (SEQ ID | MAPKKKRKVSRQEIKRINKIRRRLVKDSNTKKAGKTGPMKTLLVRV |
| | NO: 269) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | KHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGFTIT |
| | | SADYDRVLEKLKKTATGWMTTINGKELKVEGQITYYNRRKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 279) |
| CasX 491 | (SEQ ID | MAPKKKRKVSRQEIKRINKIRRRLVKDSNTKKAGKTGPMKTLLVRV |
| | NO: 270) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 280) |
| CasX 494 | (SEQ | MAPKKKRKVSRQEIKRINKIRRRLVKDSNTKKAGKTGPMKTLLVRV |
| | ID NO: 271) | MTPDLRERLENLRKKPENIPQPISNTSRANLNKLLTDYTEMKKAIL |
| | | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTRESNHPVKPLEQIGGNS |
| | | CASGPVGKALSDACMGAVASFLTKYQDIILEHQKVIKKNEKRLANL |
| | | KDIASANGLAFPKITLPPQPHTKEGIEAYNNVVAQIVIWVNLNLWQ |
| | | KLKIGRDEAKPLQRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTEM |
| | | AERQYTRMEDWLTAKLAYEGLSKTYLSKTLAQYTSKTCSNCGETIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 281) |

### Example 6: Generation of RNA Guides

For the generation of RNA single guides and spacers, templates for in vitro transcription were generated by performing PCR with Q5 polymerase (NEB M0491) according to the recommended protocol, with template oligos for each backbone and amplification primers with the T7 promoter and the spacer sequence. The DNA primer sequences for the T7 promoter, guide and spacer for guides and spacers are presented in Table 11, below. The template oligos, labeled “backbone fwd” and “backbone rev” for each scaffold, were included at a final concentration of 20 nM each, and the amplification primers (T7 promoter and the unique spacer primer) were included at a final concentration of 1 uM each. The sg2, sg32, sg64, and sg174 guides correspond to SEQ ID NOS: 5, 2104, 2106, and 2238, respectively, with the exception that sg2, sg32, and sg64 were modified with an additional 5′ G to increase transcription efficiency (compare sequences in Table 11 to Table 2). The 7.37 spacer targets beta2-microglobulin (B2M). Following PCR amplification, templates were cleaned and isolated by phenol-chloroform-isoamyl alcohol extraction followed by ethanol precipitation.

In vitro transcriptions were carried out in buffer containing 50 mM Tris pH 8.0, 30 mM MgCl₂, 0.01% Triton X-100, 2 mM spermidine, 20 mM DTT, 5 mM NTPs, 0.5 μM template, and 100 μg/mL T7 RNA polymerase. Reactions were incubated at 37° C. overnight. 20 units of DNase I (Promega #M6101)) were added per 1 mL of transcription volume and incubated for one hour. RNA products were purified via denaturing PAGE, ethanol precipitated, and resuspended in 1× phosphate buffered saline. To fold the sgRNAs, samples were heated to 70° C. for 5 min and then cooled to room temperature. The reactions were supplemented to 1 mM final MgCl₂ concentration, heated to 50° C. for 5 min and then cooled to room temperature. Final RNA guide products were stored at −80° C.

TABLE 11
Sequences for generation of guide RNA

| Primer | Primer sequence | RNA product |
|---|---|---|
| T7 promoter | GAAATTAATACGACTCACTATA (SEQ ID NO: | Used for all |
| primer | 282) | |
| sg2 backbone | GAAATTAATACGACTCACTATAGGTACTGGCGCTT | GGUACUGGCGCUUUU |
| fwd | TTATCTCATTACTTTGAGAGCCATCACCAGCGACT | AUCUCAUUACUUUGA |
| | ATGTCGTATGGGTAAAG (SEQ ID NO: 283) | GAGCCAUCACCAGCG |
| sg2 backbone | CTTTGATGCTTCTTATTTATCGGATTTCTCTCCGA | ACUAUGUCGUAUGGG |
| rev | TAAATAAGCGCTTTACCCATACGACATAGTCGCTG | UAAAGCGCUUAUUUA |
| | GTGATGGC (SEQ ID NO: 284) | UCGGAGAGAAAUCCG |
| sg2.7.37 | CGGAGCGAGACATCTCGGCCCTTTGATGCTTCTTA | AUAAAUAAGAAGCAU |
| spacer primer | TTTATCGGATTTCTCTCCG (SEQ ID NO: | CAAAGGGCCGAGAUG |
| | 285) | UCUCGCUCCG (SEQ |
| | | ID NO: 295) |
| sg32 backbone | GAAATTAATACGACTCACTATAGGTACTGGCGCTT | GGUACUGGCGCUUUU |
| fwd | TTATCTCATTACTTTGAGAGCCATCACCAGCGACT | AUCUCAUUACUUUGA |
| | ATGTCGTATGGGTAAAGCGC (SEQ ID NO: | GAGCCAUCACCAGCG |
| | 286) | ACUAUGUCGUAUGGG |
| sg32 backbone | CTTTGATGCTTCCCTCCGAAGAGGGCGCTTTACCC | UAAAGCGCCCUCUUC |
| rev | ATACGACATAG (SEQ ID NO: 287) | GGAGGGAAGCAUCAA |
| sg32.7.37 | CGGAGCGAGACATCTCGGCCCTTTGATGCTTCCCT | AGGGCCGAGAUGUCU |
| spacer primer | CCGAAGAG (SEQ ID NO: 288) | CG (SEQ ID NO: |
| | | 296) |
| sg64 backbone | GAAATTAATACGACTCACTATAGGTACTGGCGCCT | GGUACUGGCGCCUUU |
| fwd | TTATCTCATTACTTTGAGAGCCATCACCAGCGACT | AUCUCAUUACUUUGA |
| | ATGTCGTATGGGTAAAGCGC (SEQ ID NO: | GAGCCAUCACCAGCG |
| | 289) | ACUAUGUCGUAUGGG |
| sg64 backbone | CTTTGATGCTTCTTACGGACCGAAGTCCGTAAGCG | UAAAGCGCUUACGGA |
| rev | CTTTACCCATACGACATAG (SEQ ID NO: | CUUCGGUCCGUAAGA |
| | 290) | AGCAUCAAAGGGCCG |
| sg64.7.37 | CGGAGCGAGACATCTCGGCCCTTTGATGCTTCTTA | AGAUGUCUCGCUCCG |
| spacer primer | CGGACCGAAG (SEQ ID NO: 291) | (SEQ ID NO: |
| | | 297) |
| sg174 | GAAATTAATACGACTCACTATAACTGGCGCTTTTA | ACUGGCGCUUUUAUC |
| backbone fwd | TCTGATTACTTTGAGAGCCATCACCAGCGACTATG | UgAUUACUUUGAGAG |
| | TCGTAGTGGGTAAAGCT (SEQ ID NO: 292) | CCAUCACCAGCGACU |
| sg174 | CTTTGATGCTCCCTCCGAAGAGGGAGCTTTACCCA | AUGUCGUAgUGGGUA |
| backbone rev | CTACGACATAGTCGC (SEQ ID NO: 293) | AAGCUCCCUCUUCGG |
| sg174.7.37 | CGGAGCGAGACATCTCGGCCCTTTGATGCTCCCTC | AGGGAGCAUCAAAGG |
| spacer primer | C (SEQ ID NO: 294) | GCCGAGAUGUCUCGC |
| | | UCCG (SEQ ID |
| | | NO: 298) |

### Example 7: RNP Assembly

Purified wild-type and RNP of CasX and single guide RNA (sgRNA) were either prepared immediately before experiments or prepared and snap-frozen in liquid nitrogen and stored at −80° C. for later use. To prepare the RNP complexes, the CasX protein was incubated with sgRNA at 1:1.2 molar ratio. Briefly, sgRNA was added to Buffer #1 (25 mM NaPi, 150 mM NaCl, 200 mM trehalose, 1 mM MgCl2), then the CasX was added to the sgRNA solution, slowly with swirling, and incubated at 37° C. for 10 min to form RNP complexes. RNP complexes were filtered before use through a 0.22 m Costar 8160 filters that were pre-wet with 200 l Buffer #1. If needed, the RNP sample was concentrated with a 0.5 ml Ultra 100-Kd cutoff filter, (Millipore part #UFC510096), until the desired volume was obtained. Formation of competent RNP was assessed as described in Example 13.

### Example 8: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 9: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 pM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex.

### Example 10: Assessing Differential PAM Recognition In Vitro

Purified wild-type and engineered CasX variants will be complexed with single-guide RNA bearing a fixed targeting sequence. The RNP complexes will be added to buffer containing MgCl2 at a final concentration of 100 nM and incubated with 5′ Cy7.5-labeled double-stranded target DNA at a concentration of 10 nM. Separate reactions will be carried out with different DNA substrates containing different PAMs adjacent to the target nucleic acid sequence. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the rate of cleavage of the non-canonical PAMs by the CasX variants will be determined.

### Example 11: Assessing Nuclease Activity for Double-Strand Cleavage

Purified wild-type and engineered CasX variants will be complexed with single-guide RNA bearing a fixed HRS targeting sequence. The RNP complexes will be added to buffer containing MgCl2 at a final concentration of 100 nM and incubated with double-stranded target DNA with a 5′ Cy7.5 label on either the target or non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of the target and non-target strands by the wild-type and engineered variants will be determined. To more clearly differentiate between changes to target binding vs the rate of catalysis of the nucleolytic reaction itself, the protein concentration will be titrated over a range from 10 nM to 1 uM and cleavage rates will be determined at each concentration to generate a pseudo-Michaelis-Menten fit and determine the kcat* and KM*. Changes to KM* are indicative of altered binding, while changes to kcat* are indicative of altered catalysis.

### Example 12: Assessing Target Strand Loading for Cleavage

Purified wild-type and engineered CasX 491 will be complexed with single-guide RNA bearing a fixed HRS targeting sequence. The RNP complexes will be added to buffer containing MgCl2 at a final concentration of 100 nM and incubated with double-stranded target DNA with a 5′ Cy7.5 label on the target strand and a 5′ Cy5 label on the non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of both strands by the variants will be determined. Changes to the rate of target strand cleavage but not non-target strand cleavage would be indicative of improvements to the loading of the target strand in the active site for cleavage. This activity could be further isolated by repeating the assay with a dsDNA substrate that has a gap on the non-target strand, mimicking a pre-cleaved substrate. Improved cleavage of the non-target strand in this context would give further evidence that the loading and cleavage of the target strand.

### Example 13: CasX:gNA In Vitro Cleavage Assays

### 1. Determining Cleavage-Competent Fractions for Protein Variants Compared to Wild-Type Reference CasX

The ability of CasX variants to form active RNP compared to reference CasX was determined using an in vitro cleavage assay. The beta-2 microglobulin (B2M) 7.37 target for the cleavage assay was created as follows. DNA oligos with the sequence TGAAGCTGACAGCATTCGGGCCGAGATGTCTCGCTCCGTGGCCTTAGCTGTGCTCGCGCT (non-target strand, NTS (SEQ ID NO: 299)) and TGAAGCTGACAGCATTCGGGCCGAGATGTCTCGCTCCGTGGCCTTAGCTGTGCTCGCGCT (target strand, TS (SEQ ID NO: 300)) were purchased with 5′ fluorescent labels (LI-COR TRDye 700 and 800, respectively). dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), heating to 95° C. for 10 minutes, and allowing the solution to cool to room temperature.

CasX RNPs were reconstituted with the indicated CasX and guides (see graphs) at a final concentration of 1 μM with 1.5-fold excess of the indicated guide unless otherwise specified in 1× cleavage buffer (20 mM Tris HCl pH 7.5, 150 mM NaCl, 1 mM TCEP, 5% glycerol, 10 mM MgCl₂) at 37° C. for 10 min before being moved to ice until ready to use. The 7.37 target was used, along with sgRNAs having spacers complementary to the 7.37 target.

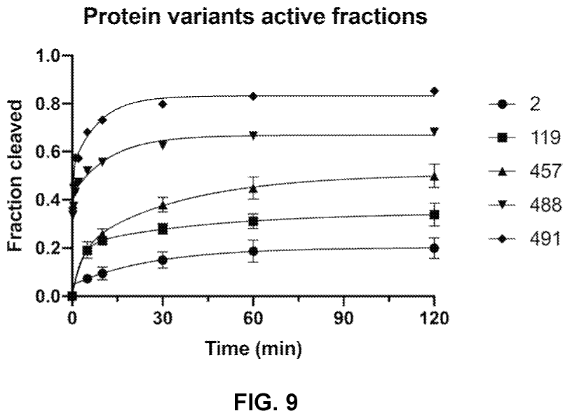

Cleavage reactions were prepared with final RNP concentrations of 100 nM and a final target concentration of 100 nM. Reactions were carried out at 37° C. and initiated by the addition of the 7.37 target DNA. Aliquots were taken at 5, 10, 30, 60, and 120 minutes and quenched by adding to 95% formamide, 20 mM EDTA. Samples were denatured by heating at 95° C. for 10 minutes and run on a 10% urea-PAGE gel. The gels were either imaged with a LI-COR Odyssey CLx and quantified using the LI-COR Image Studio software or imaged with a Cytiva Typhoon and quantified using the Cytiva IQTL software. The resulting data were plotted and analyzed using Prism. We assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. The cleavage traces were fit with a biphasic rate model, as the cleavage reaction clearly deviates from monophasic under this concentration regime, and the plateau was determined for each of three independent replicates. The mean and standard deviation were calculated to determine the active fraction (Table 12). The graph is shown in FIG. 9.

Apparent active (competent) fractions were determined for RNPs formed for CasX2+guide 174+7.37 spacer, CasX119+guide 174+7.37 spacer, CasX457+guide 174+7.37 spacer, CasX488+guide 174+7.37 spacer, and CasX491+guide 174+7.37 spacer. The determined active fractions are shown in Table 12. All CasX variants had higher active fractions than the wild-type CasX2, indicating that the engineered CasX variants form significantly more active and stable RNP with the identical guide under tested conditions compared to wild-type CasX. This may be due to an increased affinity for the sgRNA, increased stability or solubility in the presence of sgRNA, or greater stability of a cleavage-competent conformation of the engineered CasX:sgRNA complex. An increase in solubility of the RNP was indicated by a notable decrease in the observed precipitate formed when CasX457, CasX488, or CasX491 was added to the sgRNA compared to CasX2.

2. In Vitro Cleavage Assays—Determining k_{cleave} for CasX Variants Compared to Wild-Type Reference CasX

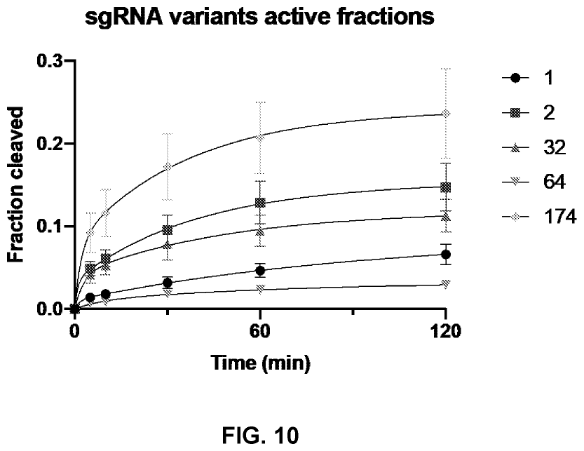

Cleavage-competent fractions were also determined using the same protocol for CasX2.2.7.37, CasX2.32.7.37, CasX2.64.7.37, and CasX2.174.7.37 to be 16±3%, 13±3%, 5±2%, and 22±5%, as shown in FIG. 10 and Table 12.

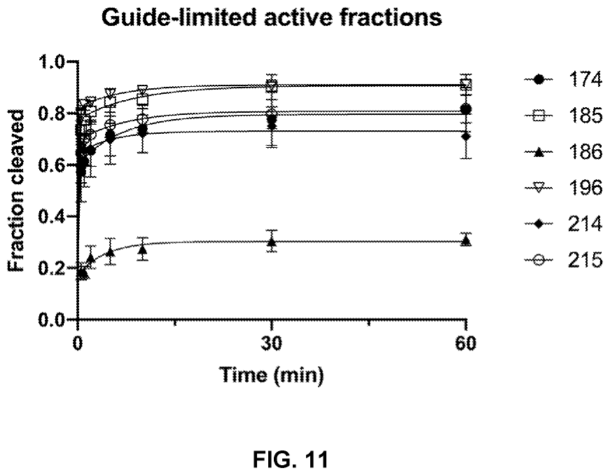

A second set of guides were tested under different conditions to better isolate the contribution of the guide to RNP formation. 174, 175, 185, 186, 196, 214, and 215 guides with 7.37 spacer were mixed with CasX491 at final concentrations of 1 μM for the guide and 1.5 μM for the protein, rather than with excess guide as before. Results are shown in FIG. 11 and Table 12. Many of these guides exhibited additional improvement over 174, with 185 and 196 achieving 91±4% and 91±1% competent fractions, respectively, compared with 80±9% for 174 under these guide-limiting conditions.

The data indicate that both CasX variants and sgRNA variants are able to form a higher degree of active RNP with guide RNA compare to wild-type CasX and wild-type sgRNA.

The apparent cleavage rates of CasX variants 119, 457, 488, and 491 compared to wild-type reference CasX were determined using an in vitro fluorescent assay for cleavage of the target 7.37.

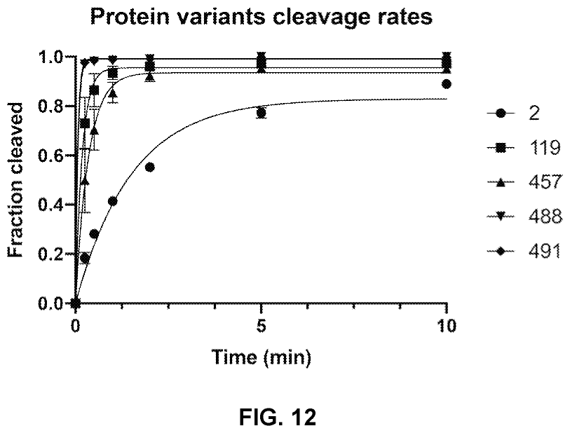

CasX RNPs were reconstituted with the indicated CasX (see FIG. 12) at a final concentration of 1 μM with 1.5-fold excess of the indicated guide in 1× cleavage buffer (20 mM Tris HCl pH 7.5, 150 mM NaCl, 1 mM TCEP, 5% glycerol, 10 mM MgCl₂) at 37° C. for 10 min before being moved to ice until ready to use. Cleavage reactions were set up with a final RNP concentration of 200 nM and a final target concentration of 10 nM. Reactions were carried out at 37° C. except where otherwise noted and initiated by the addition of the target DNA. Aliquots were taken at 0.25, 0.5, 1, 2, 5, and 10 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software or imaged with a Cytiva Typhoon and quantified using the Cytiva IQTL software. The resulting data were plotted and analyzed using Prism, and the apparent first-order rate constant of non-target strand cleavage (k_{cleave}) was determined for each CasX:sgRNA combination replicate individually. The mean and standard deviation of three replicates with independent fits are presented in Table 12, and the cleavage traces are shown in FIG. 12.

Apparent cleavage rate constants were determined for wild-type CasX2, and CasX variants 119, 457, 488, and 491 with guide 174 and spacer 7.37 utilized in each assay (see Table 12 and FIG. 12). All CasX variants had improved cleavage rates relative to the wild-type CasX2. CasX457 cleaved more slowly than 119, despite having a higher competent fraction as determined above. CasX488 and CasX491 had the highest cleavage rates by a large margin; as the target was almost entirely cleaved in the first timepoint, the true cleavage rate exceeds the resolution of this assay, and the reported k_{cleave} should be taken as a lower bound.

The data indicate that the CasX variants have a higher level of activity, with k_{cleave} rates reaching at least 30-fold higher compared to wild-type CasX2.

### 3. In Vitro Cleavage Assays: Comparison of Guide Variants to Wild-Type Guides

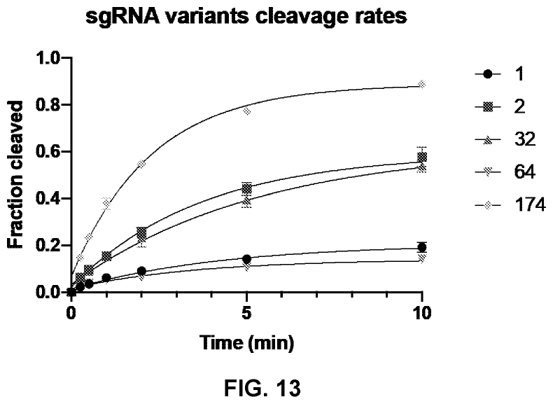

Cleavage assays were also performed with wild-type reference CasX2 and reference guide 2 compared to guide variants 32, 64, and 174 to determine whether the variants improved cleavage. The experiments were performed as described above. As many of the resulting RNPs did not approach full cleavage of the target in the time tested, we determined initial reaction velocities (V₀) rather than first-order rate constants. The first two timepoints (15 and 30 seconds) were fit with a line for each CasX:sgRNA combination and replicate. The mean and standard deviation of the slope for three replicates were determined (FIG. 13).

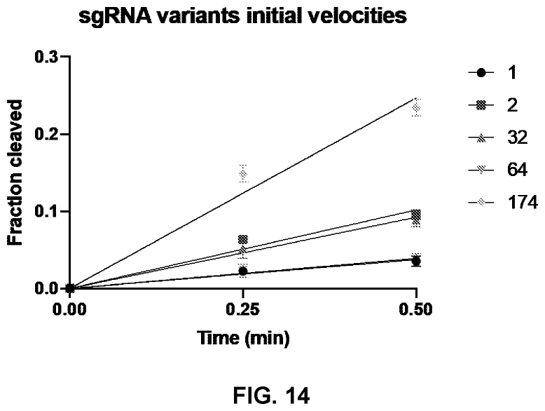

Under the assayed conditions, the V₀ for CasX2 with guides 2, 32, 64, and 174 were 20.4±1.4 nM/min, 18.4±2.4 nM/min, 7.8±1.8 nM/min, and 49.3±1.4 nM/min (see Table 12 and FIG. 13 and FIG. 14). Guide 174 showed substantial improvement in the cleavage rate of the resulting RNP (˜2.5-fold relative to 2, see FIG. 14), while guides 32 and 64 performed similar to or worse than guide 2. Notably, guide 64 supports a cleavage rate lower than that of guide 2 but performs much better in vivo (data not shown). Some of the sequence alterations to generate guide 64 likely improve in vivo transcription at the cost of a nucleotide involved in triplex formation. Improved expression of guide 64 likely explains its improved activity in vivo, while its reduced stability may lead to improper folding in vitro.

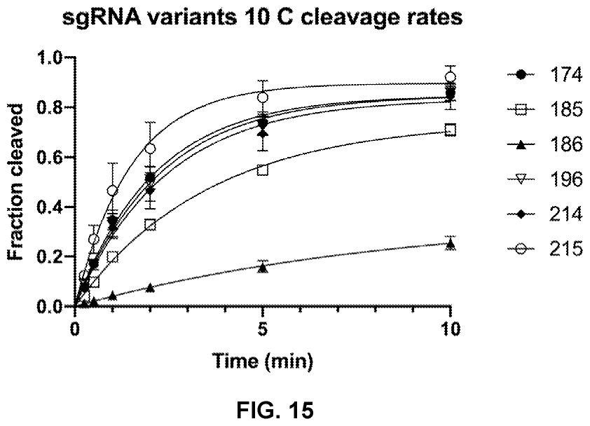

Additional experiments were carried out with guides 174, 175, 185, 186, 196, 214, and 215 with spacer 7.37 and CasX 491 to determine relative cleavage rates. To reduce cleavage kinetics to a range measurable with our assay, the cleavage reactions were incubated at 10° C. Results are in FIG. 15 and Table 12. Under these conditions, 215 was the only guide that supported a faster cleavage rate than 174.196, which exhibited the highest active fraction of RNP under guide-limiting conditions, had kinetics essentially the same as 174, again highlighting that different variants result in improvements of distinct characteristics.

The data support that, under the conditions of the assay, use of the majority of the guide variants with CasX results in RNP with a higher level of activity than one with the wild-type guide, with improvements in initial cleavage velocity ranging from ˜2-fold to >6-fold. Numbers in Table 12 indicate, from left to right, CasX variant, sgRNA scaffold, and spacer sequence of the RNP construct. In the RNP construct names in the table below, CasX protein variant, guide scaffold and spacer are indicated from left to right.

TABLE 12
Results of cleavage and RNP formation assays

| RNP | | | Competent |
|---|---|---|---|
| Construct | k_{cleave}* | Initial velocity* | fraction |
| 2.2.7.37 | | 20.4 ± 1.4 | 16 ± 3% |
| | | nM/min | |
| 2.32.7.37 | | 18.4 ± 2.4 | 13 ± 3% |
| | | nM/min | |
| 2.64.7.37 | | 7.8 ± 1.8 | 5 ± 2% |
| | | nM/min | |
| 2.174.7.37 | 0.51 ± 0.01 min⁻¹ | 49.3 ± 1.4 | 22 ± 5% |
| | | nM/min | |
| 119.174.7.37 | 6.29 ± 2.11 min⁻¹ | | 35 ± 6% |
| 457.174.7.37 | 3.01 ± 0.90 min⁻¹ | | 53 ± 7% |
| 488.174.7.37 | 15.19 min⁻¹ | | 67% |
| 491.174.7.37 | 16.59 min⁻¹/0.293 | | 83%/17% (guide- |
| | min⁻¹ (10° C.) | | limited) |
| 491.175.7.37 | 0.089 min⁻¹ (10° C.) | | 5% (guide- |
| | | | limited) |
| 491.185.7.37 | 0.227 min⁻¹ (10° C.) | | 44% (guide- |
| | | | limited) |
| 491.186.7.37 | 0.099 min⁻¹ (10° C.) | | 11% (guide- |
| | | | limited) |
| 491.196.7.37 | 0.292 min⁻¹ (10° C.) | | 46% (guide- |
| | | | limited) |
| 491.214.7.37 | 0.284 min⁻¹ (10° C.) | | 30% (guide- |
| | | | limited) |
| 491.215.7.37 | 0.398 min⁻¹ (10° C.) | | 38% (guide- |
| | | | limited) |

*Mean and standard deviation

### Example 14: Assessing Differential PAM Recognition In Vitro

In vitro cleavage assays were performed essentially as described in Example 13, using CasX2, CasX119, and CasX438 complexed with sg174.7.37. Fluorescently labeled dsDNA targets with a 7.37 spacer and either a TTC, CTC, GTC, or ATC PAM were used (sequences are in Table 13). Time points were taken at 0.25, 0.5, 1, 2, 5, 10, 30, and 60 minutes. Gels were imaged with an Cytiva Typhoon and quantified using the IQTL 8.2 software. Apparent first-order rate constants for non-target strand cleavage (k_{cleave}) were determined for each Casx:sgRNA complex on each target. Rate constants for targets with non-TTC PAM were compared to the TTC PAM target to determine whether the relative preference for each PAM was altered in a given protein variant.

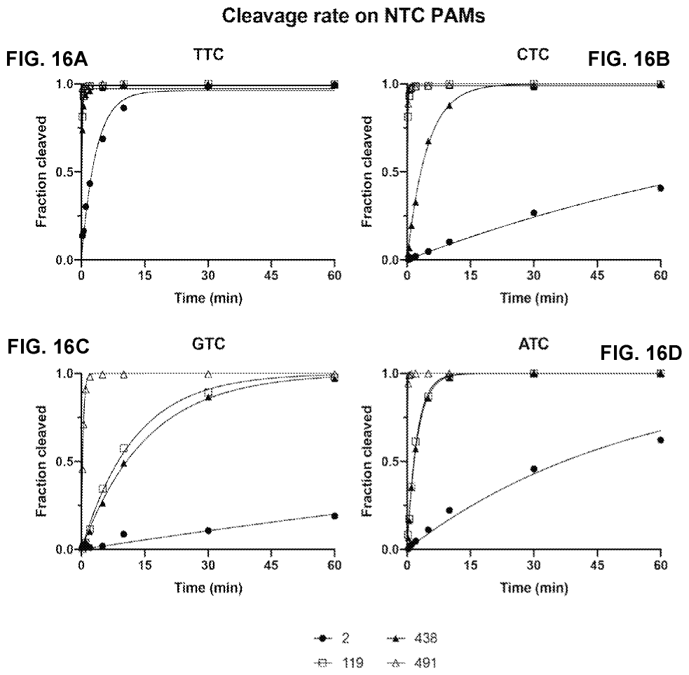

For all variants, the TTC target supported the highest cleavage rate, followed by the ATC, then the CTC, and finally the GTC target (FIGS. 16A-16D, Table 14). For each combination of CasX variant and NTC PAM, the cleavage rate k_{cleave} is shown. For all non-NTC PAMs, the relative cleavage rate as compared to the TTC rate for that variant is shown in parentheses. All non-TTC PAMs exhibited substantially decreased cleavage rates (>10-fold for all). The ratio between the cleavage rate of a given non-TTC PAM and the TTC PAM for a specific variant remained generally consistent across all variants. The CTC target supported cleavage 3.5-4.3% as fast as the TTC target; the GTC target supported cleavage 1.0-1.4% as fast; and the ATC target supported cleavage 6.5-8.3% as fast. The exception is for 491, where the kinetics of cleavage at TTC PAMs are too fast to allow accurate measurement, which artificially decreases the apparent difference between TTC and non-TTC PAMs. Comparing the relative rates of 491 on GTC, CTC, and ATC PAMs, which fall within the measurable range, results in ratios comparable to those for other variants when comparing across non-TTC PAMs, consistent with the rates increasing in tandem. Overall, differences between the variants are not substantial enough to suggest that the relative preference for the various NTC PAMs have been altered. However, the higher basal cleavage rates of the variants allow targets with ATC or CTC PAMs to be cleaved nearly completely within 10 minutes, and the apparent k_{cleaves} are comparable to or greater than the k_{cleave} of CasX2 on a TTC PAM (Table 14). This increased cleavage rate may cross the threshold necessary for effective genome editing in a human cell, explaining the apparent increase in PAM flexibility for these variants.

TABLE 13
Sequences of DNA substrates used in in vitro PAM cleavage assay.

| Guide* | DNA Sequence | SEQ ID NO |
|---|---|---|
| 7.37 TTC | AGCGCGAGCACAGCTAAGGCCACGGAGCGAGACATCTCGG | 301 |
| PAM TS | CCCGAATGCTGTCAGCTTCA | |
| 7.37 TTC | TGAAGCTGACAGCATTCGGGCCGAGATGTCTCGCTCCGTG | 302 |
| PAM NTS | GCCTTAGCTGTGCTCGCGCT | |
| 7.37 CTC | AGCGCGAGCACAGCTAAGGCCACGGAGCGAGACATCTCGG | 303 |
| PAM TS | CCCGAGTGCTGTCAGCTTCA | |
| 7.37 CTC | TGAAGCTGACAGCACTCGGGCCGAGATGTCTCGCTCCGTG | 304 |
| PAM NTS | GCCTTAGCTGTGCTCGCGCT | |
| 7.37 GTC | AGCGCGAGCACAGCTAAGGCCACGGAGCGAGACATCTCGG | 305 |
| PAM TS | CCCGACTGCTGTCAGCTTCA | |
| 7.37 GTC | TGAAGCTGACAGCAGTCGGGCCGAGATGTCTCGCTCCGTG | 305 |
| PAM NTS | GCCTTAGCTGTGCTCGCGCT | |
| 7.37 ATC | AGCGCGAGCACAGCTAAGGCCACGGAGCGAGACATCTCGG | 307 |
| PAM TS | CCCGATTGCTGTCAGCTTCA | |
| 7.37 ATC | TGAAGCTGACAGCAATCGGGCCGAGATGTCTCGCTCCGTG | 308 |
| PAM NTS | GCCTTAGCTGTGCTCGCGCT | |

*The PAM sequences for each are bolded. TS-target strand. NTS-Non-target strand.

TABLE 14
Apparent cleavage rates of CasX variants against NTC PAMs.

| Variant | TTC | | CTC | GTC | ATC |
|---|---|---|---|---|---|
| 2 | 0.267 | min⁻¹ | 9.29E−3 min⁻¹ | 3.75E−3 min⁻¹ | 1.87E−2 min⁻¹ |
| | | | (0.035) | (0.014) | (0.070) |
| 119 | 8.33 | min⁻¹ | 0.303 min⁻¹ | 8.64E−2 min⁻¹ | 0.540 min⁻¹ |
| | | | (0.036) | (0.010) | (0.065) |
| 438 | 4.94 | min⁻¹ | 0.212 min⁻¹ | 1.31E−2 min⁻¹ | 0.408 min⁻¹ |
| | | | (0.043) | (0.013) | (0.083) |
| 491 | 16.42 | min⁻¹ | 8.605 min⁻¹ | 2.447 min⁻¹ | 11.33 min⁻¹ |
| | | | (0.524) | (0.149) | (0.690) |

### Example 15: Identification of Nicking Variants

Purified modified CasX variants will be complexed with single-guide RNA bearing a fixed targeting sequence. The RNP complexes will be added to buffer containing MgCl₂ at a final concentration of 100 nM and incubated with double-stranded target DNA with a 5′ fluorescein label on the target strand and a 5′ Cy5 label on the non-target strand at a concentration of 10 nM. Aliquots of the reactions will be taken at fixed time points and quenched by the addition of an equal volume of 50 mM EDTA and 9500 formamide. The samples will be run on a denaturing polyacrylamide gel to separate cleaved and uncleaved DNA substrates. Efficient cleavage of one strand but not the other would be indicative that the variant possessed single-strand nickase activity.

### Example 16: Assessing Improved Expression and Solubility Characteristics of CasX Variants for RNP Production

Wild-type and modified CasX variants will be expressed in BL21 (DE3) E. coli under identical conditions. All proteins will be under the control of an IPTG-inducible T7 promoter. Cells will be grown to an OD of 0.6 in TB media at 37° C., at which point the growth temperature will be reduced to 16° C. and expression will be induced by the addition of 0.5 mM IPTG. Cells will be harvested following 18 hours of expression. Soluble protein fractions will be extracted and analyzed on an SDS-PAGE gel. The relative levels of soluble CasX expression will be identified by Coomassie staining. The proteins will be purified in parallel according to the protocol above, and final yields of pure protein will be compared. To determine the solubility of the purified protein, the constructs will be concentrated in storage buffer until the protein begins to precipitate. Precipitated protein will be removed by centrifugation and the final concentration of soluble protein will be measured to determine the maximum solubility for each variant. Finally, the CasX variants will be complexed with single guide RNA and concentrated until precipitation begins. Precipitated RNP will be removed by centrifugation and the final concentration of soluble RNP will be measured to determine the maximum solubility of each variant when bound to guide RNA.

### Example 17: CasX:gNA Editing of C9orf72

This example sets forth the parameters to make and test compositions capable of modifying a C9orf72 locus.

### Experimental Design:

### A) C9orf72-Modifying Spacer Selection Process:

20 bp XTC PAM spacers will be designed to target the following regions in the human genome:
(a) C9orf72 cis enhancer elements
(b) C9orf72 proximal non-coding genetic elements highly conserved across vertebrates (UCSC genome browser)
(c) C9orf72 genomic locus. The C9orf72 gene is defined as the sequence that spans chr9: 27,546,546-27,573,866 of the human genome on chromosome 9 (Homo sapiens Updated Annotation Release 109.20191205, GRCh38.p13 (NCBI). The human C9orf72 gene is described in part in the NCBI database (ncbi.nlm.nih.gov) as Reference Sequence NC_000009.12, which is incorporated by reference herein. C9orf72 targeting spacers may be similarly assembled from other genomes.

### B) Methods for Generating C9orf72 Targeting Constructs:

In order to generate C9orf72 targeting constructs, C9orf72 targeting spacers will be cloned into a base mammalian-expression plasmid construct (pStX) that is comprised of the following components: codon optimized CasX (construct CasX 491 molecule and rRNA guide 174 (491.174); see Tables for sequences)+NLS; and a mammalian selection marker, puromycin. Spacer sequence DNA will be ordered as single-stranded DNA (ssDNA) oligos from Integrated DNA Technologies (IDT) consisting of the spacer sequence and the reverse complement of this sequence. These two oligos will be annealed together and cloned into pStX individually or in bulk by Golden Gate Assembly using T4 DNA Ligase and an appropriate restriction enzyme for the plasmid. Assembled products will be transformed into chemically- or electro-competent bacterial cells, plated on Lb-Agar plates containing carbenicillin and incubated until colonies appeared. Individual colonies will be picked and miniprepped using a Qiagen Qiaprep spin Miniprep Kit (Qiagen Cat #27104), following the manufacturer's protocol. The resultant plasmids will be sequenced using Sanger sequencing to ensure correct ligation. SaCas9 and SpyCas9 control plasmids, with spacers chosen based on Cas protein-specific PAMs, will be prepared similarly to pStX plasmids described above.

### C) Methods to Generate C9orf72 Reporter Line:

A fluorescent-encoding DNA (e.g., GFP) will be knocked in at the 3′ end of the last C9orf72 exon in a HEPG2 cell line. The modified cells will be expanded by serial passage every 3-5 days and maintained in Fibroblast (FB), consisting of Dulbecco's Modified Eagle Medium (DMEM; Corning Cellgro, #10-013-CV) supplemented with 10% fetal bovine serum (FBS; Seradigm, #1500-500), or other appropriate medium, and 100 Units/ml penicillin and 100 mg/ml streptomycin (100×-Pen-Strep; GIBCO #15140-122), and can additionally include sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023). The cells will be incubated at 37° C. and 5% CO2. After 1-2 weeks, single GFP+ cells will be sorted into FB or other appropriate medium. The reporter line clones will be expanded by serial passage every 3-5 days and maintained in FB medium in an incubator at 37° C. and 5% CO2. The lines will be characterized via genomic sequencing, and functional modification of the C9orf72 locus using a C9orf72 targeting molecule. The optimal reporter lines will be identified as ones that i) had a single copy of GFP correctly integrated at the target C9orf72 locus, ii) maintained doubling times equivalent to unmodified cells, iii) resulted in reduction in GFP fluorescence upon disruption of the C9orf72 gene when assayed using the methods described, below.

### D) Methods to Assess C9orf72 Modifying Activity in C9orf72-GFP Reporter Cell Line:

C9orf72 reporter cells will be seeded at 20-40 k cells/well in a 96 well plate in 100 μl of FB (or other appropriate) medium and cultured in a 37° C. incubator with 5% CO2. The following day, confluence of seeded cells will be checked. Ideally, cells should be at ˜75% confluence at time of transfection. If cells will be at the right confluence, transfection will be carried out.

Each CasX construct (CasX 491 and guide 174, see Tables for sequences) with appropriate spacers targeting C9orf72 will be transfected at 100-500 ng per well using Lipofectamine 3000 following the manufacturer's protocol, using 3 wells per construct as replicates. SaCas9 and SpyCas9 targeting C9orf72 will be used as benchmarking controls. For each Cas protein type, a non-targeting plasmid will be used as a negative control.

After 24-48 hours of puromycin selection at 0.3-3 μg/ml, to select for successfully transfected cells, followed by 24-48 hours of recovery in FB or other appropriate medium, fluorescence in transfected cells will be analyzed via flow cytometry. In this process, cells will be gated for the appropriate forward and side scatter, selected for single cells and then gated for reporter expression (Attune Nxt Flow Cytometer, Thermo Fisher Scientific) to quantify the expression levels of fluorophores. At least 10,000 events will be collected for each sample. The data will be used to calculate the percentage of antibody-label negative (edited) cells.

A subset of cells for each sample from the example will be lysed, and genome extracted using a Quick extract solution following the manufacturer's protocol. Editing will be analyzed using a T7E1 assay. Briefly, the genomic locus at the targeted edit site will be amplified using primers (e.g., a 500 bp region around the intended target) using a PCR program on a thermocycler. The PCR amplicon will be then hybridized following a hybridization program on a thermocycler, and then treated with T7 Endonuclease for 30 minutes at 37° C. The sample will be then analyzed on a 2% agarose gel, or on a Fragment Analyzer to visualize DNA bands.

### Example 18: Methods to Assess C9orf72 Hexanucleotide Repeat Expansion (HRE) Modifying Activity in HEK293T Cells

HEK293T (Cell Culture Facility, UC Berkeley) were seeded at 30 k cells/well in a 96 well plate in 100 μl of FB medium and cultured in a 37° C. incubator with 5% C02. The following day, confluence of seeded cells will be checked. Cells that were at least ˜75% confluence at time of transfection were used for transfection.

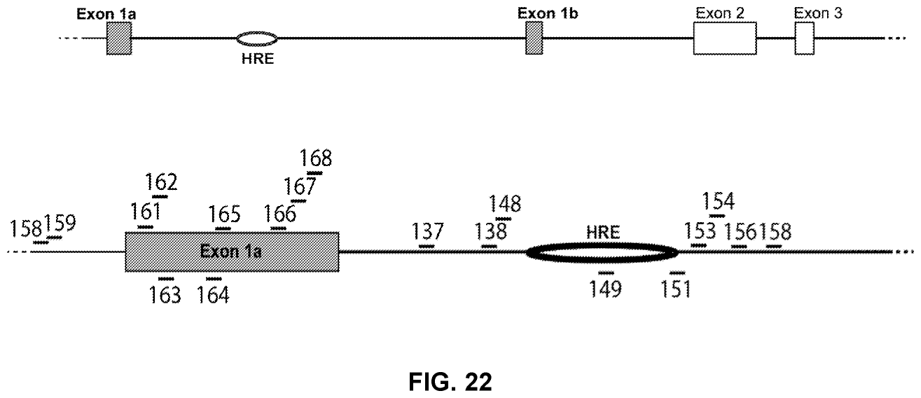

Plasmid p59.491,174,29.X, encoding CasX construct 491 with guide 174 and appropriate spacers targeting sequences 5′ to 3′ to the HRE region C9orf72 (Table 15, with targeting locations in the locus shown schematically in FIG. 22), were lipofected at 100 ng per well using Lipofectamine 3000 following the manufacturer's protocol, and placed into 3 wells per construct as replicates. A non-targeting plasmid was used as a negative control. Puromycin selection at 1-3 μg/ml was used to select for successfully transfected cells. After 4 days, samples were harvested for gDNA extraction and amplified for NGS analysis.

### Results:

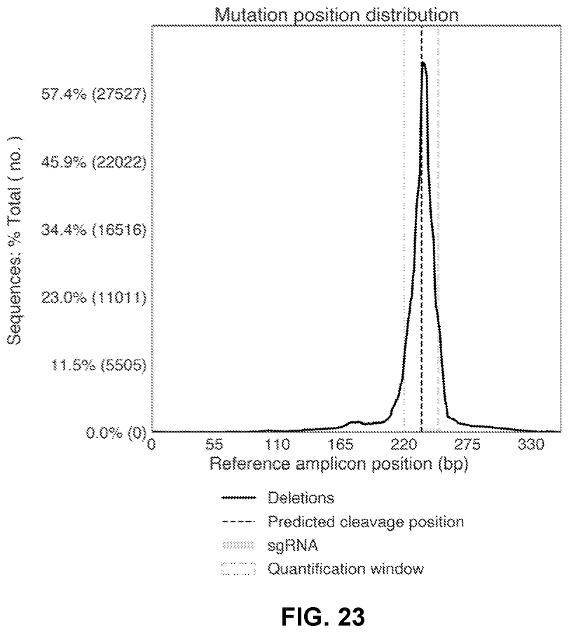

For single cuts, the percent editing is shown in Table 15 and results for a single spacer are shown in FIG. 23. Efficient editing was observed for multiple PAMs (i.e., ATC, GTC and TTC), with the highest efficiency editing seen with TTC.

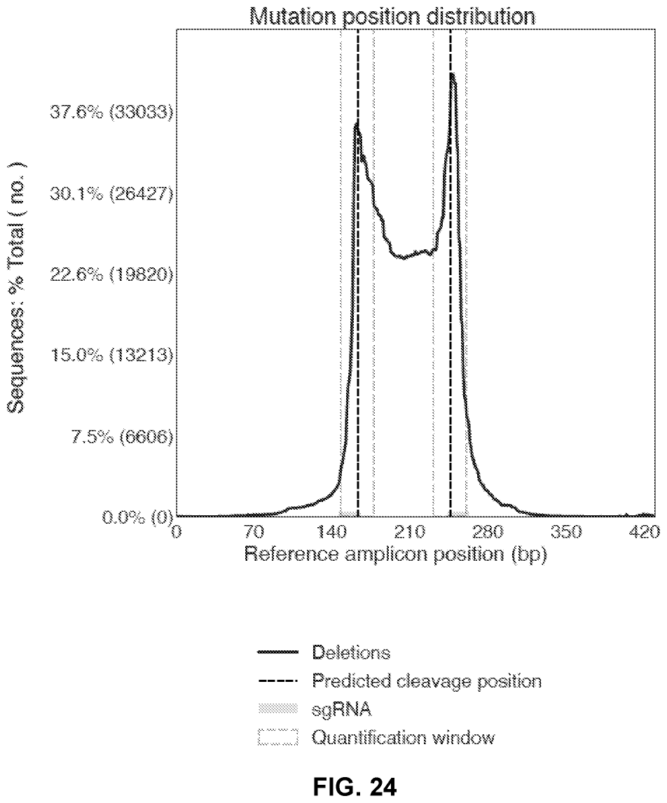

For double cuts and drop-out of the HRE region, different combinations of spacers demonstrated efficient editing, with a corresponding deletion of the intervening HRE sequence, averaging approximately 36% (Table 16). A representative graph of editing is shown in FIG. 24.

The results demonstrate that, under the conditions of the experiments, the CasX system with a single guide can be utilized to edit cis regulatory elements as well as the HRE directly, while use of two guides can be utilized to successfully excise the HRE region.

TABLE 15
C9orf72 spacers and percent editing

| Spacer | | % | SEQ |
|---|---|---|---|
| # | Spacer Sequence | Editing | ID NO |
| 137 | CUUGUCCCUGCGCCGCCGCC | 32 | 309 |
| 138 | CCACCGCCUGCGCCUCCGCC | — | 310 |
| 140 | CCUCUAGCGACUGGUGGAAU | — | 311 |
| 141 | ACCAGUCGCUAGAGGCGAAA | — | 312 |
| 142 | CUCUCAUUUCUCUGACCGAA | — | 313 |
| 143 | ACCCGCGCCUCUUCCCGGCA | 51.86 | 314 |
| 144 | GCUGCCGGGAAGAGGCGCGG | 48.05 | 315 |
| 145 | CGGCAGCCGAACCCCAAACA | 52.8 | 316 |
| 146 | UGGCGGGUGGCUGUUUGGGG | 56.23 | 317 |
| 147 | UCACUCACCCACUCGCCACC | 49.93 | 318 |
| 148 | GCCGCCGCGGGCGCAGGCAC | 36.45 | 319 |
| 149 | GGGCGGGCCCGGGGGGGGC | 20.76 | 320 |
| 151 | GGAGUCGCGCGCUAGGGGCC | 53.12 | 321 |
| 153 | UGAGUUCCAGAGCUUGCUAC | 41.34 | 322 |
| 154 | AGAGCUUGCUACAGGCUGCG | 50.55 | 323 |
| 156 | UCUGGUUAAUCUUUAUCAGG | 44.25 | 324 |
| 158 | UGUUCACCCUCAGCGAGUAC | 59.26 | 325 |
| 159 | CCCUCAGCGAGUACUGUGAG | 50.88 | 326 |
| 161 | UAAACCCACACCUGCUCUUG | 27.45 | 327 |
| 162 | UGCUAGACCCCGCCCCCAAA | — | 328 |
| 163 | CUUUUGGGGGCGGGGUCUAG | 11.03 | 329 |
| 164 | GCCCACGUAAAAGAUGACGC | 65.42 | 330 |
| 165 | UUUACGUGGGCGGAACUUGU | 12.67 | 331 |
| 166 | CUUUCCUAGCGGGACACCGU | 24.08 | 332 |
| 167 | UUCCUAGCGGGACACCGUAG | 27.93 | 333 |
| 168 | UAGCGGGACACCGUAGGUUA | 54.33 | 334 |
| 170 | AUGUGCGAUGACGUUUUCUC | — | 335 |
| 171 | CUAGCCUCGUGAGAAAACGU | — | 336 |
| 172 | CACGAGGCUAGCGAAAUGGG | — | 337 |
| 173 | UUUAUCUUAAGACCCGCUCU | — | 338 |
| 175 | UCCAGAGCGGGUCUUAAGAU | — | 339 |
| 178 | UAAGACCCGCUCUGGAGGAG | — | 340 |
| 180 | CCAGAUCCAGCAGCCUCCCC | — | 341 |
| 181 | AAACCACGAAAUCGUCUUCA | 42.86 | 342 |
| 183 | UCCACCAAUUAUGUGUUGAG | 1.2 | 343 |

TABLE 16
Deletion editing by double-cut with 2 spacers

| Spacer | % Deletion |
|---|---|
| Combinations | Editing |
| 138/151 | 45.43 |
| 138/153 | 43.88 |
| 138/154 | 33.31 |
| 138/156 | 33.87 |
| 148/151 | 32.98 |
| 148/153 | 32.94 |
| 148/154 | 26.38 |
| 148/156 | 34.7 |
| 148/158 | 40.54 |
| 149/151 | 37.94 |
| 149/153 | 31.69 |
| 149/154 | 27.83 |
| 149/156 | 37.81 |
| 149/158 | 40.15 |

### Example 19: Methods to Package C9orf72 Targeting CasX Constructs in a Lentiviral Vector

Lentiviral particles packaging C9orf72 targeting CasX:gNA constructs (e.g., CasX 491 and guide 174) targeting C9orf72 will be produced by transfecting HEK293 at a confluency of 70%-90% using polyethylenimine based transfection of transgene plasmids encoding CasX, the guide RNA, the lentiviral packaging plasmid, and the VSV-G envelope plasmids. For lentiviral particle production, media will be changed 12 hr post-transfection, and the virus will be harvested at 36-48 hr post-transfection. Viral supernatants will be filtered using 0.45 m membrane filters, and diluted in FB media (Fibroblast medium, comprised of: DMEM with Glutamax (Gibco 10566-016) supplemented with MEM-NEAA (Thermo 11140050), sodium pyruvate (Thermo 11360070), HEPES (Thermo 15630080), 2-mercaptoethanol (Gibco 21985023), penicillin/streptomycin (Thermo 15140122) with 10% volume fraction of fetal bovine serum (FBS, VWR #97068-085)), if appropriate.

### Example 20: Methods to Assess C9orf72 Modification Via a Lentiviral Screen

Lentiviral plasmids are cloned as described above and following standard cloning procedures such that each lentiviral plasmid has one spacer-guide scaffold targeting C9orf72 and one codon optimized NLS bearing CasX molecule (e.g., construct CasX 491 molecule and rRNA guide 174 (491.174) with a puromycin selection marker. The cloning is carried out such that the final titer encompasses the full library size by >100× of all possible C9orf72 spacers targeting all known PAMs and their corresponding spacer regions in the C9orf72 gene. If ˜5,000 is the library size; the libraries evaluated would be >5×10⁵.

Lentiviral particles are produced by transfecting HEK293T at a confluency of 70%-90% using polyethylenimine based transfection of plasmids containing the spacer library, the lentiviral packaging plasmid and the VSV-G envelope plasmids. For particle production, media is changed 12 hr. post-transfection, and virus harvested at 36-48 hr post-transfection.

Viral supernatants are filtered using 0.45 μm membrane filters, diluted in FB media if appropriate, and added to target cells, in this case the C9orf72-GFP reporter cell line. Supplement polyberene is added at 5-20 μg/ml to enhance transduction efficiency, if necessary. Transduced cells are selected for 24-48 hr. post-transduction using puromycin at 0.3-3 μg/ml in FB medium, and grown for 7-10 days in FB or other appropriate medium in a 37° C. incubator with 5% CO₂.

Cells are sorted on a SH-100 or MA900 SONY sorter. In this process, cells are gated for the appropriate forward and side scatter, selected for single cells and then gated for reporter expression. Different cell sorting gates are established based on fluorescence level (OFF=full KO, Med=partial disruption or knockdown (KD), High=no edit, Very High=Enhancer) to distinguish between and collect cells editing by i) highly functional C9orf72 disrupting molecules, ii) molecules that only lower expression, and iii) molecules that increase expression. This assay can also be run to identify allele specific guides if two colors are used in human patient cells. Genomic DNA are collected from each group of sorted cells using Quick Extract (Lucigen Cat #QE09050) solution following manufacturer's recommended protocol.

Spacer libraries from each collected pool are then amplified via PCR directly from the genome and collected for deep sequencing on a Miseq. Analysis of the spacers is done according to gate and abundance for a specific activity; see below for detailed methods for NGS analysis of spacer hits.

Selected guides from each sorted group are then re-cloned and individually validated in reporter cell line and in primary human cell lines for activity by flow cytometry and T7E1 assay and/or Western blotting, and indel spectrum assessed by NGS analysis. Steps followed may be similar to the description provided under Methods to assess C9orf72 modifying activity in reporter cell line.

### Methods for NGS Analysis of Spacer Hits

Provided here are methods on how to analyze the next generation sequencing (NGS) data coming from above described lentiviral screen. Spacers are each assessed for ability to disrupt a C9orf72 gene using next generation sequencing (NGS). NGS libraries are generated through specific amplification of the lentiviral backbone containing the spacer. A different library is generated for each of the sorted populations (GFP high, med, low, etc., corresponding to low, med, high C9orf72 expression), and then assessed with the Illumina Hiseq.

Sequencing reads from the Illumina Hiseq are trimmed for adapter sequences and for regions of low sequencing quality. Paired end reads are merged based on their overlap sequence to form a single consensus sequence per sequenced fragment. Consensus sequences are aligned to the designed spacer sequences using bowtie2. Reads aligning to more than one designed spacer sequence are discarded.

The ‘abundance’ of each spacer sequence is defined as the number of reads aligning to that sequence. The abundance is tabulated for each sequencing library, forming a count table giving the abundance for each spacer sequence across each of the sequencing libraries (i.e., sorted populations). Finally, the numbers of abundances are then normalized to account for the differing sequencing depth of each library by dividing by the overall read count in that library, multiplied by the mean read count across libraries. The normalized count table is used to determine the activity of each spacer in each gate (high, medium, low, etc.).

A C9orf72-GFP reporter line will be constructed by knocking in GFP at the endogenous human C9orf72 locus. A reporter (e.g., GFP reporter) coupled to a gRNA targeting sequence, complementary to the gRNA spacer, is integrated into a reporter cell line. Cells are transformed or transfected with a CasX protein and/or sgRNA variant, with the spacer motif of the sgRNA complementary to and targeting the gRNA target sequence of the reporter. The ability of the CasX:sgRNA ribonucleoprotein complex to cleave the target nucleic acid sequence is assayed by FACS. Cells that lose reporter expression indicate occurrence of CasX:sgRNA ribonucleoprotein complex-mediated cleavage and indel formation. The reporting system is based on reduced GFP fluorescence upon successful modifying (editing) of the C9orf72 locus, detected by flow cytometry.

In an initial screen, two C9orf72 spacers of the gNA will be tested. The spacers will be tested with the CasX protein (construct CasX 491 with gNA 174), using SaCas9 and SpyCas9 as controls, in the reporter cell line. The reduction in GFP fluorescence, and editing, will be evaluated in the C9orf72-GFP reporter cells, selected for successful lipofection using puromycin, and later assayed for GFP disruption via FACS. The CasX 491 and guide 174 will be expected to edit at least 5-10% of cells, demonstrating that CasX can modify the endogenous C9orf72 locus, and do so more effectively than the SaCas9 and SpyCas9 system. A T7E1 assay or Western blotting will be performed to assay gene editing in the C9orf72-GFP reporter cell line. CasX 491 and guide 174 with C9orf72 targeting spacers and non-targeting control (NT) will be lipofected into C9orf72-GFP reporter cells, selected for successful lipofection using puromycin, and later assayed for gene editing in the T7E1 assay, demonstrating successful editing of the C9orf72 locus.

### Example 21: Method to Edit the C9orf72 Gene Using CasX Using Lentivirus Construct in an Allele Specific Manner

The example shows CasX's ability to edit the C9orf72 locus. One strategy to permanently treat a C9orf72 related disease is to specifically disrupt the mutant copy of the gene while sparing the WT allele. HEK293 cells with both wild-type alleles should be editable by a WT CasX spacer, but not by a mutant CasX spacer. This example will additionally demonstrate the ability of CasX spacers to distinguish between on-target and off-target alleles that differ by a single nucleotide. HEK293 cells are seeded at 20-40 k cells/well in a 96 well plate in 100 μl of FB medium and cultured in a 37° C. incubator with 5% CO₂. The following day, confluence of seeded cells are checked to ensure that cells will be at ˜75% confluence at time of transfection. If cells are at the right confluence, transfection is carried out using the viral supernatants of Example 19 (having CasX 491 and guide 174), using 3 wells per construct as replicates. SaCas9 and SpyCas9 targeting C9orf72 are used as benchmarking controls. For each Cas protein type, a non-targeting plasmid is used as a negative control. Cells will be selected for successful transfection with puromycin at 0.3-3 μg/ml for 24-48 hours followed by 24-48 hours of recovery in FB medium. A subset of cells for each sample from the experiment will be lysed, and the genome will be extracted using a Quick extract solution following the manufacturer's protocol. Editing will be analyzed using a T7E1 assay. Briefly, the genomic locus at the targeted edit site is amplified using primers (e.g., a 500 bp region around the intended target) using a PCR program on a thermocycler. The PCR amplicon is then hybridized following a hybridization program on a thermocycler, and then treated with T7 Endonuclease for 30 minutes at 37° C. The sample is then analyzed on a 2% agarose gel, or on a Fragment Analyzer to visualize the DNA bands.

### Example 22: Method to Demonstrate Allele-Specific Editing in Autosomal Dominant C9orf72 Patient-Derived Cell Lines Bearing Hexanucleotide Repeat Expansions

Cells derived from patients with the HRS in C9orf72 will be obtained and cultured under supplier recommended conditions. Cells will be transfected with CasX construct (e.g., CasX 491 with spacer 174) using Lipofectamine 3000 following manufacturer's protocol, or nucleofected using Lonza nucleofector kit according to manufacturer's protocol, and seeded in 96 well plates for incubation and growth. Alternatively, CasX constructs may be packaged in a lentivirus, and used to transduce patient-derived cells. Cells will be selected for successful lipofection or nucleofection, or lentiviral transduction, using medium containing puromycin at 0.3-3 μg/ml for 2-4 days or longer, followed by recovery in puromycin-free medium for 2 days or longer. Editing of the C9orf72 locus may be assessed at the genomic, transcriptomic, and proteomic level. At the end of the selection and recovery period, a subset of cells for each sample from the experiment will be lysed, and genome extracted using a Quick extract (QE) solution following the manufacturer's protocol; another subset of cells will be lysed in RIPA cell lysis buffer for proteomic analysis; another subset of cells may be passaged for analysis at a later point in time. A fraction of the QE treated samples will be used to assess editing using a T7E1 assay. Briefly, the genomic locus at the targeted edit site will be amplified using primers (e.g., a 500 bp region around the intended target) using a PCR program on a thermocycler. The PCR amplicon will then be hybridized following a hybridization program on a thermocycler, and then treated with T7 Endonuclease for 30 minutes at 37° C. The sample will be then analyzed on a 2% agarose gel, or on a Fragment Analyzer to visualize DNA bands to confirm that the CasX construct can successfully edit the C9orf72 mutation. Another fraction of the QE treated samples will be used to assess editing at the C9orf72 locus using NGS.

Proteomic analysis will be performed by Western blotting. The samples lysed in RIPA buffer will first be quantified for protein content using a colorimetric protein quantification assay such as BCA (Pierce) or Bradford (BioRad) according to the manufacturer's protocol. Following quantification, dilute sample in beta-mercaptoethanol-supplemented Laemmli buffer will be used to load 2.5-20 μg of total protein per well. Samples will be heat denatured at 95-100° C. for 5-10 minutes, and then cooled to room temperature. Samples will then be loaded onto and run on a polyacrylamide gel. Once the gel has run long enough, proteins will be transferred onto a PVDF membrane, blocked for at least 1 h at room temperature, and labeled with primary antibodies against C9orf72 and an appropriate loading control. The blot will be washed three times, at five minutes per wash, with PBST (PBS supplemented with 0.1 v/v % Triton X100) on a rocker at room temperature. An appropriate reporter-conjugated secondary antibody will then be used to label the primary antibody for 1 hour at room temperature. The blot will be washed three times, at five minutes per wash, with PBST (PBS supplemented with 0.1 v/v % Triton X100) on a rocker at room temperature. Any necessary substrates will subsequently be added, quenched if required, and imaged on a gel imager. Band intensities will be quantified using appropriate software following manufacturer's protocol.

### Example 23: Method to Deliver C9orf72-Targeting Construct Via AAV: Making and Recovery of AAV with Encoded CasX System

This example describes a typical protocol followed to produce and characterize AAV vectors packaging sequences encoding CasX molecules and guide RNA.

### Materials and Methods:

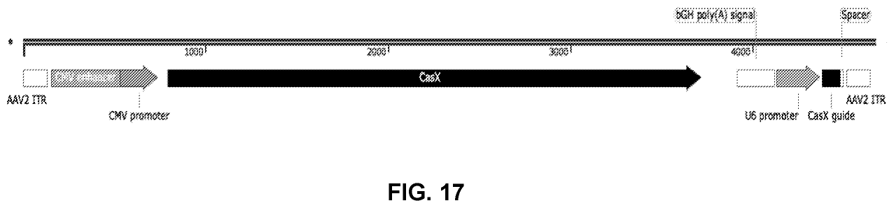

For AAV production, the tri-plasmid transfection method is used and requires three essential plasmids—pTransgene carrying the CasX:gRNA targeting the C9orf72 gene of interest to be packaged in AAV, pRC, and pHelper. DNA encoding CasX and guide RNA are cloned into an AAV transgene cassette, between the ITRs to generate the pTransgene plasmid a schematic of which is shown in FIG. 17. The constructed transgene plasmid is verified via full-length plasmid sequencing, restriction digestion, and functional tests including in vitro transfection of mammalian cells. Additional plasmids required for AAV production (pRC plasmid and pHelper plasmid) are purchased from commercial suppliers (Aldevron, Takara).

For AAV production, HEK293 cells are cultured in FB medium in a 37° C. incubator with 5% CO₂. 10-40 15 cm dishes of HEK293 cells are used in a single batch of viral production. For a single 15 cm dish, 45-60 μg plasmids are mixed together at 1:1:1 molar ratio together in 4 ml of FB medium, and complexed with Polyethyleneimine (PEI) i.e., at 3 μg PEI/μg of DNA, for 10 minutes at room temperature (Note: The ratio of the three plasmids used may be varied to optimize virus production). The PEI-DNA complex is then slowly dripped onto the 15 cm plate of HEK293 cells, and the plate of transfected cells is moved back into the incubator. The next day, the medium may be changed to FB with 2% FBS (instead of 10% FBS; Fibroblast medium, comprised of: DMEM with Glutamax (Gibco 10566-016) supplemented with MEM-NEAA (Thermo 11140050), sodium pyruvate (Thermo 11360070), HEPES (Thermo 15630080), 2-mercaptoethanol (Gibco 21985023), penicillin/streptomycin (Thermo 15140122) with 10% volume fraction of fetal bovine serum (FBS, VWR #97068-085)), if appropriate). AAV may be harvested from the supernatant, or from the cell pellet, or from a combination of the supernatant and the cell pellet, at any time between 48-120 hours after initial transfection of the plasmids.

If virus is harvested after 72 hours post-transfection, the media from the cells may be collected at this time to increase virus yields. At 2-5 days post-transfection, the medium and cells are collected (Note: The timing of harvest may be varied to optimize virus yield). The cells are pelleted by centrifugation, and the medium collected from the top. Cells are lysed in a buffer with high salt content and high-salt-active nuclease for 1 h at 37° C. (Note: The cells may also be lysed using additional methods, such as sequential freeze-thaw, or chemical lysis by detergent). The medium collected at harvest, and any medium collected at earlier time points, are treated with a 1:5 dilution of a solution containing 40% PEG8000 and 2.5M NaCl, and incubated on ice for 2 h, in order to precipitate AAV (Note: The incubation may also be carried out overnight at 4° C.). The AAV precipitate from the medium is pelleted by centrifugation, resuspended in high salt content buffer with high-salt-active nuclease and combined with the lysed cell pellet. The combined cell lysate is then clarified by centrifugation and filtration through a 0.45 m filter, and purified on an AAV Poros affinity resin column (Thermofisher Scientific). The virus is eluted from the column into a neutralizing solution (Note: At this stage, the virus may be taken through additional rounds of purification to increase the quality of the virus preparation). The eluted virus is then titered via qPCR to quantify the virus yield. For titering, a sample of virus is first digested with DNAse to remove any non-packaged viral DNA, the DNAse deactivated, and then viral capsids disrupted with Proteinase K to expose the packaged viral genomes for titering.

It is expected that ˜1×10¹² viral genomes will be obtained from one batch of virus produced using the methods as described here.

### Example 24: In Vivo Evaluation of C9orf72 Editing in a Mouse Model Having HRS Expansion

The C9-BAC mouse model (O'Rourke et al. C9orf72 BAC transgenic mice display typical pathologic features of ALS/FTD. Neuron. 88:892 (2015)) carrying the human chromosome 9 open reading frame 72 gene (C9orf72) with a hexanucleotide repeat expansion (GGGGCC) in the intron between the alternatively-spliced non-coding first exons 1a and 1b will be utilized to evaluate editing of the C9orf72 gene using the CasX:gNA systems delivered as RNP or by an AAV vector.

### Methods:

For injections of AAV or RNP containing CasX and gNA with spacers targeting the C9orf72 gene or using a non-targeting gNA as a negative control, mice will be anesthetized and placed on a rodent stereotaxic apparatus followed by injection into one of their lateral ventricles, the hippocampus, corpus striatum, primary somatosensory cortex (S1), and/or primary visual cortex V1 with varying doses of either viral particles or RNP of CasX:gNA (formulated with NLS or Lipofectamine2000 for cell delivery) in 1-5 μl volumes. A separate cohort of animals will be injected with virus or RNP intrathecally. Groups of mice will be monitored for body weight, survival and for behavioral and neuromuscular changes using assays such as the rotarod test, grip strength test, balance beam test, footprint test, and open-field test (Hao, Z., et al. Motor dysfunction and neurodegeneration in a C9orf72 mouse line expressing poly-PR. Nat. Commun. 10:2906 (2019)). Additional groups of mice will be euthanized at pre-determined intervals ranging from 1-24 months and will be transcardiacally perfused with saline. Brains will be removed from the skull, with one hemisphere processed for histological analyses, and the other hemisphere dissected and snap-frozen for biochemical and genetic analyses. Similarly, spinal cords will be dissected and snap-frozen or processed for histological analyses (cervical/thoracic). The brains and spinal cord for histology will be drop-fixed in 4% paraformaldehyde for 24 hours, then transferred to 30% sucrose for 24-48 hours and frozen in liquid nitrogen for serial sectioning followed by assay/examination for polyGP (DPR) and RNA transcript foci visualization using appropriate antibody or hybridization probes.

For polyGP (DPR) quantification, brain and spinal cord samples will be homogenized in RIPA (50 mM Tris, 150 mM NaCl, 0.5% DOC, 1% NP40, 0.1% SDS and Complete™, pH 8.0), followed by centrifugation and resuspension of the pellet in 5M guanidine-HCl. PolyGP will be quantified using the polyclonal antibody AB1358 (Millipore Sigma) for both capture and detection antibody in a 96-well format assay using PolyGP standard as a control or quantified by qPCR transcript analysis. Additionally, RIPA homogenates will be utilized for expression of C9orf72 protein, with levels determined by western blotting. Briefly, proteins from RIPA extracts will be size-fractionated by 4-12% SDS-PAGE and transferred onto PVDF membranes. To detect C9orf72, the membranes will be immunoblotted using the mouse monoclonal anti-C9orf72 antibody GT779 (Gene Tex, Irvine, CA), followed by secondary dye-conjugated antibody. Visualization will be conducted using the Odyssey/Li-Cor imaging system.

### Example 25: In Vivo Evaluation of Cognitive Behavior in a C9orf72 BAC Heterozygous Mouse Model Following Editing

C9orf72 BAC mouse models with GGGGCC repeats will be evaluated for improvements in cognitive tests following editing of the C9orf72 gene using the CasX:gNA systems described in Examples 17, 18, 22, and 23.

### Methods:

Following editing in mice of the C9orf72 gene using CasX and gNA with spacers targeting the C9orf72 gene or using a non-targeting gNA as a negative editing control, plus normal untreated mice on the same background, groups of mice will be evaluated using cognitive tests at 1, 2, and 3 months post-injection. The tests will include the Barnes maze test, the radial arm maze test, the marble burying test, and the elevated plus maze test (Jiang, J., et al. Gain of Toxicity from ALS/FTD-Linked Repeat Expansions in C9ORF72 Is Alleviated by Antisense Oligonucleotides Targeting GGGGCC-Containing RNAs. Neuron 90:535 (2016).

### Example 26: Evaluation of Spacer Length on Editing in Cells when Delivered as RNP

CasX variant 491 was purified as described above. Guide RNAs with scaffold 174 were prepared by in vitro transcription (IVT). IVT templates were generated by PCR using Q5 polymerase (NEB M0491) according to the recommended protocol, template oligos for each scaffold backbone, and amplification primers with the T7 promoter and either the 15.3 (CAAACAAATGTGTCACAAAG, SEQ ID NO: 344) or 15.5 (GGAATAATGCTGTTGTTGAA, SEQ ID NO: 345) spacer at full-length (20 nucleotides) or truncated by one or two nucleotides from the 3′ end of the respective spacer (sequences in Table 18). The sequences of the primers used to generate the IVT templates are shown in Table 17. The resulting templates were then used with T7 RNA polymerase to produce RNA guides according to standard protocols. The guides were purified using denaturing polyacrylamide gel electrophoresis and refolded prior to use. Individual RNPs were assembled by mixing protein with a 1.2-fold molar excess of guide in buffer containing 25 mM sodium phosphate buffer (pH 7.25), 300 mM NaCl, 1 mM MgCl2, and 200 mM trehalose. RNPs were incubated at 37° C. for 10 minutes and then purified via size exclusion chromatography and exchanged into buffer containing 25 mM sodium phosphate buffer (pH 7.25), 150 mM NaCl, 1 mM MgCl2, and 200 mM trehalose (Buffer 1). The concentration of the RNP was determined following purification using the Pierce 660 nm protein assay.

Purified RNPs were tested for editing at the T Cell Receptor α (TCRα) locus in Jurkat cells. RNPs were delivered by electroporation using the Lonza 4-D nucleofector system. 700,000 cells were resuspended in 20 μL of Lonza buffer SE and added to RNP diluted in Buffer 1 to the appropriate concentration and a final volume of 2 μL. Cells were electroporated using the Lonza 96-well shuttle system using the protocol CL-120. Cells were recovered at 37° C. in pre-equilibrated RPMI and then each electroporation condition was split into three wells of a 96-well plate. Cells were exchanged into fresh RPMI one day after nucleofection. On day three post-nucleofection, cells were stained with Alexa Fluor 647-labeled antibody against TCRα/β (BioLegend) and assessed for loss of surface TCRα/β using an Attune Nxt flow cytometer. A fraction of Jurkat cells does not stain positive for TCRα/β in the absence of editing. To account for this and estimate the actual percentage of cells that had TCRα knocked out via editing, the formula TCR_{KO}=(TCR-_{obs}−TCR-_{neg})/(1−TCR-_{neg}) was applied, where TCR_{KO} is the estimated knockout rate of TCRα, TCR-_{obs} is the observed fraction of cells negative for TCR staining in the experimental sample, and TCR-_{neg} is the fraction of cells negative for TCR staining in the no RNP control sample. This formula assumes that cells that do and do not express TCRα/β are edited at equal rates. The corrected fraction of TCRα knockout cells were plotted against the concentration of RNP using Prism. For each spacer, the three spacer lengths were fit with dose response curves using shared parameters except for the EC50. The reported p-values are the probability that the dose curve for the 20-nt spacer and that of the compared truncated spacer can be modeled with the same EC50 parameter.

TABLE 17
Oligonucleotides used to generate IVT templates

| Construct | DNA sequence | SEQ ID NO |
|---|---|---|
| Scaffold 174 | GAAATTAATACGACTCACTATAACTGGCGCTTTTATC | 346 |
| template fwd | TGATTACTTTGAGAGCCATCACCAGCGACTATGTCGT | |
| | AGTGGGTAAAGCT | |
| Scaffold 174 | CTTTGATGCTCCCTCCGAAGAGGGAGCTTTACCCACT | 347 |
| template rev | ACGACATAGTCGC | |
| T7 | GAAATTAATACGACTCACTATA | 348 |
| amplification | | |
| primer | | |
| 15.3 20-nt | CTTTGTGACACATTTGTTTGCTTTGATGCTCCCTCC | 349 |
| spacer primer | | |
| 15.3 19-nt | TTTGTGACACATTTGTTTGCTTTGATGCTCCCTCC | 350 |
| spacer primer | | |
| 15.3 18-nt | TTGTGACACATTTGTTTGCTTTGATGCTCCCTCC | 351 |
| spacer primer | | |
| 15.5 20-nt | TTCAACAACAGCATTATTCCCTTTGATGCTCCCTCC | 352 |
| spacer primer | | |
| 15.5 19-nt | TCAACAACAGCATTATTCCCTTTGATGCTCCCTCC | 353 |
| spacer primer | | |
| 15.5 18-nt | CAACAACAGCATTATTCCCTTTGATGCTCCCTCC | 354 |
| spacer primer | | |

TABLE 18
Spacer Sequences

| | | SEQ |
|---|---|---|
| Construct | RNA sequence | ID NO |
| 15.3 20-nt spacer | CAAACAAAUGUGUCACAAAG | 355 |
| 15.3 19-nt spacer | CAAACAAAUGUGUCACAAA | 356 |
| 15.3 18-nt spacer | CAAACAAAUGUGUCACAA | 357 |
| 15.5 20-nt spacer | GGAAUAAUGCUGUUGUUGAA | 358 |
| 15.5 19-nt spacer | GGAAUAAUGCUGUUGUUGA | 359 |
| 15.5 18-nt spacer | GGAAUAAUGCUGUUGUUG | 360 |

### Results

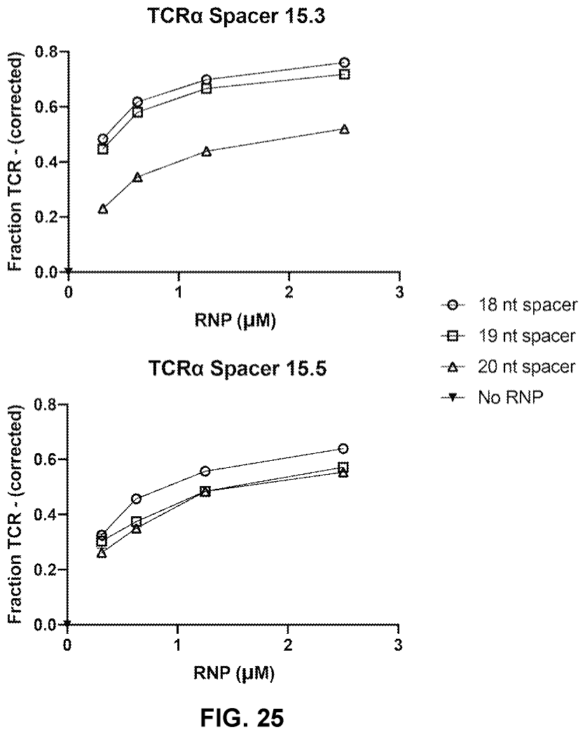

CasX RNPs were assembled using CasX variant 491 and guides composed of scaffold 174 with either spacer 15.3 or 15.5, both of which target the constant region of the TCRα gene. Guides with full-length 20-nt spacers as well as truncated 19- and 18-nt spacers were tested to determine whether use of shorter spacers supported increased editing when pre-assembled RNPs are nucleofected for ex vivo editing. RNPs were tested at 2-fold dilutions ranging from 0.3125 μM to 2.5 μM in a 22 μL nucleofection reaction. Editing was assessed by flow cytometry three days after nucleofection. For both spacer sequences, the RNPs with truncated spacers largely edited more efficiently than those with 20-nt spacers across the dose range (FIG. 25, dose-response curves). For spacer 15.3, the 18-nt and 19-nt spacers had EC50 values of 0.225 μM and 0.299 μM, respectively, as compared to 1.414 μM for the 20-nt spacer (p<0.0001 for both truncations; extra-sum-of-squares F test). For spacer 15.5, the 18-nt spacer had an EC50 of 0.519 μM vs 0.938 μM for the 20-nt spacer (p=0.0001), while the 19-nt spacer was more similar to the 20-nt spacer, with an EC50 of 0.808 μM (p=0.0762). Despite the fact that the 19-nt 15.3 spacer had editing similar to the 18-nt spacer while the 19-nt 15.5 spacer more closely resembles the corresponding 20-nt spacer, for both spacers tested, however, the direction of the trend remained consistent and suggests that using guides with 18-nt spacers may be a generalizable strategy for increasing editing when the CasX editing molecule is delivered as a pre-assembled RNP. Additional experiments with cell-based assays will be conducted to confirm these findings.

1. A system comprising a CasX variant protein and a guide nucleic acid (gNA), wherein the CasX variant protein comprises a sequence having at least 70% sequence identity to SEQ ID NO: 138 and exhibits one or more improved characteristics relative to a CasX reference protein of any one of SEQ ID NOS: 1-3, wherein the CasX variant protein is capable of forming a ribonuclear protein complex (RNP) with the qNA, and wherein the gNA comprises a targeting sequence complementary to a target nucleic acid sequence comprising a chromosome 9 open reading frame 72 (C9orf72) gene.

2. The system of claim 1, wherein the C9orf72 gene comprises one or more mutations, or wherein the C9orf72 gene mutation comprises more than 30, more than 100, more than 500, more than 700, more than 1000, or more than 1600 copies of a hexanucleotide repeat sequence GGGGCC in a hexanucleotide repeat sequence expansion (HRS).

3-10. (canceled)

11. The system of claim 1, wherein:
(a) the qNA is a single-molecule guide RNA (gRNA) comprising a scaffold sequence and a targeting sequence, and wherein the scaffold sequence comprises a scaffold stem loop sequence of CCAGCGACUAUGUCGUAGUGG (SEQ ID NO: 32) or a sequence with at least 1, 2, 3, 4, or 5 mismatches thereto;
(b) the targeting sequence of the gNA comprises a sequence selected from the group consisting of SEQ ID NOS: 309-343, 363-2100 and 2295-21835, or a sequence having at least about 65%, at least about 75%, at least about 85%, or at least about 95% identity thereto;
(c) the qNA has a scaffold comprising a sequence having at least 70% sequence identity to the sequence of SEQ ID NO: 2238; and/or
(d) wherein the qNA is chemically modified.

12-27. (canceled)

28. The system of claim 1, further comprising a second gNA, wherein the second gNA has a targeting sequence complementary to a different or overlapping portion of the target nucleic acid sequence compared to the targeting sequence of the gNA.

29. The system of claim 28, wherein the qNA is complementary to a sequence that is 5′ to the HRS and the targeting sequence of the second gNA is complementary to a sequence that is 5′ or 3′ to the HRS.

30-40. (canceled)

41. The system of claim 1, wherein the CasX variant protein further comprises one or more nuclear localization signals (NLS), wherein the one or more NLS are selected from the group of sequences consisting of PKKKRKV (SEQ ID NO: 165), KRPAATKKAGQAKKKK (SEQ ID NO: 166), PAAKRVKLD (SEQ ID NO: 167), RQRRNELKRSP (SEQ ID NO: 168), NQSSNFGPMKGGNFGGRSSGPYGGGGQYFAKPRNQGGY (SEQ ID NO: 169), RMRIZFKNKGKDTAELRRRRVEVSVELRKAKKDEQILKRRNV (SEQ ID NO: 170), VSRKRPRP (SEQ ID NO: 171), PPKKARED (SEQ ID NO: 172), PQPKKKPL (SEQ ID NO: 173), SALIKKKKKMAP (SEQ ID NO: 174), DRLRR (SEQ ID NO: 175), PKQKKRK (SEQ ID NO: 176), RKLKKKIKKL (SEQ ID NO: 177), REKKKFLKRR (SEQ ID NO:178) KRKGDEVDGVDEVAKKK SKK (SEQ ID NO: 179), RKCLQAGMNLEARKTKK (SEQ ID NO: 180), PRPRKIPR (SEQ ID NO: 181), PPRKKRTVV (SEQ ID NO: 182), NLSKKKKRKREK (SEQ ID NO: 183), RRPSRPFRKP (SEQ ID NO: 184), KRPRSPSS (SEQ ID NO: 185), KRGINDRNFWRGENERKTR (SEQ ID NO: 186), PRPPKMARYDN (SEQ ID NO: 187), KRSFSKAF (SEQ ID NO: 188), KLKIKRPVK (SEQ ID NO: 189), PKTRRRPRRS QRKRPPT (SEQ ID NO: 191), RRKKRRPRRKKRR (SEQ ID NO: 194), PKKKSRKPKKKSRK (SEQ ID NO: 195), HKKKHPDASVNFSEFSK (SEQ ID NO: 196), QRPGPYDRPQRPGPYDRP (SEQ ID NO: 197), LSPSLSPLLSPSLSPL (SEQ ID NO: 198), RGKGGKGLGKGGAKRHRK (SEQ ID NO: 199), PKRGRGRPKRGRGR (SEQ ID NO: 200), MSRRRKANPTKLSENAKKLAKEVEN (SEQ ID NO: 192), PKKKRKVPPPPAAKRVKLD (SEQ ID NO: 190), PKKKRKVPPPPKKKRKV (SEQ ID NO: 201), PAKRARRGYKC (SEQ ID NO: 202), KLGPRKATGRW (SEQ ID NO: 203), and PRRKREE (SEQ ID NO: 204), and wherein:
(i) the one or more NLS are at or near a C-terminus of the CasX variant protein,
(ii) the one or more NLS are at or near a N-terminus of the CasX variant protein, and/or
(iii) the CasX variant protein comprises at least two NLS that are at or near the N-terminus and the C-terminus of the CasX variant protein.

42-45. (canceled)

46. The system of claim 1, wherein the Cas X variant protein is capable of forming a ribonuclear protein complex (RNP) with the gNA.

47. The system of claim 46, wherein the CasX variant protein and the gNA exhibit at least one or more improved characteristics as compared to the reference CasX protein of any one of SEQ ID NOS: 1-3 and a reference gNA of any one of SEQ ID NOS: 4-16, and
wherein the improved characteristic is selected from the group consisting of improved folding of the CasX variant protein, improved binding affinity to the qNA, improved binding affinity to a target DNA, improved ability to utilize a greater spectrum of one or more protospacer adjacent motif (PAM) sequences including ATC, CTC, GTC or TTC in the editing of target DNA, improved unwinding of the target DNA, increased editing activity, improved editing efficiency, improved editing specificity, increased nuclease activity, increased target strand loading for double strand cleavage, decreased target strand loading for single strand nicking, decreased off-target cleavage, improved binding of non-target DNA strand, improved protein stability, improved protein solubility, improved protein:gNA RNP stability, improved protein:gNA complex solubility, improved protein yield, improved protein expression, and improved fusion characteristics, and
wherein the improved characteristic of the RNP of the CasX variant protein is at least 1.1 to 100,000-fold improved relative to the reference CasX protein of any one of SEQ ID NOS: 1-3 and the reference gRNA comprising any one of SEQ ID NOS: 4-16.

48-51. (canceled)

52. The system of claim 47, wherein;
(a) the RNP comprising the CasX variant protein and the gNA exhibits greater editing efficiency and/or binding of a target sequence in the target DNA when any one of the PAM sequences TTC, ATC, GTC or CTC is located 1 nucleotide 5′ to the non-target strand sequence having identity with the targeting sequence of the gNA in a cellular assay system compared to the editing efficiency and/or binding of an RNP comprising a reference CasX protein and a reference gNA in a comparable assay system, and
(i) the PAM sequence is TTC and the targeting sequence of the qNA comprises a sequence selected from the group consisting of SEQ ID NOs: 5427-12893,
(ii) the PAM sequence is ATC and the targeting sequence of the qNA comprises a sequence selected from the group consisting of SEQ ID NOs: 363-2100 and 2295-5426,
(iii) the PAM sequence is CTC and the targeting sequence of the qNA comprises a sequence selected from the group consisting of SEQ ID NOS: 16203-21835,
(iv) the PAM sequence is GTC and the targeting sequence of the qNA comprises a sequence selected from the group consisting of SEQ ID NOS: 12894-16202; and/or
(b) the RNP has at least a 5%, at least a 10%, at least a 15%, or at least a 20% higher percentage of cleavage-competent RNP compared to an RNP of the reference CasX protein of any one of SEQ ID NOS: 1-3 and the reference qNA comprising any one of SEQ ID NOS: 4-16, or the RNP has at least a 2-fold, at least 5-fold, or at least a 10-fold increased cleavage rate in an in vitro assay compared to a RNP of the reference CasX protein of any one of SEQ ID NOS: 1-3.

53-64. (canceled)

65. The system of claim 1, wherein the CasX variant protein comprises a nuclease domain having double-stranded cleavage activity.

66-78. (canceled)

79. A nucleic acid comprising a sequence encoding:
(a) a gNA comprising a targeting sequence complementary to a target nucleic acid sequence comprising a chromosome 9 open reading frame 72 (C9orf72) gene; and
(b) a CasX variant protein comprising a sequence having at least 70% sequence identity to SEQ ID NO: 138 and exhibits one or more improved characteristics relative to a CasX reference protein of any one of SEQ ID NOS: 1-3, wherein the CasX variant protein is capable of forming a ribonuclear protein complex (RNP) with the gRNA, and wherein the CasX variant protein further comprises one or more nuclear localization signals (NLS).

80. (canceled)

81. A vector comprising the nucleic acid of claim 79, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, and an RNA vector.

82-83. (canceled)

84. The vector of claim 81, wherein the vector is an AAV vector selected from the group consisting of AAV1, AAV2, AAV3, AAV4, AAV5, AAV6, AAV7, AAV8, AAV9, AAV10, AAV11, AAV12, AAV44.9, AAV-Rh74, and AAVRh10.

85-92. (canceled)

93. A host cell comprising the vector of claim 81, wherein the host cell is selected from the group consisting of BHK, HEK293, HEK293T, NS0, SP2/0, YO myeloma cells, P3X63 mouse myeloma cells, PER, PER.C6, NIH3T3, COS, HeLa, CHO, and yeast cells.

94. (canceled)

95. A method of modifying a chromosome 9 open reading frame 72 (C9orf72) gene target nucleic acid sequence in a population of cells, the method comprising the steps of,
introducing into the population of cells:
a. the system of claim 1;
b. the nucleic acid of claim 79;
c. the vector of claim 81; or
d. a combination thereof; and
modifying the C9orf72 gene target nucleic acid sequence in the population of cells targeted by the gNA and the CasX variant protein, wherein the modifying comprises:
(i) introducing a double-stranded break in the C9orf72 target nucleic acid sequence; and/or
(ii) introducing an insertion, deletion, substitution, duplication, or inversion of one or more nucleotides in the C9orf72 target nucleic acid sequence.

96-111. (canceled)

112. The method of claim 95, wherein the cell is a eukaryotic cell selected from the group consisting of a rodent cell, a mouse cell, a rat cell, a pig cell, a primate cell, and a non-human primate cell.

113-114. (canceled)

115. The method of claim 112, wherein the population of cells is selected from the group consisting of Purkinje cell, frontal cortex neuron, motor cortex neuron, hippocampus neuron, cerebellum neuron, upper motor neuron, spinal cord neuron, spinal cord motor neuron, glial cell, and astrocytes.

116-146. (canceled)

147. A population of cells modified by:
a. the system of claim 1;
b. the nucleic acid of claim 79;
c. the vector of claim 81; or
d. a combination thereof,
wherein the population of cells once modified include at least one of the following characteristics relative to a population of unmodified cells:
(i) at least 10%, at least about 20%, at least about 30%, at least about 40%, at least about 50%, at least about 60%, at least about 70%, at least about 80%, or at least about 90% do not express a detectable level of dipeptide repeat protein (DPR),
(ii) expression of a functional C9orf72 protein is increased by at least about 10%, at least about 20%, at least about 30%, at least about 40%, at least about 50%, at least about 60%, at least about 70%, at least about 80%, or at least about 90%, and/or
(iii) mutation of the C9orf72 gene is corrected, resulting in expression of a functional C9orf72 protein,
wherein cells of the population of cells are selected from the group consisting of Purkinje cell, frontal cortex neuron, motor cortex neuron, hippocampus neuron, cerebellum neuron, upper motor neuron, spinal cord neuron, spinal cord motor neuron, qlial cell, and astrocytes.

148-192. (canceled)

193. A composition for use in a method of treating a chromosome 9 open reading frame 72 (C9orf721-related disorder in a subject in need thereof, the method comprising the step of:
modifying a C9orf72 gene in cells of the subject by contacting said cells with a therapeutically effective dose of:
a. the system of claim 1;
b. the nucleic acid of claim 79;
c. the vector of claim 81; or
d. combinations thereof,
wherein the C9orf72 gene of the cells targeted by the gNA is modified by the CasX variant protein.
